(19) United States
(12) Patent Application Publication
(10) Pub. No.: US 2023/0227849 A1
(43) Pub. Date: Jul. 20, 2023

(54) METHODS OF IDENTIFYING AND CHARACTERIZING ANELLOVIRUSES AND USES THEREOF

(71) Applicant: FLAGSHIP PIONEERING INNOVATIONS V, INC., Cambridge, MA (US)

(72) Inventors: Avak Kahvejian, Lexington, MA (US); Erica Gabrielle Weinstein, Newton, MA (US); Roger Joseph Hajjar, Lexington, MA (US); Nathan Lawrence Yozwiak, Newton, MA (US); Simon Delagrave, Sudbury, MA (US); Yann Paul Guy Régis Echelard, Jamaica Plain, MA (US); Simeon Ungerleider Springer, Lexington, MA (US); Cesar A. Arze, Boston, MA (US); Kristian Graugaard Andersen, Cambridge, MA (US)

(21) Appl. No.: 18/010,674

(22) PCT Filed: Jun. 17, 2021
(86) PCT No.: PCT/US2021/037828

§ 371 Date: Dec. 15, 2022

Related U.S. Application Data
(60) Provisional application No. 63/040,371, filed on Jun. 17, 2020.
(60) Provisional application No. 63/130,074, filed on Dec. 23, 2020.
(60) Provisional application No. 63/147,029, filed on Feb. 8, 2021.

(51) Int. Cl. C12N 15/86; C12Q 1/70; A61K 48/00

(52) U.S. Cl. CPC C12N 15/86; C12Q 1/701; A61K 48/0066; A61K 48/0041; C12N 2750/00043; C12N 2750/00071

(57) ABSTRACT

This invention relates generally to compositions and methods for administering an anellovector (e.g., a synthetic anellovector) that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). Also provided are methods for amplifying circular nucleic acids comprising Anellovirus sequences.

### CROSS-REFERENCE TO RELATED APPLICATIONS

This application claims the benefit of U.S. Provisional Application Nos. 63/040,371, filed Jun. 17, 2020; 63/130,074, filed Dec. 23, 2020; and 63/147,029, filed Feb. 8, 2021. The contents of the aforementioned applications are hereby incorporated by reference in their entirety.

### BACKGROUND

There is an ongoing need to develop compositions and methods for making suitable vectors to deliver therapeutic effectors to patients.

### SUMMARY

The present disclosure provides compositions and methods for administering an anellovector (e.g., a synthetic anellovector) that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). Described herein are, for example, are methods of delivering an effector, comprising administering to a subject a first plurality of anellovectors and then a second plurality of anellovectors. In some embodiments, the second plurality of anellovectors comprise the same proteinaceous exterior as the anellovectors of the first plurality. In some embodiments, the second plurality of anellovectors comprises a proteinaceous exterior with at least one surface epitope in common with the anellovectors of the first plurality of anellovectors. Without wishing to be bound by theory, certain viral vectors for gene therapy result in an immune response (e.g., neutralizing antibodies), against the viral proteins, making those viral vectors unsuitable for repeated delivery to a subject. As shown, e.g., in Example 1 herein, Anellovectors do not seem to trigger a neutralizing immune response, and are thus suitable for administration in multiple doses.

The disclosure further provides methods for amplifying nucleic acid molecules comprising Anellovirus sequences, and compositions relating to such methods (e.g., reaction mixtures and products thereof). The methods generally involve providing a sample comprising a nucleic acid molecule (e.g., a circular nucleic acid molecule), which is contacted with a primer (e.g., with degenerate primers or a primer specific to an Anellovirus sequence, e.g., as described herein) and a DNA polymerase (e.g., a DNA-dependent DNA polymerase). Generally, the interaction of the nucleic acid molecule with the primer and the DNA polymerase results in rolling circle amplification of the nucleic acid molecule, if it comprises an Anellovirus sequence (e.g., an Anellovirus sequence comprising a target site recognized by the primer). In some instances, the primer is part of a plurality of primers (e.g., a plurality of degenerate primers, wherein the non-degenerate nucleotides of the primer are largely identical; or a plurality of Anellovirus-specific primers, wherein the Anellovirus-specific primers each comprise an identical sequence that binds to an Anellovirus sequence, e.g., as described herein). In some instances, the primer comprises a sequence as listed in Table A. In certain embodiments, the plurality of primers all comprise a sequence as listed in a single row of Table A.

The present disclosure further provides methods for determining the sequences of nucleic acid molecules amplified according to the amplification methods described herein, as well as methods of analyzing sequencing data obtained for a plurality of such amplified nucleic acid molecule. In some instances, the sequences of amplified nucleic acid molecules are determined by deep sequencing methods (also referred to as next-generation sequencing methods), e.g., as described herein. In some instances, the sequencing data are analyzed by computational methods, e.g., as described herein, for example, to identify Anellovirus sequences from nucleic acid molecules amplified as described herein.

The present disclosure additionally provides compositions and methods relating to anellovectors (e.g., synthetic anellovectors), e.g., anellovectors comprising a genetic element comprising an Anellovirus sequence identified or isolated according to the methods described herein; and/or anellovectors comprising one or more components (e.g., a capsid protein, e.g., an ORF1 molecule) encoded by an Anellovirus sequence identified or isolated according to the methods described herein.

An anellovector and components thereof that can be used in the methods for delivering an effector described herein (e.g., produced using a composition or method as described herein) generally comprise a genetic element (e.g., a genetic element comprising or encoding an effector, e.g., an exogenous or endogenous effector, e.g., a therapeutic effector) encapsulated in a proteinaceous exterior (e.g., a proteinaceous exterior comprising an Anellovirus capsid protein, e.g., an Anellovirus ORF1 protein or a polypeptide encoded by an Anellovirus ORF1 nucleic acid, e.g., as described herein), which is capable of introducing the genetic element into a cell (e.g., a mammalian cell, e.g., a human cell). In some embodiments, the anellovector is an infectious vehicle or particle comprising a proteinaceous exterior comprising a polypeptide encoded by an Anellovirus ORF1 nucleic acid (e.g., an ORF1 nucleic acid of Alphatorquevirus, Betatorquevirus, or Gammatorquevirus, e.g., an ORF1 of Alphatorquevirus clade 1, Alphatorquevirus clade 2, Alphatorquevirus clade 3, Alphatorquevirus clade 4, Alphatorquevirus clade 5, Alphatorquevirus clade 6, or Alphatorquevirus clade 7, e.g., as described herein). The genetic element of an anellovector of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some embodiments, the genetic element of an anellovector is produced using a composition or method, as described herein.

In some instances, the anellovectors that can be used in the methods of delivering an effector described herein comprise a genetic element which comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some embodiments, the effector is an endogenous effector or an exogenous effector, e.g., to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell (e.g., as described in Examples 3 and 4 of PCT/US19/65995). In another example, the anellovector can deliver and express an effector, e.g., an exogenous protein, in vivo (e.g., as described in Examples 10 and 14 of PCT/US19/65995). Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject.

In some embodiments, the compositions and methods described herein can be used to produce the genetic element of a synthetic anellovector to be used in the methods of administering anellovectors described herein, e.g., in a host cell. A synthetic anellovector has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors include an exogenous genetic element enclosed within a proteinaceous exterior, which can be used for delivering the genetic element, or an effector (e.g., an exogenous effector or an endogenous effector) encoded therein (e.g., a polypeptide or nucleic acid effector), into eukaryotic (e.g., human) cells. In embodiments, the anellovector does not cause a detectable and/or an unwanted immune or inflammatory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector may be substantially non-immunogenic to the target cell, tissue or subject.

In some embodiments, the compositions and methods described herein can be used to produce the genetic element of an anellovector, e.g. an anellovector that can be used in the methods of delivering an effector described herein, comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the anellovector is capable of delivering the genetic element into a eukaryotic (e.g., mammalian, e.g., human) cell. In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined by quantitative gel purification assay of genomic DNA separated from free vector, e.g., as described in Wang et al. (2004, Gene Therapy 11: 711-721, incorporated herein by reference in its entirety). In some embodiments, the genetic element is enclosed within the proteinaceous exterior. In some embodiments, the anellovector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as described herein). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Anellovirus sequence as described herein). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In some embodiments, the compositions and methods described herein can be used to produce the genetic element of an infectious (e.g., to a human cell) anellovector, vehicle, or particle comprising a capsid (e.g., a capsid comprising an Anellovirus ORF, e.g., ORF1, polypeptide) encapsulating a genetic element comprising a protein binding sequence that binds to the capsid and a heterologous (to the Anellovirus) sequence encoding a therapeutic effector that can be used in the methods of administering an anellovector described herein. In embodiments, the anellovector is capable of delivering the genetic element into a mammalian, e.g., human, cell. In some embodiments, the genetic element has less than about 6% (e.g., less than 10%, 9.5%, 9%, 8%, 7%, 6%, 5.5%, 5%, 4.5%, 4%, 3.5%, 3%, 2.5%, 2%, 1.5%, or less) identity to a wild type Anellovirus genome sequence. In some embodiments, the genetic element has no more than 1.5%, 2%, 2.5%, 3%, 3.5%, 4%, 4.5%, 5%, 5.5% or 6% identity to a wild type Anellovirus genome sequence. In some embodiments, the genetic element has at least about 2% to at least about 5.5% (e.g., 2 to 5%, 3% to 5%, 4% to 5%) identity to a wild type Anellovirus. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell (e.g., by comparing integration frequency into genomic DNA relative to genetic element sequences from cell lysates). In some embodiments, integration frequency is determined by quantitative gel purification assay of genomic DNA separated from free vector, e.g., as described in Wang et al. (2004, Gene Therapy 11: 711-721, incorporated herein by reference in its entirety).

In some embodiments, Anelloviruses or anellovectors, administered according to the methods described herein, can be used as effective delivery vehicles for introducing an agent, such as an effector described herein, to a target cell, e.g., a target cell in a subject to be treated therapeutically or prophylactically.

In some embodiments, the compositions and methods described herein can be used to produce the genetic element of an anellovector that can be used in the methods of administration described herein, comprising a proteinaceous exterior comprising a polypeptide (e.g., a synthetic polypeptide, e.g., an ORF1 molecule) comprising (e.g., in series):

(i) a first region comprising an arginine-rich region, e.g., a sequence of at least about 40 amino acids comprising at least 60%, 70%, or 80% basic residues (e.g., arginine, lysine, or a combination thereof),

(ii) a second region comprising a jelly-roll domain, e.g., a sequence comprising at least 6 beta strands,

(iii) a third region comprising an N22 domain sequence described herein,

(iv) a fourth region comprising an Anellovirus ORF1 C-terminal domain (CTD) sequence described herein, and

(v) optionally wherein the polypeptide has an amino acid sequence having less than 100%, 99%, 98%, 95%, 90%, 85%, 80% sequence identity to a wild type Anellovirus ORF1 protein, e.g., as described herein.

In an aspect, the invention features a method of amplifying a circular nucleic acid molecule comprising an Anellovirus sequence, the method comprising: (a) providing a sample comprising a circular nucleic acid molecule comprising an Anellovirus sequence and a primer having at least 7, 8, or 9 complementary to a portion of the Anellovirus sequence; and (b) contacting the circular nucleic acid molecule with a DNA-dependent DNA polymerase molecule; wherein the contacting results in linear amplification (e.g., rolling circle amplification or multiple strand displacement amplification) of the nucleic acid molecule, or a portion thereof.

In an aspect, the invention features a method of amplifying a circular nucleic acid molecule comprising an Anellovirus sequence, the method comprising: (a) providing a sample comprising a circular nucleic acid molecule comprising an Anellovirus sequence; and (b) contacting the circular nucleic acid molecule with a plurality of primers, wherein a first primer of said plurality has at least 7, 8, or 9 nucleotides complementary to a portion of the Anellovirus sequence, in the presence of a DNA-dependent DNA polymerase molecule; wherein the contacting results in linear amplification (e.g., rolling circle amplification or multiple strand displacement amplification) of the nucleic acid molecule, or a portion thereof.

In an aspect, the invention features a method of amplifying a circular nucleic acid molecule, the method comprising: (a) providing a sample comprising a circular nucleic acid molecule and a first primer and a second primer, wherein the first primer has at least 70%, 75%, 80%, 85%, 90%, 95%, 96%, 97%, 98%, 99%, or 100% sequence identity to the second primer, and wherein the first primer and the second primer are not identical; and (b) contacting the circular nucleic acid molecule with a DNA-dependent DNA polymerase molecule; wherein the contacting results in linear amplification (e.g., rolling circle amplification or multiple strand displacement amplification) of the nucleic acid molecule, or a portion thereof.

In an aspect, the invention features a method of amplifying a circular nucleic acid molecule, the method comprising: (a) providing a sample comprising a circular nucleic acid molecule and a plurality of distinct primers, wherein each of the plurality of primers share the same orientation relative to the nucleic acid molecule; and (b) contacting the circular nucleic acid molecule with a DNA-dependent DNA polymerase molecule; wherein the contacting results in linear amplification (e.g., rolling circle amplification or multiple strand displacement amplification) of the nucleic acid molecule, or a portion thereof.

In an aspect, the invention features a method of amplifying a circular nucleic acid molecule comprising an Anellovirus sequence, the method comprising: (a) providing a sample comprising a circular nucleic acid molecule and a plurality of primers each complementary to a portion of the Anellovirus sequence; and (b) contacting the circular nucleic acid molecule with a DNA-dependent DNA polymerase molecule; wherein: (i) the circular nucleic acid molecule comprises a plurality of sequences recognized by the one or more primers; (ii) the plurality of primers are all positive-strand primers or all negative-strand primers; (iii) the plurality of primers are all same-strand primers; (iv) the plurality of primers all comprise at least 3, 4, 5, 6, 7, 8, 9, or 10 contiguous nucleotides in common; and/or (v) the plurality of primers comprises at least 2, 3, 4, 5, 10, 15, 20, 25, 30, 35, 40, 45, 50, 60, 70, 80, 90, 100, or more different primers.

In an aspect, the invention features a method of amplifying a circular nucleic acid molecule comprising an Anellovirus sequence, the method comprising: (a) providing a sample comprising a circular nucleic acid molecule comprising an Anellovirus sequence and one or more primers complementary to a portion of the Anellovirus sequence; and (b) contacting the circular nucleic acid molecule with a DNA-dependent DNA polymerase molecule; wherein: (i) the circular nucleic acid molecule comprises a plurality of sequences recognized by the one or more primers; (ii) the one or more primers are all positive-strand primers or all negative-strand primers; (iii) the one or more primers are all same-strand primers; (iv) the one or more primers all comprise at least 3, 4, 5, 6, 7, 8, 9, or 10 contiguous nucleotides in common; and/or (v) the one or more primers comprises at least 2, 3, 4, 5, 10, 15, 20, 25, 30, 35, 40, 45, 50, 60, 70, 80, 90, 100, or more different primers

In an aspect, the invention features a method of amplifying a circular nucleic acid molecule comprising an Anellovirus sequence, the method comprising: (a) providing a sample comprising a circular nucleic acid molecule comprising an Anellovirus sequence and a plurality of primers complementary to a portion of the Anellovirus sequence; and (b) contacting the circular nucleic acid molecule with a DNA-dependent DNA polymerase molecule; wherein the contacting results in rolling circle amplification of the nucleic acid molecule, or a portion thereof; and wherein the sequences of the primers of the plurality are at least 75%, 80%, 85%, 90%, 95%, 96%, 97%, 98%, 99%, or 100% identical to each other.

In an aspect, the invention features a primer comprising a nucleic acid sequence according to any of SEQ ID NOs: 1-24, e.g., any of SEQ ID NOs: 1, 3, 4, 6, 8, 10, 12, 14, 17, 19, 21, or 23.

In an aspect, the invention features a mixture comprising a plurality of different primers, wherein each of the plurality of primers binds to a nucleic acid molecule comprising one or more sequences recognized by a primer having a sequence as listed in Table A.

In an aspect, the invention features a kit or a mixture comprising a plurality of different primers, wherein each of the plurality of primers binds to a nucleic acid molecule having a sequence of any of SEQ ID NOs: 1-24, e.g., any of SEQ ID NOs: 2, 5, 7, 9, 11, 13, 15, 16, 18, 20, 22, or 24.

In an aspect, the invention features a kit or a mixture comprising a nucleic acid sequence according to any 2, 3, 4, 5, 6, 7, 8, 9, 10, 11, or 12 or more of SEQ ID NOs: 1-24, e.g., any of SEQ ID NOs: 1, 3, 4, 6, 8, 10, 12, 14, 17, 19, 21, or 23.

In an aspect, the invention features an isolated nucleic acid molecule having a sequence of any of SEQ ID NOs: 13-24.

In an aspect, the invention features an isolated nucleic acid molecule (e.g., a circular nucleic acid molecule, e.g., a circular DNA molecule) comprising a thiophosphate-comprising primer sequence comprising a sequence according to any of SEQ ID NOs: 1-12 and at least 100, 200, 300, 400, 500, 600, 700, 800, 900, 1000, 1500, 2000, 2500, 3000, 3500, or 4000 contiguous nucleotides of an Anellovirus sequence.

In an aspect, the invention features an isolated nucleic acid molecule (e.g., a circular nucleic acid molecule, e.g., a circular DNA molecule) comprising a plurality of Anellovirus sequences, or fragments thereof each comprising at least 100, 200, 300, 400, 500, 600, 700, 800, 900, 1000, 1500, 2000, 2500, 3000, 3500, or 4000 contiguous nucleotides of the Anellovirus sequence; wherein the Anellovirus sequences or fragments thereof each comprise (e.g., at one end) a thiophosphate-comprising primer sequence comprising a sequence according to any of SEQ ID NOs: 1-12.

In an aspect, the invention features an isolated nucleic acid molecule (e.g., a nucleic acid construct) comprising the sequence of a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus, e.g., as disclosed herein. In embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined by quantitative gel purification assay of genomic DNA separated from free vector, e.g., as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element is capable of directing expression of the effector in a eukaryotic (e.g., mammalian, e.g., human) cell.

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein).

In an aspect, the invention features a host cell comprising: (a) a nucleic acid molecule comprising a sequence encoding one or more of an ORF1 molecule, an ORF2 molecule, or an ORF3 molecule (e.g, a sequence encoding an Anellovirus ORF1 polypeptide described herein), e.g., wherein the nucleic acid molecule is a plasmid, is a viral nucleic acid, or is integrated into a chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 polypeptide (e.g., an ORF1 protein). For example, the host cell comprises (a) and (b) either in cis (both part of the same nucleic acid molecule) or in trans (each part of a different nucleic acid molecule). In embodiments, the genetic element of (b) is a circular, single-stranded DNA. In some embodiments, the host cell is a manufacturing cell line, e.g., as described herein. In some embodiments, the host cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an anellovector (e.g., a synthetic anellovector), e.g., an anellovector that can be administered by the methods described herein. In embodiments, the pharmaceutical composition further comprises a pharmaceutically acceptable carrier or excipient. In embodiments, the pharmaceutical composition comprises a unit dose comprising about 10⁵-10¹⁴ (e.g., about 10⁶-10¹³, 10⁷-10¹², 10⁸-10¹¹, or 10⁹-10¹⁰) genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In embodiments, the payload is a nucleic acid. In embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

In an aspect, the invention features a method of making an anellovector, e.g., a synthetic anellovector that can be used in a method of administrating an anellovector described herein. The method includes:

(a) providing a host cell comprising:
(i) a first nucleic acid molecule comprising the nucleic acid sequence of a genetic element of an anellovector, e.g., as described herein; and
(ii) a second nucleic acid molecule encoding an Anellovirus ORF1 polypeptide, or one or more of an amino acid sequence chosen from ORF1, ORF2, ORF2/2, ORF2/3, ORF1/1, or ORF1/2, e.g., as described herein, or an amino acid sequence having at least 70% (e.g., at least 70%, 75%, 80%, 85%, 90%, 95%, 96%, 97%, 98%, 99%, or 100%) sequence identity thereto; and

(b) incubating the host cell under conditions suitable for replication (e.g., rolling circle replication) of the nucleic acid sequence of the genetic element, thereby producing a genetic element; and

optionally (c) incubating the host cell under conditions suitable for enclosure of the genetic element in a proteinaceous exterior (e.g., comprising a polypeptide encoded by the second nucleic acid molecule).

In another aspect, the invention features a method of manufacturing an anellovector composition, e.g., an anellovector composition that can be used in the methods of administration described herein, the composition comprising one or more of (e.g., all of) (a), (b), and (c):

a) providing a host cell comprising, e.g., expressing one or more components (e.g., all of the components) of an anellovector, e.g., a synthetic anellovector, e.g., as described herein;

b) culturing the host cell under conditions suitable for producing a preparation of anellovectors from the host cell, wherein the anellovectors of the preparation comprise a proteinaceous exterior (e.g., comprising an Anellovector ORF1 polypeptide) encapsulating the genetic element (e.g., as described herein), thereby making a preparation of anellovectors; and

optionally, c) formulating the preparation of anellovectors, e.g., as a pharmaceutical composition suitable for administration to a subject.

For example, the host cell provided in this method of manufacturing comprises (a) a nucleic acid comprising a sequence encoding an Anellovirus ORF1 polypeptide described herein, wherein the nucleic acid is a plasmid, is a viral nucleic acid or genome, or is integrated into a helper cell chromosome; and (b) a nucleic acid construct capable of producing a genetic element (e.g., comprising a genetic element sequence and/or genetic element region, e.g., as described herein), e.g., wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging sequence) that binds the polypeptide of (a), wherein the host cell comprises (a) and (b) either in cis or in trans. In embodiments, the genetic element of (b) is circular, single-stranded DNA. In some embodiments, the host cell is a manufacturing cell line.

In some embodiments, the components of the anellovector are introduced into the host cell at the time of production (e.g., by transient transfection). In some embodiments, the host cell stably expresses the components of the anellovector (e.g., wherein one or more nucleic acids encoding the components of the anellovector are introduced into the host cell, or a progenitor thereof, e.g., by stable transfection).

In an aspect, the invention features a method of manufacturing an anellovector composition, comprising: a) providing a plurality of anellovectors described herein, or a preparation of anellovectors described herein; and b) formulating the anellovectors or preparation thereof, e.g., as a pharmaceutical composition suitable for administration to a subject.

In an aspect, the invention features a method of making a host cell, e.g., a first host cell or a producer cell (e.g., as shown in FIG. 12 of PCT/US19/65995), e.g., a population of first host cells, comprising an anellovector, the method comprising introducing a nucleic acid construct capable of producing a genetic element, e.g., as described herein, to a host cell and culturing the host cell under conditions suitable for production of the anellovector. In embodiments, the method further comprises introducing a helper, e.g., a helper virus, to the host cell. In embodiments, the introducing comprises transfection (e.g., chemical transfection) or electroporation of the host cell with the anellovector.

In an aspect, the invention features a method of making an anellovector, comprising providing a host cell, e.g., a first host cell or producer cell (e.g., as shown in FIG. 12 of PCT/US19/65995), comprising an anellovector, e.g., as described herein, and purifying the anellovector from the host cell. In some embodiments, the method further comprises, prior to the providing step, contacting the host cell with a nucleic acid construct or an anellovector, e.g., as described herein, and incubating the host cell under conditions suitable for production of the anellovector. In embodiments, the host cell is the first host cell or producer cell described in the above method of making a host cell. In embodiments, purifying the anellovector from the host cell comprises lysing the host cell.

In some embodiments, the method further comprises a second step of contacting the anellovector produced by the first host cell or producer cell with a second host cell, e.g., a permissive cell (e.g., as shown in FIG. 12 of PCT/US19/65995), e.g., a population of second host cells. In some embodiments, the method further comprises incubating the second host cell inder conditions suitable for production of the anellovector. In some embodiments, the method further comprises purifying an anellovector from the second host cell, e.g., thereby producing an anellovector seed population. In embodiments, at least about 2-100-fold more of the anellovector is produced from the population of second host cells than from the population of first host cells. In embodiments, purifying the anellovector from the second host cell comprises lysing the second host cell. In some embodiments, the method further comprises a second step of contacting the anellovector produced by the second host cell with a third host cell, e.g., permissive cells (e.g., as shown in FIG. 12 of PCT/US19/65995), e.g., a population of third host cells. In some embodiments, the method further comprises incubating the third host cell inder conditions suitable for production of the anellovector. In some embodiments, the method further comprises purifying a anellovector from the third host cell, e.g., thereby producing an anellovector stock population. In embodiments, purifying the anellovector from the third host cell comprises lysing the third host cell. In embodiments, at least about 2-100-fold more of the anellovector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of anellovectors by the host cell. In some embodiments, anellovectors produced by a host cell separated from the host cell (e.g., by lysing the host cell) prior to contact with a second host cell. In some embodiments, anellovectors produced by a host cell are contacted with a second host cell without an intervening purification step.

In an aspect, the invention features a method of making a pharmaceutical anellovector preparation, e.g., a preparation to be used in the methods of administration described herein. The method comprises (a) making an anellovector preparation as described herein, (b) evaluating the preparation (e.g., a pharmaceutical anellovector preparation, anellovector seed population or the anellovector stock population) for one or more pharmaceutical quality control parameters, e.g., identity, purity, titer, potency (e.g., in genomic equivalents per anellovector particle), and/or the nucleic acid sequence, e.g., from the genetic element comprised by the anellovector, and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the anellovector, e.g., the sequence encoding the effector. In some embodiments, evaluating purity comprises evaluating the amount of an impurity, e.g., Mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates. In some embodiments, evaluating titer comprises evaluating the ratio of functional versus non-functional (e.g., infectious vs non-infectious) anellovectors in the preparation (e.g., as evaluated by HPLC). In some embodiments, evaluating potency comprises evaluating the level of anellovector function (e.g., expression and/or function of an effector encoded therein or genomic equivalents) detectable in the preparation.

In embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anellovectors can be produced in a single batch. In embodiments, the levels of the anellovectors produced in the batch can be evaluated (e.g., individually or together).

In an aspect, the invention features a host cell comprising:

(i) a first nucleic acid molecule comprising a nucleic acid construct as described herein, and

(ii) optionally, a second nucleic acid molecule encoding one or more of an amino acid sequence chosen from ORF1, ORF2, ORF2/2, ORF2/3, ORF1/1, or ORF1/2, e.g., as described herein, or an amino acid sequence having at least about 70% (e.g., at least about 70, 80, 90, 95, 96, 97, 98, 99, or 100%) sequence identity thereto.

In an aspect, the invention features a reaction mixture comprising an anellovector described herein and a helper virus that can be used in the methods of administration described herein, wherein the helper virus comprises a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an anellovector (e.g., a synthetic anellovector) is isolated, e.g., isolated from a host cell and/or isolated from other constituents in a solution (e.g., a supernatant). In some embodiments, an anellovector (e.g., a synthetic anellovector) is purified, e.g., from a solution (e.g., a supernatant). In some embodiments, an anellovector is enriched in a solution relative to other constituents in the solution.

In some embodiments of any of the aforesaid anellovectors, compositions or methods, providing an anellovector comprises separating (e.g., harvesting) an anellovector from a composition comprising an anellovector-producing cell, e.g., as described herein. In other embodiments, providing an anellovector comprises obtaining an anellovector or a preparation thereof, e.g., from a third party.

In some embodiments of any of the aforesaid anellovectors, compositions or methods, the genetic element comprises an anellovector genome, e.g., as identified according to the methods described herein. In embodiments, the anellovector genome comprises a TTV-tth8 nucleic acid sequence, e.g., a TTV-tth8 nucleic acid, e.g., having deletions of at least 10%, 20%, 30%, 40%, 50%, 60%, 70%, 80%, 90%, 95%, 99%, or 100% of nucleotides 3436-3707 of the TTV-tth8 nucleic acid sequence. In embodiments, the anellovector genome comprises a TTMV-LY2 nucleic acid sequence, e.g., a TTMV-LY2 nucleic acid sequence, e.g., having deletions of at least 10%, 20%, 30%, 40%, 50%, 60%, 70%, 80%, 90%, 95%, 99%, or 100% of nucleotides 574-1371, 1432-2210, 574-2210, and/or 2610-2809 of the TTMV-LY2 nucleic acid sequence. In embodiments, the genetic element is capable of self-replication and/or self-amplification. In embodiments, the genetic element is not capable of self-replication and/or self-amplification. In embodiments, the genetic element is capable of replicating and/or being amplified in trans, e.g., in the presence of a helper, e.g., a helper virus.

Additional features of any of the aforesaid anellovectors, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### ENUMERATED EMBODIMENTS

1. A method of delivering an effector to a human subject who has previously been administered a first plurality of anellovectors, said method comprising:
administering to the subject a second plurality of anellovectors, wherein:

(i) the first plurality of anellovectors, comprises:
(a) a proteinaceous exterior that comprises an ORF1 molecule;
(b) a genetic element comprising a promoter element and a nucleic acid sequence (e.g., a DNA sequence) encoding an effector (e.g., an exogenous effector or an endogenous effector), and

(ii) the second plurality of anellovectors comprises:
(a) the same proteinaceous exterior as the anellovectors of the first plurality,
a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or
a proteinaceous exterior with at least one surface epitope in common with the anellovectors of the first plurality, and
(b) a genetic element comprising a promoter element and a nucleic acid sequence (e.g., a DNA sequence) encoding an effector (e.g., the effector of (i)(b) or a second effector, e.g., a second exogenous or endogenous effector),

thereby delivering the effector to the subject.

2. The method of embodiment 1, which comprises administering the first plurality of anellovectors to the subject.

3. A method of delivering an effector to a human subject, comprising:
(i) administering to the subject a first plurality of anellovectors comprising:
(a) a proteinaceous exterior that comprises an ORF1 molecule;
(b) a genetic element comprising a promoter element and a nucleic acid sequence (e.g., a DNA sequence) encoding an effector (e.g., an exogenous effector or an endogenous effector), and (ii) subsequently administering to the subject a second plurality of anellovectors comprising:
(a) the same proteinaceous exterior as the anellovectors of the first plurality,
a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or
a proteinaceous exterior with at least one surface epitope in common with the anellovectors of the first plurality, and
(b) a genetic element comprising a promoter element and a nucleic acid sequence (e.g., a DNA sequence) encoding the effector (e.g., the effector of (i)(b) or a second effector, e.g., a second exogenous or endogenous effector),

thereby delivering the effector to the subject.

4. A method of selecting a human subject to receive an effector,
wherein the subject previously received, or was identified as having received, a first plurality of anellovectors comprising:
(a) a proteinaceous exterior that comprises an ORF1 molecule;
(b) a genetic element comprising a promoter element and a nucleic acid sequence (e.g., a DNA sequence) encoding the effector (e.g., an exogenous effector or an endogenous effector),

said method comprising selecting the subject to receive a second plurality of anellovectors comprising:

the same proteinaceous exterior as the anellovectors of the first plurality,

a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or

a proteinaceous exterior with at least one surface epitope in common with the anellovectors of the first plurality.

5. A method of identifying a human subject suitable to receive a second plurality of anellovectors, comprising:
identifying the subject as having received a first plurality of anellovectors comprising:
(a) a proteinaceous exterior that comprises an ORF1 molecule;
(b) a genetic element comprising a promoter element and a nucleic acid sequence (e.g., a DNA sequence) encoding the effector (e.g., an exogenous effector or an endogenous effector),

wherein the subject being identified as having received the first plurality of anellovectors is indicative that the subject is suitable to receive the second plurality of anellovectors, wherein the second plurality of anellovectors comprises:

the same proteinaceous exterior as the anellovectors of the first plurality,

a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or
a proteinaceous exterior with at least one surface epitope in common with the anellovectors of the first plurality.

6. The method of embodiment 4 or 5, wherein the subject is selected on the basis of having received the first plurality of anellovectors.

7. The method of embodiment 6, wherein the subject received the first plurality of anellovectors in a blood transfusion.

8. The method of embodiment 4 or 5, wherein the subject is evaluated between the administration of the first and second pluralities of anellovectors, e.g., for the presence of an immune response, e.g., antibodies, against one or more anellovectors of the first plurality.

9. The method of embodiment 8, wherein the second plurality of anellovectors is administered if the presence of an immune response is not detected.

10. The method of embodiment 8, wherein the second plurality of anellovectors is administered if the presence of an immune response is detected.

11. The method of embodiment 5 or 6, wherein the subject is evaluated between the administration of the first and second pluralities of anellovectors, e.g., for the presence (e.g., persistence) of anellovectors from the first plurality, or progeny thereof.

12. The method of embodiment 11, wherein the second plurality of anellovectors is administered if the presence of anellovectors from the first plurality, or the progeny thereof, are not detected.

13. The method of embodiment 11, wherein the second plurality of anellovectors is administered if the presence of anellovectors from the first plurality, or the progeny thereof, are detected.

14. A composition for use as a medicament for treating a human subject,
wherein the subject has previously been administered a first plurality of anellovectors comprising:
(a) a proteinaceous exterior that comprises an ORF1 molecule;
(b) a genetic element comprising a promoter element and a nucleic acid sequence (e.g., a DNA sequence) encoding the effector (e.g., an exogenous effector or an endogenous effector), said composition for use comprising a second plurality of anellovectors comprising:

(a) the same proteinaceous exterior as the anellovectors of the first plurality,

a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or
a proteinaceous exterior with at least one surface epitope in common with the anellovectors of the first plurality and
(b) a genetic element comprising a promoter element and a nucleic acid sequence (e.g., a DNA sequence) encoding an effector (e.g., an exogenous effector or an endogenous effector).

15. The method or composition for use of any of the preceding embodiments, wherein the first and the second plurality comprise about the same dosage of anellovectors, e.g., wherein the first plurality and the second plurality of anellovectors comprise about the same quantity and/or concentration of anellovectors.

16. The method or composition for use of any of the preceding embodiments, wherein the second plurality of anellovectors comprises about the same number of anellovectors as the first plurality of anellovectors, e.g., the second plurality comprises 90-110%, e.g., 95-105% of the number of anellovectors in the first plurality.

17. The method or composition for use of any of the preceding embodiments, wherein the second plurality of anellovectors comprises about the same number of anellovectors as the first plurality of anellovectors when normalized to body mass of the subject at the time of administration, e.g., the second plurality comprises 90-110%, e.g., 95-105% of the number of anellovectors in the first plurality when normalized to body mass of the subject at the time of administration.

18. The method or composition for use of any of the preceding embodiments, wherein the first plurality comprises a greater dosage of anellovectors than the second plurality, e.g., wherein the first plurality comprises a greater quantity and/or concentration of anellovectors relative to the second plurality.

19. The method or composition for use of any of the preceding embodiments, wherein the first plurality comprises a lower dosage of anellovectors than the second plurality, e.g., wherein the first plurality comprises a lower quantity and/or concentration of anellovectors relative to the second plurality.

20. The method or composition for use of any of the preceding embodiments, wherein the second plurality of anellovectors is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality of anellovectors to the subject.

21. The method or composition for use of any of the preceding embodiments, wherein the second plurality of anellovectors is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality of anellovectors to the subject.

22. The method or composition for use of any of the preceding embodiments, which further comprises assessing, after administration of the first plurality of anellovectors and before administration of the second plurality of anellovectors, one or more of:

a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);

b) the level or activity of the anellovector of the first plurality in the subject (e.g., by detecting the level of the ORF1 of the anellovector);

c) the presence, severity, progression, or a sign or symptom of a disease in the subject that the anellovector was administered to treat.

23. The method or composition for use of any of the preceding embodiments, which further comprises administering to the subject a third, fourth, fifth, and/or further plurality of anellovectors comprising:
(a) the same proteinaceous exterior as the anellovectors of the first plurality,
a proteinaceous exterior comprising a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality, or
a proteinaceous exterior with at least one surface epitope in common with the anellovectors of the first plurality and
(b) a genetic element comprising a promoter element and a nucleic acid sequence (e.g., a DNA sequence) encoding an effector (e.g., an exogenous effector or an endogenous effector).

24. The method or composition for use of any of the preceding embodiments, which comprises administering a repeated dose of anellovectors over the course of at least 1, 2, 3, 4, or 5 years.

25. The method or composition for use of embodiment 24, wherein the repeated dose is administered about every least 1, 2, 3, or 4 weeks, or 1, 2, 3, 4, 5, 6, 7, 8, 9, 10, 11, or 12 months.

26. The method or composition for use of any of the preceding embodiments, wherein the first plurality and the second plurality are administered via the same route of administration, e.g., intravenous administration.

27. The method or composition for use of any of embodiments 1-25, wherein the first plurality and the second plurality are administered via different routes of administration.

28. The method or composition for use of any of the preceding embodiments, wherein the first and the second pluralities of anellovectors are administered by the same entity (e.g., the same health care provider).

29. The method or composition for use of any of the embodiments 1-28, wherein the first and the second pluralities of anellovectors are administered by different entities (e.g., different health care providers).

30. The method or composition for use of any of the preceding embodiments, wherein wherein the subject is evaluated for the presence of an immune response, e.g., antibodies, against an Anellovirus, e.g., wherein the subject is evaluated before administration of the first plurality, before administration of the second plurality, or after administration of the second plurality.

31. The method or composition for use of any of the preceding embodiments, wherein the subject is administered an immune suppressant with the first and/or second plurality of anellovectors (e.g., administered simultaneously, or administered before or after such that the immune suppressant is active in the subject when the anellovectors are present in the subject).

32. The method or composition for use of any of embodiments 1-30, wherein the subject is not administered an immune suppressant with the first and/or second plurality of anellovectors.

33. The method or composition for use of any of the preceding embodiments, wherein the second plurality of anellovectors comprises a proteinaceous exterior with at least one surface epitope in common with the anellovectors of the first plurality.

34. The method or composition for use of any of the preceding embodiments, wherein the second plurality of anellovectors comprises the same proteinaceous exterior as the anellovectors of the first plurality.

35. The method or composition for use of any of the preceding embodiments, wherein the second plurality of anellovectors comprises an ORF1 molecule having the same amino acid sequence as the ORF1 molecule comprised by the anellovectors of the first plurality.

36. The method or composition for use of any of the preceding embodiments, wherein the second plurality of anellovectors comprises a proteinaceous exterior with at least 70%, 75%, 80%, 85%, 90%, 95%, 96%, 97%, 98%, 99%, or 100% amino acid sequence identity to the proteinaceous exterior of the anellovectors of the first plurality.

37. The method or composition for use of any of the preceding embodiments, wherein the second plurality of anellovectors comprises an ORF1 molecule having at least 70%, 75%, 80%, 85%, 90%, 95%, 96%, 97%, 98%, 99%, or 100% amino acid sequence identity to the ORF1 molecule of the anellovectors of the first plurality.

38. The method or composition for use of embodiment 37, wherein the proteinaceous exterior of the second plurality of anellovectors comprises one or more amino acid sequence difference (e.g., a conservative mutation) from the proteinaceous exterior of the first plurality of anellovectors.

39. The method or composition for use of embodiment 18, wherein the proteinaceous exterior of the second plurality of anellovectors comprises the same tertiary structure as the proteinaceous exterior of the first plurality of anellovectors (e.g., a calculated root-mean-square-deviation (RMSD) of about 0, e.g., 0).

40. The method or composition for use of embodiment 37, wherein an antibody that binds the proteinaceous exterior of the first plurality of anellovectors also binds to the proteinaceous exterior of the second plurality of anellovectors.

41. The method or composition for use of embodiment 40, wherein the antibody is comprised in the subject.

42. The method or composition for use of embodiment 40 or 41, wherein the antibody binds with about the same affinity (e.g., having a K_{D} of about 90-110%, e.g., 95-105%) to the proteinaceous exterior of the first plurality of anellovectors as to the proteinaceous exterior of the second plurality of anellovectors.

43. The method or composition for use of any of the preceding embodiments, wherein the second plurality of anellovectors delivers more copies (e.g., at least 2, 3, 4, 5, 6, 7, 8, 9, 10, 20, 30, 40, 50, 60, 70, 80, 90, 100, 500, or 1000-fold as many copies) of the effector to the subject than the first plurality of anellovectors.

44. The method or composition for use of any of the preceding embodiments, wherein the second plurality of anellovectors delivers about the same number of copies of the effector to the subject as the first plurality of anellovectors.

45. The method or composition for use of any of the preceding embodiments, wherein the second plurality of anellovectors delivers the effector to the subject at a level of at least about 50%, 55%, 60%, 65%, 70%, 75%, 80%, 85%, 90%, 95%, 96%, 97%, 98%, 99%, or 100% of copies of the effector delivered to the subject by the first plurality of anellovectors (e.g., wherein the effector delivered by the first plurality may be the same or different form the effector delivered by the second plurality).

46. The method or composition for use of any of the preceding embodiments, wherein the effector of the first and/or second plurality of anellovectors is an exogenous effector.

47. The method or composition for use of any of the preceding embodiments, wherein the Anellovectors of the first and/or second plurality are synthetic Anellovectors.

48. The method or composition for use of any of the preceding embodiments, wherein the Anellovectors of the first and/or second plurality are recombinant Anellovectors.

49. The method or composition for use of any of the preceding embodiments, wherein the effector of the first plurality of anellovectors is an endogenous effector and the effector of the second plurality is an exogenous effector.

50. The method or composition for use of any of the preceding embodiments, wherein the effector of the first and/or second plurality of anellovectors comprises growth hormone (e.g., human growth hormone (hGH)).

51. The method or composition for use of any of the preceding embodiments, wherein the effector of the first and/or second plurality of anellovectors comprises erythropoietin (EPO), e.g., human EPO.

52. The method or composition for use of any of the preceding embodiments, wherein the effector of the second plurality of anellovectors is the same as the effector of the first plurality of anellovectors.

53. The method or composition for use of any of the preceding embodiments, wherein the genetic element of the second plurality of anellovectors is the same as the genetic element of the first plurality of anellovectors, or wherein the genetic element of the first plurality of anellovectors has at least 70%, 75%, 80%, 85%, 90%, 95%, 96%, 97%, 98%, 99%, or 100% nucleic acid sequence identity to the genetic element of the second plurality of anellovectors.

54. The method or composition for use of any of embodiments 1-51, wherein the effector of the second plurality of anellovectors is different from the effector of the first plurality of anellovectors.

55. The method or composition for use of any of embodiments 1-51 and 54, wherein the genetic element of the second plurality of anellovectors is different from the genetic element of the first plurality of anellovectors.

56. The method or composition for use of any of embodiments 1-51 and 54-55, wherein the effector of the first plurality of anellovectors is a first exogenous effector, and the exogenous effector of the second plurality of anellovectors is a second exogenous effector.

57. The method or composition for use of any of embodiments 1-51 and 54-56, wherein:

the first plurality of anellovectors is administered to treat a first disease or condition in the subject, and

the second plurality of anellovectors is administered to treat a second disease or condition in the subject.

58. The method or composition for use of any of embodiments 1-51, wherein:

the first plurality of anellovectors is administered to treat a first disease or condition in the subject, and

the second plurality of anellovectors is administered to treat the first disease or condition in the subject.

59. A method of delivering an exogenous effector to a human subject, comprising:
(i) administering to the subject a first plurality of anellovectors comprising:
   (a) a proteinaceous exterior that comprises an ORF1 molecule;
   (b) a genetic element comprising a promoter element and a nucleic acid sequence (e.g., a DNA sequence) encoding an exogenous effector, and

(ii) subsequently administering to the subject a second plurality of anellovectors comprising:
(a) a proteinaceous exterior comprising an ORF1 molecule having the same sequence to the ORF1 molecule in the proteinaceous exterior of the first plurality, and
(b) a genetic element having the same nucleic acid sequence as the genetic element of the first plurality of anellovectors;

thereby delivering the exogenous effector to the subject.

60. The method or composition for use of any of the preceding embodiments, wherein the subject has hemophilia.

61. The method or composition for use of any of the preceding embodiments, wherein the subject has received a blood transfusion.

62. The method or composition for use of any of the preceding embodiments, wherein the effector of the first and/or second plurality of anellovectors is an endogenous effector.

63. The method or composition for use of any of the preceding embodiments, wherein the anellovectors of the first plurality are packaging deficient and/or replication deficient.

64. The method or composition for use of any of the preceding embodiments, wherein the anellovectors of the second plurality are packaging deficient and/or replication deficient.

65. The method or composition for use of any of the preceding embodiments, wherein the first plurality of anellovectors comprise a mixture of active and inactive particles.

66. The method or composition for use of any of the preceding embodiments, wherein the second plurality of anellovectors comprise a mixture of active and inactive particles.

67. The method or composition for use of any of the preceding embodiments, wherein a genetic element comprised in the anellovectors of the first plurality is detectable in the subject at least 50, 60, 70, 80, 90, 100, 110, 120, 130, 140, or 150 days after administration thereof, e.g., by a high-resolution melting (HRM) assay, e.g., as described in Example 1.

68. The method or composition for use of any of the preceding embodiments, wherein a genetic element comprised in the anellovectors of the second plurality is detectable in the subject at least 50, 60, 70, 80, 90, 100, 110, 120, 130, 140, or 150 days after administration thereof, e.g., by a high-resolution melting (HRM) assay, e.g., as described in Example 1.

69. The method or composition for use of any of the preceding embodiments, wherein the first and/or second plurality of anellovectors was isolated from a producer cell.

70. The method or composition for use of any of the preceding embodiments, wherein the first and/or second plurality of anellovectors was not obtained from a biological sample (e.g., blood) obtained from the subject.

71. The method or composition for use of any of the preceding embodiments, wherein the first plurality of anellovectors is administered to the subject as part of a first pharmaceutical composition.

72. The method or composition for use of any of the preceding embodiments, wherein the second plurality of anellovectors is administered to the subject as part of a second pharmaceutical composition.

73. The method or composition for use of embodiment 71 or 72, wherein at least 70%, 80%, 90%, 95%, or 100% of the genetic elements of anellovectors in the first pharmaceutical composition are identical to each other.

74. The method or composition for use of embodiment 71 or 72, wherein at least 70%, 80%, 90%, 95%, or 100% of the genetic elements of anellovectors in the first pharmaceutical composition have at least 70%, 75%, 80%, 85%, 90%, 95% or 100% sequence identity to a desired genetic element sequence.

75. The method or composition for use of embodiment 71 or 72, wherein at least 70%, 80%, 90%, 95%, or 100% of the genetic elements of anellovectors in the second pharmaceutical composition are identical to each other.

76. The method or composition for use of embodiment 71 or 72, wherein at least 70%, 80%, 90%, 95%, or 100% of the genetic elements of anellovectors in the second pharmaceutical composition have at least 70%, 75%, 80%, 85%, 90%, 95%, or 100% sequence identity to a desired genetic element sequence.

77. The method or composition for use of embodiment 71 or 72, wherein the first and/or second pharmaceutical compositions do not comprise red blood cells.

78. The method or composition for use of embodiment 71 or 72, wherein the first and/or second pharmaceutical compositions do not comprise cells.

79. The method or composition for use of any of the preceding embodiments, wherein the genetic element of the first and/or second plurality of anellovectors comprises an Anellovirus 5′ UTR, or a nucleic acid sequence having at least 80%, 85%, 90%, 95%, 96%, 97%, 98%, 99%, or 100% sequence identity thereto.

80. The method or composition for use of any of the preceding embodiments, wherein the genetic element of the first and/or second plurality of anellovectors comprises the nucleic acid sequence of nucleotides 323-393 of SEQ ID NO: 41, or a nucleic acid sequence having at least 80%, 85%, 90%, 95%, 96%, 97%, 98%, 99%, or 100% sequence identity thereto.

81. The method or composition for use of any of the preceding embodiments, wherein the genetic element of the first and/or second plurality of anellovectors comprises a sequence of at least 100 nucleotides in length, which consists of G or C at at least 80% of the positions.

82. The method or composition for use of any of the preceding embodiments, wherein the genetic element of the first and/or second plurality of anellovectors comprises a sequence having a GC-rich region nucleotide sequence of:

CGGCGGX₁GGX₂GX₃X₄X₅CGCGCTX₆CGCGCGCX₇X₈X₉X₁₀CX₁₁X₁₂X₁₃X₁₄GGGGX₁₅X₁₆X₁₇X₁₈X₁₉X₂₀X₂₁GCX₂₂X₂₃X₂₄X₂₅CCCCCCCX₂₆CGCGCATX₂₇X₂₈GCX₂₉CGGGX₃₀CCCCCCCCCX₃₁X₃₂X₃₃GGGGGGCTCCGX₃₄CCCCCCGGCCCCCC, wherein:

X₁=G or C

X₂=G, C, or absent

X₃=C or absent

X₄=G or C

X₅=G or C

X₆=T, G, or A

X₇=G or C

X₈=G or absent

X₉=C or absent

X₁₀=C or absent

X₁₁=G, A, or absent

X₁₂=G or C

X₁₃=C or T

X₁₄=G or A

X₁₅=G or A

X₁₆=A, G, T, or absent

X₁₇=G, C, or absent

X₁₈=G, C, or absent

X₁₉=C, A, or absent

X₂₀=C or A

X₂₁=T or A

X₂₂=G or C

X₂₃=G, T, or absent

X₂₄=C or absent

X₂₅=G, C, or absent

X₂₆=G or C

X₂₇=G or absent

X₂₈=C or absent

X₂₉=G or A

X₃₀=G or T

X₃₁=C, T, or absent

X₃₂=G, C, A, or absent

X₃₃=G or C

X₃₄=C or absent (SEQ ID NO: 743).

83. The method or composition for use of any of the anellovectors comprises the amino acid sequence of SEQ ID NO: 45, or an amino acid sequence having at least 80%, 85%, 90%, 95%, 96%, 97%, 98%, 99%, or 100% sequence identity thereto.

84. The method or composition for use of any of the preceding embodiments, wherein the first and/or second plurality of anellovectors comprises one or more polypeptides comprising one or more of an amino acid sequence chosen from an Anellovirus ORF2, ORF2/2, ORF2/3, ORF1, ORF1/1, or ORF1/2, or an amino acid sequence having at least 80%, 85%, 90%, 95%, 96%, 97%, 98%, 99%, or 100% sequence identity thereto.

85. The method or composition for use of any of the preceding embodiments, wherein the first and/or second plurality of anellovectors comprises a nucleic acid sequence encoding an amino acid sequence chosen from ORF1, ORF2, ORF2/2, ORF2/3, ORF1/1, or ORF1/2 of Table 12, or an amino acid sequence having at least 80%, 85%, 90%, 95%, 96%, 97%, 98%, 99%, or 100% sequence identity thereto.

86. The method or composition for use of any of the preceding embodiments, wherein the first and/or second plurality of anellovectors does not comprise a polypeptide having at least 80%, 85%, 90%, 95%, 96%, 97%, 98%, 99%, or 100% sequence identity to an Anellovirus ORF2, ORF2/2, ORF2/3, ORF1/1, or ORF1/2.

87. The method or composition for use of any of the preceding embodiments, wherein the genetic element of the first and/or second plurality of anellovectors is circular, single stranded DNA.

88. The method or composition for use of any of the preceding embodiments, wherein the genetic element of the first and/or second plurality of anellovectors integrates at a frequency of less than 1% of the anellovectors that enters a cell of the subject.

89. The method or composition for use of any of the preceding embodiments, wherein the first and/or second plurality of anellovectors do not comprise a polynucleotide encoding one or both of a replication factor and a capsid protein.

90. The method or composition for use of any of the preceding embodiments, wherein the anellovectors of the first and/or second plurality are replication defective.

91. The method or composition for use of any of the preceding embodiments, wherein the effector comprises:

(i) an intracellular polypeptide other than nano-luciferase;

(ii) an intracellular nucleic acid (e.g., an miRNA or siRNA);

(iii) a secreted polypeptide chosen from an antibody molecule, an enzyme, a hormone, a cytokine molecule, a complement inhibitor, a growth factor, or a growth factor inhibitor, or a functional variant of any of the foregoing; or

(iv) a polypeptide that, when mutated, causes a human disease, or a functional variant of said polypeptide.

92. The method or composition for use of any of the preceding embodiments, wherein the anellovectors of the first plurality, the second plurality, or both of the first and second pluralities, were made by a method comprising:

a) providing a nucleic acid construct that comprises:
i) a first Anellovirus genetic element comprising a sequence encoding an exogenous effector; and
ii) a second Anellovirus genetic element or fragment thereof, placed in tandem with the first Anellovirus genetic element; and
iii) optionally, a spacer sequence situated between (i) and (ii); and

b) contacting a cell (e.g., a mammalian host cell) with the nucleic acid construct under conditions that allow the Anellovirus genetic element of the nucleic acid construct to be replicated or amplified;

thereby manufacturing the anellovector genetic element.

93. The method or composition for use of embodiment 92, wherein the second Anellovirus genetic element or fragment thereof has a length of less than 2800, 2700, 2600, 2500, 2000, 1500, 1000, 900, 800, 700, 600, or 500 nucleotides.

94. The method or composition for use of embodiment 92 or 93, wherein the second Anellovirus genetic element or fragment thereof is positioned 3′ relative to the first Anellovirus genome.

95. The method or composition for use of any of embodiments 92-94, wherein the second Anellovirus genetic element or fragment thereof is positioned 5′ relative to the first Anellovirus genome.

96. The method or composition for use of any of embodiments 92-95, wherein the nucleic acid construct comprises the spacer sequence, wherein optionally the spacer sequence has a length of 1, 2, 3, 4, 5, 6, 7, 8, 9, 10, 11, 12, 13, 14, 15, 16, 17, 18, 19, 20, or more amino acids, or a length between 1-5, 5-10, 10-15, or 15-20 amino acids.

97. The method or composition for use of any of embodiments 92-96, wherein the nucleic acid construct does not comprise the spacer sequence.

98. The method or composition for use of any of the preceding embodiments, wherein the anellovectors of the first plurality, the second plurality, or both of the first and second pluralities, were made by a method comprising:

(i) providing an insect cell comprising:
a) an Anellovirus genetic element comprising a promoter operably linked to a sequence encoding an exogenous effector, and
b) an Anellovirus ORF1 molecule;

(ii) incubating the insect cell under conditions suitable for enclosure of the Anellovirus genetic element in a proteinaceous exterior comprising the Anellovirus ORF1 molecule.

99. The method or composition for use of embodiment 98, wherein providing the insect cell comprises introducing into the insect cell a nucleic acid construct encoding Anellovirus ORF1 molecule

100. The method or composition for use of embodiment 99, wherein the nucleic acid comprises a backbone region suitable for replication of the nucleic acid construct in insect cells (e.g., a Baculovirus backbone region), optionally wherein the backbone region is also suitable for replication of the nucleic acid construct in bacterial cells.

101. The method or composition for use of any of embodiments 98-100, wherein providing the insect cell comprises introducing into the insect cell the Anellovirus genetic element.

102. A method of amplifying a circular DNA molecule comprising an Anellovirus sequence, the method comprising:

(a) providing a sample comprising a circular DNA molecule comprising an Anellovirus sequence and a first primer having at least 7, 8, or 9 nucleotides complementary to a portion of the Anellovirus sequence; and

(b) contacting the circular DNA molecule with a polymerase molecule (e.g., a DNA-dependent DNA polymerase molecule);

wherein the contacting results in linear amplification (e.g., rolling circle amplification or multiple strand displacement amplification) of the DNA molecule, or a portion thereof.

103. The method of embodiment 102, wherein (a) comprises contacting the circular DNA molecule with the primer.

104. A method of amplifying a circular DNA molecule comprising an Anellovirus sequence, the method comprising:

(a) providing a sample comprising a circular DNA molecule comprising an Anellovirus sequence; and

(b) contacting the circular DNA molecule with a plurality of primers, wherein a first primer of said plurality has at least 7, 8, or 9 nucleotides complementary to a portion of the Anellovirus sequence, in the presence of a polymerase (e.g., a DNA-dependent DNA polymerase molecule);

wherein the contacting results in rolling circle amplification of the DNA molecule, or a portion thereof.

105. The method of embodiment 104, wherein (b) comprises contacting the circular DNA molecule with the polymerase molecule.

106. The method of any of embodiments 102-105, wherein the sample comprises a plurality of primers having at least 7, 8, or 9 nucleotides complementary to a portion of the Anellovirus sequence.

107. The method of any of embodiments 102-106, wherein the first primer has at least 50%, 55%, 60%, 65%, 70%, 75%, 80%, 85%, 90%, 95%, 96%, 97%, 98%, 99%, or 100% sequence identity to a second primer of the plurality, and wherein the first primer and the second primer are not identical.

108. The method of any of embodiments 102-107, wherein each of the plurality of primers share the same orientation relative to the circular DNA molecule.

109. The method of any of embodiments 102-108, wherein:
(i) the circular DNA molecule comprises a plurality of sequences recognized by the plurality of primers;
(ii) the plurality of primers are all positive-strand primers or all negative-strand primers;
(iii) the plurality of primers are all same-strand primers;
(iv) the plurality of primers all comprise at least 3, 4, 5, 6, 7, 8, 9, or 10 contiguous nucleotides in common; and/or

(v) the plurality of primers comprises at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more different primers.

110. The method of any of embodiments 102-109, wherein the first primer and the second primer differ at 1, 2, 3, or 4 positions, wherein optionally the first primer and the second primer are each 9 nucleotides in length.

111. The method of any of embodiments 102-110, further comprising, prior to the contacting step, enriching the sample for one or more constituents of interest.

112. The method of embodiment 111, wherein the one or more constituents of interest comprises nucleic acid molecules.

113. The method of embodiment 112, wherein the one or more constituents of interest comprises non-chromosomal nucleic acid molecules, e.g., circular non-chromosomal nucleic acid molecules and/or viral nucleic acid molecules (e.g., Anellovirus nucleic acid molecules, e.g., Anellovirus genomes).

114. The method of any of embodiments 102-113, further comprising, prior to the contacting step, denaturing the circular DNA molecule, e.g., by exposing the circular DNA molecule to a temperature of at least about 80, 85, 90, 91, 92, 93, 94, 95, 96, 97, 98, or 99° C., e.g., for at least about 1, 2, 3, 4, or 5 minutes.

115. The method of embodiment 114, further comprising, after the denaturing step, cooling the circular DNA molecule, e.g., to about 2, 3, 4, 5, 6, or 7° C.

116. The method of any of embodiments 102-115, further comprising, after the contacting step, incubating the sample, e.g., at about 25, 26, 27, 28, 29, 30, 31, 32, 33, 34, or 35° C., e.g., for at least about 10, 15, 16, 17, 18, 19, 20, 21, 22, 23, 24, 25, or 30 hours.

117. The method of embodiment 116, further comprising, after the incubating step, incubating the sample under conditions suitable to inactivate the polymerase molecule (e.g., incubating the sample at about 60, 61, 62, 63, 64, 65, 66, 67, 68, 69, or 70° C., e.g., for at least 5, 6, 7, 8, 9, 10, 11, 12, 13, 14, or 15 minutes).

118. The method of any of embodiments 102-117, wherein the amplified nucleic acid molecule is validated by PCR, e.g., using one or more pan-Anellovirus primers, e.g., as described in Ninomiya et al. 2008 (J. Clin. Microbiol. 46: 507-514; incorporated herein by reference with respect to the pan-Anellovirus primers and methods relating to the same).

119. The method of any of embodiments 102-118, wherein the amplified nucleic acid molecule is assessed by library quality control (QC) techniques, e.g., as described herein, e.g., in Example 36.

120. The method of any of embodiments 102-119, wherein the contacting of (b) occurs in a mixture having one or more of the following characteristics:

(i) a concentration of the primer or primers of about 0.1, 0.2, 0.3, 0.4, 0.5, 0.6, 0.7, or 0.8 μM per primer, or 0.1-0.2, 0.2-0.3, 0.3-0.4, 0.4-0.5, 0.5-0.6, 0.6-0.7, or 0.7-0.8 μM per primer;

(ii) a polymerase (e.g., a DNA polymerase) buffer suitable for the polymerase molecule (e.g., the DNA-dependent DNA polymerase molecule) to synthesize DNA (e.g., a Phi29 DNA polymerase buffer);

(iii) comprising bovine albumin serum, e.g., at a concentration of about 100, 150, 160, 170, 180, 190, 200, 210, 220, 230, 240, 250, or 300 ng/μL, or about 100-150, 150-175, 175-190, 190-200, 200-210, 210-225, 225-250, or 250-300 ng/μL;

(iii) comprising dNTPs, e.g., at a concentration of about 0.5, 0.6, 0.7, 0.8, 0.9, 1.0, 1.1, 1.2, 1.3, 1.4, 1.5, or 2 mM, or about 0.5-0.7, 0.7-0.9, 0.9-1.0, 1.0-1.1, 1.1-1.3, 1.3-1.5, or 1.5-2 mM; and/or

(iv) wherein the polymerase molecule (e.g., the DNA-dependent DNA polymerase molecule) comprises Phi29 polymerase, e.g., at a concentration of about 1, 1.5, 2, 2.5, or 3 U/μL, or 1-1.5, 1.5-2, 2-2.5, or 2.5-3 U/μL.

121. The method of any of embodiments 102-120, wherein the method does not comprise thermocycling, e.g., wherein the method is performed isothermically.

122. The method of any of embodiments 102-121, wherein the amplification comprises displacement (e.g., partial or full displacement) of the strand synthesized by the polymerase molecule (e.g., the DNA-dependent DNA polymerase molecule) from the circular DNA molecule.

123. The method of any of embodiments 102-122, wherein the strand synthesized by the polymerase molecule (e.g., the DNA-dependent DNA polymerase molecule) is released into the surrounding solution.

124. The method of embodiment 123, wherein the polymerase molecule (e.g., the DNA-dependent DNA polymerase molecule) nicks the synthesized strand, thereby releasing the synthesized strand.

125. The method of any of embodiments 102-124, wherein the polymerase molecule (e.g., the DNA-dependent DNA polymerase molecule) synthesizes a product strand comprising a plurality of copies of the sequence of the circular DNA molecule, or a fragment thereof comprising at least 1000, 2000, 2500, 3000, 3500, or 4000 contiguous nucleotides thereof.

126. The method of embodiment 125, wherein the plurality of copies of the sequence of the circular DNA molecule, or the fragment thereof, are arranged in tandem within the product strand.

127. The method of any of embodiments 102-124, wherein the polymerase molecule (e.g., the DNA-dependent DNA polymerase molecule) synthesizes a product strand comprising one copy of the sequence of the circular DNA molecule, or a fragment thereof comprising at least 1000, 2000, 2500, 3000, 3500, or 4000 contiguous nucleotides thereof.

128. The method of any of embodiments 102-127, further comprising sequencing the amplified circular DNA molecules.

129. The method of embodiment 128, wherein the sequencing comprises next-generation sequencing (e.g., sequencing by synthesis (e.g., Illumina sequencing), pyrosequencing, reversible terminator sequencing, sequencing by ligation, or nanopore sequencing, or any combination thereof).

130. The method of embodiment 128, wherein the sequencing comprises Sanger sequencing.

131. The method of any of embodiments 128-130, further comprising computational analysis of the sequencing results.

132. The method of embodiment 131, wherein the computational analysis comprises identifying one or more Anellovirus sequences represented in the sequences of the amplified nucleic acid molecules.

133. The method of embodiment 131 or 132, wherein the computational analysis comprises determining sequence similarity of the genome sequence or one or more elements comprised and/or encoded therein within a plurality (e.g., at least 2, 3, 4, 5, 6, 7, 8, 9, 10, 15, 20, 30, 40, 50, 60, 70, 80, 90, 100, 200, 300, 400, 500, 600, 700, 800, 900, 1000, 1100, 1200, 1300, 1400, or 1500) of distinct sequences of the amplified nucleic acid molecules.

134. The method of any of embodiments 131-133, wherein the computational analysis comprises determining the Anellovirus sequences present in each sample, each subject, each tissue or cell type, and/or each time point.

135. The method of any of embodiments 131-134, wherein the computational analysis comprises determining the unique Anellovirus lineages present in each sample, each subject, each tissue or cell type, and/or each time point.

136. The method of any of embodiments 131-135, wherein the computational analysis comprises comparing the sequences present in one sample to another sample.

137. The method of any of embodiments 131-136, wherein the computational analysis comprises comparing the sequences present in one subject to another subject.

138. The method of any of embodiments 131-137, wherein the computational analysis comprises comparing the sequences present in one tissue or cell type to another tissue or cell type.

139. The method of any of embodiments 131-138, wherein the computational analysis comprises comparing the sequences present at one time point to the sequences present at another time point.

140. The method of any of embodiments 131-139, wherein the computational analysis comprises multidimensional scaling (MDS) of the sequences, or portions thereof (e.g., portions comprising or encoding one or more of: a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, and/or GC rich region).

141. The method of any of embodiments, 131-140, wherein the computational analysis comprises phylogenetic analysis.

142. The method of embodiment 133, wherein the one or more elements comprised and/or encoded in the genome sequence of the Anellovirus comprises one or more of: a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, and/or GC rich region.

143. The method of any of embodiments 102-142, wherein the sample is obtained from a subject (e.g., a human subject, e.g., a healthy or asymptomatic human subject).

144. The method of embodiment 143, wherein the sample is a biological sample.

145. The method of embodiment 144, wherein the biological sample comprises blood or serum.

146. The method of any of embodiments 102-145, wherein the sample comprises at least 2, 3, 4, 5, 6, 7, 8, 9, or 10 different circular DNA molecules (e.g., comprising at least 2, 3, 4, 5, 6, 7, 8, 9, or 10 different Anellovirus sequences).

147. The method of any of embodiments 102-146, wherein the method is performed on a plurality of samples (e.g., at least 5, 10, 15, 20, 25, 30, 40, 50, 60, 70, 80, 90, 100, 110, 120, 125, 126, 127, 128, 129, 130, 140, 150, 160, 170, 180, 190, 200, 250, 300, 400, 500, 600, 700, 800, 900, or 1000 samples), e.g., in parallel.

148. The method of embodiment 147, wherein the plurality of samples is obtained from a plurality of subjects (e.g., human subjects), e.g., at least 5, 10, 15, 20, 25, 30, 40, 50, 60, 70, 80, 90, 100, 110, 120, 125, 126, 127, 128, 129, 130, 140, 150, 160, 170, 180, 190, 200, 250, 300, 400, 500, 600, 700, 800, 900, or 1000 subjects, e.g., serially or in parallel.

149. The method of embodiment 147 or 148, wherein the plurality of samples is obtained from a plurality of time points (e.g., a plurality of samples obtained from the same subject at multiple time points, or a plurality of samples obtained from a plurality of subjects at multiple time points).

150. The method of any of embodiments 147-149, wherein the plurality of samples is obtained from a plurality of tissue or cell types, e.g., at least 5, 10, 15, 20, 25, 30, 40, 50, 60, 70, 80, 90, or 100 different tissue or cell types.

151. A primer comprising a nucleic acid sequence according to any of SEQ ID NOs: 1-24, e.g., any of SEQ ID NOs: 1, 3, 4, 6, 8, 10, 12, 14, 17, 19, 21, or 23.

152. The primer of embodiment 151, which is 9, 10, 11, 12, 13, 14, or 15 nucleotides in length.

153. A kit or a mixture comprising a plurality of different primers,

wherein each of the plurality of primers binds to a nucleic acid molecule having a sequence of any of SEQ ID NOs: 1-24, e.g., any of SEQ ID NOs: 2, 5, 7, 9, 11, 13, 15, 16, 18, 20, 22, or 24.

154. A kit or a mixture comprising a plurality of different primers comprising a nucleic acid sequence according to any 2, 3, 4, 5, 6, 7, 8, 9, 10, 11, or 12 or more of SEQ ID NOs: 1-24, e.g., any of SEQ ID NOs: 1, 3, 4, 6, 8, 10, 12, 14, 17, 19, 21, or 23.

155. The kit or mixture of any of embodiments 153-154, wherein or more primers of the plurality comprises a nucleic acid sequence according to CGAATGGYW (SEQ ID NO: 1), e.g., wherein primers in the plurality comprise a nucleic acid sequence according to any combination of 2, 3, or all of CGAATGGCA, CGAATGGCT, CGAATGGTA, or CGAATGGTT.

156. The kit or mixture of any of embodiments 153-155, wherein or more primers of the plurality comprises a nucleic acid sequence according to YTGYGGBTG (SEQ ID NO: 3), e.g., wherein primers in the plurality comprise a nucleic acid sequence according to any combination of 2, 3, 4, 5, 6, 7, 8, 9, 10, 11, or all of CTGCGGCTG, CTGCGGGTG, CTGCGGTTG, CTGTGGCTG, CTGTGGGTG, CTGTGGTTG, TTGCGGCTG, TTGCGGGTG, TTGCGGTTG, TTGTGGCTG, TTGTGGGTG, or TTGTGGTTG.

157. The kit or mixture of any of embodiments 153-156, wherein or more primers of the plurality comprises a nucleic acid sequence according to YAGAMACMM (SEQ ID NO: 4), e.g., wherein primers in the plurality comprise a nucleic acid sequence according to any combination of 2, 3, 4, 5, 6, 7, 8, 9, 10, 11, 12, 13, 14, 15, or all of CAGAAACAA, CAGAAACAC, CAGAAACCA, CAGAAACCC, CAGACACAA, CAGACACAC, CAGACACCA, CAGACACCC, TAGAAACAA, TAGAAACAC, TAGAAACCA, TAGAAACCC, TAGACACAA, TAGACACAC, TAGACACCA, or TAGACACCC.

158. The kit or mixture of any of embodiments 153-157, wherein or more primers of the plurality comprises a nucleic acid sequence according to GTACCAYTTR (SEQ ID NO: 17), e.g., wherein primers in the plurality comprise a nucleic acid sequence according to any combination of 2, 3, or all of GTACCACTTA, GTACCACTTG, GTACCATTTA, GTACCATTTG.

159. The kit or mixture of any of embodiments 153-158, wherein or more primers of the plurality comprises a nucleic acid sequence according to SACCACWAAC (SEQ ID NO: 6), e.g., wherein primers in the plurality comprise a nucleic acid sequence according to any combination of 2, 3, or all of GACCACAAAC, GACCACTAAC, CACCACAAAC, or CACCACTAAC.

160. The kit or mixture of any of embodiments 153-159, wherein or more primers of the plurality comprises a nucleic acid sequence according to CACCGACVA (SEQ ID NO: 19), e.g., wherein primers in the plurality comprise a nucleic acid sequence according to any combination of 2 or all of CACCGACAA, CACCGACCA, or CACCGACGA.

161. The kit of any of embodiments 153-160, wherein optionally each primer is in a separate container.

162. The mixture of any of embodiments 153-161.

163. The mixture of any of embodiments 153-162, which further comprises one or both of a polymerase molecule (e.g., a DNA-dependent DNA-polymerase molecule) or a circular nucleic acid molecule comprising an Anellovirus sequence.

164. An isolated nucleic acid molecule comprising one or more sequences having a sequence of any of SEQ ID NOs: 13-24.

165. A method of amplifying a circular nucleic acid molecule, the method comprising:

(a) providing a sample comprising a circular nucleic acid molecule of embodiment 63 and the mixture of any of embodiments 153-163 or the primer of embodiment 153 or 154;

(b) contacting the circular nucleic acid molecule with the polymerase molecule (e.g., the DNA-dependent DNA polymerase molecule);

wherein the contacting results in linear amplification (e.g., rolling circle amplification or multiple strand displacement amplification) of the nucleic acid molecule, or a portion thereof.

166. A circular DNA molecule comprising a thiophosphate-comprising primer sequence comprising a sequence according to any of SEQ ID NOs: 1-12 and at least 100, 200, 300, 400, 500, 600, 700, 800, 900, 1000, 1500, 2000, 2500, 3000, 3500, or 4000 contiguous nucleotides of an Anellovirus sequence.

167. The circular DNA molecule of embodiment 166, wherein the primer sequence comprises one or more (e.g., 1 or 2) thiophosphate linkages.

168. The circular DNA molecule of embodiment 167, which comprises 1 or 2 thiophosphate linkages, wherein optionally all the other linkages in the circular DNA molecule are phosphate linkages.

169. A DNA molecule comprising a plurality of Anellovirus sequences, or fragments thereof each comprising at least 100, 200, 300, 400, 500, 600, 700, 800, 900, 1000, 1500, 2000, 2500, 3000, 3500, or 4000 contiguous nucleotides of the Anellovirus sequence;

wherein the Anellovirus sequences or fragments thereof each comprise (e.g., at one end) a thiophosphate-comprising primer sequence comprising a sequence according to any of SEQ ID NOs: 1-12.

170. The DNA molecule of embodiment 169, wherein the Anellovirus sequences or fragments thereof are arranged in tandem.

171. The DNA molecule of embodiment 169 or 170, wherein the primer sequences each comprise one or more (e.g., 1 or 2) thiophosphate linkages.

172. The DNA molecule of any of embodiments 169-171, which comprises one or more (e.g., 1, 2, 3, 4, 5, 6, 7, 8, 9, 10, 11, 12, 13, or 14) sequences each having at least 75%, 80%, 85%, 90%, 95%, 96%, 97%, 98%, 99%, or 100% sequence identity to an Anellovirus element as listed in any one of Tables A1, A3, A5, A7, A9, A11, B1-B5, 1, 3, 5, 7, 9, 11, 13, 15, or 17 of PCT/US2019/065995.

173. The DNA molecule of embodiment 172, wherein the sequences have at least 75%, 80%, 85%, 90%, 95%, 96%, 97%, 98%, 99%, or 100% sequence identity to a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, or GC rich region as listed in any one of Tables A1, A3, A5, A7, A9, A11, B1-B5, 1, 3, 5, 7, 9, 11, 13, 15, or 17 of PCT/US2019/065995.

174. The primer, method, mixture, or nucleic acid molecule of any of embodiments 102-173, wherein the primer comprises at least 5, 6, 7, 8, 9, 10, 11, 12, 13, 14, 15, 16, 17, 18, 19, or 20 nucleotides.

175. The method, mixture, or nucleic acid molecule of any of embodiments 102-174, wherein each primer of the plurality is independently selected from 9, 10, 11, 12, 13, 14, or 15 nucleotides in length.

176. The method, mixture, or nucleic acid molecule of any of embodiments 102-175, wherein each primer of the plurality is the same length in nucleotides.

177. The method, mixture, or nucleic acid molecule of any of embodiments 102-176, wherein each primer of the plurality is 9 nucleotides in length.

178. The method or mixture of any of embodiments 102-177, wherein the polymerase molecule is a DNA-dependent DNA polymerase molecule, e.g., a Phi29 DNA polymerase molecule.

179. The method or mixture of any of embodiments 102-178, wherein the polymerase molecule (e.g., the DNA-dependent DNA polymerase molecule) can synthesize a DNA product of at least 1, 2, 3, 4, 5, 10, 20, 30, 40, 50, 60, or 70 kb.

180. The method, mixture, or nucleic acid molecule of any of embodiments 102-179, wherein each primer comprises one or more (e.g., 1 or 2) thiophosphate linkages.

181. The method, mixture, or nucleic acid molecule of embodiment 180, wherein the one or more thiophosphate modifications are each positioned between two of the three 3′-most nucleotides in the primer.

182. The method, mixture, or nucleic acid molecule of embodiment 181, wherein one thiophosphate modification is positioned between the first and second nucleotides at the 3′ end of the primer.

183. The method, mixture, or nucleic acid molecule of embodiment 181 or 182, wherein one thiophosphate modification is positioned between the second and third nucleotides at the 3′ end of the primer.

184. The method, mixture, or nucleic acid molecule of any of embodiments 102-183, wherein the circular DNA molecule is single-stranded.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

FIG. 1 is a graph showing the relative time of each blood draw for all recipient patients over the days post-transfusion.

FIGS. 2A and 2B depict similarity of Anellovirus capsid protein in donor strains versus recipient strains pre-transfusion (A). Strains circled in red are strains that are categorized as re-dosing candidates. These strains were observed both pre-transfusion and at one or more time points post-transfusion (B).

FIG. 3 is a series of graphs showing persistence of re-dosed Anelloviruses in patients, as determined by High-Resolution Melting (HRM) assay. Recipient patients were tested for Anellovirus profiles at 24, 82, 110, and 167 days post-transfusion, and the resultant profiles were compared to the Anellovirus profiles of the recipient patient at day 0 versus the Anellovirus profile of the donor.

FIG. 4 depicts a schematic of a kanamycin vector encoding the LY1 strain of TTMiniV (“Anellovector 1”).

FIG. 5 depicts a schematic of a kanamycin vector encoding the LY2 strain of TTMiniV (“Anellovector 2”).

FIG. 6 depicts transfection efficiency of synthetic anellovectors in 293T and A549 cells.

FIGS. 7A and 7B depict quantitative PCR results that illustrate successful infection of 293T cells by synthetic anellovectors.

FIGS. 8A and 8B depict quantitative PCR results that illustrate successful infection of A549 cells by synthetic anellovectors.

FIGS. 9A and 9B depict quantitative PCR results that illustrate successful infection of Raji cells by synthetic anellovectors.

FIGS. 10A and 10B depict quantitative PCR results that illustrate successful infection of Jurkat cells by synthetic anellovectors.

FIGS. 11A and 11B depict quantitative PCR results that illustrate successful infection of Chang cells by synthetic anellovectors.

FIG. 12 is a schematic showing an exemplary workflow for production of anellovectors (e.g., replication-competent or replication-deficient anellovectors as described herein).

FIG. 13 is a graph showing fold change in miR-625 expression in HEK293T cells transfected with the indicated plasmid.

FIG. 14 is a diagram showing infection of Raji B cells with anellovectors encoding a miRNA targeting n-myc interacting protein (NMI). Shown is quantification of genome equivalents of anellovectors detected after infection of Raji B cells (arrow) or control cells with NMI miRNA-encoding anellovectors.

FIG. 15 is a diagram showing infection of Raji B cells with anellovectors encoding a miRNA targeting n-myc interacting protein (NMI). The Western blot shows that anellovectors encoding the miRNA against NMI reduced NMI protein expression in Raji B cells, whereas Raji B cells infected with anellovectors lacking the miRNA showed comparable NMI protein expression to controls.

FIG. 16 is a series of graphs showing quantification of anellovector particles generated in host cells after infection with an anellovector comprising an endogenous miRNA-encoding sequence and a corresponding anellovector in which the endogenous miRNA-encoding sequence was deleted.

FIGS. 17A-17B are a series of diagrams showing constructs used to produce anellovectors expressing nano-luciferase (A) and a series of anellovector/plasmid combinations used to transfect cells (B)

FIGS. 18A-18C are a series of diagrams showing nano-luciferase expression in mice injected with anellovectors. (A) Nano-luciferase expression in mice at days 0-9 after injection. (B) Nano-luciferase expression in mice injected with various anellovector/plasmid construct combinations, as indicated. (C) Quantification of nano-luciferase luminescence detected in mice after injection. Group A received a TTMV-LY2 vector±nano-luciferase. Group B received a nano-luciferase protein and TTMV-LY2 ORFs.

FIG. 19A is a gel electrophoresis image showing circularization of TTMV-LY2 plasmids pVL46-063 and pVL46-240.

FIG. 19B is a chromatogram showing copy numbers for linear and circular TTMV-LY2 constructs, as determined by size exclusion chromatography (SEC).

FIG. 19C is a schematic showing the domains of an Anellovirus ORF1 molecule and the hypervariable region to be replaced with a hypervariable domain from a different Anellovirus.

FIG. 19D is a schematic showing the domains of ORF1 and the hypervariable region that will be replaced with a protein or peptide of interest (POI) from a non-anellovirus source.

FIG. 20 is a graph showing that anellovectors based on tth8 or LY2, engineered to contain a sequence encoding human erythropoietin (hEpo), could deliver a functional transgene to mammalian cells.

FIGS. 21A and 21B are a series of graphs showing that engineered anellovectors administered to mice were detectable seven days after intravenous injection.

FIG. 22 is a graph showing that hGH mRNA was detected in the cellular fraction of whole blood seven days after intravenous administration of an engineered anellovector encoding hGH.

FIG. 23 is a graph showing the ability of an in vitro circularized (IVC) TTV-tth8 genome (IVC TTV-tth8) compared to a TTV-tth8 genome in a plasmid to yield TTV-tth8 genome copies at the expected density in HEK293T cells.

FIG. 24 is a series of graphs showing the ability of an in vitro circularized (IVC) LY2 genome (WT LY2 IVC) and a wild-type LY2 genome in plasmid (WT LY2 Plasmid) to yield LY2 genome copies at the expected density in Jurkat cells.

FIG. 25 is a diagram showing an alignment of secondary structure of the jelly roll domain of Anellovirus ORF1 proteins from Alphatorquevirus, Betatorquevirus, and Gammatorquevirus (SEQ ID NOs: 950-975). These secondary structural elements are highly conserved.

FIGS. 26A-26C are a series of diagrams showing that a tandem Anellovirus plasmid can increase Anellovirus production. (A) Plasmid map for an exemplary tandem Anellovirus plasmid. (B) Transfection of MOLT-4 cells with a tandem Anellovirus plasmid resulted in recovery of wild-type sized anellovirus genomes. (C) Anellovirus genomes produced in MOLT-4 cells from tandem anellovirus plasmid migrate at the expected density for encapsidated viral particles. GCR=GC-rich region. Bacterial SM=bacterial selection marker. Bacterial ori=bacterial origin of replication. ORFs=open reading frames. Prom.=promoter. 5CD=5′ untranslated region conserved domain.

FIGS. 27A-27E are a series of diagrams showing exemplary tandem constructs based on the Ring2 genome. (A) Tandem constructs comprising a first copy of a genetic element and a full or partial second copy of the genetic element positioned 3′ relative to the first copy. Each successive construct includes a greater truncation of the 3′ end of the second copy. The constructs may include a downstream replication-facilitating sequence (dRFS), e.g., comprising the 5CD (5′ UTR conserved domain), as indicated. (B) Tandem constructs comprising a first copy of a genetic element and a full or partial second copy of the genetic element positioned 5′ relative to the first copy. Each successive construct includes a greater truncation of the 5′ end of the second copy. (C) Tandem constructs comprising a partial first copy of a genetic element (e.g., comprising an uRFS) and a partial second copy (e.g., comprising a dRFS) of the genetic element positioned 5′ relative to the first copy. Each successive construct includes a greater truncation of the 5′ end of the first copy and a greater proportion of the 3′ end of the second copy. (D) Southern blot on total DNA harvested from MOLT-4 cells transfected with constructs shown in 2A and 2B, demonstrating recovery of wild-type length anellovirus genomes. (E) DNase-protection qPCR of anelloviral genomes from CsCl density gradients, demonstrating enclosure of anelloviral genomes produced in MOLT-4 cells with constructs shown in 2A and 2B.

FIG. 27F is a series of diagrams showing long RNA reads for full-length Ring1 ORF1 mRNA from Jurkat cells transfected with a variety of Ring1 constructs (as indicated), including a tandem Ring1 construct encoding, in the first copy of the Ring1 backbone, a sequence encoding an eGFP-ORF1 fusion protein.

FIG. 27G is a series of diagrams showing detection of ORF1 protein expression in MOLT-4 cells into which Ring2 tandem constructs had been introduced by nucleofection.

FIG. 27H is a diagram showing an exemplary baculovirus construct comprising two Ring2 genomes arranged in tandem.

FIG. 27I is a series of diagrams showing delivery of tandem Ring2 genomes to Sf9 cells via baculovirus.

FIG. 28 depicts expression of Ring2 ORF1 with a C-terminal His tag in insect cells.

FIG. 29 depicts expression of Ring1 ORF1 and ORF1/1 with a C-terminal His tag in insect cells.

FIG. 30 depicts expression of Ring2 ORF1 with an N-terminal His-tag, with or without PreScission cleavage sequence, in insect cells.

FIG. 31 depicts expression of Ring1 ORFs 1/1, 1/2, 2, 2/2, and 2/3 as C-terminal His-tagged recombinant proteins in insect cells.

FIG. 32 depicts expression of individual Ring2 ORFs in insect cells. Two exposures of the same blot are shown in the middle and right panels. The left panel shows the structures of Ring2 constructs tested as indicated.

FIG. 33 depicts baculovirus-mediated co-expression of Ring2 ORF1+“FullORF”, ORF1+ORF2, ORF1+ORF2/2, and ORF1+ORF2/3 in insect cells.

FIG. 34 depicts simultaneous co-expression of multiple Ring2 proteins in insect cells using baculovirus.

FIG. 35 depicts expression of ORFs from Anellovirus genome delivered into insect cells by baculovirus and by transfection.

FIG. 36 shows that expression of Ring1 ORF2 is independent of the polyhedron promoter (arrow labeled pH) in Sf9 cells.

FIG. 37 depicts co-delivery of Ring2 ORF1-His and Ring2 genomic DNA into Sf9 cells, followed by incubation and fractionation on a CsCl linear density gradient. An anti-His tag Western blot of fractions is shown at the top of the figure, as well as a qPCR assay of each fraction. Bottom panels show transmission electron microscopy images of two individual fractions and a pool of fractions, as indicated by boxes on the Western blot. The inset in the middle panel is a zoomed-in view showing proteasome-like structures.

FIG. 38 depicts characterization of Sf9 isopycnic fractions by immunogold electron microscopy.

FIG. 39 depicts expression of ORF1 from additional Anellovirus strains.

FIG. 40 depicts plots of the sequence read counts of Anelloviruses in subjects of Example 36. The total number of reads are presented for reads derived from donor samples and those derived from transfusion recipient samples. Bars in shades of blue represent total reads while bars in shades of red indicate reads identified as Anellovirus reads. Light blue bars=donor total reads; light red bars=donor Anellovirus reads; dark blue bars=recipient total reads; dark red bars=recipient Anellovirus reads.

FIG. 41 illustrates mapping of the extent of Anellovirus diversity. Panel A of FIG. 41 depicts the maximum-likelihood phylogeny of Anellovirus ORF1 amino acid sequences (n=1575). Tips are colored based on agglomerative clustering of pairwise amino acid distances to produce 10 arbitrary clusters. Grey branches connect previously published sequences to the root and black branches represent sequences reported in this study. Black dashes to the right of the tree indicate the positions and volume of new sequences. Panel B of FIG. 41 depicts multidimensional scaling (MDS) analysis of 1575 Anellovirus ORF1 amino acid sequences (points are colored as in Panel A) compared to eight other viral surface proteins: 2627 Human papillomavirus (HPV) L1, 86 Adeno-associated virus (AAV) capsid, 3000 Human immunodeficiency virus 1 (HIV1) env, 3000 Dengue virus envelope, 425 Middle East-associated respiratory syndrome coronavirus (MERS-CoV) Spike, 3000 Influenza A virus HA (group 2, subtypes H3, H4, H7, H10, and H14), 172 Ebolavirus (genus-wide) GP, 632 Lassa fever virus GPC protein sequences. MDS plots for all viruses are shown on the same scale; scale bar equals 0.2 amino acid substitutions per site in MDS projection space.

FIG. 42A is a schematic showing motif locations on an exemplary Anellovirus genome. Shown are the layouts of open reading frame (ORF) locations and their corresponding identified motifs on a theoretical Anellovirus genome.

FIG. 42B is a diagram showing conserved motifs in Anellovirus ORF3 sequences. A third open reading frame (ORF3) was predicted in addition to ORF1 and ORF2 near the 3′ end of 471 Anellovirus genomes in the TTVS dataset. Two novel and highly conserved motifs were identified near the 3′ end of ORF3: Motif 1 (a) was observed in 467 out of the 471 sequences (99%); Motif 2 (b) was observed in 463 out of the 471 sequences (98%).

FIG. 42C depicts plots of the percentage pairwise-identities across Anellovirus lineages. Sequences were binned into four groups (full contigs, ORF1 capsid proteins, ORF2, and 5′ UTR) to evaluate the similarities across each region.

FIG. 43 illustrates site diversity of the viral proteins. The plots in FIG. 43 depict the number of unique amino acids at each site in the viral protein sequence. Anellovirus ORF1 sequences belonging to Alphatorquevirus (yellow), Betatorquevirus (green), and Gammatorquevirus (red) are shown on the left, HIV-1 env, Influenza virus group 2 HA, and adeno-associated virus capsid sequences on the right for comparison. The number of unique amino acids in each of the viral protein alignments with a smoothed average (50 amino acid long window) is shown in black. Alignment columns comprised of at least 90% gaps were excluded. The first 150 amino acids of Anellovirus alignment are highlighted with a transparent grey box to indicate the approximate position of Anellovirus ORF1 sequences that significantly resemble circovirus capsid sequences according to HHpred.

FIG. 44 illustrates phylogenetic analysis of the 5′ untranslated region of Anellovirus sequences. The phylogenetic tree on the left shows the relationship between the three anellovirus genera (Alphatorquevirus in red, Betatorquevirus in blue, and Gammatorquevirus in purple) in the 5′ untranslated region. To the right of the phylogenetic tree is the 73-nucleotide alignment (adenine in red, cytidine in blue, thymidine in green, guanine in yellow, and gaps and ambiguous nucleotides in grey). A group of five Gammatorqueviruses (classified as such based on the entire genome) appear to be more closely related to Betatorqueviruses than to other Gammatorqueviruses.

FIGS. 45A-45C illustrates characterization of personal anellomes (i.e., the set of Anellovirus sequences and, in some instances, their relative frequency, present in a single subject, such as a human patient). Panel A of FIG. 45 provides results of pan-Anellovirus PCR testing. Fifteen transfusion recipients were paired with one or more blood donors and received a blood transfusion following surgery. Recipient samples were collected post-transfusion over a period of 280 days. Pan-Anellovirus PCR-positive samples are shown in red. Panel B of FIG. 45 depicts a plot of the number of unique Anellovirus lineages identified per individual. Panel C of FIG. 45 depicts Anellovirus diversity in each transfusion recipient. MDS analysis demonstrated Anellovirus diversity within study subjects that spans the space of overall known Anellovirus diversity. Convex hulls depict the amount of the diversity space encompassed in each subject set. Numbers presented above each facet indicate the fraction of area occupied by the convex hull of the patient's Anelloviruses compared to the area of the convex hull of all Anelloviruses sampled.

FIG. 46 depicts plots of the average amino acid identity (AAI) within subjects. Average amino acid identity was computed between Anellovirus lineages found in each transfusion-recipient subject. The dotted vertical line in each panel represents the mean AAI in each subject.

FIGS. 47A-47B illustrates the transmission of Anellovirus lineages via blood transfusion. FIG. 47A depicts stream graphs showing relative abundance of Anellovirus lineages in transfusion-recipients longitudinally following blood transfusion. Lineages colored in shades of red denote transmitted strains from the donor(s) while shades of blue indicate Anellovirus lineages endemic to the recipient. FIG. 47B depicts a comparison of pairwise-distances between different subsets of Anelloviruses isolated from transfusion subjects. Similarity of Anelloviruses between donors and those in the recipient prior to transfusion did not predict transmissibility.

FIG. 48 illustrates the impact of Anellovirus recombination on Anellovirus diversity. Panel A of FIG. 48 depicts plots of the tangled chain of midpoint-rooted phylogenies inferred from 500 nucleotide fragments of Anellovirus ORF1 with the position of each lineage in successive phylogenies shown with lines colored by their relative position in the first phylogeny. Unlinked evolution across the Anellovirus genome is evidence of recombination. Panel B of FIG. 48 presents evidence of recombination in Anelloviruses through homoplasies. Ancestral sequence reconstruction between sequences within 80% identity of each other at nucleotide level show numerous repeat mutations—each line connects identical mutations that occurred on different branches, with fractions along the length of the branch indicating the relative position of the mutation in the Anellovirus genome. Ticks on branches indicate mutations that occurred uniquely on the branch in question. Branches are colored by the fraction of all mutations that were homoplasies with highest values (all mutations are homoplasies/no unique mutations) highlighted in white. Panel C of FIG. 48 depicts plots of linkage disequilibrium (measured as χ²_{df}, which is equivalent to r² but also applies to sites with more than two alleles) decay as a function of physical distance between polymorphic sites. Each dot corresponds to a pair of polymorphic ORF1 sites where both sites were more than 10% non-gap and non-ambiguous characters. Red line indicates the local LD average in 100 nt-long windows.

FIG. 49 illustrates the long recombination tracts identified in the non-coding genomic regions of Alphatorqueviruses. Recombination tracts depicted comprise at least three mutations within a 10-nucleotides span that occurring at least twice in the phylogenetic tree. Each putative recombination tract is outlined in black. The nucleotide state of all sequences descended from a branch with a putative recombination tract are shown as colored boxes (adenine in red, cytidine in blue, thymidine in green, guanine in yellow) with nucleotide indicated. Identical tracts are connected with grey boxes to highlight similarities.

FIG. 50 illustrates the phylogenetic positions of the recombination tracts identified among the non-coding genomic regions of Alphatorqueviruses. Recombination tracts span the entirety of Alphatorquevirus diversity and suggest that minimal barriers exist to genetic material exchange even between distantly related genomes.

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

### DETAILED DESCRIPTION OF THE INVENTION

### Definitions

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

The term “amplification,” as used herein, refers to replication of a nucleic acid molecule or a portion thereof, to produce one or more additional copies of the nucleic acid molecule or a portion thereof (e.g., a genetic element or a genetic element region). In some embodiments, amplification results in partial replication of a nucleic acid sequence. In some embodiments, amplification occurs via rolling circle replication.

As used herein, the term “anellovector” refers to a vehicle comprising a genetic element, e.g., a circular DNA, enclosed in a proteinaceous exterior, e.g, the genetic element is substantially protected from digestion with DNAse I by a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the anellovector is purified, e.g., it is separated from its original source and/or substantially free (>50%, >60%, >70%, >80%, >90%) of other components. In some embodiments, the anellovector is capable of introducing the genetic element into a target cell (e.g., via infection). In some embodiments, the anellovector is an infective synthetic Anellovirus viral particle.

As used herein, the term “Anellovirus sequence” refers to a sequence of a naturally occurring Anellovirus or fragment thereof. The term includes Anellovirus sequences that have been identified as of the filing date as well as other Anellovirus sequences that have not yet been identified or sequenced. In some instances, the term “Anellovirus sequence,” as used herein with respect to nucleic acid sequences, refers to a nucleic acid molecule comprising a nucleic acid sequence of at least about 100, 200, 300, 400, 500, 600, 700, 800, 900, 1000, 1200, 1400, 1600, 1800, 2000, 2500, 3000, 3500 or 4000 nucleotides, wherein the nucleic acid sequence has at least about 50%, 60%, 70%, 80%, 85%, 90%, 95%, 96%, 97%, 98%, 99%, or 100% sequence identity with a contiguous sequence of the same length comprised in a known Anellovirus genome, e.g., as described herein. An Anellovirus sequence may comprise, in some instances, a complete viral (e.g., Anellovirus) genome sequence. In other instances, an Anellovirus sequence may comprise a partial viral (e.g., Anellovirus) genome sequence. In some instances, an Anellovirus sequence comprises the nucleic acid sequence of one or more of a TATA box, cap site, initiator element, transcriptional start site, 5′ UTR conserved domain, ORF1-encoding sequence, ORF1/1-encoding sequence, ORF1/2-encoding sequence, ORF2-encoding sequence, ORF2/2-encoding sequence, ORF2/3-encoding sequence, ORF2t/3-encoding sequence, three open-reading frame region, poly(A) signal, GC-rich region, or any combination thereof, of a naturally-occurring (e.g., wild-type) Anellovirus (e.g., an Anellovirus having a sequence as annotated, or as encoded by, a sequence listed in any of the Tables provided herein). In some instances, an Anellovirus sequence comprises at least one difference (e.g., a point mutation, substitution, deletion, insertion, or modification relative thereto) from a known Anellovirus genome, e.g., as described herein.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

As used herein, the term “complementary” when used to describe a first nucleotide sequence in relation to a second nucleotide sequence, refers to the ability of the first and second nucleotide sequences to hybridize and form a duplex structure through matching base pairs under specified conditions. Such conditions can be, for example, stringent hybridization conditions such as in 1×phi29 DNA polymerase buffer (NEB). Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides. Two sequences that are complementary may be perfectly complementary (100% matched base pairs) or may contain one or more mismatches (e.g., 1, 2, 3, 4, 5 mismatches, or up to about 1%, 2%, or 5% mismatches).

As used herein, a nucleic acid “encoding” refers to a nucleic acid sequence encoding an amino acid sequence or a polynucleotide, e.g., an mRNA or functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid molecule that is or can be enclosed within (e.g., protected from DNAse I digestion by) a proteinaceous exterior, e.g., to form an anellovector as described herein. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

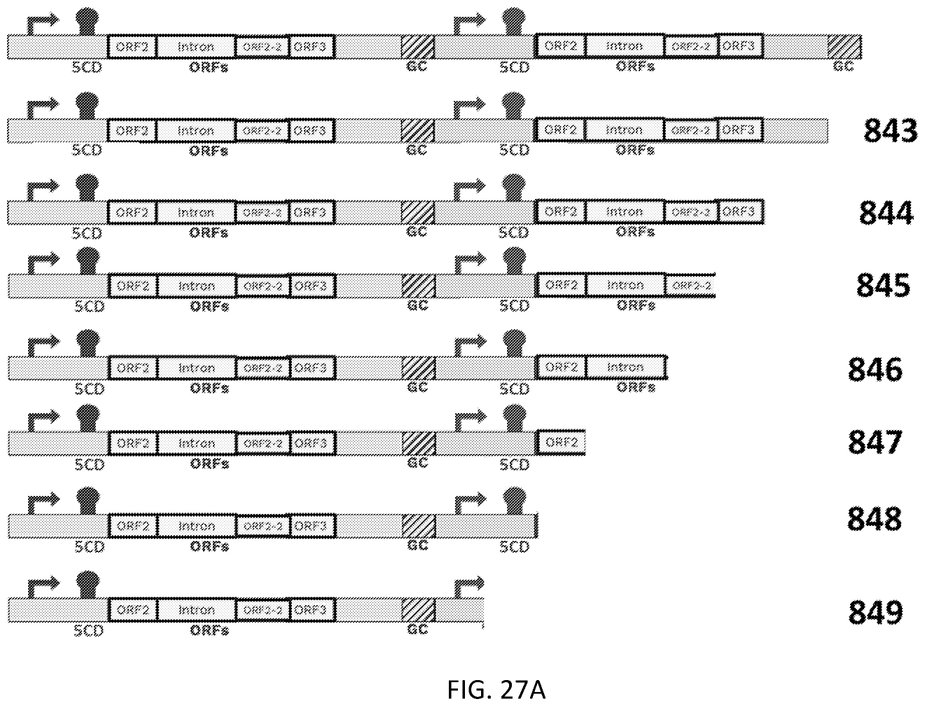

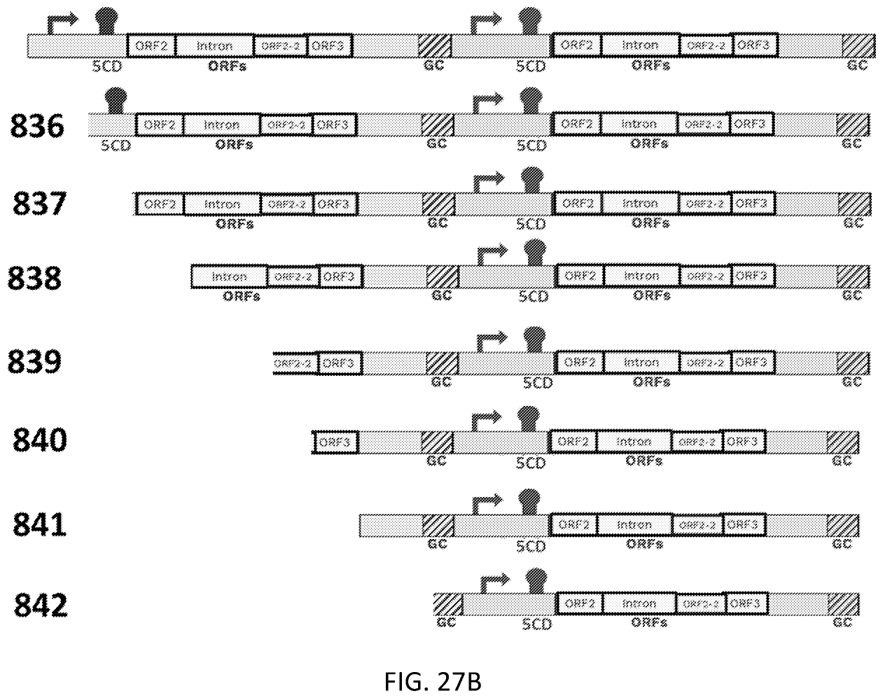

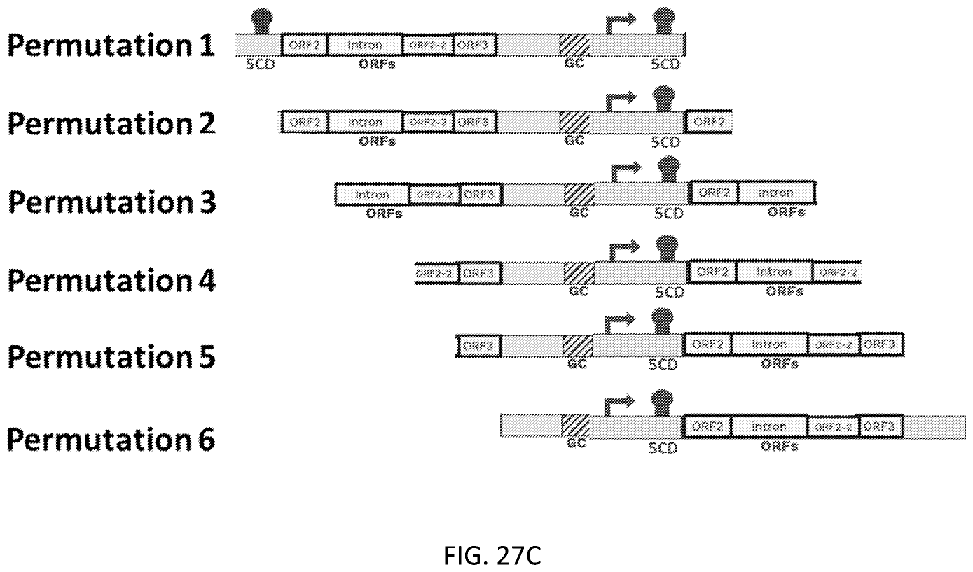

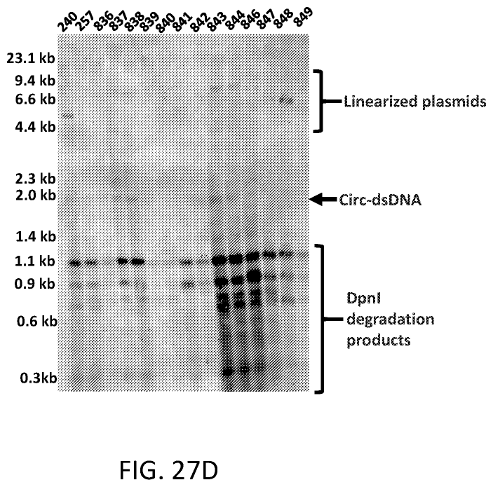

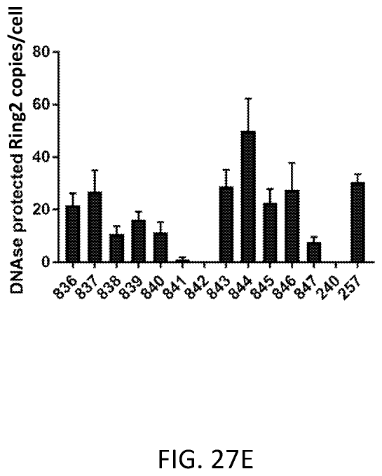

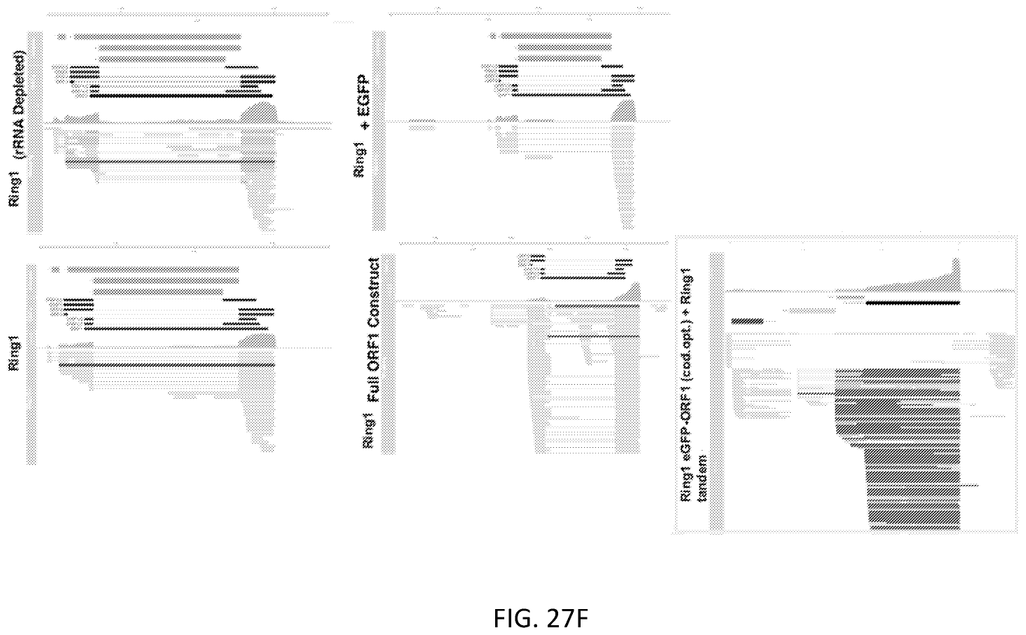

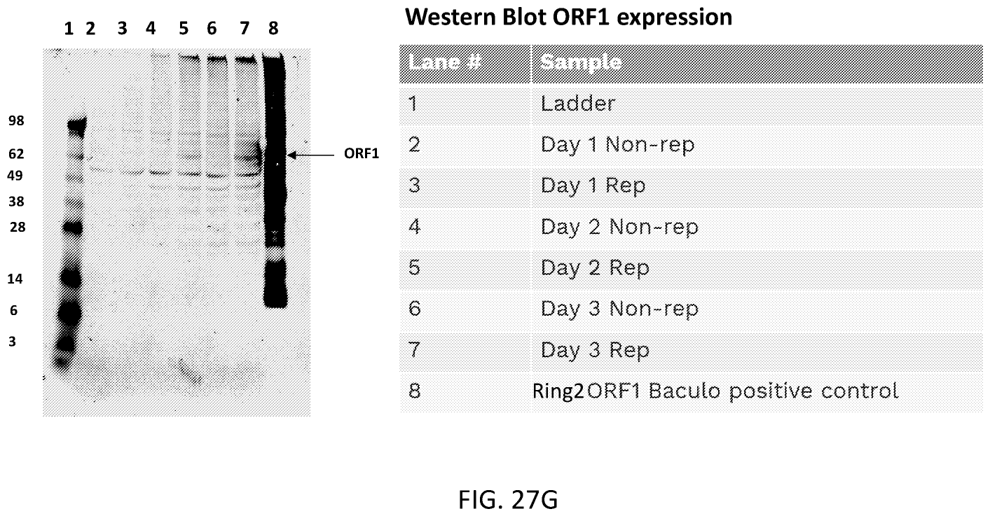

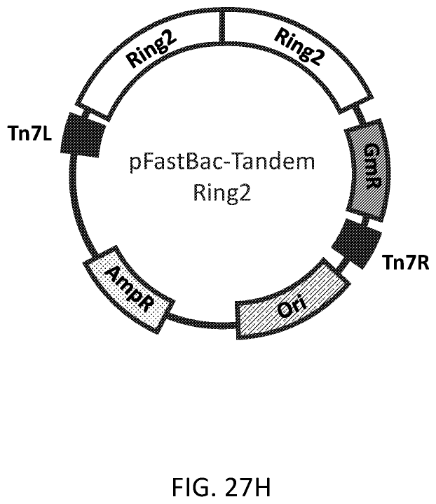

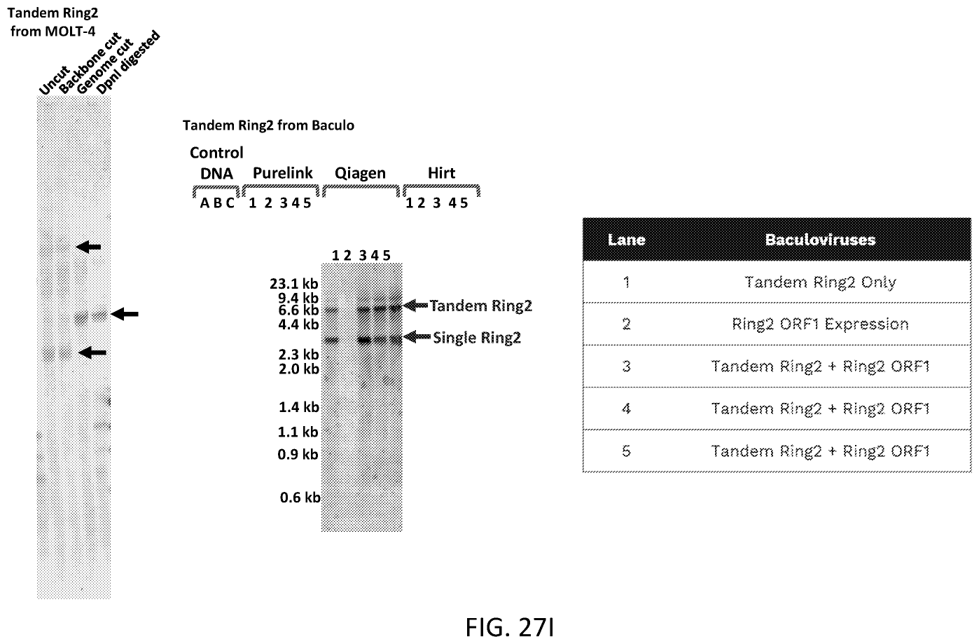

As used herein, “genetic element construct” refers to a nucleic acid construct (e.g., a plasmid, bacmid, cosmid, or minicircle) comprising at least one (e.g., two) genetic element sequence(s), or fragment thereof. In some embodiments, a genetic element construct comprises at least one full length genetic element sequence. In some embodiments, a genetic element comprises a full length genetic element sequence and a partial genetic element sequence. In some embodiments, a genetic element comprises two or more partial genetic element sequences (e.g., in 5′ to 3′ order, a 5′-truncated genetic element sequence arranged in tandem with a 3′-truncated genetic element sequence, e.g., as shown in FIG. 27C).

The term “genetic element region,” as used herein, refers to a region of a construct that comprises the sequence of a genetic element. In some embodiments, the genetic element region comprises a sequence having sufficient identity to a wild-type Anellovirus sequence, or a fragment thereof, to be enclosed by a proteinaceous exterior, thereby forming an anellovector (e.g., a sequence having at least 70%, 75%, 80%, 85%, 90%, 95%, 96%, 97%, 98%, 99%, or 100% sequence identity to the wild-type Anellovirus sequence or fragment thereof). In embodiments, the genetic element region comprises a protein binding sequence, e.g., as described herein (e.g., a 5′ UTR, 3′ UTR, and/or a GC-rich region as described herein, or a sequence having at least 70%, 75%, 80%, 85%, 90%, 95%, 96%, 97%, 98%, 99%, or 100% sequence identity thereto). In some embodiments, the genetic element region can undergo rolling circle replication. In some embodiments, the genetic element comprises a Rep protein binding site. In some embodiments, the genetic element comprises a Rep protein displacement site. In some embodiments, the construct comprising a genetic element region is not enclosed in a proteinaceous exterior, but a genetic element produced from the construct can be enclosed in a proteinaceous exterior. In some embodiments, the construct comprising the genetic element region further comprises a vector backbone.

As used herein, the term “mutant” when used with respect to a genome (e.g., an Anellovirus genome), or a fragment thereof, refers to a sequence having at least one change relative to a corresponding wild-type Anellovirus sequence. In some embodiments, the mutant genome or fragment thereof comprises at least one single nucleotide polymorphism, addition, deletion, or frameshift relative to the corresponding wild-type Anellovirus sequence. In some embodiments, the mutant genome or fragment thereof comprises a deletion of at least one Anellovirus ORF (e.g., one or more of ORF1, ORF2, ORF2/2, ORF2/3, ORF1/1, and/or ORF1/2) relative to the corresponding wild-type Anellovirus sequence. In some embodiments, the mutant genome or fragment thereof comprises a deletion of all Anellovirus ORFs (e.g., all of ORF1, ORF2, ORF2/2, ORF2/3, ORF1/1, and ORF1/2) relative to the corresponding wild-type Anellovirus sequence. In some embodiments, the mutant genome or fragment thereof comprises a deletion of at least one Anellovirus noncoding region (e.g., one or more of a 5′ UTR, 3′ UTR, and/or GC-rich region) relative to the corresponding wild-type Anellovirus sequence. In some embodiments, the mutant genome or fragment thereof comprises or encodes an exogenous effector.

“ORF molecule” refers to a polypeptide having an activity and/or a structural feature of an Anellovirus ORF protein (e.g., an Anellovirus ORF1, ORF2, ORF2/2, ORF2/3, ORF1/1, and/or ORF1/2 protein), or a functional fragment thereof. When used generically (i.e., “ORF molecule”), the polypeptide may comprise an activity and/or structural feature of any of the Anellovirus ORFs described herein (e.g., an Anellovirus ORF1, ORF2, ORF2/2, ORF2/3, ORF1/1, and/or ORF1/2), or a functional fragment thereof. When used with a modifier to indicate a particular open reading frame (e.g., “ORF1 molecule,” “ORF2 molecule,” “ORF2/2 molecule,” “ORF2/3 molecule,” “ORF1/1 molecule,” or “ORF1/2 molecule”), it is generally meant that the polypeptide comprises an activity and/or structural feature of the corresponding Anellovirus ORF protein, or a functional fragment thereof (for example, as defined below for “ORF1 molecule”). For example, an “ORF2 molecule” comprises an activity and/or structural feature of an Anellovirus ORF2 protein, or a functional fragment thereof.

As used herein, the term “ORF1 molecule” refers to a polypeptide having an activity and/or a structural feature of an Anellovirus ORF1 protein (e.g., an Anellovirus ORF1 protein as described herein, or a functional fragment thereof. An ORF1 molecule may, in some instances, comprise one or more of (e.g., 1, 2, 3 or 4 of): a first region comprising at least 60% basic residues (e.g., at least 60% arginine residues), a second region comprising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid. An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein).

As used herein, the term “ORF2 molecule” refers to a polypeptide having an activity and/or a structural feature of an Anellovirus ORF2 protein (e.g., an Anellovirus ORF2 protein as described herein, or a functional fragment thereof. An “Anellovirus ORF2 protein,” as used herein, refers to an ORF2 protein encoded by an Anellovirus genome (e.g., a wild-type Anellovirus genome, e.g., as described herein).

As used herein, the term “primer” refers to a nucleic acid sequence that can bind to a template nucleic acid and allow for polymerization of a complementary strand in the presence of appropriate enzymes and buffer conditions. In some embodiments, a primer comprises DNA. In some embodiments, a primer has a length of between 8 and 15 nucleotides, e.g., between 9 and 13 nucleotides, e.g., more than 4 but less than 30, 25, 20, 15, or 10 nucleotides.

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “Rep” or “replication protein” refers to a protein, e.g., a viral protein, that promotes viral genome replication. In some embodiments, the replication protein is an Anellovirus Rep protein.

As used herein, the term “Rep binding site” refers to a nucleic acid sequence within a nucleic acid molecule that is recognized and bound by a Rep protein (e.g., an Anellovirus Rep protein). In some embodiments, a Rep binding site comprises a 5′ UTR (e.g., comprising a hairpin loop). In some embodiments, a Rep binding site comprises an origin of replication (ORI).

As used herein, the term “Rep displacement site” refers to a nucleic acid sequence within a nucleic acid molecule that is capable of causing a Rep protein (e.g., an Anellovirus Rep protein) associated with (e.g., bound to) the nucleic acid molecule to release the nucleic acid molecule upon reaching the Rep displacement site. In some embodiments, a Rep displacement site comprises a 5′ UTR (e.g., comprising a hairpin loop). In some embodiments, a Rep displacement site comprises an origin of replication (ORI).

As used herein, a “substantially non-pathogenic” organism, particle, or component, refers to an organism, particle (e.g., a virus or an anellovector, e.g., as described herein), or component thereof that does not cause or induce unacceptable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce unacceptable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization. In some embodiments, integration frequency is determined by quantitative gel purification assay of genomic DNA separated from free vector, e.g., as described in Wang et al. (2004, Gene Therapy 11: 711-721, incorporated herein by reference in its entirety).

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In embodiments, the substantially non-immunogenic organism, particle, or component does not produce a clinically significant immune response. In embodiments, the substantially non-immunogenic anellovector does not produce a clinically significant immune response against a protein comprising an amino acid sequence or encoded by a nucleic acid sequence of an Anellovirus or anellovector genetic element. In embodiments, an immune response (e.g., an undesired or untargeted immune response) is detected by assaying antibody (e.g., neutralizing antibody) presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies (e.g., neutralizing antibody) against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subsequence comprising an exogenous sequence or a sequence heterologous to the remainder of the larger sequence, e.g., a corresponding subsequence from a different Anellovirus).

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

This invention relates generally to methods of administration of anellovectors, and uses thereof. The present disclosure provides anellovectors, compositions comprising anellovectors, and methods of making or using anellovectors. Anellovectors are generally useful as delivery vehicles, e.g., for delivering a therapeutic agent to a eukaryotic cell. Generally, an anellovector will include a genetic element comprising a nucleic acid sequence (e.g., encoding an effector, e.g., an exogenous effector or an endogenous effector) enclosed within a proteinaceous exterior. An anellovector may include one or more deletions of sequences (e.g., regions or domains as described herein) relative to an Anellovirus sequence (e.g., as described herein). Anellovectors can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells. This invention further relates generally to methods of amplifying nucleic acid molecules comprising Anellovirus sequences, methods of sequencing such amplified nucleic acid molecules, methods of analyzing sequence data obtained for such amplified nucleic acid molecules, and compositions for use in such methods. The Anellovirus sequences determined using methods described herein can, in some instances, be used to produce anellovectors, e.g., synthetic anellovectors, e.g., be included in a genetic element of an anellovector as described herein.

### Table of Contents

### I. Compositions and Methods for Making Anellovectors

A. Components and Assembly of Anellovectors
i. ORF1 molecules for assembly of anellovectors
ii. ORF2 molecules for assembly of anellovectors

B. Genetic Element Constructs
i. Plasmids
ii. Circular nucleic acid constructs
iii. In vitro circularization
iv. Cis/trans constructs
v. Expression cassettes
vi. Design and production of a genetic element construct

C. Effectors

D. Host Cells
i. Introduction of genetic elements into host cells
ii. Methods for providing Anellovirus protein(s) in cis or trans
iii. Helpers
iv. Exemplary cell types

E. Culture Conditions

F. Harvest

### I. Compositions and Methods for Making Anellovectors

A. Components and Assembly of Anellovectors
i. ORF1 molecules for assembly of anellovectors
ii. ORF2 molecules for assembly of anellovectors

B. Genetic Element Constructs
i. Plasmids
ii. Circular nucleic acid constructs
iii. In vitro circularization
iv. Cis/trans constructs
v. Expression cassettes
vi. Design and production of a genetic element construct

C. Effectors

D. Host Cells
i. Introduction of genetic elements into host cells
ii. Methods for providing Anellovirus protein(s) in cis or trans
iii. Helpers
iv. Exemplary cell types

E. Culture Conditions

F. Harvest

G. Enrichment and Purification

### II. Anellovectors

A. Anelloviruses

B. ORF1 molecules

C. ORF2 molecules

D. Genetic elements

E. Protein binding sequences

F. 5′ UTR Regions

G. GC-rich regions

H. Effectors

I. Regulatory Sequences

J. Replication Proteins

K. Other Sequences

L. Proteinaceous exterior

### III. Nucleic Acid Constructs

### IV. Compositions

V. Host cells

### VI. Methods of Use

### VII. Administration/Delivery

VIII. Methods of amplifying anellovirus sequences

A. DNA amplification
a. Rolling circle amplification
b. Primers
c. Samples and target sequences

B. Sequencing

C. Computational Analysis

### I. Compositions and Methods for Making Anellovectors

The present disclosure provides, in some aspects, anellovectors and methods thereof for delivering effectors. In some embodiments, the anellovectors or components thereof can be made as described below. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell. In some embodiments, the anellovectors or components thereof can be made using a tandem construct, e.g., as described in U.S. Provisional Application 63/038,483, which is incorporated herein by reference in its entirety. In some embodiments, the anellovectors or components thereof can be made using a bacmid/insect cell system, e.g., as described in U.S. Provisional Application No. 63/038,603, which is incorporated herein by reference in its entirety.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an anellovector.

Components and Assembly of Anellovectors

The compositions and methods herein can be used to produce anellovectors. As described herein, an anellovector generally comprises a genetic element (e.g., a single-stranded, circular DNA molecule, e.g., comprising a 5′ UTR region as described herein) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, the genetic element is enclosed within the proteinaceous exterior in a host cell (e.g., as described herein). In some embodiments, the host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, the host cell comprises the genetic element and/or a nucleic acid construct comprising the sequence of the genetic element. In some embodiments, the nucleic acid construct is selected from a plasmid, viral nucleic acid, minicircle, bacmid, or artificial chromosome. In some embodiments, the genetic element is excised from the nucleic acid construct and, optionally, converted from a double-stranded form to a single-stranded form (e.g., by denaturation). In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein. In other embodiments, the nucleic acid construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

ORF1 Molecules, e.g., for Assembly of Anellovectors

An anellovector can be made, for example, by enclosing a genetic element within a proteinaceous exterior. The proteinaceous exterior of an Anellovector generally comprises a polypeptide encoded by an Anellovirus ORF1 nucleic acid (e.g., an Anellovirus ORF1 molecule or a splice variant or functional fragment thereof, e.g., as described herein). An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 arginine-rich region, jelly-roll region, N22 domain, hypervariable region, and/or C-terminal domain. In some embodiments, the proteinaceous exterior comprises an Anellovirus ORF1 jelly-roll region (e.g., as described herein). In some embodiments, the proteinaceous exterior comprises an Anellovirus ORF1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein, e.g., produced using a composition or construct as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer.

In some embodiments, a first plurality of anellovectors comprising an ORF1 molecule as described herein is administered to a subject. In some embodiments, a second plurality of anellovectors comprising an ORF1 molecule described herein, is subsequently administered to the subject following administration of the first plurality. In some embodiments, the second plurality of anellovectors comprises an ORF1 molecule having the same amino acid sequence as the ORF1 molecule comprised by the anellovectors of the first plurality. In some embodiments, the second plurality of anellovectors comprises an ORF1 molecule having at least 70%, 75%, 80%, 85%, 90%, 95%, 96%, 97%, 98%, 99%, or 100% amino acid sequence identity to the ORF1 molecule comprised by the anellovectors of the first plurality.

ORF2 Molecules, e.g., for Assembly of Anellovectors

Producing an anellovector using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector does not comprise an ORF2 molecule, or a splice variant or functional fragment thereof, and/or a nucleic acid encoding an ORF2 molecule, or a splice variant or functional fragment thereof. In some embodiments, producing the anellovector comprises expression of an ORF2 molecule, or a splice variant or functional fragment thereof, but the ORF2 molecule is not incorporated into the anellovector.

Genetic Element Constructs, e.g., for Assembly of Anellovectors

The genetic element of an anellovector as described herein may be produced from a genetic element construct that comprises a genetic element region and optionally other sequence such as vector backbone. Generally, the genetic element construct comprises an Anellovirus 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the anellovector as described herein (e.g., in a cell culture system), which may comprise one or more of the following steps: (a) introducing (e.g., transfecting) a genetic element (e.g., linearized) into a cell line sensitive to anellovector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization (IVC), e.g., as described in Example 15.

Generally, in vitro circularized DNA constructs can be produced by digesting a genetic element construct (e.g., a plasmid comprising the sequence of a genetic element) to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

Cis/Trans Constructs

In some embodiments, a genetic element construct as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell).

In some embodiments, the genetic element construct comprises a sequence encoding an Anellovirus ORF1 molecule, or a splice variant or functional fragment thereof (e.g., a jelly-roll region, e.g., as described herein). In some embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In some embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Effectors

The compositions and methods described herein can be used to produce a genetic element of an anellovector comprising a sequence encoding an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

Host Cells

The anellovectors described herein can be produced, for example, in a host cell. Generally, a host cell is provided that comprises an anellovector genetic element and the components of an anellovector proteinaceous exterior (e.g., a polypeptide encoded by an Anellovirus ORF1 nucleic acid or an Anellovirus ORF1 molecule). The host cell is then incubated under conditions suitable for enclosure of the genetic element within the proteinaceous exterior (e.g., culture conditions as described herein). In some embodiments, the host cell is further incubated under conditions suitable for release of the anellovector from the host cell, e.g., into the surrounding supernatant. In some embodiments, the host cell is lysed for harvest of anellovectors from the cell lysate. In some embodiments, an anellovector may be introduced to a host cell line grown to a high cell density.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an anellovector comprising the genetic element

In some embodiments, the genetic element construct is capable of replication once introduced into the host cell. In some embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the anellovector. To this end, cell lines that express an anellovector polymerase protein may be utilized as appropriate host cells. Host cells may be similarly engineered to provide other viral functions or additional functions.

To prepare the anellovector disclosed herein, a genetic element construct may be used to transfect cells that provide anellovector proteins and functions required for replication and production. Alternatively, cells may be transfected with a second construct (e.g., a virus) providing anellovector proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the anellovector. In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into anellovectors using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

Methods for Providing Anellovirus Protein(s) in Cis or Trans

In some embodiments (e.g., cis embodiments described herein), the genetic element construct further comprises one or more expression cassettes comprising a coding sequence for an Anellovirus ORF (e.g., an Anellovirus ORF1, ORF2, ORF2/2, ORF2/3, ORF1/1, or ORF1/2, or a functional fragment thereof). In some embodiments, the genetic element construct comprises an expression cassette comprising a coding sequence for an Anellovirus ORF1, or a splice variant or functional fragment thereof. Such genetic element constructs, which comprise expression cassettes for the effector as well as the one or more Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more Anellovirus ORFs (e.g., an Anellovirus ORF1, ORF2, ORF2/2, ORF2/3, ORF1/1, or ORF1/2, or a functional fragment thereof). In some embodiments, the genetic element construct does not comprise an expression cassette comprising a coding sequence for an Anellovirus ORF1, or a splice variant or functional fragment thereof. Such genetic element constructs, which comprise expression cassettes for the effector but lack expression cassettes for one or more Anellovirus ORFs (e.g., Anellovirus ORF1 or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells comprising such genetic element constructs are incapable of enclosure of the genetic elements within proteinaceous exteriors in the absence of an additional nucleic construct encoding an Anellovirus ORF1 molecule. In other words, such genetic element constructs may be used for trans anellovector production methods in host cells, e.g., as described herein.

Helpers

In some embodiments, a helper construct is introduced into a host cell (e.g., a host cell comprising a genetic element construct or a genetic element as described herein). In some embodiments, the helper construct is introduced into the host cell prior to introduction of the genetic element construct. In some embodiments, the helper construct is introduced into the host cell concurrently with the introduction of the genetic element construct. In some embodiments, the helper construct is introduced into the host cell after introduction of the genetic element construct.

Exemplary Cell Types

Exemplary host cells suitable for production of anellovectors include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell, a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell. In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In an aspect, the present disclosure provides a method of manufacturing an anellovector comprising a genetic element enclosed in a proteinaceous exterior, the method comprising providing a MOLT-4 cell comprising an anellovector genetic element, and incubating the MOLT-4 cell under conditions that allow the anellovector genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the anellovector genetic element is produced in the MOLT-4 cell, e.g., from a genetic element construct (e.g., as described herein). In some embodiments, the method further comprises introducing the anellovector genetic element construct into the MOLT-4 cell.

In an aspect, the present disclosure provides a method of manufacturing an anellovector comprising a genetic element enclosed in a proteinaceous exterior, the method comprising providing a MOLT-3 cell comprising an anellovector genetic element, and incubating the MOLT-3 cell under conditions that allow the anellovector genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the anellovector genetic element is produced in the MOLT-3 cell, e.g., from a genetic element construct (e.g., as described herein). In some embodiments, the method further comprises introducing the anellovector genetic element construct into the MOLT-3 cell.

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the anellovector is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

Culture Conditions

Host cells comprising a genetic element and components of a proteinaceous exterior can be incubated under conditions suitable for enclosure of the genetic element within the proteinaceous exterior, thereby producing an anellovector. Suitable culture conditions include those described, e.g., in any of Examples 4, 5, 7, 8, 9, 10, 11, or 15. In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release anellovectors produced therein into the surrounding supernatant.

The production of anellovector-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

Harvest

Anellovectors produced by host cells can be harvested, e.g., according to methods known in the art. For example, anellovectors released into the surrounding supernatant by host cells in culture can be harvested from the supernatant (e.g., as described in Example 4). In some embodiments, the supernatant is separated from the host cells to obtain the anellovectors. In some embodiments, the host cells are lysed before or during harvest. In some embodiments, the anellovectors are harvested from the host cell lysates (e.g., as described in Example 10). In some embodiments, the anellovectors are harvested from both the host cell lysates and the supernatant. In some embodiments, the purification and isolation of anellovectors is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the anellovector may be harvested and/or purified by separation of solutes based on biophysical properties, e.g., ion exchange chromatography or tangential flow filtration, prior to formulation with a pharmaceutical excipient.

Enrichment and Purification

Harvested anellovectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested anellovectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### II. Anellovectors

In some aspects, the invention described herein comprises compositions and methods of using and making an anellovector, anellovector preparations, and therapeutic compositions. In some embodiments, the anellovectors are made using compositions and methods as described herein. In some embodiments, the anellovector comprises one or more nucleic acids or polypeptides comprising a sequence, structure, and/or function that is based on an Anellovirus (e.g., an Anellovirus as described herein), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety. In some embodiments the anellovector comprises an anellovector, or a component thereof (e.g., a genetic element, e.g., comprising a sequence encoding an effector, and/or a proteinaceous exterior), e.g., as described in PCT Application No. PCT/US19/65995, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as described herein); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:

a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and

b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In embodiments, the promoter element comprises a TATA box. In embodiments, the promoter element is endogenous to a wild-type Anellovirus, e.g., as described herein.

In some embodiments, the genetic element comprises one or more of the following characteristics: single-stranded, circular, negative strand, and/or DNA. In embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The anellovectors, compositions comprising anellovectors, methods using such anellovectors, etc., as described herein are, in some instances, based in part on the examples which illustrate how different effectors, for example miRNAs (e.g. against IFN or miR-625), shRNA, etc and protein binding sequences, for example, DNA sequences that bind to capsid protein such as Q99153, are combined with proteinaceous exteriors, for example a capsid disclosed in Arch Virol (2007) 152: 1961-1975, to produce anellovectors which can then be used to deliver an effector to cells (e.g., animal cells, e.g., human cells or non-human animal cells such as pig or mouse cells). In some embodiments, the effector can silence expression of a factor such as an interferon. The examples further describe how anellovectors can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specific Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In some embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, introduction of the anellovector, or genetic element comprised therein, decreases level of interferon produced by the cell. In some embodiments, introduction of the anellovector, or genetic element comprised therein, into a cell modulates (e.g., increases or decreases) a function of the cell. In some embodiments, introduction of the anellovector, or genetic element comprised therein, into a cell modulates (e.g., increases or decreases) the viability of the cell. In some embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

Anelloviruses

In some embodiments, an anellovector, e.g., as described herein, comprises sequences or expression products derived from an Anellovirus. In some embodiments, an anellovector includes one or more sequences or expression products that are exogenous relative to the Anellovirus. In some embodiments, an anellovector includes one or more sequences or expression products that are endogenous relative to the Anellovirus. In some embodiments, an anellovector includes one or more sequences or expression products that are heterologous relative to one or more other sequences or expression products in the anellovector. Anelloviruses generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234).

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of an Anellovirus, e.g., as described herein. In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein. In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (or a splice variant or functional fragment thereof) or a polypeptide encoded by an Anellovirus ORF1 nucleic acid.

In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table A1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table A1.

In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table B1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table B1.

In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleic acid sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TAIP nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus initiator element nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table C1. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table C1.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the anellovector comprises a nucleic acid sequence selected from a sequence as shown in any of Tables A1-M2, or a sequence having at least 70%, 75%, 80%, 85%, 90%, 95%, 96%, 97%, 98%, 99%, or 100% sequence identity thereto. In embodiments, the anellovector comprises a polypeptide comprising a sequence as shown in any of Tables Tables A2-M2, or a sequence having at least 70%, 75%, 80%, 85%, 90%, 95%, 96%, 97%, 98%, 99%, or 100% sequence identity thereto.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any of Tables A-M). In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any of Tables A-M). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any of Tables A2-M2, or an ORF1 or ORF2 amino acid sequence encoded by a nucleic acid sequence as shown in any of Tables A1-M1). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (e.g., an ORF1 amino acid sequence as shown in any of Tables A2-M2, or an ORF1 amino acid sequence encoded by a nucleic acid sequence as shown in any of Tables A1-M1).

In some embodiments, an anellovector as described herein is a chimeric anellovector. In some embodiments, a chimeric anellovector further comprises one or more elements, polypeptides, or nucleic acids from a virus other than an Anellovirus.

In some embodiments, the chimeric anellovector comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein). For example, a chimeric anellovector may comprise an ORF1 molecule from one Anellovirus (e.g., a Ring1 ORF1 molecule, or an ORF1 molecule having at least 75%, 80%, 85%, 90%, 95%, 96%, 97%, 98%, or 99% amino acid sequence identity thereto) and an ORF2 molecule from a different Anellovirus (e.g., a Ring2 ORF2 molecule, or an ORF2 molecule having at least 75%, 80%, 85%, 90%, 95%, 96%, 97%, 98%, or 99% amino acid sequence identity thereto). In another example, a chimeric anellovector may comprise a first ORF1 molecule from one Anellovirus (e.g., a Ring1 ORF1 molecule, or an ORF1 molecule having at least 75%, 80%, 85%, 90%, 95%, 96%, 97%, 98%, or 99% amino acid sequence identity thereto) and a second ORF1 molecule from a different Anellovirus (e.g., a Ring2 ORF1 molecule, or an ORF1 molecule having at least 75%, 80%, 85%, 90%, 95%, 96%, 97%, 98%, or 99% amino acid sequence identity thereto).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In some embodiments, the anellovector comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In some embodiments, the chimeric ORF1 molecule comprises an ORF1 jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto.

In some embodiments, the anellovector comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In some embodiments, the anellovector comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In some embodiments, the anellovector comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In some embodiments, the anellovector comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In some embodiments, the anellovector comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In some embodiments, the anellovector comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

Additional exemplary Anellovirus genomes, for which sequences or subsequences comprised therein can be utilized in the compositions and methods described herein (e.g., to form a genetic element of an anellovector, e.g., as described herein) are described, for example, in PCT Application Nos. PCT/US2018/037379 and PCT/US19/65995 (incorporated herein by reference in their entirety). In some embodiments, the exemplary Anellovirus sequences comprise a nucleic acid sequence as listed in any of Tables A1, A3, A5, A7, A9, A11, B1-B5, 1, 3, 5, 7, 9, 11, 13, 15, or 17 of PCT/US19/65995, incorporated herein by reference. In some embodiments, the exemplary Anellovirus sequences comprise an amino acid sequence as listed in any of Tables A2, A4, A6, A8, A10, A12, C1-C5, 2, 4, 6, 8, 10, 12, 14, 16, or 18 of PCT/US19/65995, incorporated herein by reference. In some embodiments, the exemplary Anellovirus sequences comprise an ORF1 molecule sequence, or a nucleic acid sequence encoding same, e.g., as listed in any of Tables 21, 23, 25, 27, 29, 31, 33, 35, D2, D4, D6, D8, D10, or 37A-37C of PCT/US19/65995, incorporated herein by reference.

TABLE Al
Exemplary Anellovirus nucleic acid sequence {Alphatorquevirus, Clade 3)

Name Ring1
Genus/Clade Alphatorquevirus, Clade 3
Accession Number AJ620231.1
Full Sequence: 3753 bp
1 10 20 30 40 50
| | | | | |
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
TTTGCGTGCCAGCAGCTAAGAAAAAACCAACTGCTATGAGCTTCTGGAAA
CCTCCGGTACACAATGTCACGGGGATCCAACGCATGTGGTATGAGTCCTT
TCACCGTGGCCACGCTTCTTTTTGTGGTTGTGGGAATCCTATACTTCACA
TTACTGCACTTGCTGAAACATATGGCCATCCAACAGGCCCGAGACCTTCT
GGGCCACCGGGAGTAGACCCCAACCCCCACATCCGTAGAGCCAGGCCTGC
CCCGGCCGCTCCGGAGCCCTCACAGGTTGATTCGAGACCAGCCCTGACAT
GGCATGGGGATGGTGGAAGCGACGGAGGCGCTGGTGGTTCCGGAAGCGGT
GGACCCGTGGCAGACTTCGCAGACGATGGCCTCGATCAGCTCGTCGCCGC
CCTAGACGACGAAGAGTAAGGAGGCGCAGACGGTGGAGGAGGGGGAGACG
AAAAACAAGGACTTACAGACGCAGGAGACGCTTTAGACGCAGGGGACGAA
AAGCAAAACTTATAATAAAACTGTGGCAACCTGCAGTAATTAAAAGATGC
AGAATAAAGGGATACATACCACTGATTATAAGTGGGAACGGTACCTTTGC
CACAAACTTTACCAGTCACATAAATGACAGAATAATGAAAGGCCCCTTCG
GGGGAGGACACAGCACTATGAGGTTCAGCCTCTACATTTTGTTTGAGGAG
CACCTCAGACACATGAACTTCTGGACCAGAAGCAACGATAACCTAGAGCT
AACCAGATACTTGGGGGCTTCAGTAAAAATATACAGGCACCCAGACCAAG
ACTTTATAGTAATATACAACAGAAGAACCCCTCTAGGAGGCAACATCTAC
ACAGCACCCTCTCTACACCCAGGCAATGCCATTTTAGCAAAACACAAAAT
ATTAGTACCAAGTTTACAGACAAGACCAAAGGGTAGAAAAGCAATTAGAC
TAAGAATAGCACCCCCCACACTCTTTACAGACAAGTGGTACTTTCAAAAG
GACATAGCCGACCTCACCCTTTTCAACATCATGGCAGTTGAGGCTGACTT
GCGGTTTCCGTTCTGCTCACCACAAACTGACAACACTTGCATCAGCTTCC
AGGTCCTTAGTTCCGTTTACAACAACTACCTCAGTATTAATACCTTTAAT
AATGACAACTCAGACTCAAAGTTAAAAGAATTTTTAAATAAAGCATTTCC
AACAACAGGCACAAAAGGAACAAGTTTAAATGCACTAAATACATTTAGAA
CAGAAGGATGCATAAGTCACCCACAACTAAAAAAACCAAACCCACAAATA
AACAAACCATTAGAGTCACAATACTTTGCACCTTTAGATGCCCTCTGGGG
AGACCCCATATACTATAATGATCTAAATGAAAACAAAAGTTTGAACGATA
TCATTGAGAAAATACTAATAAAAAACATGATTACATACCATGCAAAACTA
AGAGAATTTCCAAATTCATACCAAGGAAACAAGGCCTTTTGCCACCTAAC
AGGCATATACAGCCCACCATACCTAAACCAAGGCAGAATATCTCCAGAAA
TATTTGGACTGTACACAGAAATAATTTACAACCCTTACACAGACAAAGGA
ACTGGAAACAAAGTATGGATGGACCCACTAACTAAAGAGAACAACATATA
TAAAGAAGGACAGAGCAAATGCCTACTGACTGACATGCCCCTATGGACTT
TACTTTTTGGATATACAGACTGGTGTAAAAAGGACACTAATAACTGGGAC
TTACCACTAAACTACAGACTAGTACTAATATGCCCTTATACCTTTCCAAA
ATTGTACAATGAAAAAGTAAAAGACTATGGGTACATCCCGTACTCCTACA
AATTCGGAGCGGGTCAGATGCCAGACGGCAGCAACTACATACCCTTTCAG
TTTAGAGCAAAGTGGTACCCCACAGTACTACACCAGCAACAGGTAATGGA
GGACATAAGCAGGAGCGGGCCCTTTGCACCTAAGGTAGAAAAACCAAGCA
CTCAGCTGGTAATGAAGTACTGTTTTAACTTTAACTGGGGCGGTAACCCT
ATCATTGAACAGATTGTTAAAGACCCCAGCTTCCAGCCCACCTATGAAAT
ACCCGGTACCGGTAACATCCCTAGAAGAATACAAGTCATCGACCCGCGGG
TCCTGGGACCGCACTACTCGTTCCGGTCATGGGACATGCGCAGACACACA
TTTAGCAGAGCAAGTATTAAGAGAGTGTCAGAACAACAAGAAACTTCTGA
CCTTGTATTCTCAGGCCCAAAAAAGCCTCGGGTCGACATCCCAAAACAAG
AAACCCAAGAAGAAAGCTCACATTCACTCCAAAGAGAATCGAGACCGTGG
GAGACCGAGGAAGAAAGCGAGACAGAAGCCCTCTCGCAAGAGAGCCAAGA
GGTCCCCTTCCAACAGCAGTTGCAGCAGCAGTACCAAGAGCAGCTCAAGC
TCAGACAGGGAATCAAAGTCCTCTTCGAGCAGCTCATAAGGACCCAACAA
GGGGTCCATGTAAACCCATGCCTACGGTAGGTCCCAGGCAGTGGCTGTTT
CCAGAGAGAAAGCCAGCCCCAGCTCCTAGCAGTGGAGACTGGGCCATGGA
GTTTCTCGCAGCAAAAATATTTGATAGGCCAGTTAGAAGCAACCTTAAAG
ATACCCCTTACTACCCATATGTTAAAAACCAATACAATGTCTACTTTGAC
CTTAAATTTGAATAAACAGCAGCTTCAAACTTGCAAGGCCGTGGGAGTTT
CACTGGTCGGTGTCTACCTCTAAAGGTCACTAAGCACTCCGAGCGTAAGC
GAGGAGTGCGACCCTCCCCCCTGGAACAACTTCTTCGGAGTCCGGCGCTA
CGCCTTCGGCTGCGCCGGACACCTCAGACCCCCCCTCCACCCGAAACGCT
TGCGCGTTTCGGACCTTCGGCGTCGGGGGGGTCGGGAGCTTTATTAAACG
GACTCCGAAGTGCTCTTGGACACTGAGGGGGTGAACAGCAACGAAAGTGA
GTGGGGCCAGACTTCGCCATAAGGCCTTTATCTTCTTGCCATTTGTCAGT
GTCCGGGGTCGCCATAGGCTTCGGGCTCGTTTTTAGGCCTTCCGGACTAC
AAAAATCGCCATTTTGGTGACGTCACGGCCGCCATCTTAAGTAGTTGAGG
CGGACGGTGGCGTGAGTTCAAAGGTCACCATCAGCCACACCTACTCAAAA
TGGTGGACAATTTCTTCCGGGTCAAAGGTTACAGCCGCCATGTTAAAACA
CGTGACGTATGACGTCACGGCCGCCATTTTGTGACACAAGATGGCCGACT
TCCTTCCTCTTTTTCAAAAAAAAGCGGAAGTGCCGCCGCGGCGGCGGGGG
GCGGCGCGCTGCGCGCGCCGCCCAGTAGGGGGAGCCATGCGCCCCCCCCC
GCGCATGCGCGGGGCCCCCCCCCGCGGGGGGCTCCGCCCCCCGGCCCCCC
CCG (SEQ ID NO: 16)

Annotations:

| Putative Domain | Base range |
|---|---|
| TATA Box | 83-88 |
| Cap Site | 104-111 |
| Transcriptional Start Site | 111 |
| 5′ UTR Conserved Domain | 170-240 |
| ORF2 | 336-719 |
| ORF2/2 | 336-715; 2363-2789 |
| ORF2/3 | 336-715; 2565-3015 |
| ORF2t/3 | 336-388; 2565-3015 |
| ORF1 | 599-2830 |
| ORF1/1 | 599-715; 2363-2830 |
| ORF1/2 | 599-715; 2565-2789 |
| Three open-reading frame region | 2551-2786 |
| Poly(A) Signal | 3011-3016 |
| GC-rich region | 3632-3753 |

TABLE A2
Exemplary Anellovirus amino acid sequences (Alphatorquevirus, Clade 3)
Ring1 (Alphatorquevirus Clade 3)

| | |
|---|---|
| ORF2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEE (SEQ ID NO: 17) |
| ORF2/2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEELLKTPASSPPMKYPVPVTSLEEYKSSTRGSWDRTTRSGHGT |
| | CADTHLAEQVLRECQNNKKLLTLYSQAQKSLGSTSQNKKPKKKAHIHSKENRDRG |
| | RPRKKARQKPSRKRAKRSPSNSSCSSSTKSSSSSDRESKSSSSSS (SEQ ID NO: 18) |
| ORF2/3 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEEPKKASGRHPKTRNPRRKLTFTPKRIETVGDRGRKRDRSPLA |
| | REPRGPLPTAVAAAVPRAAQAQTGNQSPLRAAHKDPTRGPCKPMPTVGPRQWLFP |
| | ERKPAPAPSSGDWAMEFLAAKIFDRPVRSNLKDTPYYPYVKNQYNVYFDLKFE |
| | (SEQ ID NO: 19) |
| ORF2t/3 | MSFWKPPVHNVTGIQRMWPKKASGRHPKTRNPRRKLTFTPKRIETVGDRGRKRDR |
| | SPLAREPRGPLPTAVAAAVPRAAQAQTGNQSPLRAAHKDPTRGPCKPMPTVGPRQ |
| | WLFPERKPAPAPSSGDWAMEFLAAKIFDRPVRSNLKDTPYYPYVKNQYNVYFDLK |
| | FE (SEQ ID NO: 20) |
| ORF1 | MAWGWWKRRRRWWFRKRWTRGRLRRRWPRSARRRPRRRRVRRRRRWRRGRRK |
| | TRTYRRRRRFRRRGRKAKLIIKLWQPAVIKRCRIKGYIPLIISGNGTFATNFTSHINDR |
| | IMKGPFGGGHSTMRFSLYILFEEHLRHMNFWTRSNDNLELTRYLGASVKIYRHPDQ |
| | DFIVIYNRRTPLGGNIYTAPSLHPGNAILAKHKILVPSLQTRPKGRKAIRLRIAPPTLFT |
| | DKWYFQKDIADLTLFNIMAVEADLRFPFCSPQTDNTCISFQVLSSVYNNYLSINTFN |
| | NDNSDSKLKEFLNKAFPTTGTKGTSLNALNTFRTEGCISHPQLKKPNPQINKPLESQ |
| | YFAPLDALWGDPIYYNDLNENKSLNDIIEKILIKNMITYHAKLREFPNSYQGNKAFC |
| | HLTGIYSPPYLNQGRISPEIFGLYTEIIYNPYTDKGTGNKVWMDPLTKENNIYKEGQS |
| | KCLLTDMPLWTLLFGYTDWCKKDTNNWDLPLNYRLVLICPYTFPKLYNEKVKDY |
| | GYIPYSYKFGAGQMPDGSNYIPFQFRAKWYPTVLHQQQVMEDISRSGPFAPKVEKP |
| | STQLVMKYCFNFNWGGNPIIEQIVKDPSFQPTYEIPGTGNIPRRIQVIDPRVLGPHYSF |
| | RSWDMRRHTFSRASIKRVSEQQETSDLVFSGPKKPRVDIPKQETQEESSHSLQRESR |
| | PWETEEESETEALSQESQEVPFQQQLQQQYQEQLKLRQGIKVLFEQLIRTQQGVHV |
| | NPCLR (SEQ ID NO: 21) |
| ORF1/1 | MAWGWWKRRRRWWFRKRWTRGRLRRRWPRSARRRPRRRRIVKDPSFQPTYEIPG |
| | TGNIPRRIQVIDPRVLGPHYSFRSWDMRRHTFSRASIKRVSEQQETSDLVFSGPKKPR |
| | VDIPKQETQEESSHSLQRESRPWETEEESETEALSQESQEVPFQQQLQQQYQEQLKL |
| | RQGIKVLFEQLIRTQQGVHVNPCLR (SEQ ID NO: 22) |
| ORF1/2 | MAWGWWKRRRRWWFRKRWTRGRLRRRWPRSARRRPRRRRAQKSLGSTSQNKK |
| | PKKKAHIHSKENRDRGRPRKKARQKPSRKRAKRSPSNSSCSSSTKSSSSSDRESKSSS |
| | SSS (SEQ ID NO: 23) |

TABLE B1
Exemplary Anellovirus nucleic acid sequence
(Betatorquevirus)

| | |
|---|---|
| Name | Ring2 |
| Genus/Clade | Betatorquevirus |
| Accession Number | JX134045.1 |

Full Sequence: 2797 bp
1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)

Annotations:

| Putative Domain | Base range |
|---|---|
| TATA Box | 237-243 |
| Cap Site | 260-267 |
| Transcriptional Start Site | 267 |
| 5’ UTR Conserved Domain | 323-393 |
| ORF2 | 424-723 |
| ORF2/2 | 424-719; 2274-2589 |
| ORF2/3 | 424-719; 2449-2812 |
| ORF1 | 612-2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE B2
Exemplary Anellovirus amino acid sequences (Betatorquevirus)
Ring2 (Betatorquevirus)

| | |
|---|---|
| ORF2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEG (SEQ ID NO: 55) |
| ORF2/2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGFNIPYPVTSMKQLRY |
| | RVQGKPQNPSYTPSTIDTGTTQQQLCHELAKTGHLKTLFLKLQSQIDSNCSNKPSNA |
| | CKSRKKRRRKKKKKYSSSSATSDSSSSCTESE (SEQ ID NO: 56) |
| ORF2/3 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGARSTATAQTSPRMP |
| | ANLGRNAGEKRKRSTAAHQQPQTAAAAVQRANNIIIKGPITFNCVKKVKLFDDKPK |
| | NRRFTPEEFETELQIAKWLKRPPRSFVNDPPFYPWLPPEPVVNFKLNFTE (SEQ ID |
| | NO: 57) |
| ORF1 | MPYYYRRRRYNYRRPRWYGRGWIRRPFRRRFRRKRRVRPTYTTIPLKQWQPPYKR |
| | TCYIKGQDCLIYYSNLRLGMNSTMYEKSIVPVHWPGGGSFSVSMLTLDALYDIHKL |
| | CRNWWTSTNQDLPLVRYKGCKITFYQSTFTDYIVRIHTELPANSNKLTYPNTHPLM |
| | MMMSKYKHIIPSRQTRRKKKPYTKIFVKPPPQFENKWYFATDLYKIPLLQIHCTACN |
| | LQNPFVKPDKLSNNVTLWSLNTISIQNRNMSVDQGQSWPFKILGTQSFYFYFYTGA |
| | NLPGDTTQIPVADLLPLTNPRINRPGQSLNEAKITDHITFTEYKNKFTNYWGNPFNK |
| | HIQEHLDMILYSLKSPEAIKNEWTTENMKWNQLNNAGTMALTPFNEPIFTQIQYNP |
| | DRDTGEDTQLYLLSNATGTGWDPPGIPELILEGFPLWLIYWGFADFQKNLKKVTNID |
| | TNYMLVAKTKFTQKPGTFYLVILNDTFVEGNSPYEKQPLPEDNIKWYPQVQYQLEA |
| | QNKLLQTGPFTPNIQGQLSDNISMFYKFYFKWGGSPPKAINVENPAHQIQYPIPRNE |
| | HETTSLQSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID NO: 58) |
| ORF1/1 | MPYYYRRRRYNYRRPRWYGRGWIRRPFRRRFRRKRRIQYPIPRNEHETTSLQSPGE |
| | APESILYSFDYRHGNYTTTALSRISQDWALKDTVSKITEPDRQQLLKQALECLQISEE |
| | TQEKKEKEVQQLISNLRQQQQLYRERIISLLKDQ (SEQ ID NO: 59) |
| ORF1/2 | MPYYYRRRRYNYRRPRWYGRGWIRRPFRRRFRRKRRSQIDSNCSNKPSNACKSRK |
| | KRRRKKKKKYSSSSATSDSSSSCTESE (SEQ ID NO: 60) |

TABLE C1
Exemplary Anellovirus nucleic acid sequence
(Gammatorquevirus)

| | |
|---|---|
| Name | Ring4 |
| Genus/Clade | Gammatorquevirus |
| Accession Number | |

Full Sequence: 3176 bp
1 10 20 30 40 50
| | | | | |
TAAAATGGCGGGAGCCAATCATTTTATACTTTCACTTTCCAATTAAAAAT
GGCCACGTCACAAACAAGGGGTGGAGCCATTTAAACTATATAACTAAGTG
GGGTGGCGAATGGCTGAGTTTACCCCGCTAGACGGTGCAGGGACCGGATC
GAGCGCAGCGAGGAGGTCCCCGGCTGCCCATGGGCGGGAGCCGAGGTGAG
TGAAACCACCGAGGTCTAGGGGCAATTCGGGCTAGGGCAGTCTAGCGGAA
CGGGCAAGAAACTTAAAACAATATTTGTTTTACAGATGGTTAGTATATCC
TCAAGTGATTTTTTTAAGAAAACGAAATTTAATGAGGAGACGCAGAACCA
AGTATGGATGTCTCAAATTGCTGACTCTCATGATAATATCTGCAGTTGCT
GGCATCCATTTGCTCACCTTCTTGCTTCCATATTTCCTCCTGGCCACAAA
GATCGTGATCTTACTATTAACCAAATTCTTCTAAGAGATTATAAAGAAAA
ATGCCATTCTGGTGGAGAAGAAGGAGAAAATTCTGGACCAACAACAGGTT
TAATTACACCAAAAGAAGAAGATATAGAAAAAGATGGCCCAGAAGGCGCC
GCAGAAGAAGACCATACAGACGCCCTGTTCGCCGCCGCCGTAGAAAACTT
CGAAAGGTAAAGAGAAAAAAAAAATCTTTAATTGTTAGACAATGGCAACC
AGACAGTATAAGAACTTGTAAAATTATAGGACAGTCAGCTATAGTTGTTG
GGGCTGAAGGAAAGCAAATGTACTGTTATACTGTCAATAAGTTAATTAAT
GTGCCCCCAAAAACACCATATGGGGGAGGCTTTGGAGTAGACCAATACAC
ACTGAAATACTTATATGAAGAATACAGATTTGCACAAAACATTTGGACAC
AATCTAATGTACTGAAAGACTTATGCAGATACATAAATGTTAAGCTAATA
TTCTACAGAGACAACAAAACAGACTTTGTCCTTTCCTATGACAGAAACCC
ACCTTTTCAACTAACAAAATTTACATACCCAGGAGCACACCCACAACAAA
TCATGCTTCAAAAACACCACAAATTCATACTATCACAAATGACAAAGCCT
AATGGAAGACTAACAAAAAAACTCAAAATTAAACCTCCTAAACAAATGCT
TTCTAAATGGTTCTTTTCAAAACAATTCTGTAAATACCCTTTACTATCTC
TTAAAGCTTCTGCACTAGACCTTAGGCACTCTTACCTAGGCTGCTGTAAT
GAAAATCCACAGGTATTTTTTTATTATTTAAACCATGGATACTACACAAT
AACAAACTGGGGAGCACAATCCTCAACAGCATACAGACCTAACTCCAAGG
TGACAGACACAACATACTACAGATACAAAAATGACAGAAAAAATATTAAC
ATTAAAAGCCATGAATACGAAAAAAGTATATCATATGAAAACGGTTATTT
TCAATCTAGTTTCTTACAAACACAGTGCATATATACCAGTGAGCGTGGTG
AAGCCTGTATAGCAGAAAAACCACTAGGAATAGCTATTTACAATCCAGTA
AAAGACAATGGAGATGGTAATATGATATACCTTGTAAGCACTCTAGCAAA
CACTTGGGACCAGCCTCCAAAAGACAGTGCTATTTTAATACAAGGAGTAC
CCATATGGCTAGGCTTATTTGGATATTTAGACTACTGTAGACAAATTAAA
GCTGACAAAACATGGCTAGACAGTCATGTACTAGTAATTCAAAGTCCTGC
TATTTTTACTTACCCAAATCCAGGAGCAGGCAAATGGTATTGTCCACTAT
CACAAAGTTTTATAAATGGCAATGGTCCGTTTAATCAACCACCTACACTG
CTACAAAAAGCAAAGTGGTTTCCACAAATACAATACCAACAAGAAATTAT
TAATAGCTTTGTAGAATCAGGACCATTTGTTCCCAAATATGCAAATCAAA
CTGAAAGCAACTGGGAACTAAAATATAAATATGTTTTTACATTTAAGTGG
GGTGGACCACAATTCCATGAACCAGAAATTGCTGACCCTAGCAAACAAGA
GCAGTATGATGTCCCCGATACTTTCTACCAAACAATACAAATTGAAGATC
CAGAAGGACAAGACCCCAGATCTCTCATCCATGATTGGGACTACAGACGA
GGCTTTATTAAAGAAAGATCTCTTAAAAGAATGTCAACTTACTTCTCAAC
TCATACAGATCAGCAAGCAACTTCAGAGGAAGACATTCCCAAAAAGAAAA
AGAGAATTGGACCCCAACTCACAGTCCCACAACAAAAAGAAGAGGAGACA
CTGTCATGTCTCCTCTCTCTCTGCAAAAAAGATACCTTCCAAGAAACAGA
GACACAAGAAGACCTCCAGCAGCTCATCAAGCAGCAGCAGGAGCAGCAGC
TCCTCCTCAAGAGAAACATCCTCCAGCTCATCCACAAACTAAAAGAGAAT
CAACAAATGCTTCAGCTTCACACAGGCATGTTACCTTAACCAGATTTAAA
CCTGGATTTGAAGAGCAAACAGAGAGAGAATTAGCAATTATATTTCATAG
GCCCCCTAGAACCTACAAAGAGGACCTTCCATTCTATCCCTGGCTACCAC
CTGCACCCCTTGTACAATTTAACCTTAACTTCAAAGGCTAGGCCAACAAT
GTACACTTAGTAAAGCATGTTTATTAAAGCACAACCCCCAAAATAAATGT
AAAAATAAAAAAAAAAAAAAAAAAATAAAAAATTGCAAAAATTCGGCGCT
CGCGCGCATGTGCGCCTCTGGCGCAAATCACGCAACGCTCGCGCGCCCGC
GTATGTCTCTTTACCACGCACCTAGATTGGGGTGCGCGCGCTAGCGCGCG
CACCCCAATGCGCCCCGCCCTCGTTCCGACCCGCTTGCGCGGGTCGGACC
ACTTCGGGCTCGGGGGGGCGCGCCTGCGGCGCTTTTTTACTAAACAGACT
CCGAGCCGCCATTTGGCCCCCTAAGCTCCGCCCCCCTCATGAATATTCAT
AAAGGAAACCACATAATTAGAATTGCCGACCACAAACTGCCATATGCTAA
TTAGTTCCCCTTTTACAAAGTAAAAGGGGAAGTGAACATAGCCCCACACC
CGCAGGGGCAAGGCCCCGCACCCCTACGTCACTAACCACGCCCCCGCCGC
CATCTTGGGTGCGGCAGGGCGGGGGC (SEQ ID NO: 886)

Annotations:

| Putative Domain | Base range |
|---|---|
| TATA Box | 87-93 |
| Cap Site | 110-117 |
| Transcriptional Start Site | 117 |
| 5′ UTR Conserved Domain | 185-254 |
| ORF2 | 286-660 |
| ORF2/2 | 286-656; 1998-2442 |
| ORF2/3 | 286-656; 2209-2641 |
| TAIP | 385-484 |
| ORF1 | 501-2489 |
| ORF1/1 | 501-656; 1998-2489 |
| ORF1/2 | 501-656; 2209-2442 |
| Three open-reading frame region | 2209-2439 |
| Poly(A) Signal | 2672-2678 |
| GC-rich region | 3076-3176 |

TABLE C2
Exemplary Anellovirus amino acid sequences (Gammatorquevirus)
Ring4 (Gammatorquevirus)

| | |
|---|---|
| ORF2 | MVSISSSDFFKKTKFNEETQNQVWMSQIADSHDNICSCWHPFAHLLASIFPP |
| | GHKDRDLTINQILLR |
| | DYKEKCHSGGEEGENSGPTTGLITPKEEDIEKDGPEGAAEEDHTDALFAAAV |
| | ENFER (SEQ ID NO: 887) |
| ORF2/2 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFESGVDHNSMNQKLLTLANKSSMMSPILSTKQYKL |
| | KIQKDKTPDLSSMIGTTDEALLKKDLLKECQLTSQLIQISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 888) |
| ORF2/3 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFERSASNFRGRHSQKEKENWTPTHSPTTKRRGDTV |
| | MSPLSLQKRYLPRNRDTRRPPAAHQAAAGAAAPPQEKHPPAHPQTKRESTN |
| | ASASHRHVTLTRFKPGFEEQTERELAIIFHRPPRTYKEDLPFYPWLPPAPLVQ |
| | FNLNFKG (SEQ ID NO: 889) |
| TAIP | MRRRRTKYGCLKLLTLMIISAVAGIHLLTFLLPYFLLATKIVILLLTKFF (SEQ |
| | ID NO: 890) |
| ORF1 | MPFWWRRRRKFWTNNRFNYTKRRRYRKRWPRRRRRRRPYRRPVRRRRRK |
| | LRKVKRKKKSLIVRQWQPDSIRTCKIIGQSAIVVGAEGKQMYCYTVNKLINV |
| | PPKTPYGGGFGVDQYTLKYLYEEYRFAQNIWTQSNVLKDLCRYINVKLIFY |
| | RDNKTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNENPQ |
| | VFFYYLNHGYYTITNWGAQSSTAYRPNSKVTDTTYYRYKNDRKNINIKSHE |
| | YEKSISYENGYFQSSFLQTQCIYTSERGEACIAEKPLGIAIYNPVKDNGDGNM |
| | IYLVSTLANTWDQPPKDSAILIQGVPIWLGLFGYLDYCRQIKADKTWLDSHV |
| | LVIQSPAIFTYPNPGAGKWYCPLSQSFINGNGPFNQPPTLLQKAKWFPQIQYQ |
| | QEIINSFVESGPFVPKYANQTESNWELKYKYVFTFKWGGPQFHEPEIADPSK |
| | QEQYDVPDTFYQTIQIEDPEGQDPRSLIHDWDYRRGFIKERSLKRMSTYFST |
| | HTDQQATSEEDIPKKKKRIGPQLTVPQQKEEETLSCLLSLCKKDTFQETETQE |
| | DLQQLIKQQQEQQLLLKRNILQLIHKLKENQQMLQLHTGMLP (SEQ ID NO: |
| | 891) |
| ORF1/1 | MPFWWRRRRKFWTNNRFNYTKRRRYRKRWPRRRRRRRPYRRPVRRRRRK |
| | LRKWGGPQFHEPEIADPSKQEQYDVPDTFYQTIQIEDPEGQDPRSLIHDWDY |
| | RRGFIKERSLKRMSTYFSTHTDQQATSEEDIPKKKKRIGPQLTVPQQKEEETL |
| | SCLLSLCKKDTFQETETQEDLQQLIKQQQEQQLLLKRNILQLIHKLKENQQM |
| | LQLHTGMLP (SEQ ID NO: 892) |
| ORF1/2 | MPFWWRRRRKFWTNNRFNYTKRRRYRKRWPRRRRRRRPYRRPVRRRRRK |
| | LRKISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 893) |

In some embodiments, an anellovector comprises a nucleic acid comprising a sequence listed in PCT Application No. PCT/US2018/037379, incorporated herein by reference in its entirety. In some embodiments, an anellovector comprises a polypeptide comprising a sequence listed in PCT Application No. PCT/US2018/037379, incorporated herein by reference in its entirety. In some embodiments, an anellovector comprises a nucleic acid comprising a sequence listed in PCT Application No. PCT/US 19/65995, incorporated herein by reference in its entirety. In some embodiments, an anellovector comprises a polypeptide comprising a sequence listed in PCT Application No. PCT/US19/65995, incorporated herein by reference in its entirety.

ORF1 Molecules

In some embodiments, the anellovector comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein). In some embodiments, the ORF1 molecule comprises a truncation relative to an Anellovirus ORF1 protein (e.g., an Anellovirus ORF1 protein as described herein). An ORF1 molecule may be capable of binding to other ORF1 molecules, e.g., to form a proteinaceous exterior (e.g., as described herein), e.g., a capsid. In some embodiments, the proteinaceous exterior may enclose a nucleic acid molecule (e.g., a genetic element as described herein). In some embodiments, a plurality of ORF1 molecules may form a multimer, e.g., to form a proteinaceous exterior. In some embodiments, the multimer may be a homomultimer. In other embodiments, the multimer may be a heteromultimer.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) β-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

N22 Domain

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

Hypervariable Region (HVR)

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

Exemplary ORF1 Sequences

Exemplary Anellovirus ORF1 amino acid sequences, and the sequences of exemplary ORF1 domains, are provided in the tables below. In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z). In some embodiments, an anellovector described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables N-Z.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed in any of Tables N-Z), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses (e.g., any combination of ORF1 subsequences selected from the Alphatorquevirus Clade 1-7 subsequences listed in Tables N-Z). In embodiments, the ORF1 molecule comprises one or more of an Arg-rich domain, a jelly-roll domain, an N22 domain, and a CTD from one Anellovirus, and an HVR from another. In embodiments, the ORF1 molecule comprises one or more of a jelly-roll domain, an HVR, an N22 domain, and a CTD from one Anellovirus, and an Arg-rich domain from another. In embodiments, the ORF1 molecule comprises one or more of an Arg-rich domain, an HVR, an N22 domain, and a CTD from one Anellovirus, and a jelly-roll domain from another. In embodiments, the ORF1 molecule comprises one or more of an Arg-rich domain, a jelly-roll domain, an HVR, and a CTD from one Anellovirus, and an N22 domain from another. In embodiments, the ORF1 molecule comprises one or more of an Arg-rich domain, a jelly-roll domain, an HVR, and an N22 domain from one Anellovirus, and a CTD from another.

Additional exemplary Anelloviruses for which the ORF1 molecules, or splice variants or functional fragments thereof, can be utilized in the compositions and methods described herein (e.g., to form the proteinaceous exterior of an anellovector, e.g., by enclosing a genetic element) are described, for example, in PCT Application Nos. PCT/US2018/037379 and PCT/US19/65995 (incorporated herein by reference in their entirety).

TABLE N
Exemplary Anellovirus ORF1 amino acid subsequence
(Alphatorquevirus, Clade 3)

| | |
|---|---|
| Name | Ring1 |
| Genus/Clade | Alphatorquevirus, Clade3 |
| Accession Number | AJ620231.1 |
| Protein Accession Number | CAF05750.1 |

Full Sequence: 743 AA
1 10 20 30 40 50
| | | | | |
MAWGWWKRRRRWWFRKRWTRGRLRRRWPRSARRRPRRRRVRRRRRWRRGR
RKTRTYRRRRRFRRRGRKAKLIIKLWQPAVIKRCRIKGYIPLIISGNGTF
ATNFTSHINDRIMKGPFGGGHSTMRFSLYILFEEHLRHMNFWTRSNDNLE
LTRYLGASVKIYRHPDQDFIVIYNRRTPLGGNIYTAPSLHPGNAILAKHK
ILVPSLQTRPKGRKAIRLRIAPPTLFTDKWYFQKDIADLTLFNIMAVEAD
LRFPFCSPQTDNTCISFQVLSSVYNNYLSINTFNNDNSDSKLKEFLNKAF
PTTGTKGTSLNALNTFRTEGCISHPQLKKPNPQINKPLESQYFAPLDALW
GDPIYYNDLNENKSLNDIIEKILIKNMITYHAKLREFPNSYQGNKAFCHL
TGIYSPPYLNQGRISPEIFGLYTEIIYNPYTDKGTGNKVWMDPLTKENNI
YKEGQSKCLLTDMPLWTLLFGYTDWCKKDTNNWDLPLNYRLVLICPYTFP
KLYNEKVKDYGYIPYSYKFGAGQMPDGSNYIPFQFRAKWYPTVLHQQQVM
EDISRSGPFAPKVEKPSTQLVMKYCFNFNWGGNPIIEQIVKDPSFQPTYE
IPGTGNIPRRIQVIDPRVLGPHYSFRSWDMRRHTFSRASIKRVSEQQETS
DLVFSGPKKPRVDIPKQETQEESSHSLQRESRPWETEEESETEALSQESQ
EVPFQQQLQQQYQEQLKLRQGIKVLFEQLIRTQQGVHVNPCLR
(SEQ ID NO: 185)

Annotations:

| Putative Domain | AA range |
|---|---|
| Arg-Rich Region | 1-68 |
| Jelly-roll domain | 69-280 |
| Hypervariable Region | 281-413 |
| N22 | 414-579 |
| C-terminal Domain | 580-743 |

TABLE O
Exemplary Anellovirus ORF1 amino acid subsequence (Alphatorquevirus, Clade 3)
Ring1 ORF1 (Alphatorquevirus Clade 3)

| | |
|---|---|
| Arg-Rich | MAWGWWKRRRRWWFRKRWTRGRLRRRWPRSARRRPRRRRVRRRR |
| Region | RWRRGRRKTRTYRRRRRFRRRGRK (SEQ ID NO: 186) |
| Jelly-roll | AKLIIKLWQPAVIKRCRIKGYIPLIISGNGTFATNFTSHINDRIMKGPFGG |
| Domain | GHSTMRFSLYILFEEHLRHMNFWTRSNDNLELTRYLGASVKIYRHPDQ |
| | DFIVIYNRRTPLGGNIYTAPSLHPGNAILAKHKILVPSLQTRPKGRKAIRL |
| | RIAPPTLFTDKWYFQKDIADLTLFNIMAVEADLRFPFCSPQTDNTCISFQ |
| | VLSSVYNNYLSI (SEQ ID NO: 187) |
| Hypervariable | NTFNNDNSDSKLKEFLNKAFPTTGTKGTSLNALNTFRTEGCISHPQLKK |
| domain | PNPQINKPLESQYFAPLDALWGDPIYYNDLNENKSLNDIIEKILIKNMIT |
| | YHAKLREFPNSYQGNKAFCHLTGIYSPPYLNQGR (SEQ ID NO: 188) |
| N22 | ISPEIFGLYTEIIYNPYTDKGTGNKVWMDPLTKENNIYKEGQSKCLLTD |
| | MPLWTLLFGYTDWCKKDTNNWDLPLNYRLVLICPYTFPKLYNEKVKD |
| | YGYIPYSYKFGAGQMPDGSNYIPFQFRAKWYPTVLHQQQVMEDISRSG |
| | PFAPKVEKPSTQLVMKYCFNFN (SEQ ID NO: 189) |
| C-terminal | WGGNPIIEQIVKDPSFQPTYEIPGTGNIPRRIQVIDPRVLGPHYSFRSWD |
| domain | MRRHTFSRASIKRVSEQQETSDLVFSGPKKPRVDIPKQETQEESSHSLQR |
| | ESRPWETEEESETEALSQESQEVPFQQQLQQQYQEQLKLRQGIKVLFEQ |
| | LIRTQQGVHVNPCLR (SEQ ID NO: 190) |

TABLE P
Exemplary Anellovirus ORF1 amino acid subsequence
(Betatorquevirus)

| | |
|---|---|
| Name | Ring2 |
| Genus/Clade | Betatorquevirus |
| Accession Number | JX134045.1 |
| Protein Accession Number | AGG91484.1 |

Full Sequence: 666 AA
1 10 20 30 40 50
| | | | | |
MPYYYRRRRYNYRRPRWYGRGWIRRPFRRRFRRKRRVRPTYTTIPLKQWQ
PPYKRTCYIKGQDCLIYYSNLRLGMNSTMYEKSIVPVHWPGGGSFSVSML
TLDALYDIHKLCRNWWTSTNQDLPLVRYKGCKITFYQSTFTDYIVRIHTE
LPANSNKLTYPNTHPLMMMMSKYKHIIPSRQTRRKKKPYTKIFVKPPPQF
ENKWYFATDLYKIPLLQIHCTACNLQNPFVKPDKLSNNVTLWSLNTISIQ
NRNMSVDQGQSWPFKILGTQSFYFYFYTGANLPGDTTQIPVADLLPLTNP
RINRPGQSLNEAKITDHITFTEYKNKFTNYWGNPFNKHIQEHLDMILYSL
KSPEAIKNEWTTENMKWNQLNNAGTMALTPFNEPIFTQIQYNPDRDTGED
TQLYLLSNATGTGWDPPGIPELILEGFPLWLIYWGFADFQKNLKKVTNID
TNYMLVAKTKFTQKPGTFYLVILNDTFVEGNSPYEKQPLPEDNIKWYPQV
QYQLEAQNKLLQTGPFTPNIQGQLSDNISMFYKFYFKWGGSPPKAINVEN
PAHQIQYPIPRNEHETTSLQSPGEAPESILYSFDYRHGNYTTTALSRISQ
DWALKDTVSKITEPDRQQLLKQALECLQISEETQEKKEKEVQQLISNLRQ
QQQLYRERIISLLKDQ (SEQ ID NO: 215)

Annotations:

| Putative Domain | AA range |
|---|---|
| Arg-Rich Region | 1-38 |
| Jelly-roll domain | 39-246 |
| Hypervariable Region | 247-374 |
| N22 | 375-537 |
| C-terminal Domain | 538-666 |

TABLE Q
Exemplary Anellovirus ORF1 amino acid subsequence (Betatorquevirus)
Ring2 ORF1 (Betatorquevirus)

| | |
|---|---|
| Arg-Rich | MPYYYRRRRYNYRRPRWYGRGWIRRPFRRRFRRKRRVR (SEQ ID NO: |
| Region | 216) |
| Jelly-roll | PTYTTIPLKQWQPPYKRTCYIKGQDCLIYYSNLRLGMNSTMYEKSIVPV |
| Domain | HWPGGGSFSVSMLTLDALYDIHKLCRNWWTSTNQDLPLVRYKGCKIT |
| | FYQSTFTDYIVRIHTELPANSNKLTYPNTHPLMMMMSKYKHIIPSRQTR |
| | RKKKPYTKIFVKPPPQFENKWYFATDLYKIPLLQIHCTACNLQNPFVKP |
| | DKLSNNVTLWSLNT (SEQ ID NO: 217) |
| Hypervariable | ISIQNRNMSVDQGQSWPFKILGTQSFYFYFYTGANLPGDTTQIPVADLL |
| domain | PLTNPRINRPGQSLNEAKITDHITFTEYKNKFTNYWGNPFNKHIQEHLD |
| | MILYSLKSPEAIKNEWTTENMKWNQLNNAG (SEQ ID NO: 218) |
| N22 | TMALTPFNEPIFTQIQYNPDRDTGEDTQLYLLSNATGTGWDPPGIPELIL |
| | EGFPLWLIYWGFADFQKNLKKVTNIDTNYMLVAKTKFTQKPGTFYLVI |
| | LNDTFVEGNSPYEKQPLPEDNIKWYPQVQYQLEAQNKLLQTGPFTPNI |
| | QGQLSDNISMFYKFYFK (SEQ ID NO: 219) |
| C-terminal | WGGSPPKAINVENPAHQIQYPIPRNEHETTSLQSPGEAPESILYSFDYRH |
| domain | GNYTTTALSRISQDWALKDTVSKITEPDRQQLLKQALECLQISEETQEK |
| | KEKEVQQLISNLRQQQQLYRERIISLLKDQ (SEQ ID NO: 220) |

TABLE R
Exemplary Anellovirus ORF1 amino acid subsequence
(Gammatorquevirus)

| | |
|---|---|
| Name | Ring4 |
| Genus/Clade | Gammatorquevirus |
| Accession Number | |
| Protein Accession Number | |

Full Sequence: 662 AA
1 10 20 30 40 50
| | | | | |
MPFWWRRRRKFWTNNRFNYTKRRRYRKRWPRRRRRRRPYRRPVRRRRRKL
RKVKRKKKSLIVRQWQPDSIRTCKIIGQSAIVVGAEGKQMYCYTVNKLIN
VPPKTPYGGGFGVDQYTLKYLYEEYRFAQNIWTQSNVLKDLCRYINVKLI
FYRDNKTDFVLSYDRNPPFQLTKFTYPGAHPQQIMLQKHHKFILSQMTKP
NGRLTKKLKIKPPKQMLSKWFFSKQFCKYPLLSLKASALDLRHSYLGCCN
ENPQVFFYYLNHGYYTITNWGAQSSTAYRPNSKVTDTTYYRYKNDRKNIN
IKSHEYEKSISYENGYFQSSFLQTQCIYTSERGEACIAEKPLGIAIYNPV
KDNGDGNMIYLVSTLANTWDQPPKDSAILIQGVPIWLGLFGYLDYCRQIK
ADKTWLDSHVLVIQSPAIFTYPNPGAGKWYCPLSQSFINGNGPFNQPPTL
LQKAKWFPQIQYQQEIINSFVESGPFVPKYANQTESNWELKYKYVFTFKW
GGPQFHEPEIADPSKQEQYDVPDTFYQTIQIEDPEGQDPRSLIHDWDYRR
GFIKERSLKRMSTYFSTHTDQQATSEEDIPKKKKRIGPQLTVPQQKEEET
LSCLLSLCKKDTFQETETQEDLQQLIKQQQEQQLLLKRNILQLIHKLKEN
OQMLQLHTGMLP (SEQ ID NO: 925)

Annotations:

| Putative Domain | AA range |
|---|---|
| Arg-Rich Region | 1-58 |
| Jelly-roll domain | 59-260 |
| Hypervariable Region | 261-339 |
| N22 | 340-499 |
| C-terminal Domain | 500-662 |

TABLE S
Exemplary Anellovirus ORF1 amino acid subsequence (Gammatorquevirus)
Ring4 (Gammatorquevirus)

| | |
|---|---|
| Arg-Rich | MPFWWRRRRKFWTNNRFNYTKRRRYRKRWPRRRRRRRPYRRPVRRR |
| Region | RRKLRKVKRKKK (SEQ ID NO: 926) |
| Jelly-roll | SLIVRQWQPDSIRTCKIIGQSAIVVGAEGKQMYCYTVNKLINVPPKTPY |
| Domain | GGGFGVDQYTLKYLYEEYRFAQNIWTQSNVLKDLCRYINVKLIFYRDN |
| | KTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNE |
| | NPQVFFYYL (SEQ ID NO: 927) |
| Hypervariable | NHGYYTITNWGAQSSTAYRPNSKVTDTTYYRYKNDRKNINIKSHEYEK |
| domain | SISYENGYFQSSFLQTQCIYTSERGEACIAE (SEQ ID NO: 928) |
| N22 | KPLGIAIYNPVKDNGDGNMIYLVSTLANTWDQPPKDSAILIQGVPIWLG |
| | LFGYLDYCRQIKADKTWLDSHVLVIQSPAIFTYPNPGAGKWYCPLSQSF |
| | INGNGPFNQPPTLLQKAKWFPQIQYQQEIINSFVESGPFVPKYANQTESN |
| | WELKYKYVFTFK (SEQ ID NO: 929) |
| C-terminal | WGGPQFHEPEIADPSKQEQYDVPDTFYQTIQIEDPEGQDPRSLIHDWDY |
| domain | RRGFIKERSLKRMSTYFSTHTDQQATSEEDIPKKKKRIGPQLTVPQQKE |
| | EETLSCLLSLCKKDTFQETETQEDLQQLIKQQQEQQLLLKRNILQLIHKL |
| | KENQQMLQLHTGMLP (SEQ ID NO: 930) |

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization signal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

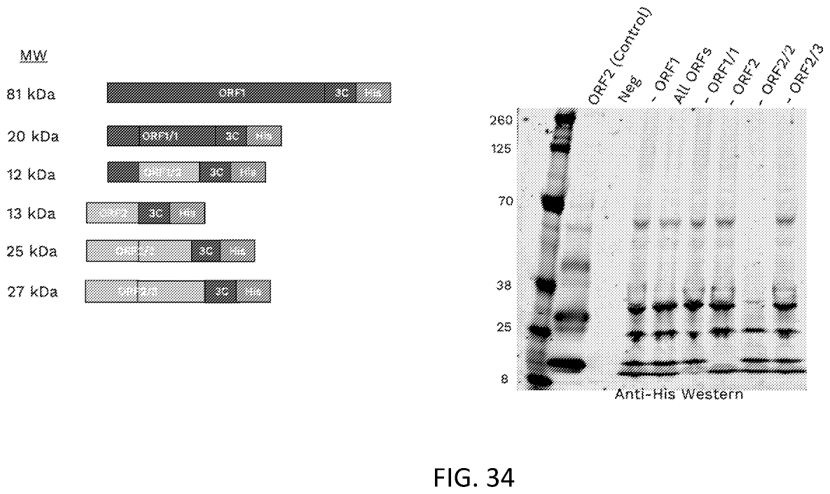

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34 of PCT/US19/65995). In some embodiments, the conserved motifs may show 60, 70, 80, 85, 90, 95, or 100% sequence identity to an ORF1 protein of one or more wild-type Anellovirus clades (e.g., Alphatorquevirus, clade 1; Alphatorquevirus, clade 2; Alphatorquevirus, clade 3; Alphatorquevirus, clade 4; Alphatorquevirus, clade 5; Alphatorquevirus, clade 6; Alphatorquevirus, clade 7; Betatorquevirus; and/or Gammatorquevirus). In embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule comprises at least one difference (e.g., a mutation, chemical modification, or epigenetic alteration) relative to a wild-type ORF1 protein, e.g., as described herein.

Conserved ORF1 Motif in N22 Domain

In some embodiments, a polypeptide (e.g., an ORF1 molecule) described herein comprises the amino acid sequence YNPX²DXGX²N (SEQ ID NO: 829), wherein Xⁿ is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

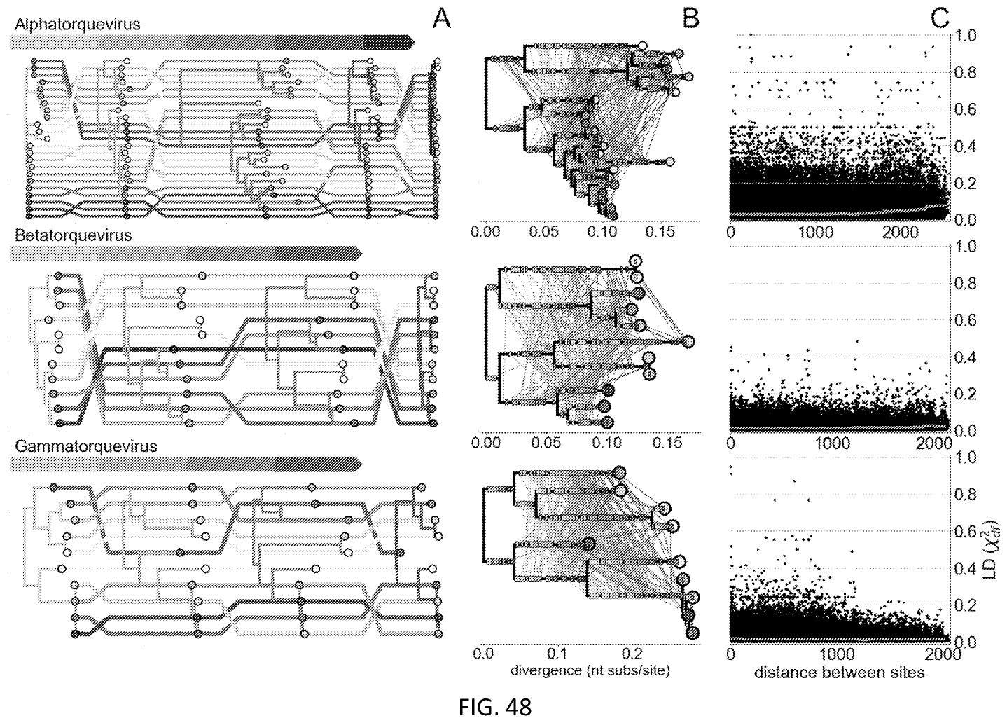

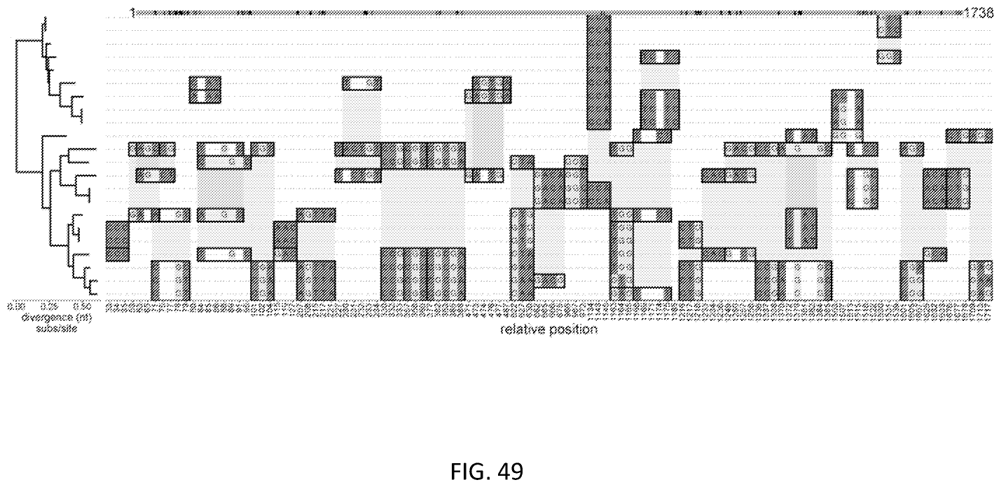

Exemplary YNPX²DXGX²N (SEQ ID NO: 829) motif-flanking secondary structures are described in Example 47 and FIG. 48 of PCT/US19/65995; incorporated herein by reference in its entirety. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT/US19/65995, flanking a YNPX²DXGX²N (SEQ ID NO: 829) motif (e.g., as described herein).

Conserved Secondary Structural Motif in ORF1 Jelly-Roll Domain

In some embodiments, a polypeptide (e.g., an ORF1 molecule) described herein comprises one or more secondary structural elements comprised by an Anellovirus ORF1 protein (e.g., as described herein). In some embodiments, an ORF1 molecule comprises one or more secondary structural elements comprised by the jelly-roll domain of an Anellovirus ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

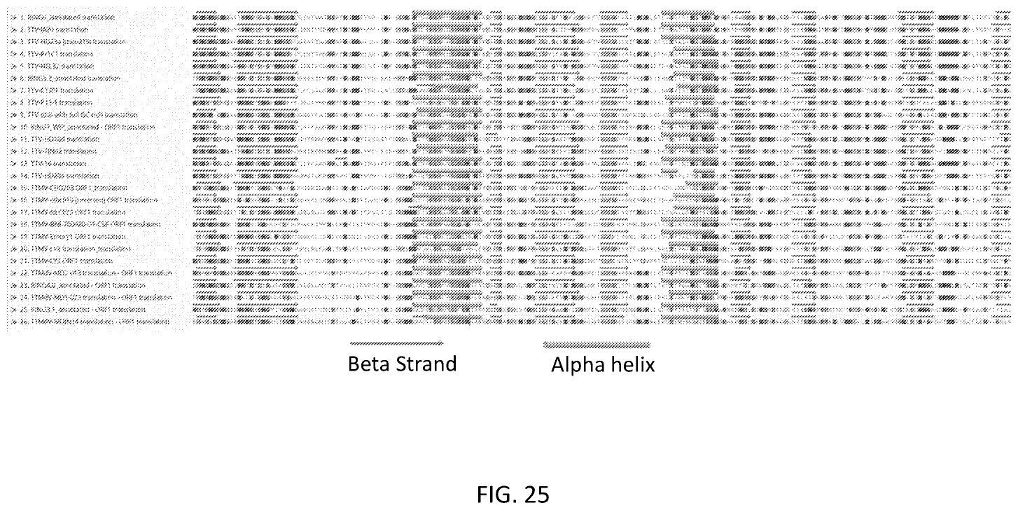

Exemplary jelly-roll domain secondary structures are described in Example 47 of PCT/US19/65995 and FIG. 25 herein. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 25 herein.

Consensus ORF1 Domain Sequences

In some embodiments, an ORF1 molecule, e.g., as described herein, comprises one or more of a jelly-roll domain, N22 domain, and/or C-terminal domain (CTD). In some embodiments, the jelly-roll domain comprises an amino acid sequence having a jelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(X_{a-b})” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYATHS | 227 |
| | DDTIQKGPFGGGMSTTTFSLRVLYDEYQRFMNRWTYSNED | |
| | LDLARYLGCKFTFYRHPDXDFIVQYNTNPPFKDTKLTAPSIH | |
| | P(X₁₋₅)GMLMLSKRKILIPSLKTRPKGKHYVKVRIGPPKLFED | |
| | KWYTQSDLCDVPLVXLYATAADLQHPFGSPQTDNPCVTFQ | |
| | VLGSXYNKHLSISP; | |
| | wherein X = any amino acid. | |
| N22 | SNFEFPGAYTDITYNPLTDKGVGNMVWIQYLTKPDTIXDKT | 228 |
| | QS(X₀₋₃)KCLIEDLPLWAALYGYVDFCEKETGDSAIIXNXGRV | |
| | LIRCPYTKPPLYDKT(X₀₋₄)NKGFVPYSTNFGNGKMPGGSGY | |
| | VPIYWRARWYPTLFHQKEVLEDIVQSGPFAYKDEKPSTQLV | |
| | MKYCFNFN; | |
| | wherein X = any amino acid. | |
| CTD | WGGNPISQQVVRNPCKDSG(X₀₋₃)SGXGRQPRSVQVVDPKY | 229 |
| | MGPEYTFHSWDWRRGLFGEKAIKRMSEQPTDDEIFTGGXPK | |
| | RPRRDPPTXQXPEE(X₁₋₄)QKESSSFR(X₂₋₁₄)PWESSSQEXESES | |
| | QEEEE(X₀₋₃₀)EQTVQQQLRQQLREQRRLRVQLQLLFQQLLKT | |
| | (X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESIVPH | 230 |
| | HEPGGGGWSIQQFTLGALYEEHLKLRNWWTKSNDGLPLVR | |
| | YLGCTIKLYRSEDTDYIVTYQRCYPMTATKLTYLSTQPSRM | |
| | LMNKHKIIVPSKXT(X₁₋₄)NKKKKPYKKIFIKPPSQMQNKWYF | |
| | QQDIANTPLLQLTXTACSLDRMYLSSDSISNNITFTSLNTNFF | |
| | QNPNFQ; | |
| | wherein X = any amino acid. | |
| N22 | (X₄₋₁₀)TPLYFECRYNPFKDKGTGNKVYLVSNN(X₁₋₈)TGWDPP | 231 |
| | TDPDLIIEGFPLWLLLWGWLDWQKKLGKIQNIDTDYILVIQS | |
| | XYYIPP(X₁₋₃)KLPYYVPLDXD(X₀₋₂)FLHGRSPY(X₃₋₁₆)PSDKQH | |
| | WHPKVRFQXETINNIALTGPGTPKLPNQKSIQAHMKYKFYF | |
| | K; | |
| | wherein X = any amino acid. | |
| CTD | WGGCPAPMETITDPCKQPKYPIPNNLLQTTSLQXPTTPIETYL | 232 |
| | YKFDERRGLLTKKAAKRIKKDXTTETTLFTDTGXXTSTTLPT | |
| | XXQTETTQEEXTSEEE(X₀₋₅)ETLLQQLQQLRRKQKQLRXRIL | |
| | QLLQLLXLL(X₀₋₂₆)*; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNEKDE | 233 |
| | YTPPKAPGGGGFGVELFSLEYLYEQWKARNNIWTKSNXYK | |
| | DLCRYTGCKITFYRHPTTDFIVXYSRQPPFEIDKXTYMXXHP | |
| | QXLLLRKHKKIILSKATNPKGKLKKKIKIKPPKQMLNKWFF | |
| | QKQFAXYGLVQLQAAACBLRYPRLGCCNENRLITLYYLN; | |
| | wherein X = any amino acid. | |
| N22 | LPIVVARYNPAXDTGKGNKXWLXSTLNGSXWAPPTTDKDL | 234 |
| | IIEGLPLWLALYGYWSYJKKVKKDKGILQSHMFVVKSPAIQP | |
| | LXTATTQXTFYPXIDNSFIQGKXPYDEPJTXNQKKLWYPTLE | |
| | HQQETINAIVESGPYVPKLDNQKNSTWELXYXYTFYFK; | |
| | wherein X = any amino acid. | |
| CTD | WGGPQIPDQPVEDPKXQGTYPVPDTXQQTIQIXNPLKQKPE | 235 |
| | TMFHDWDYRRGIITSTALKRMQENLETDSSFXSDSEETP(X₀₋₂) | |
| | KKKKRLTXELPXPQEETEEIQSCLLSLCEESTCQEE(X₁₋₆)ENL | |
| | QQLIHQQQQQQQQLKHNILKLLSDLKZKQRLLQLQTGILE | |
| | (X₁₋₁₀)*; | |
| | wherein X = any amino acid. | |

In some embodiments, the jelly-roll domain comprises a jelly-roll domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 21, 23, 25, 27, 29, 31, 33, 35, D2, D4, D6, D8, D10, or 37A-37C, or an amino acid sequence having at least 70%, 75%, 80%, 8%, 90%, 95%, 96%, 97%, 98%, 99%, or 100% sequence identity thereto.

Identification of ORF1 Protein Sequences

In some embodiments, an Anellovirus ORF1 protein sequence, or a nucleic acid sequence encoding an ORF1 protein, can be identified from the genome of an Anellovirus (e.g., a putative Anellovirus genome identified, for example, by nucleic acid sequencing techniques, e.g., deep sequencing techniques). In some embodiments, an ORF1 protein sequence is identified by one or more (e.g., 1, 2, or all 3) of the following selection criteria:

(i) Length Selection: Protein sequences (e.g., putative Anellovirus ORF1 sequences passing the criteria described in (ii) or (iii) below) may be size-selected for those greater than about 600 amino acid residues to identify putative Anellovirus ORF1 proteins. In some embodiments, an Anellovirus ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an Anellovirus ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.

(ii) Presence of ORF1 motif: Protein sequences (e.g., putative Anellovirus ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN. In some embodiments, a putative Anellovirus ORF1 sequence comprises the sequence Y[NCS]PXXDX[GASKR]XX[NTSVAK].

(iii) Presence of arginine-rich region: Protein sequences (e.g., putative Anellovirus ORF1 sequences passing the criteria described in (i) and/or (ii) above) may be filtered for those that include an arginine-rich region (e.g., as described herein). In some embodiments, a putative Anellovirus ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative Anellovirus ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative Anellovirus ORF1 protein. In some embodiments, the arginine-rich region is positioned at least about 50 amino acids downstream of the start codon of the putative Anellovirus ORF1 protein.

ORF2 Molecules

In some embodiments, the anellovector comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in any of Tables A2, A4, A6, A8, A10, A12, C1-C5, 2, 4, 6, 8, 10, 12, 14, 16, or 18), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in any of Tables A2, A4, A6, A8, A10, A12, C1-C5, 2, 4, 6, 8, 10, 12, 14, 16, or 18.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule comprises at least one difference (e.g., a mutation, chemical modification, or epigenetic alteration) relative to a wild-type ORF2 protein, e.g., as described herein (e.g., as shown in any of Tables A2, A4, A6, A8, A10, A12, C1-C5, 2, 4, 6, 8, 10, 12, 14, 16, or 18).

Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein Xⁿ is a contiguous sequence of any n amino acids. In embodiments, X⁷ indicates a contiguous sequence of any seven amino acids. In embodiments, X³ indicates a contiguous sequence of any three amino acids. In embodiments, X¹ indicates any single amino acid. In embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein Xⁿ is a contiguous sequence of any n amino acids.

Genetic Element

In some embodiments, the anellovector comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein.

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb. In some embodiments, the genetic element has a length between about 3.6-3.9 kb. In some embodiments, the genetic element has a length between about 2.8-2.9 kb. In some embodiments, the genetic element has a length between about 2.0-3.2 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences.

In some embodiments, the genetic element was produced from a double-stranded circular DNA (e.g., produced by in vitro circularization). In some embodiments, the genetic element was produced by rolling circle replication from the double-stranded circular DNA. In some embodiments, the rolling circle replication occurs in a cell (e.g., a host cell, e.g., a mammalian cell, e.g., a human cell, e.g., a HEK293T cell, an A549 cell, or a Jurkat cell). In some embodiments, the genetic element can be amplified exponentially by rolling circle replication in the cell. In some embodiments, the genetic element can be amplified linearly by rolling circle replication in the cell. In some embodiments, the double-stranded circular DNA or genetic element is capable of yielding at least 2, 4, 8, 16, 32, 64, 128, 256, 518, 1024 or more times the original quantity by rolling circle replication in the cell. In some embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8 of PCT/US19/65995.

In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence, e.g., as described herein.

In some embodiments, the protein binding sequence has at least about 70%, 75%, 80%, 85%, 90%, 95%, 96%, 97%, 98%, 99%, or 100% sequence identity to an Anellovirus 5′ UTR conserved domain nucleotide sequence, e.g., as described herein.

5′ UTR Regions

In some embodiments, a nucleic acid molecule as described herein (e.g., a genetic element, genetic element construct, or genetic element region) comprises a 5′ UTR sequence, e.g., a 5′ UTR conserved domain sequence as described herein (e.g., in any of Tables A1, B1, or C1), or a sequence having at least 75%, 80%, 85%, 90%, 95%, 96%, 97%, 98%, or 99% sequence identity thereto.

In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGGGX₁CAGTCT, or a nucleic acid sequence having no more than 1, 2, 3, 4, 5, 6, 7, 8, 9, or 10 nucleotide differences (e.g., substitutions, deletions, or additions) relative thereto. In embodiments, X₁ is A. In some embodiments, X₁ is absent.

In some embodiments, the 5′ UTR sequence comprises the nucleic acid sequence of the 5′ UTR of an Alphatorquevirus (e.g., Ring1), or a sequence having at least 75%, 80%, 85%, 90%, 95%, 96%, 97%, 98%, or 99% sequence identity thereto. In some embodiments, the 5′ UTR sequence comprises the nucleic acid sequence of the 5′ UTR conserved domain listed in Table A1, or a sequence having at least 75%, 80%, 85%, 90%, 95%, 96%, 97%, 98%, or 99% sequence identity thereto. In some embodiments, the nucleic acid molecule comprises a nucleic acid sequence having at least 95% sequence identity to the 5′ UTR conserved domain listed in Table A1. In some embodiments, the nucleic acid molecule comprises a nucleic acid sequence having at least 95.775% sequence identity to the 5′ UTR conserved domain listed in Table A1. In some embodiments, the nucleic acid molecule comprises a nucleic acid sequence having at least 97% sequence identity to the 5′ UTR conserved domain listed in Table A1. In some embodiments, the nucleic acid molecule comprises a nucleic acid sequence having at least 97.183% sequence identity to the 5′ UTR conserved domain listed in Table A1. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTTTACACACCGCAGTCAAGGGGCAATTCGGGCTCGGGACTGGC, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of an Betatorquevirus (e.g., Ring2), or a sequence having at least 75%, 80%, 85%, 86%, 87%, 88%, 89%, 90%, 91%, 92%, 93%, 94%, 95%, 96%, 97%, 98%, or 99% sequence identity thereto. In some embodiments, the 5′ UTR sequence comprises the nucleic acid sequence of the 5′ UTR conserved domain listed in Table B1, or a sequence having at least 75%, 80%, 85%, 86%, 87%, 88%, 89%, 90%, 91%, 92%, 93%, 94%, 95%, 96%, 97%, 98%, or 99% sequence identity thereto. In some embodiments, the nucleic acid molecule comprises a nucleic acid sequence having at least 85% sequence identity to the 5′ UTR conserved domain listed in Table B1. In some embodiments, the nucleic acid molecule comprises a nucleic acid sequence having at least 87% sequence identity to the 5′ UTR conserved domain listed in Table B1. In some embodiments, the nucleic acid molecule comprises a nucleic acid sequence having at least 87.324% sequence identity to the 5′ UTR conserved domain listed in Table B1. In some embodiments, the nucleic acid molecule comprises a nucleic acid sequence having at least 88% sequence identity to the 5′ UTR conserved domain listed in Table B1. In some embodiments, the nucleic acid molecule comprises a nucleic acid sequence having at least 88.732% sequence identity to the 5′ UTR conserved domain listed in Table B1. In some embodiments, the nucleic acid molecule comprises a nucleic acid sequence having at least 91% sequence identity to the 5′ UTR conserved domain listed in Table B1. In some embodiments, the nucleic acid molecule comprises a nucleic acid sequence having at least 91.549% sequence identity to the 5′ UTR conserved domain listed in Table B1. In some embodiments, the nucleic acid molecule comprises a nucleic acid sequence having at least 92% sequence identity to the 5′ UTR conserved domain listed in Table B1. In some embodiments, the nucleic acid molecule comprises a nucleic acid sequence having at least 92.958% sequence identity to the 5′ UTR conserved domain listed in Table B1. In some embodiments, the nucleic acid molecule comprises a nucleic acid sequence having at least 94% sequence identity to the 5′ UTR conserved domain listed in Table B1. In some embodiments, the nucleic acid molecule comprises a nucleic acid sequence having at least 94.366% sequence identity to the 5′ UTR conserved domain listed in Table B1. In some embodiments, the nucleic acid molecule comprises a nucleic acid sequence having at least 95% sequence identity to the 5′ UTR conserved domain listed in Table B1. In some embodiments, the nucleic acid molecule comprises a nucleic acid sequence having at least 95.775% sequence identity to the 5′ UTR conserved domain listed in Table B1. In some embodiments, the nucleic acid molecule comprises a nucleic acid sequence having at least 97% sequence identity to the 5′ UTR conserved domain listed in Table B1. In some embodiments, the nucleic acid molecule comprises a nucleic acid sequence having at least 97.183% sequence identity to the 5′ UTR conserved domain listed in Table B1. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAAGTCAAGGGGCAATTCGGGCTAGATCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the 5′ UTR sequence comprises the nucleic acid sequence of the 5′ UTR of an Gammatorquevirus (e.g., Ring4), or a sequence having at least 75%, 80%, 85%, 90%, 95%, 96%, 97%, 98%, or 99% sequence identity thereto. In some embodiments, the 5′ UTR sequence comprises the nucleic acid sequence of the 5′ UTR conserved domain listed in Table C1, or a sequence having at least 75%, 80%, 85%, 90%, 95%, 96%, 97%, 98%, or 99% sequence identity thereto. In some embodiments, the nucleic acid molecule comprises a nucleic acid sequence having at least 97% sequence identity to the 5′ UTR conserved domain listed in Table C1. In some embodiments, the nucleic acid molecule comprises a nucleic acid sequence having at least 97.183% sequence identity to the 5′ UTR conserved domain listed in Table C1. In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having at least 85%, 90%, 95%, 96%, 97%, 98%, or 99% sequence identity thereto. In some embodiments, the 5′ UTR sequence comprises the nucleic acid sequence AGGTGAGTGAAACCACCGAGGTCTAGGGGCAATTCGGGCTAGGGCAGTCT, or a nucleic acid sequence having no more than 1, 2, 3, 4, 5, 6, 7, 8, 9, or 10 nucleotide differences (e.g., substitutions, deletions, or additions) relative thereto.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Anellovirus 5′ UTR sequence, e.g., a nucleic acid sequence shown in Table 38. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

TABLE 38
Exemplary 5’ UTR sequences from Anelloviruses

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = G or A | |
| | X₄ = T or C | |
| | X₅ = A, C, or T | |
| Exemplary TTV | CGGGTGCCGGAGGTGAGTTTACACACCGCAGTC | 106 |
| Sequence | AAGGGGCAATTCGGGCTCGGGACTGGCCGGGCT | |
| | WTGGG | |
| TTV-CT30F | CGGGTGCCGTAGGTGAGTTTACACACCGCAGTC | 107 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 108 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 109 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 110 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 111 |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 112 |
| Consensus 5’ UTR | AAGGGGCAATTCGGGCTCGGGACTGGCCGGGC | |
| | X₁X₂TGGG; wherein X₁ comprises T or C, and | |
| | wherein X₂ comprises A, C, or T. | |
| Alphatorquevirus | CGGGTGCCGTAGGTGAGTTTACACACCGCAGTC | 113 |
| Clade 1 5’ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 114 |
| Clade 2 5’ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 115 |
| Clade 3 5’ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 116 |
| Clade 4 5’ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 117 |
| Clade 5 5’ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 118 |
| Clade 6 5’ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 119 |
| Clade 7 5’ UTR (e.g., | AAGGGGCAATTCGGGCTCGGGACTGGCCGGGCT | |
| TTV-HD16d) | ATGGG | |

Identification of 5′ UTR Sequences

In some embodiments, an Anellovirus 5′ UTR sequence can be identified within the genome of an Anellovirus (e.g., a putative Anellovirus genome identified, for example, by nucleic acid sequencing techniques, e.g., deep sequencing techniques). In some embodiments, an Anellovirus 5′ UTR sequence is identified by one or both of the following steps:

(i) Identification of circularization junction point: In some embodiments, a 5′ UTR will be positioned near a circularization junction point of a full-length, circularized Anellovirus genome. A circularization junction point can be identified, for example, by identifying overlapping regions of the sequence. In some embodiments, a overlapping region of the sequence can be trimmed from the sequence to produce a full-length Anellovirus genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anellovirus genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anellovirus, e.g., as described herein).

(ii) Identification of 5′ UTR sequence: Once a putative Anellovirus genome sequence has been obtained, the sequence (or portions thereof, e.g., having a length between about 40-50, 50-60, 60-70, 70-80, 80-90, or 90-100 nucleotides) can be compared to one or more Anellovirus 5′ UTR sequences (e.g., as described herein) to identify sequences having substantial homology thereto. In some embodiments, a putative Anellovirus 5′ UTR region has at least 50%, 60%, 70%, 75%, 80%, 85%, 90%, 95%, 96%, 97%, 98%, 99%, or 100% sequence identity to an Anellovirus 5′ UTR sequence as described herein.

GC-Rich Regions

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In some embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39. In embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39.

In some embodiments, the 36-nucleotide GC-rich sequence is selected from:
(i) CGCGCTGCGCGCGCCGCCCAGTAGGGGGAGCCATGC (SEQ ID NO: 160),
(ii) GCGCTX₁CGCGCGCGCGCCGGGGGGCTGCGCCCCCCC (SEQ ID NO: 164), wherein X₁ is selected from T, G, or A;
(iii) GCGCTTCGCGCGCCGCCCACTAGGGGGCGTTGCGCG (SEQ ID NO: 165);
(iv) GCGCTGCGCGCGCCGCCCAGTAGGGGGCGCAATGCG (SEQ ID NO: 166);
(v) GCGCTGCGCGCGCGGCCCCCGGGGGAGGCATTGCCT (SEQ ID NO: 167);
(vi) GCGCTGCGCGCGCGCGCCGGGGGGGCGCCAGCGCCC (SEQ ID NO: 168);
(vii) GCGCTTCGCGCGCGCGCCGGGGGGCTCCGCCCCCCC (SEQ ID NO: 169);
(viii) GCGCTTCGCGCGCGCGCCGGGGGGCTGCGCCCCCCC (SEQ ID NO: 170);
(ix) GCGCTACGCGCGCGCGCCGGGGGGCTGCGCCCCCCC (SEQ ID NO: 171); or
(x) GCGCTACGCGCGCGCGCCGGGGGGCTCTGCCCCCCC (SEQ ID NO: 172).
   In some embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In some embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CGCGC | 120 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X | |
| | | ₃₃GGGGGGCTCCGX₃₄CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 121 |
| Sequence | | DCGCGCGCSNNNCRCCRGGGGGNNNNCWG | |
| | | CSNCNCCCCCCCCCGCGCATGCGCGGGKCC | |
| | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 122 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 123 |
| | | CG | |
| | Fragment 3 | CCCCCCGGCCCCCCCCCGTGCTAAACCCAC | 124 |
| | | CGCGCATGCGCGACCACGCCCCCGCCGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 126 |
| | Fragment 2 | GGGGGCG | 127 |
| | Fragment 3 | GCCGCG | 128 |
| | Fragment 4 | TTCGCGCGCCGCCCACCAGGGGGTG | 129 |
| | Fragment 5 | CTGCG | 130 |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCCCCC | 133 |
| | | GTGCTAAACCCACCGCGCATGCGCGACCAC | |
| | | GCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | GCCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGCGCCAGCG | 137 |
| | Fragment 4 | CCCCCCCCCCCGCGCAT | 138 |
| | Fragment 5 | GCACGGGTCCCCCCCCCCACGGGGGGCTCC | 139 |
| | | G | |
| | Fragment 6 | CCCCCCGGCCCCCCCCC | 140 |
| TTV-JA20 | Full sequence | CCGTCGGCGGGGGGGCCGCGCGCTGCGCG | 141 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 142 |
| | | CGCGGCCC | |
| | Fragment 2 | CCGGGGGAGGCACAGCCTCCCCCCCCCGCG | 143 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG-- | 144 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | 148 |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTAGG | |
| | | GGGAGCCATGCG---CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTAGG | 155 |
| | | GGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGGGA | 160 |
| | | GCCATGC | |
| | Fragment 8 | CCGCCATCTTAAGTAGTTGAGGCGGACGGT | 161 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 162 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional GC- | 36-nucleotide | CGCGCTGCGCGCGCCGCCCAGTAGGGGGA | 163 |
| rich | consensus GC- | GCCATGC | |
| Sequences | rich region | | |
| | sequence 1 | | |
| | 36-nucleotide | GCGCTX₁CGCGCGCGCGCCGGGGGGCTGCG | 164 |
| | region | CCCCCCC, wherein X₁ is selected | |
| | consensus | from T, G, or A | |
| | sequence 2 | | |
| | TTV Clade 1 | GCGCTTCGCGCGCCGCCCACTAGGGGGCGT | 165 |
| | 36-nucleotide | TGCGCG | |
| | region | | |
| | TTV Clade 3 | GCGCTGCGCGCGCCGCCCAGTAGGGGGCG | 166 |
| | 36-nucleotide | CAATGCG | |
| | region | | |
| | TTV Clade 3 | GCGCTGCGCGCGCGGCCCCCGGGGGAGGC | 167 |
| | isolate GH1 36- | ATTGCCT | |
| | nucleotide | | |
| | region | | |
| | TTV Clade 3 | GCGCTGCGCGCGCGCGCCGGGGGGGCGCC | 168 |
| | sle1932 36- | AGCGCCC | |
| | nucleotide | | |
| | region | | |
| | TTV Clade 4 | GCGCTTCGCGCGCGCGCCGGGGGGCTCCGC | 169 |
| | ctdc002 36- | CCCCCC | |
| | nucleotide | | |
| | region | | |
| | TTV Clade 5 | GCGCTTCGCGCGCGCGCCGGGGGGCTCCGC | 170 |
| | 36-nucleotide | CCCCCC | |
| | region | | |
| | TTV Clade 6 | GCGCTACGCGCGCGCGCCGGGGGGCTGCG | 171 |
| | 36-nucleotide | CCCCCCC | |
| | region | | |
| | TTV Clade 7 | GCGCTACGCGCGCGCGCCGGGGGGCTCTGC | 172 |
| | 36-nucleotide | CCCCCC | |
| | region | | |
| Additional | TTV-CT30F | GCGGCGGGGGGGCGGCCGCGTTCGCGCGC | 801 |
| Alpha- | | CGCCCACCAGGGGGTGCTGCGCGCCCCCCC | |
| torquevirus | | CCGCGCATGCGCGGGGCCCCCCCCCGGGG | |
| GC-rich region | | GGGCTCCGCCCCCCCGGCCCCCCCCCGTGC | |
| sequences | | TAAACCCACCGCGCATGCGCGACCACGCCC | |
| | | CCGCCGCC | |
| | TTV-P13-1 | CCGAGCGTTAGCGAGGAGTGCGACCCTACC | 802 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 803 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 804 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 805 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 806 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 807 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

Effectors

In some embodiments, the genetic element may include one or more sequences that encode an effector, e.g., a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described herein. In some embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In some embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF as described herein, e.g., an ORF1, ORF1/1, ORF1/2, ORF2, ORF2/2, ORF2/3, and/or ORF2t/3).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described herein.

Regulatory Nucleic Acids

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In some embodiments, the regulatory nucleic acid encodes an miRNA. In some embodiments, the regulatory nucleic acid is endogenous to a wild-type Anellovirus. In some embodiments, the regulatory nucleic acid is exogenous to a wild-type Anellovirus.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein, e.g., in Table 40.

TABLE 40
Examples of regulatory nucleic acids, e.g., miRNAs.

| Accession | Exemplary | | SEQ | miRNA_ | SEQ | miRNA_ | SEQ |
|---|---|---|---|---|---|---|---|
| number of | subsequence | | ID | 5prime_per_ | ID | 3prime_per_ | ID |
| strain | nucleotides | Pre_miRNA | NO: | MiRdup | NO: | MiRdup | NO: |
| AB008394.1 | AB008394_ | GCCAUUUUAAGUA | 300 | AGUAGCUGAC | 395 | CAUCCUCGGC | 490 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AB008394.1 | AB008394_ | GCGUACGUCACAA | 301 | CAAGUCACGU | 396 | GGCCCCGUCA | 491 |
| | 3579_3657 | GUCACGUGGAGGG | | GGAGGGGACC | | CGUGACUUAC | |
| | | GACCCGCUGUAAC | | CG(5′) | | CAC(3′) | |
| | | CCGGAAGUAGGCC | | | | | |
| | | CCGUCACGUGACU | | | | | |
| | | UACCACGUGUGUA | | | | | |
| AB017613.1 | AB017613_ | GCCAUUUUAAGUA | 302 | AAGUAGCUGA | 397 | UCAUCCUCGG | 492 |
| | 3462_3539 | GCUGACGUCAAGG | | CGUCAAGGAU | | CGGAAGCUAC | |
| | | AUUGACGUGAAGG | | UGACG(5′) | | ACAA(3′) | |
| | | UUAAAGGUCAUCC | | | | | |
| | | UCGGCGGAAGCUA | | | | | |
| | | CACAAAAUGGUG | | | | | |
| AB017613.1 | AB017613_ | GCACACGUCAUAA | 303 | AUAAGUCACG | 398 | GGCCCCGUCA | 493 |
| | 3566_3644 | GUCACGUGGUGGG | | UGGUGGGGAC | | CGUGAUUUGU | |
| | | GACCCGCUGUAAC | | CCG(5′) | | CAC(3′) | |
| | | CCGGAAGUAGGCC | | | | | |
| | | CCGUCACGUGAUU | | | | | |
| | | UGUCACGUGUGUA | | | | | |
| AB025946.1 | AB025946_ | CUUCCGGGUCAUA | 304 | UGGGGAGGGU | 399 | CCGGGUCAUA | 494 |
| | 3534_3600 | GGUCACACCUACG | | UGGCGUAUAG | | GGUCACACCU | |
| | | UCACAAGUCACGU | | CCCGGA(3′) | | ACGUCAC(5′) | |
| | | GGGGAGGGUUGGC | | | | | |
| | | GUAUAGCCCGGAA | | | | | |
| | | G | | | | | |
| AB025946.1 | AB025946_ | GCCGGGGGGCUGC | 305 | CCCCCCCCGG | 400 | GGCUGCCGCC | 495 |
| | 3730_3798 | CGCCCCCCCCGGG | | GGGGGGGUUU | | CCCCCCGGGG | |
| | | GAAAGGGGGGGGC | | GCCC(3′) | | AAAGGGGG(5′) | |
| | | CCCCCCCGGGGGG | | | | | |
| | | GGGUUUGCCCCCC | | | | | |
| | | GGC | | | | | |
| AB028668.1 | AB028668_ | AUACGUCAUCAGU | 306 | AUCAGUCACG | 401 | AUCCUCGUCC | 496 |
| | 3537_3615 | CACGUGGGGGAAG | | UGGGGGAAGG | | ACGUGACUGU | |
| | | GCGUGCCUAAACC | | CGUGC(5′) | | GA(3′) | |
| | | CGGAAGCAUCCUC | | | | | |
| | | GUCCACGUGACUG | | | | | |
| | | UGACGUGUGUGGC | | | | | |
| AB028669.1 | AB028669_ | CAUUUUAAGUAAG | 307 | AAGUAAGGCG | 402 | GAGCACUUCC | 497 |
| | 3440_3513 | GCGGAAGCAGCUC | | GAAGCAGCUC | | GGCUUGCCCA | |
| | | GGCGUACACAAAA | | GG(5′) | | A(3′) | |
| | | UGGCGGCGGAGCA | | | | | |
| | | CUUCCGGCUUGCC | | | | | |
| | | CAAAAUGG | | | | | |
| AB028669.1 | AB028669_ | GUCACAAGUCACG | 308 | AGUCACGUGG | 403 | CAAUCCUCUU | 498 |
| | 3548_3619 | UGGGGAGGGUUGG | | GGAGGGUUGG | | ACGUGGCCUG | |
| | | CGUUUAACCCGGA | | C(5′) | | (3′) | |
| | | AGCCAAUCCUCUU | | | | | |
| | | ACGUGGCCUGUCA | | | | | |
| | | CGUGAC | | | | | |
| AB037926.1 | AB037926_ | CGACCGCGUCCCG | 309 | CCCGAAGGCG | 404 | CGAGGUUAAG | 499 |
| | 162_232 | AAGGCGGGUACCC | | GGUACCCGAG | | GGCCAAUUCG | |
| | | GAGGUGAGUUUAC | | GU(5′) | | GGCU(3′) | |
| | | ACACCGAGGUUAA | | | | | |
| | | GGGCCAAUUCGGG | | | | | |
| | | CUUGG | | | | | |
| AB037926.1 | AB037926_ | CGCGGUAUCGUAG | 310 | UAUCGUAGCC | 405 | GGGCCCCCGC | 500 |
| | 3454_3513 | CCGACGCGGACCC | | GACGCGGACC | | GGGGCUCUCG | |
| | | CGUUUUCGGGGCC | | CCG(5′) | | GCG(3′) | |
| | | CCCGCGGGGCUCU | | | | | |
| | | CGGCGCG | | | | | |
| AB037926.1 | AB037926_ | CGCCAUUUUGUGA | 311 | AUUUUGUGAU | 406 | GCGGGGCGUG | 501 |
| | 3531_3609 | UACGCGCGUCCCC | | ACGCGCGUCC | | GCCGUAUCAG | |
| | | UCCCGGCUUCCGU | | CCUCCC(5′) | | AAAAUGG(3′) | |
| | | ACAACGUCAGGCG | | | | | |
| | | GGGCGUGGCCGUA | | | | | |
| | | UCAGAAAAUGGCG | | | | | |
| AB037926.1 | AB037926_ | GCUACGUCAUAAG | 312 | AAGUCACGUG | 407 | CCUCGGUCAC | 502 |
| | 3637_3714 | UCACGUGACUGGG | | ACUGGGCAGG | | GUGGCCUGU(3′) | |
| | | CAGGUACUAAACC | | U(5′) | | | |
| | | CGGAAGUAUCCUC | | | | | |
| | | GGUCACGUGGCCU | | | | | |
| | | GUCACGUAGUUG | | | | | |
| AB038621.1 | AB038621_ | GGCUSUGAGGUCA | 313 | UGACGUCAAA | 408 | CCUCGUCACG | 503 |
| | 3511_3591 | AAGUCACGUGGGR | | GUCACGUGGG | | UGACCUGACG | |
| | | AGGGUGGCGUUAA | | RAGGGU(5′) | | UCACAG(3′) | |
| | | ACCCGGAAGUCAU | | | | | |
| | | CCUCGUCACGUGA | | | | | |
| | | CCUGACGUCACAG | | | | | |
| | | CC | | | | | |
| AB038622.1 | AB038622_ | GCCCGUCCGCGGC | 314 | GAUCGAGCGU | 409 | CCGUCCGCGG | 504 |
| | 227_293 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGAAGG | | | | | |
| | | U | | | | | |
| AB038622.1 | AB038622_ | GGUUGUGACGUCA | 315 | UGACGUCAAA | 410 | AUCCUCGUCA | 505 |
| | 3510_3591 | AAGUCACGUGGGG | | GUCACGUGGG | | CGUGACCUGA | |
| | | AGGGCGGCGUUAA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB038623.1 | AB038623_ | GCCCGUCCGCGGC | 316 | GAUCGAGCGU | 411 | CCGUCCGCGG | 506 |
| | 228_295 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB038624.1 | AB038624_ | GCCCGUCCGCGGC | 317 | GAUCGAGCGU | 412 | CCGUCCGCGG | 507 |
| | 228_295 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB038624.1 | AB038624_ | GGCUGUGACGUCA | 318 | UGACGUCAAA | 413 | AUCCUCGUCA | 508 |
| | 3511_3592 | AAGUCACGUGGGG | | GUCACGUGGG | | CGUGACCUGA | |
| | | AGGGCGGCGUUAA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB041957.1 | AB041957_ | AGACCACGUGGUA | 319 | ACGUGGUAAG | 414 | CUGACCCGCG | 509 |
| | 3414_3493 | AGUCACGUGGGGG | | UCACGUGGGG | | UGACUGGUCA | |
| | | CAGCUGCUGUAAA | | GCAGCU(5′) | | CGUGA(3′) | |
| | | CCCGGAAGUAGCU | | | | | |
| | | GACCCGCGUGACU | | | | | |
| | | GGUCACGUGACCU | | | | | |
| | | G | | | | | |
| AB049608.1 | AB049608_ | CGCCAUUUUAUAA | 320 | AUUUUAUAAU | 415 | CGGGGCGUGG | 510 |
| | 3199_3277 | UACGCGCGUCCCC | | ACGCGCGUCC | | CCGUAUUAGA | |
| | | UCCCGGCUUCCGU | | CCUCC(5′) | | AAAUGG(3′) | |
| | | ACUACGUCAGGCG | | | | | |
| | | GGGCGUGGCCGUA | | | | | |
| | | UUAGAAAAUGGUG | | | | | |
| AB050448.1 | AB050448_ | UAAGUAAGGCGGA | 321 | AAGGGACAGC | 416 | AGUAAGGCGG | 511 |
| | 3393_3465 | ACCAGGCUGUCAC | | CUUCCGGCUU | | AACCAGGCUG | |
| | | CCUGUGUCAAAGG | | GC(3′) | | UCACCCUGU(5′) | |
| | | UCAAGGGACAGCC | | | | | |
| | | UUCCGGCUUGCAC | | | | | |
| | | AAAAUGG | | | | | |
| AB054647.1 | AB054647_ | UGCCUACGUCAUA | 322 | GAUAAGUCAC | 417 | UAGCUGACCC | 512 |
| | 3537_3615 | AGUCACGUGGGGA | | GUGGGGACGG | | GCGUGACUUG | |
| | | CGGCUGCUGUAAA | | CUGCU(5′) | | UCAC(3′) | |
| | | CACGGAAGUAGCU | | | | | |
| | | GACCCGCGUGACU | | | | | |
| | | UGUCAGGUGAGCA | | | | | |
| AB054648.1 | AB054648_ | UUGUGUAAGGCGG | 323 | UAAGGCGGAA | 418 | GGUCAGCCUC | 513 |
| | 3439_3511 | AACAGGCUGACAC | | CAGGCUGACA | | CGCUUUGCA(3′) | |
| | | CCCGUGUCAAAGG | | CCCC(5′) | | | |
| | | UCAGGGGUCAGCC | | | | | |
| | | UCCGCUUUGCACC | | | | | |
| | | AAAUGGU | | | | | |
| AB054648.1 | AB054648_ | UACCUACGUCAUAA | 324 | UACGUCAUAA | 419 | GCUGACCCGC | 514 |
| | 3538_3617 | GUCACGUGGGAAG | | GUCACGUGGG | | GUGGCUUGUC | |
| | | AGCUGCUGUGAAC | | AAGAGCUG(5′) | | ACGUGAGU(3′) | |
| | | CUGGAAGUAGCUG | | | | | |
| | | ACCCGCGUGGCUU | | | | | |
| | | GUCACGUGAGUGC | | | | | |
| AB064595.1 | AB064595_ | UUUUCCUGGCCCG | 325 | UCGGGCGUCC | 420 | GGCCCGUCCG | 515 |
| | 116_191 | UCCGCGGCGAGAG | | CGAGGGCGGG | | CGGCGAGAGC | |
| | | CGCGAGCGAAGCG | | UG(3′) | | GCGAG(5′) | |
| | | AGCGAUCGGGCGU | | | | | |
| | | CCCGAGGGCGGGU | | | | | |
| | | GCCGGAGGUG | | | | | |
| AB064595.1 | AB064595_ | AAAGUGAGUGGGG | 326 | AAAGUGAGUG | 421 | UCCGGGUGCG | 516 |
| | 3283_3351 | CCAGACUUCGCCA | | GGGCCAGACU | | UCUGGGGGCC | |
| | | UAGGGCCUUUAAC | | UCGCC(5′) | | GCCAUUU(3′) | |
| | | UUCCGGGUGCGUC | | | | | |
| | | UGGGGGCCGCCAU | | | | | |
| | | UUU | | | | | |
| AB064595.1 | AB064595_ | GUGACGUUACUCU | 327 | CUCUCACGUG | 422 | AUCCUCGACC | 517 |
| | 3427_3500 | CACGUGAUGGGGG | | AUGGGGGCGU | | AGGUGAGUGU | |
| | | CGUGCUCUAACCC | | GC(5′) | | G(3′) | |
| | | GGAAGCAUCCUCG | | | | | |
| | | ACCACGUGACUGU | | | | | |
| | | GAOGUCAC | | | | | |
| AB064595.1 | AB064595_ | AGCGUCUACUACG | 328 | UCUACUACGU | 423 | AUAAACCAGA | 518 |
| | 41_116 | UACACUUCCUGGG | | ACACUUCCUG | | GGGGUGACGA | |
| | | GUGUGUCCUGCCA | | GGGUGUGU(5′) | | AUGGUAGAGU(3′) | |
| | | CUGUAUAUAAACCA | | | | | |
| | | GAGGGGUGACGAA | | | | | |
| | | UGGUAGAGU | | | | | |
| AB064596.1 | AB064596_ | GUGACGUCAAAGU | 329 | UGGCUGUUGU | 424 | CAAAGUCACG | 519 |
| | 3424_3497 | CAGGUGGUGACGG | | CAGGUGACUU | | UGGUGACGGC | |
| | | CCAUUUUAACCCG | | GA(3′) | | CAU(5′) | |
| | | GAAGUGGCUGUUG | | | | | |
| | | UCACGUGACUUGA | | | | | |
| | | CGUCACGG | | | | | |
| AB064597.1 | AB064597_ | GCUUUAGACGCCA | 330 | AGACGCCAUU | 425 | GUAGGCGCGU | 520 |
| | 3191_3253 | UUUUAGGCCCUCG | | UUAGGCCCUC | | UUUAAUGACG | |
| | | CGGGCACCCGUAG | | GCGG(5′) | | UCACGG(3′) | |
| | | GCGCGUUUUAAUG | | | | | |
| | | ACGUCACGGC | | | | | |
| AB064597.1 | AB064597_ | CACCCGUAGGCGC | 331 | UGUCGUGACG | 426 | UAGGCGCGUU | 521 |
| | 3221_3294 | GUUUUAAUGACGU | | UUUGAGACAC | | UUAAUGACGU | |
| | | CACGGCAGCCAUU | | GUGAU(3′) | | CACGGCAG(5′) | |
| | | UUGUCGUGACGUU | | | | | |
| | | UGAGACACGUGAU | | | | | |
| | | GGGGGCGU | | | | | |
| AB064597.1 | AB064597_ | GUCGUGACGUUUG | 332 | UGACGUUUGA | 427 | AUCCCUGGUC | 522 |
| | 3262_3342 | AGACACGUGAUGG | | GACACGUGAU | | ACGUGACUCU | |
| | | GGGCGUGCCUAAA | | GGGGGCGUGC | | GACGUCACG(3′) | |
| | | CCCGGAAGCAUCC | | (5′) | | | |
| | | CUGGUCACGUGAC | | | | | |
| | | UCUGACGUCACGG | | | | | |
| | | CG | | | | | |
| AB064598.1 | AB064598_ | CGAAAGUGAGUGG | 333 | AGUGAGUGGG | 428 | GCGUGUGGGG | 523 |
| | 3179_3256 | GGCCAGACUUCGC | | GCCAGACUUC | | GCCGCCAUUU | |
| | | CAUAAGGCCUUUA | | GC(5′) | | UAGCUU(3′) | |
| | | ACUUCCGGGUGCG | | | | | |
| | | UGUGGGGGCCGCC | | | | | |
| | | AUUUUAGCUUCG | | | | | |
| AB064598.1 | AB064598_ | CUGUGACGUCAAA | 334 | UGUGAGGUCA | 429 | UCAUCCUCGU | 524 |
| | 3323_3399 | GUCACGUGGGGAG | | AAGUCACGUG | | CACGUGACCU | |
| | | GGCGGCGUGUAAC | | GGGAGGGCGG | | GACGUCACG(3′) | |
| | | CCGGAAGUCAUCC | | (5′) | | | |
| | | UCGUCACGUGACC | | | | | |
| | | UGACGUCACGG | | | | | |
| AB064598.1 | AB064598_ | CUGUCCGCCAUCU | 335 | AAAAGAGGAA | 430 | CGCCAUCUUG | 525 |
| | 3412_3485 | UGUGACUUCCUUC | | GUAUGACGUA | | UGACUUCCUU | |
| | | CGCUUUUUCAAAAA | | GCGGCGG(3′) | | CCGCUUUUU(5′) | |
| | | AAAAGAGGAAGUAU | | | | | |
| | | GACGUAGCGGCGG | | | | | |
| | | GGGGGC | | | | | |
| AB064599.1 | AB064599_ | GGUAGAGUUUUUU | 336 | AGCGAGCGGC | 431 | UAGAGUUUUU | 526 |
| | 108_175 | CCGCCCGUCCGCA | | CGAGCGACCC | | UCCGCCCGUC | |
| | | GCGAGGACGCGAG | | G(3′) | | CG(5′) | |
| | | CGCAGCGAGCGGC | | | | | |
| | | CGAGCGACCCGUG | | | | | |
| | | GG | | | | | |
| AB064599.1 | AB064599_ | GCUGUGACGUUUC | 337 | UUCAGUCACG | 432 | GUCCCUGGUC | 527 |
| | 3389_3469 | AGUCACGUGGGGA | | UGGGGAGGGA | | ACGUGAUUGU | |
| | | GGGAACGCCUAAA | | ACGC(5′) | | GAC(3′) | |
| | | CCCGGAAGCGUCC | | | | | |
| | | CUGGUCACGUGAU | | | | | |
| | | UGUGACGUCACGG | | | | | |
| | | CC | | | | | |
| AB064599.1 | AB064599_ | CCGCCAUUUUGUG | 338 | AAAAGAGGAA | 433 | CAUUUUGUGA | 528 |
| | 3483_3546 | ACUUCCUUCCGCU | | GUGUGACGUA | | CUUCCUUCCG | |
| | | UUUUCAAAAAAAAA | | GCGG(3′) | | CUUUUU(5′) | |
| | | GAGGAAGUGUGAC | | | | | |
| | | GUAGCGGCGG | | | | | |
| AB064600.1 | AB064600_ | GACUGUGACGUCA | 339 | UGUGACGUCA | 434 | UCAUCCUCGU | 529 |
| | 3378_3456 | AAGUCACGUGGGG | | AAGUCACGUG | | CACGUGACCU | |
| | | AGGGCGGCGUGUA | | GGGAGGGCGG | | GACGUCACG(3′) | |
| | | ACCCGGAAGUCAU | | (5′) | | | |
| | | CCUCGUCACGUGA | | | | | |
| | | CCUGACGUCACGG | | | | | |
| AB064600.1 | AB064600_ | CUGUCCGCCAUCU | 340 | AAAAGAGGAA | 435 | CCGCCAUCUU | 530 |
| | 3469_3542 | UGUGACUUCCUUC | | GUAUGACGUG | | GUGACUUCCU | |
| | | CGCUUUUUCAAAAA | | GCGG(3′) | | UCCGCUUUUU | |
| | | AAAAGAGGAAGUAU | | | | (5′) | |
| | | GACGUGGCGGCGG | | | | | |
| | | GGGGGC | | | | | |
| AB064601.1 | AB064601_ | GGUUGUGACGUCA | 341 | UGACGUCAAA | 436 | AUCCUCGUCA | 531 |
| | 3318_3398 | AAGUCACGUGGGG | | GUCACGUGGG | | CGUGACCUGA | |
| | | AGGGCGGCGUGUA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB064601.1 | AB064601_ | CCCGCCAUCUUGU | 342 | AAAAAAGAGG | 437 | CGCCAUCUUG | 532 |
| | 3412_3477 | GACUUCCUUCCGC | | AAGUGUGACG | | UGACUUCCUU | |
| | | UUUUUCAAAAAAAA | | UAGCGGCGG | | CCGCUUUUUC | |
| | | AGAGGAAGUGUGA | | (3′) | | (5′) | |
| | | CGUAGCGGCGGG | | | | | |
| AB064602.1 | AB064602_ | GCCCGUCCGCGGC | 343 | GAUCGAGCGU | 438 | CCGUCCGCGG | 533 |
| | 125_192 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB064602.1 | AB064602_ | GACUGUGACGUCA | 344 | UGUGACGUCA | 439 | UCAUCCUCGU | 534 |
| | 3368_3446 | AAGUCACGUGGGG | | AAGUCACGUG | | CACGUGACCU | |
| | | AGGAGGGCGUGUA | | GGGAGGAGGG | | GACGUCACG(3′) | |
| | | ACCCGGAAGUCAU | | (5′) | | | |
| | | CCUCGUCACGUGA | | | | | |
| | | CCUGACGUCACGG | | | | | |
| AB064603.1 | AB064603_ | UCGCGUCUUAGUG | 345 | UUGGUCCUGA | 440 | CUUAGUGACG | 535 |
| | 3385_3447 | ACGUCACGGCAGC | | CGUCACUGUC | | UCACGGCAGC | |
| | | CAUCUUGGUCCUG | | A(3′) | | CAU(5′) | |
| | | AGGUCACUGUCAC | | | | | |
| | | GUGGGGAGGG | | | | | |
| AB064603.1 | AB064603_ | UGACGUCACUGUC | 346 | CGUCACUGUC | 441 | GUCCCUGGUC | 536 |
| | 3422_3498 | ACGUGGGGAGGGA | | ACGUGGGGAG | | ACGUGACAUG | |
| | | ACACGUGAACCCG | | GGAACAC(5′) | | ACGUC(3′) | |
| | | GAAGUGUCCCUGG | | | | | |
| | | UCACGUGACAUGA | | | | | |
| | | CGUCACGGCCG | | | | | |
| AB064604.1 | AB064604_ | CGCCAUUUUAAGU | 347 | UAAGUAAGCA | 442 | CACAGCCGGU | 537 |
| | 3436_3514 | AAGCAUGGCGGGC | | UGGCGGGCGG | | CAUGCUUGCA | |
| | | GGUGAUGUCAAAU | | UGAU(5′) | | CAAA(3′) | |
| | | GUUAAAGGUCACA | | | | | |
| | | GCCGGUCAUGCUU | | | | | |
| | | GCACAAAAUGGCG | | | | | |
| AB064605.1 | AB064605_ | CGCCAUUUUAAGU | 348 | AAGUAAGCAU | 443 | ACAGCCUGUC | 538 |
| | 3440_3518 | AAGCAUGGCGGGC | | GGCGGGCGGU | | AUGCUUGCAC | |
| | | GGUGACGUGCAAU | | GA(5′) | | AA(3′) | |
| | | GUCAAAGGUCACA | | | | | |
| | | GCCUGUCAUGCUU | | | | | |
| | | GCACAAAAUGGCG | | | | | |
| AB064606.1 | AB064606_ | CCAUCUUAAGUAG | 349 | UAAGUAGUUG | 444 | CACCAUCAGC | 539 |
| | 3377_3449 | UUGAGGCGGACGG | | AGGCGGACGG | | CACACCUACU | |
| | | UGGCGUCGGUUCA | | UGGC(5′) | | CAAA(3′) | |
| | | AAGGUCACCAUCA | | | | | |
| | | GCCACACCUACUC | | | | | |
| | | AAAAUGG | | | | | |
| AB064607.1 | AB064607_ | GCCUGUCAUGCUU | 350 | UCAUGCUUGC | 445 | CGGGUCGCCG | 540 |
| | 3502_3569 | GCACAAAAUGGCG | | ACAAAAUGGC | | CCAUAUUUGG | |
| | | GACUUCCGCUUCC | | GGACUUCCG | | UCACGUGA(3′) | |
| | | GGGUCGCCGCCAU | | (5′) | | | |
| | | AUUUGGUCACGUG | | | | | |
| | | AC | | | | | |
| AF079173.1 | AF079173_ | GCCAUUUUAAGUA | 351 | AGUAGCUGAC | 446 | CAUCCUCGGC | 541 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF116842.1 | AF116842_ | GCCAUUUUAAGUA | 352 | AGUAGCUGAC | 447 | CAUCCUCGGC | 542 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF116842.1 | AF116842_ | GCAUAGGUCACAA | 353 | ACAAGUCACG | 448 | GGCCCCGUCA | 543 |
| | 3579_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCAGGUGUGUA | | | | | |
| AF122913.1 | AF122913_ | GCCAUUUUAAGUA | 354 | AAGUAGCUGA | 449 | UCAUCCUCGG | 544 |
| | 3475_3551 | GCUGACGUCAAGG | | CGUGAAGGAU | | CGGAAGCUAC | |
| | | AUUGACGUGAAGG | | UGACG(5′) | | ACAA(3′) | |
| | | UUAAAGGUCAUCC | | | | | |
| | | UCGGCGGAAGCUA | | | | | |
| | | CACAAAAUGGU | | | | | |
| AF122913.1 | AF122913_ | GCACACGUCAUAA | 355 | AUAAGUCACG | 450 | GGCCCCGUCA | 545 |
| | 3579_3657 | GUCACGUGGUGGG | | UGGUGGGGAC | | CGUGAUUUGU | |
| | | GACCCGCUGUAAC | | CCG(5′) | | CAC(3′) | |
| | | CCGGAAGUAGGCC | | | | | |
| | | CCGUCACGUGAUU | | | | | |
| | | UGUCACGUGUGUA | | | | | |
| AF122914.1 | AF122914_ | GCCAUUUUAAGUC | 356 | AAGUCAGCUC | 451 | GUCAUCCUCA | 546 |
| | 3476_3552 | AGCUCUGGGGAGG | | UGGGGAGGCG | | CCAUAACUGG | |
| | | CGUGACUUCCAGU | | UGACUU(5′) | | CACAA(3′) | |
| | | UCAAAGGUCAUCC | | | | | |
| | | UCACCAUAACUGG | | | | | |
| | | CACAAAAUGGC | | | | | |
| AF122915.1 | AF122915_ | GCCAUUUUAAGUA | 357 | AGUAGCUGAC | 452 | CAUCCUCGGC | 547 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF122915.1 | AF122915_ | GCAUAGGUCACAA | 358 | CAAGUCACGU | 453 | GGCCCCGUCA | 548 |
| | 3579_3657 | GUCACGUGGAGGG | | GGAGGGGACA | | CGUGACUUAC | |
| | | GACACGCUGUAAC | | CG(5′) | | CAC(3′) | |
| | | CCGGAAGUAGGCC | | | | | |
| | | CCGUCACGUGACU | | | | | |
| | | UACCAGGUGUGUA | | | | | |
| AF122916.1 | AF122916_ | GCGCCAUGUUAAG | 359 | UGUUAAGUGG | 454 | AUCCUCGACG | 549 |
| | 3458_3537 | UGGCUGUCGCCGA | | CUGUCGCCGA | | GUAACCGCAA | |
| | | GGAUUGACGUCAC | | GGAUUGA(5′) | | ACAUG(3′) | |
| | | AGUUCAAAGGUCA | | | | | |
| | | UCCUCGACGGUAA | | | | | |
| | | CCGCAAACAUGGC | | | | | |
| | | G | | | | | |
| AF122916.1 | AF122916_ | CAUGCGUCAUAAG | 360 | UAAGUCACAU | 455 | GGCCCCGACA | 550 |
| | 3565_3641 | UCACAUGACAGGG | | GACAGGGGUC | | UGUGACUCGU | |
| | | GUCCACUUAAACAC | | CA(5′) | | C(3′) | |
| | | GGAAGUAGGCCCC | | | | | |
| | | GACAUGUGACUCG | | | | | |
| | | UCACGUGUGU | | | | | |
| AF122916.1 | AF122916_ | UGGCAGCACUUCC | 361 | CGGAGAGGGA | 456 | AGCACUUCCG | 551 |
| | 91_164 | GAAUGGCUGAGUU | | GCCACGGAGG | | AAUGGCUGAG | |
| | | UUCCACGCCCGUC | | UG(3′) | | UUUUCCA(5′) | |
| | | CGCGGAGAGGGAG | | | | | |
| | | CCACGGAGGUGAU | | | | | |
| | | CCCGAACG | | | | | |
| AF122917.1 | AF122917_ | GCCAUUUUAAGUC | 362 | AAGUCAGCGC | 457 | AUCCUCACCG | 552 |
| | 3369_3447 | AGCGCUGGGGAGG | | UGGGGAGGCA | | GAACUGACAC | |
| | | CAUGACUGUAAGU | | UGA(5′) | | AA(3′) | |
| | | UCAAAGGUCAUCC | | | | | |
| | | UCACCGGAACUGA | | | | | |
| | | CACAAAAUGGCCG | | | | | |
| AF122918.1 | AF122918_ | GCCAUCUUAAGUG | 363 | UCUUAAGUGG | 458 | CAUCCUCGGC | 553 |
| | 3460_3540 | GCUGUCGCCGAGG | | CUGUCGCCGA | | GGUAACCGCA | |
| | | AUUGACGUCACAG | | GGAUUGAC(5′) | | AAGAUG(3′) | |
| | | UUCAAAGGUCAUC | | | | | |
| | | CUCGGCGGUAACC | | | | | |
| | | GCAAAGAUGGCGG | | | | | |
| | | UC | | | | | |
| AF122918.1 | AF122918_ | AUACGUCAUAAGU | 364 | AAGUCACAUG | 459 | UAGGCCCCGA | 554 |
| | 3566_3642 | CACAUGUCUAGGG | | UCUAGGGGUC | | CAUGUGACUC | |
| | | GUCCACUUAAACAC | | CACU(5′) | | GU(3′) | |
| | | GGAAGUAGGCCCC | | | | | |
| | | GACAUGUGACUCG | | | | | |
| | | UCACGUGUGU | | | | | |
| AF122919.1 | AF122919_ | CCAUUUUAAGUAA | 365 | AAGUAAGGCG | 460 | ACAGCCUUCC | 555 |
| | 3370_3447 | GGCGGAAGCAGCU | | GAAGCAGCUG | | GCUUUGCACA | |
| | | GUCCCUGUAACAA | | UCC(5′) | | A(3′) | |
| | | AAUGGCGGCGACA | | | | | |
| | | GCCUUCCGCUUUG | | | | | |
| | | CACAAAAUGGAG | | | | | |
| AF122920.1 | AF122920_ | GCCAUCUUAAGUG | 366 | AUCUUAAGUG | 461 | CAUCCUCGGC | 556 |
| | 3460_3540 | GCUGUCGCUGAGG | | GCUGUCGCUG | | GGUAACCGCA | |
| | | AUUGACGUCACAG | | AGGAUUGAC | | AAGAUGG(3′) | |
| | | UUCAAAGGUCAUC | | (5′) | | | |
| | | CUCGGCGGUAACC | | | | | |
| | | GCAAAGAUGGCGG | | | | | |
| | | UC | | | | | |
| AF122920.1 | AF122920_ | CAUACGUCAUAAG | 367 | UAAGUCACAU | 462 | UAGGCCCCGA | 557 |
| | 3565_3641 | UCACAUGACAGGA | | GACAGGAGUC | | CAUGUGACUC | |
| | | GUCCACUUAAACAC | | CACU(5′) | | GUC(3′) | |
| | | GGAAGUAGGCCCC | | | | | |
| | | GACAUGUGACUCG | | | | | |
| | | UCACGUGUGU | | | | | |
| AF122921.1 | AF122921_ | CGCCAUCUUAAGU | 368 | AAGUGGCUGU | 463 | UCCUCGGCGG | 558 |
| | 3459_3540 | GGCUGUCGCCGAG | | CGCCGAGGAU | | UAACCGCAAA | |
| | | GAUUGGCGUCACA | | UG(5′) | | (3′) | |
| | | GUUCAAAGGUCAU | | | | | |
| | | CCUCGGCGGUAAC | | | | | |
| | | CGCAAAGAUGGCG | | | | | |
| | | GU | | | | | |
| AF122921.1 | AF122921_ | CAUACGUCAUAAG | 369 | UAAGUCACAU | 464 | GGCCCCGACA | 559 |
| | 3565_3641 | UCACAUGACAGGG | | GACAGGGGUC | | UGUGACUCGU | |
| | | GUCCACUUAAACAC | | CA(5′) | | C(3′) | |
| | | GGAAGUAGGCCCC | | | | | |
| | | GACAUGUGACUCG | | | | | |
| | | UCACGUGUGU | | | | | |
| AF129887.1 | AF129887_ | GCAUACGUCACAA | 370 | ACAAGUCACG | 465 | GGCCCCGUCA | 560 |
| | 3579_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCACGUGGUGU | | | | | |
| AF247137.1 | AF247137_ | CCGCCAUUUUAGG | 371 | AUUUUAGGCU | 466 | UCAAACACCC | 561 |
| | 3453_3530 | CUGUUGCCGGGCG | | GUUGCCGGGC | | AGCGACACCA | |
| | | UUUGACUUCCGUG | | GUUUGACU(5′) | | AAAAAUGG(3′) | |
| | | UUAAAGGUCAAACA | | | | | |
| | | CCCAGCGACACCA | | | | | |
| | | AAAAAUGGCCG | | | | | |
| AF247137.1 | AF247137_ | CUACGUCAUAAGU | 372 | AUAAGUCACG | 467 | CCUCGCCCAC | 562 |
| | 3559_3636 | CACGUGACAGGGA | | UGACAGGGAG | | GUGACUUACC | |
| | | GGGGCGACAAACC | | GGG(5′) | | AC(3′) | |
| | | CGGAAGUCAUCCU | | | | | |
| | | CGCCCACGUGACU | | | | | |
| | | UACCACGUGGUG | | | | | |
| AF247138.1 | AF247138_ | GCCAUUUUAAGUA | 373 | AAGUAGGUGA | 468 | CCUCGGCGGA | 563 |
| | 3455_3532 | GGUGACGUCCAGG | | CGUCCAGGAC | | ACCUAUACAA | |
| | | ACUGACGUAAAGU | | U(5′) | | (3′) | |
| | | UCAAAGGUCAUCC | | | | | |
| | | UCGGCGGAACCUA | | | | | |
| | | UACAAAAUGGCG | | | | | |
| AF247138.1 | AF247138_ | CUACGUCAUAAGU | 374 | CAUAAGUCAC | 469 | GCCCCGUCAC | 564 |
| | 1356_3637 | CACGUGGGGACGG | | GUGGGGACGG | | GUGAUUUACC | |
| | | CUGUACUUAAACAC | | CUGU(5′) | | AC(3′) | |
| | | GGAAGUAGGCCCC | | | | | |
| | | GUCACGUGAUUUA | | | | | |
| | | CCACGUGGUG | | | | | |
| AF261761.1 | AF261761_ | GCCAUUUUAAGUA | 375 | UAAGUAAGGC | 470 | GCGGCGGAGC | 565 |
| | 3431_3504 | AGGCGGAAGAGCU | | GGAAGAGCUC | | ACUUCCGCUU | |
| | | CUAGCUAUACAAAA | | UAGCUA(5′) | | UGCCCAAA(3′) | |
| | | UGGCGGCGGAGCA | | | | | |
| | | CUUCCGCUUUGCC | | | | | |
| | | CAAAAUG | | | | | |
| AF351132.1 | AF351132_ | GCCAUUUUAAGUA | 376 | AGUAGCUGAC | 471 | CAUCCUCGGC | 566 |
| | 3475_3552 | GCUGACGUCAAGG | | GUCAAGGAUU | | GGAAGCUACA | |
| | | AUUGACGUAGAGG | | GAC(5′) | | CAA(3′) | |
| | | UUAAAGGUCAUCC | | | | | |
| | | UCGGCGGAAGCUA | | | | | |
| | | CACAAAAUGGUG | | | | | |
| AF351132.1 | AF351132_ | GCAUACGUCACAA | 377 | ACAAGUCACG | 472 | GGCCCCGUCA | 567 |
| | 3579_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCACGUGUGUA | | | | | |
| AF435014.1 | AF435014_ | GGCGCCAUUUUAA | 378 | UAAGUAAGCA | 473 | CACCGCACUU | 568 |
| | 3344_3426 | GUAAGCAUGGCGG | | UGGCGGGCGG | | CCGUGCUUGC | |
| | | GCGGCGACGUCAC | | CGAC(5′) | | ACAAA(3′) | |
| | | AUGUCAAAGGUCA | | | | | |
| | | CCGCACUUCCGUG | | | | | |
| | | CUUGCACAAAAUG | | | | | |
| | | GC | | | | | |
| AF435014.1 | AF435014_ | UGCUACGUCAUCG | 379 | AUCGAGACAC | 474 | UCGCUGACAC | 569 |
| | 3345_3526 | AGACACGUGGUGC | | GUGGUGCCAG | | ACGUGUCUUG | |
| | | CAGCAGCUGUAAA | | CAGCU(5′) | | UCAC(3′) | |
| | | CCCGGAAGUCGCU | | | | | |
| | | GACACACGUGUCU | | | | | |
| | | UGUCACGU | | | | | |
| AJ620212.1 | AJ620212_ | GCCAUUUUAAGUA | 380 | UCAUCCUCAG | 475 | CAUUUUAAGU | 570 |
| | 3360_3438 | AGCACCGCCUAGG | | CCGGAACUUA | | AAGCACCGCC | |
| | | GAUGACGUAUAAG | | CACAAAAUGG | | UAGGGAUGAC | |
| | | UUCAAAGGUCAUC | | (3′) | | (5′) | |
| | | CUCAGCCGGAACU | | | | | |
| | | UACACAAAAUGGU | | | | | |
| AJ620212.1 | AJ620212_ | ACGUCAUAUGUCA | 381 | AUAUGUCACG | 476 | GUAGGCCCCG | 571 |
| | 3470_3542 | CGUGGGGAGGCCC | | UGGGGAGGCC | | UCACGUGUCA | |
| | | UGCUGCGCAAACG | | CUGCUG(5′) | | UACCAC(3′) | |
| | | CGGAAGUAGGCCC | | | | | |
| | | CGUCACGUGUCAU | | | | | |
| | | ACCACGU | | | | | |
| AJ620218.1 | AJ620218_ | CCAUUUUAAGUAA | 382 | AAGUAAGGCG | 477 | GGCGGGGCAC | 572 |
| | 3381_3458 | GGCGGAAGCAGCU | | GAAGCAGCUC | | UUCCGGCUUG | |
| | | CCACUUUCUCACAA | | CACUUU(5′) | | CCCAA(3′) | |
| | | AAUGGCGGCGGGG | | | | | |
| | | CACUUCCGGCUUG | | | | | |
| | | CCCAAAAUGGC | | | | | |
| AJ620226.1 | AJ620226_ | CCAUUUUAAGUAA | 383 | AAGUAAGGCG | 478 | CGGCGGAGCA | 573 |
| | 3451_3523 | GGCGGAAGUUUCU | | GAAGUUUCUC | | CUUCCGGCUU | |
| | | CCACUAUACAAAAU | | CACU(5′) | | GCCCAA(3′) | |
| | | GGCGGCGGAGCAC | | | | | |
| | | UUCCGGCUUGCCC | | | | | |
| | | AAAAUG | | | | | |
| AJ620227.1 | AJ620227_ | CCAUCUUAAGUAG | 384 | UAAGUAGUUG | 479 | CACCAUCAGC | 574 |
| | 3379_3451 | UUGAGGCGGACGG | | AGGCGGACGG | | CACACCUACU | |
| | | UGGCGUGAGUUCA | | UGGC(5′) | | CAAA(3′) | |
| | | AAGGUCACCAUCA | | | | | |
| | | GCCACACCUACUC | | | | | |
| | | AAAAUGG | | | | | |
| AJ620231.1 | AJ620231_ | CGCCAUCUUAAGU | 385 | UAAGUAGUUG | 480 | ACCAUCAGCC | 575 |
| | 3429_3505 | AGUUGAGGCGGAC | | AGGCGGACGG | | ACACCUACUC | |
| | | GGUGGCGUGAGUU | | UGG(5′) | | AAA(3′) | |
| | | CAAAGGUCACCAU | | | | | |
| | | CAGCCACACCUAC | | | | | |
| | | UCAAAAUGGUG | | | | | |
| AY666122.1 | AY666122_ | UUUCGGACCUUCG | 386 | GACCUUCGGC | 481 | GACUCCGAGA | 576 |
| | 3163_3236 | GCGUCGGGGGGGU | | GUCGGGGGG | | UGCCAUUGGA | |
| | | CGGGGGCUUUACU | | GUCGGGGG(5′) | | CACUGAGG(3′) | |
| | | AAACAGACUCCGA | | | | | |
| | | GAUGCCAUUGGAC | | | | | |
| | | ACUGAGGG | | | | | |
| AY666122.1 | AY666122_ | CCAUUUUAAGUAG | 387 | AUCCUCGGCG | 482 | AGUAGGUGCC | 577 |
| | 3388_3464 | GUGCCGUCCAGCA | | GAACCUAUA | | GUCCAGCA(5′) | |
| | | CUGCUGUUCCGGG | | (3′) | | | |
| | | UUAAAGGGCAUCC | | | | | |
| | | UCGGCGGAACCUA | | | | | |
| | | UACAAAAUGGC | | | | | |
| AY666122.1 | AY666122_ | CUACGUCAUCGAU | 388 | AUCGAUGACG | 483 | AAGUAGGCCC | 578 |
| | 3494_3567 | GACGUGGGGAGGC | | UGGGGAGGCG | | CGCUACGUCA | |
| | | GUACUAUGAAACG | | UACUAU(5′) | | UCAUCAC(3′) | |
| | | CGGAAGUAGGCCC | | | | | |
| | | CGCUACGUCAUCA | | | | | |
| | | UCACGUGG | | | | | |
| AY823988.1 | AY823988_ | CCAUUUUAAGUAA | 389 | UGGCGGAGGA | 484 | AAGGCGGAAG | 579 |
| | 3452_3525 | GGCGGAAGAGCUG | | GCACUUCCGG | | AGCUGCUCUA | |
| | | CUCUAUAUACAAAA | | CUUG(3′) | | UAU(5′) | |
| | | UGGCGGAGGAGCA | | | | | |
| | | CUUCCGGCUUGCC | | | | | |
| | | CAAAAUG | | | | | |
| AY823988.1 | AY823988_ | UGCCUACGUAACA | 390 | AACAAGUCAC | 485 | CAAUCCUCCC | 580 |
| | 3554_3629 | AGUCACGUGGGGA | | GUGGGGAGGG | | ACGUGGCCUG | |
| | | GGGUUGGCGUAUA | | UUGGC(5′) | | UCAC(3′) | |
| | | ACCCGGAAGUCAA | | | | | |
| | | UCCUCCCACGUGG | | | | | |
| | | CCUGUCACGU | | | | | |
| AY823989.1 | AY823989_ | UAAGUAAGGCGGA | 391 | AGGGGUCAGC | 486 | AAGGCGGAAC | 581 |
| | 3551_3623 | ACCAGGCUGUCAC | | CUUCCGCUUU | | CAGGCUGUCA | |
| | | CCCGUGUCAAAGG | | A(3′) | | CCCCGU(5′) | |
| | | UCAGGGGUCAGCC | | | | | |
| | | UUCCGCUUUACAC | | | | | |
| | | AAAAUGG | | | | | |
| AY823989.1 | AY823989_ | UAAGUAAGGCGGA | 392 | AGGGGUCAGC | 487 | AAGGCGGAAC | 582 |
| | 3551_3623 | ACCAGGCUGUCAC | | CUUCCGCUUU | | CAGGCUGUCA | |
| | | CCCGUGUCAAAGG | | A(3′) | | CCCCGU(5′) | |
| | | UCAGGGGUCAGCC | | | | | |
| | | UUCCGCUUUACAC | | | | | |
| | | AAAAUGG | | | | | |
| DQ361268.1 | DQ361268_ | GCAGCCAUUUUAA | 393 | UAAGUCAGCU | 488 | CAUCCUCACC | 583 |
| | 3413_3494 | GUCAGCUUCGGGG | | UCGGGGAGGG | | GGAACUGGUA | |
| | | AGGGUCACGCAAA | | UCAC(5′) | | CAAA(3′) | |
| | | GUUCAAAGGUCAU | | | | | |
| | | CCUCACCGGAACU | | | | | |
| | | GGUACAAAAUGGC | | | | | |
| | | CG | | | | | |
| DQ361268.1 | DQ361268_ | UGCUACGUCAUAA | 394 | UCAUAAGUGA | 489 | UAGGCCCCGC | 584 |
| | 3519_3593 | GUGACGUAGCUGG | | CGUAGCUGGU | | CACGUCACUU | |
| | | UGUCUGCUGUAAA | | GUCUGCU(5′) | | GUCACG(3′) | |
| | | CACGGAAGUAGGC | | | | | |
| | | CCCGCCACGUCAC | | | | | |
| | | UUGUCACGU | | | | | |

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polypeptide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

Exemplary Secreted Polypeptide Effectors

Exemplary secreted therapeutics are described herein, e.g., in the tables below.

TABLE 50
Exemplary cytokines and cytokine receptors

| Cytokine | Cytokine receptor(s) | Entrez Gene ID | UniProt ID |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type 1, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| e | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBB, or | IL-12Rβ2/gp130; IL- | 10148 | Q14213 |
| a heterodimer thereof) | 12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TPR-I and TBR-II | 7046, 7048 | P36897, P37173 |
| TNF-α | TNFR1, TNFR2 | 7132, 7133 | P19438, P20333 |

In some embodiments, an effector described herein comprises a cytokine of Table 50, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 50 or a functional variant or fragment thereof) and a second, heterologous region. In some embodiments, the first region is a first cytokine polypeptide of Table 50. In some embodiments, the second region is a second cytokine polypeptide of Table 50, wherein the first and second cytokine polypeptides form a cytokine heterodimer with each other in a wild-type cell. In some embodiments, the polypeptide of Table 50 or functional variant thereof comprises a signal sequence, e.g., a signal sequence that is endogenous to the effector, or a heterologous signal sequence. In some embodiments, an anellovector encoding a cytokine of Table 50, or a functional variant thereof, is used for the treatment of a disease or disorder described herein.

In some embodiments, an effector described herein comprises an antibody molecule (e.g., an scFv) that binds a cytokine of Table 50. In some embodiments, an effector described herein comprises an antibody molecule (e.g., an scFv) that binds a cytokine receptor of Table 50. In some embodiments, the antibody molecule comprises a signal sequence.

Exemplary cytokines and cytokine receptors are described, e.g., in Akdis et al., “Interleukins (from IL-1 to IL-38), interferons, transforming growth factor β, and TNF-α: Receptors, functions, and roles in diseases” October 2016 Volume 138, Issue 4, Pages 984-1010, which is herein incorporated by reference in its entirety, including Table I therein.

TABLE 51
Exemplary polypeptide hormones and receptors

| Hormone | Receptor | Entrez Gene ID | UniProt ID |
|---|---|---|---|
| Natriuretic Peptide, e.g.. Atrial | NPRA, NPRB, NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | OHR | 2690 | P10912 |
| Human growth hormone (hGH) | hGH receptor (human | 2690 | P10912 |
| | GHR) | | |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Anticiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 51, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 51 or functional variant thereof comprises a signal sequence, e.g., a signal sequence that is endogenous to the effector, or a heterologous signal sequence. In some embodiments, an anellovector encoding a hormone of Table 51, or a functional variant thereof, is used for the treatment of a disease or disorder described herein.

In some embodiments, an effector described herein comprises an antibody molecule (e.g., an scFv) that binds a hormone of Table 51. In some embodiments, an effector described herein comprises an antibody molecule (e.g., an scFv) that binds a hormone receptor of Table 51. In some embodiments, the antibody molecule comprises a signal sequence.

TABLE 52
Exemplary growth factors

| | | |
|---|---|---|
| Growth Factor | Entrez Gene ID | UniProt ID |

PDGF family

| PDGF(e.g., PDGF-1, | PDGF receptor, e.g., | 5156 | P16234 |
|---|---|---|---|
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |
| VEGF family | | | |
| VEGF (e.g., isoforms | VEGFR-1, VEGFR- | 2321 | P17948 |
| VEGF 121, VEGF 165, | 2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR-3 | | |
| P1GF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, | P11487, P08620, |
| FGF-7, FGF-8, FGF-9 | | 2254 | P12034, P10767, |
| | | | P21781, P55075, P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 52, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 52 or functional variant thereof comprises a signal sequence, e.g., a signal sequence that is endogenous to the effector, or a heterologous signal sequence. In some embodiments, an anellovector encoding a growth factor of Table 52, or a functional variant thereof, is used for the treatment of a disease or disorder described herein.

In some embodiments, an effector described herein comprises an antibody molecule (e.g., an scFv) that binds a growth factor of Table 52. In some embodiments, an effector described herein comprises an antibody molecule (e.g., an scFv) that binds a growth factor receptor of Table 52. In some embodiments, the antibody molecule comprises a signal sequence.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety.

TABLE 53
Clotting-associated factors

| Effector | Indication | Entrez Gene ID | UniProt ID |
|---|---|---|---|
| Factor 1 | Afibrinogenomia | 2243, 2266, 2244 | P02671, P02679, |
| (fibrinogen) | | | P02675 |
| Factor II | Factor II Deficiency | 2147 | P00734 |
| Factor IX | Hemophilia B | 2158 | P00740 |
| Factor V | Owren’s disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

In some embodiments, an effector described herein comprises a polypeptide of Table 53, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 53 or functional variant thereof comprises a signal sequence, e.g., a signal sequence that is endogenous to the effector, or a heterologous signal sequence. In some embodiments, an anellovector encoding a polypeptide of Table 53, or a functional variant thereof is used for the treatment of a disease or disorder of Table 53.

Exemplary Protein Replacement Therapeutics

Exemplary protein replacement therapeutics are described herein, e.g., in the tables below.

TABLE 54
Exemplary enzymatic effectors and corresponding indications

| Effector | deficiency | Entrez Gene ID | UniProt ID |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenic Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383,384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, | Q8TDN7, |
| | (lipogranulomatosis) | 55331 | Q5QJU3, Q9NUN7 |
| Cystathionine B | Homocystinuria | 875 | P35520 |
| synthase | | | |
| Dolichol-P-mannose | Congenital disorders of N- | 8813, 54344 | 060762, Q9P2X0 |
| synthase | glycosylation CDG le | | |
| Dolicho-P- | Congenital disorders of N- | 84920 | Q5BKT4 |
| Glc:Man9GlcNAc2-PP- | glycosylation CDG ic | | |
| dolichol | | | |
| glucosyltransferase | | | |
| Dolicho-P- | Congenital disorders of N- | 10195 | Q92685 |
| Man:Man5GlcNAc2- | glycosylation CDG Id | | |
| PP-dolichol | | | |
| mannosyltransferase | | | |
| Dolichyl-P-glucose: Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose: Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren’s disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GMI gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, 8372, | Q12794, Q12891, |
| | IX (hyaluronidase deficiency) | 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransf | | | |
| erase | | | |
| Metalloproteinase-2 | Winchester syndrome | 4313 | P08253 |
| methylmalonyl-CoA | Methylmalonic acidemia | 4594 | P22033 |
| mutase | (vitamin bl2 non-responsive) | | |
| N-Acetyl | Mucopolysaccharidosis MPS | 411 | P15848 |
| galactosamine a-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl-1- | Mucolipidosis ML III | 79158 | Q3T906 |
| phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl-1- | Mucolipidosis ML III | 84572 | Q9UJJ9 |
| phosphotransferase, | (pseudo-Hurler's | | |
| substrate-recognition | poly dystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc:dolichyl- | Congenital disorders of N- | 1798 | Q9H3H5 |
| P NAcGIc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type 1 (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-l-Iduronidase | Mucopolysaccharidosis MPS | 2517 | P04066 |
| | I H/S (Hurler-Scheie | | |
| | syndrome) | | |
| α-l-Iduronidase | Mucopolysaccharidosis MPS | 3425 | P35475 |
| | I-H (Hurler's syndrome) | | |
| α-l-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoffs disease | 3073 | P06865 |

In some embodiments, an effector described herein comprises an enzyme of Table 54, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, an anellovector encoding an enzyme of Table 54, or a functional variant thereof is used for the treatment of a disease or disorder of Table 54. In some embodiments, an anellovector is used to deliver uridine diphosphate glucuronyl-transferase or a functional variant thereof to a target cell, e.g., a liver cell. In some embodiments, an anellovector is used to deliver OCA1 or a functional variant thereof to a target cell, e.g., a retinal cell.

TABLE 55
Exemplary non-enzymatic effectors and corresponding indications

| Effector | Indication | Entrez Gene ID | UniProt ID |
|---|---|---|---|
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

In some embodiments, an effector described herein comprises an erythropoietin (EPO), e.g., a human erythropoietin (hEPO), or a functional variant thereof. In some embodiments, an anellovector encoding an erythropoietin, or a functional variant thereof is used for stimulating erythropoiesis. In some embodiments, an anellovector encoding an erythropoietin, or a functional variant thereof is used for the treatment of a disease or disorder, e.g., anemia. In some embodiments, an anellovector is used to deliver EPO or a functional variant thereof to a target cell, e.g., a red blood cell.

In some embodiments, an effector described herein comprises a polypeptide of Table 55, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, an anellovector encoding a polypeptide of Table 55, or a functional variant thereof is used for the treatment of a disease or disorder of Table 55. In some embodiments, an anellovector is used to deliver SMN or a functional variant thereof to a target cell, e.g., a cell of the spinal cord and/or a motor neuron. In some embodiments, an anellovector is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3; 26(10):2337-2356. doi: 10.1016/j.ymthe.2018.07.011. Epub 2018 Jul. 17.

In some embodiments, an effector described herein comprises a clotting factor, e.g., a clotting factor listed in Table 54 or Table 55 herein. In some embodiments, an effector described herein comprises a protein that, when mutated, causes a lysosomal storage disorder, e.g., a protein listed in Table 54 or Table 55 herein. In some embodiments, an effector described herein comprises a transporter protein, e.g., a transporter protein listed in Table 55 herein.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polypeptide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| Target | Gene accession # | Protein accession # |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pm |
| | | c/articles/PMC2732113/figure/F1 |
| | | / |
| miR-222 | MI0000299 | |
| miR-302-367 | MIR302A And | https://www.ncbi.nlm.nih.gov/pm |
| | MIR367 | c/articles/PMC4400607/ |

Transformation Factors

Therapeutic polypeptides described herein also include transformation factors, e.g., protein factors that transform fibroblasts into differentiated cell e.g., factors disclosed in Table 57 or functional variants thereof, e.g., a protein having at least 80%, 85%, 90%, 95%, 967%, 98%, 99% identity to a protein sequence disclosed in Table 57 by reference to its UniProt ID.

TABLE 57
Exemplary transformation factors

| | | Gene | Protein |
|---|---|---|---|
| Target | Indication | accession # | accession # |
| MESP1 | Organ Repair by | Gene ID: | EAX02066 |
| | transforming fibroblasts | 55897 | |
| ETS2 | Organ Repair by | Gene ID: | NP_005230 |
| | transforming fibroblasts | 2114 | |
| HAND2 | Organ Repair by | Gene ID: | NP_068808 |
| | transforming fibroblasts | 9464 | |
| MYOCARDIN | Organ Repair by | Gene ID: | NP_001139784 |
| | transforming fibroblasts | 93649 | |
| ESRRA | Organ Repair by | Gene ID: | AAH92470 |
| | transforming fibroblasts | 2101 | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | Gene ID: | NP_000651.3 |
| | transforming fibroblasts | 7040 | |
| WNT | Organ Repair by | Gene ID: | NP_005421 |
| | transforming fibroblasts | 7471 | |
| JAK | Organ Repair by | Gene ID: | XP_001308784 |
| | transforming fibroblasts | 3716 | |
| NOTCH | Organ Repair by | Gene ID: | XP_011517019 |
| | transforming fibroblasts | 4851 | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| Target | Gene accession # | Protein accession # |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or anellovector described herein includes a polypeptide linked to a ligand that is capable of targeting a specific location, tissue, or cell.

Gene Editing Components

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningitidis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, e.g., provided either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

Replication Proteins

In some embodiments, the genetic element of the anellovector, e.g., synthetic anellovector, may include sequences that encode one or more replication proteins. In some embodiments, the anellovector may replicate by a rolling-circle replication method, e.g., synthesis of the leading strand and the lagging strand is uncoupled. In such embodiments, the anellovector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element may comprise one or more sequences or a fragment of a sequence from a substantially non-pathogenic virus having at least about 60%, 70% 80%, 85%, 90% 95%, 96%, 97%, 98% and 99% nucleotide sequence identity to any one of the nucleotide sequences described herein, e.g., Table 41.

TABLE 41
Examples of Anelloviruses and their sequences. Accessions numbers and related sequence
information may be obtained at www.ncbi.nlm.nih.gov/genbank/, as referenced on Dec. 11, 2018.

| Accession # | Description |
|---|---|
| AB017613.1 | Torque teno virus 16 DNA, complete genome, isolate: TUS01 |
| AB026345.1 | TT virus genes for ORF1 and ORF2, complete cds, isolate: TRM1 |
| AB026346.1 | TT virus genes for ORF1 and ORF2, complete cds, isolate: TK16 |
| AB026347.1 | TT virus genes for ORF1 and ORF2, complete cds, isolate: TP1-3 |
| AB028669.1 | TT virus gene for ORF1 and ORF2, complete genome, isolate: TJN02 |
| AB030487.1 | TT virus gene for pORF2a, pORF2b, pORF1, complete cds, clone: JaCHCTC19 |
| AB030488.1 | TT virus gene for pORF2a, pORF2b, pORF1, complete cds, clone: JaBD89 |
| AB030489.1 | TT virus gene for pORF2a, pORF2b, pORF1, complete cds, clone: JaBD98 |
| AB038340.1 | TT virus genes for ORF2s, ORF1, ORF3, complete cds |
| AB038622.1 | TT virus genes for ORF2, ORF1, ORF3, complete cds, isolate: TTVyon-LC011 |
| AB038623.1 | TT virus genes for ORF2, ORF1, ORF3, complete cds, isolate: TTVyon-KC186 |
| AB038624.1 | TT virus genes for ORF2, ORF1, ORF3, complete cds, isolate: TTVyon-KC197 |
| AB041821.1 | TT virus mRNA for VP1, complete cds |
| AB050448.1 | Torque teno virus genes for ORF1, ORF2, ORF3, ORF4, complete cds, isolate: |
| | TYM9 |
| AB060592.1 | Torque teno virus gene for ORF1, ORF2, ORF3, ORF4, clone: SAa-39 |
| AB060593.1 | Torque teno virus gene for ORF1, ORF2, ORF3, ORF4, complete cds, clone: |
| | SAa-38 |
| AB060595.1 | TT virus gene for ORF1, ORF2, ORF3, ORF4, complete cds, clone: SAj-30 |
| AB060596.1 | TT virus gene for ORF1, ORF2, ORF3, ORF4, complete cds, clone: SAf-09 |
| AB064596.1 | Torque teno virus DNA, complete genome, isolate: CT25F |
| AB064597.1 | Torque teno virus DNA, complete genome, isolate: CT30F |
| AB064599.1 | Torque teno virus DNA, complete genome, isolate: JT03F |
| AB064600.1 | Torque teno virus DNA, complete genome, isolate: JT05F |
| AB064601.1 | Torque teno virus DNA, complete genome, isolate: JT14F |
| AB064602.1 | Torque teno virus DNA, complete genome, isolate: JT19F |
| AB064603.1 | Torque teno virus DNA, complete genome, isolate: JT41F |
| AB064604.1 | Torque teno virus DNA, complete genome, isolate: CT39F |
| AB064606.1 | Torque teno virus DNA, complete genome, isolate: JT33F |
| AB290918.1 | Torque teno midi virus 1 DNA, complete genome, isolate: MD1-073 |
| AF079173.1 | TT virus strain TTVCHN1, complete genome |
| AF116842.1 | TT virus strain BDH1, complete genome |
| AF122914.3 | TT virus isolate JA20, complete genome |
| AF122917.1 | TT virus isolate JA4, complete genome |
| AF122919.1 | TT virus isolate JA10 unknown genes |
| AF129887.1 | TT virus TTVCHN2, complete genome |
| AF247137.1 | TT virus isolate TUPB, complete genome |
| AF254410.1 | TT virus ORF2 protein and ORF1 protein genes, complete cds |
| AF298585.1 | TT virus Polish isolate P/1C1, complete genome |
| AF315076.1 | TTV-like virus DXL1 unknown genes |
| AF315077.1 | TTV-like virus DXL2 unknown genes |
| AF345521.1 | TT virus isolate TCHN-G1 Orf2 and Orf 1 genes, complete cds |
| AF345522.1 | TT virus isolate TCHN-E Orf2 and Orf 1 genes, complete cds |
| AF345525.1 | TT virus isolate TCHN-D2 Orf2 and Orf 1 genes, complete cds |
| AF345527.1 | TT virus isolate TCHN-C2 Orf2 and Orf 1 genes, complete cds |
| AF345528.1 | TT virus isolate TCHN-F Orf2 and Orf 1 genes, complete cds |
| AF345529.1 | TT virus isolate TCHN-G2 Orf2 and Orf 1 genes, complete cds |
| AF371370.1 | TT virus ORF1, ORF3, and ORF2 genes, complete cds |
| AJ620212.1 | Torque teno virus, isolate tth6, complete genome |
| AJ620213.1 | Torque teno virus, isolate tth10, complete genome |
| AJ620214.1 | Torque teno virus, isolate tth11g2, complete genome |
| AJ620215.1 | Torque teno virus, isolate tth18, complete genome |
| AJ620216.1 | Torque teno virus, isolate tth20, complete genome |
| AJ620217.1 | Torque teno virus, isolate tth21, complete genome |
| AJ620218.1 | Torque teno virus, isolate tth3, complete genome |
| AJ620219.1 | Torque teno virus, isolate tth9, complete genome |
| AJ620220.1 | Torque teno virus, isolate tth16, complete genome |
| AJ620221.1 | Torque teno virus, isolate tth17, complete genome |
| AJ620222.1 | Torque teno virus, isolate tth25, complete genome |
| AJ620223.1 | Torque teno virus, isolate tth26, complete genome |
| AJ620224.1 | Torque teno virus, isolate tth27, complete genome |
| AJ620225.1 | Torque teno virus, isolate tth31, complete genome |
| AJ620226.1 | Torque teno virus, isolate tth4, complete genome |
| AJ620227.1 | Torque teno virus, isolate tth5, complete genome |
| AJ620228.1 | Torque teno virus, isolate tth14, complete genome |
| AJ620229.1 | Torque teno virus, isolate tth29, complete genome |
| AJ620230.1 | Torque teno virus, isolate tth7, complete genome |
| AJ620231.1 | Torque teno virus, isolate tth8, complete genome |
| AJ620232.1 | Torque teno virus, isolate tth13, complete genome |
| AJ620233.1 | Torque teno virus, isolate tth19, complete genome |
| AJ620234.1 | Torque teno virus, isolate tth22g4, complete genome |
| AJ620235.1 | Torque teno virus, isolate tth23, complete genome |
| AM711976.1 | TT virus sle1957 complete genome |
| AM712003.1 | TT virus sle1931 complete genome |
| AM712004.1 | TT virus sle1932 complete genome |
| AM712030.1 | TT virus sle2057 complete genome |
| AM712031.1 | TT virus sle2058 complete genome |
| AM712032.1 | TT virus sle2072 complete genome |
| AM712033.1 | TT virus sle2061 complete genome |
| AM712034.1 | TT virus sle2065 complete genome |
| AY026465.1 | TT virus isolate L01 ORF2 and ORF1 genes, complete cds |
| AY026466.1 | TT virus isolate L02 ORF2 and ORF1 genes, complete cds |
| DQ003341.1 | Torque teno virus clone P2-9-02 ORF2 (ORF2), ORF1A (ORF1A), and ORF1B |
| | (ORF1B) genes, complete cds |
| DQ003342.1 | Torque teno virus clone P2-9-07 ORF2 (ORF2), ORF1A (ORF1A), and ORF1B |
| | (ORF1B) genes, complete cds |
| DQ003343.1 | Torque teno virus clone P2-9-08 ORF2 (ORF2), ORF1A (ORF1A), and ORF1B |
| | (ORF1B) genes, complete cds |
| DQ003344.1 | Torque teno virus clone P2-9-16 ORF2 (ORF2), ORF1A (ORF1A), and ORF1B |
| | (ORF1B) genes, complete cds |
| DQ186994.1 | Torque teno virus clone P601 ORF2 (ORF2) and ORF1 (ORF1) genes, complete |
| | cds |
| DQ186995.1 | Torque teno virus clone P605 ORF2 (ORF2) and ORF1 (ORF1) genes, complete |
| | cds |
| DQ186996.1 | Torque teno virus clone BM1A-02 ORF2 (ORF2) and ORF1 (ORF1) genes, |
| | complete cds |
| DQ186997.1 | Torque teno virus clone BM1A-09 ORF2 (ORF2) and ORF1 (ORF1) genes, |
| | complete cds |
| DQ186998.1 | Torque teno virus clone BM1A-13 ORF2 (ORF2) and ORF1 (ORF1) genes, |
| | complete cds |
| DQ186999.1 | Torque teno virus clone BM1B-05 ORF2 (ORF2) and ORF1 (ORF1) genes, |
| | complete cds |
| DQ187000.1 | Torque teno virus clone BM1B-07 ORF2 (ORF2) and ORF1 (ORF1) genes, |
| | complete cds |
| DQ187001.1 | Torque teno virus clone BM1B-11 ORF2 (ORF2) and ORF1 (ORF1) genes, |
| | complete cds |
| DQ187002.1 | Torque teno virus clone BM1 B-14 ORF2 (ORF2) and ORF1 (ORF1) genes, |
| | complete cds |
| DQ187003.1 | Torque teno virus clone BM1B-08 ORF2 (ORF2) gene, complete cds; and |
| | nonfunctional ORF1 (ORF1) gene, complete sequence |
| DQ187004.1 | Torque teno virus clone BM1C-16 ORF2 (ORF2) and ORF1 (ORF1) genes, |
| | complete cds |
| DQ187005.1 | Torque teno virus clone BM1C-10 ORF2 (ORF2) and ORF1 (ORF1) genes, |
| | complete cds |
| DQ187007.1 | Torque teno virus clone BM2C-25 ORF2 (ORF2) gene, complete cds; and |
| | nonfunctional ORF1 (ORF1) gene, complete sequence |
| DQ361268.1 | Torque teno virus isolate ViPi04 ORF1 gene, complete cds |
| EF538879.1 | Torque teno virus isolate CSC5 ORF2 and ORF1 genes, complete cds |
| EU305675.1 | Torque teno virus isolate LTT7 ORF1 gene, complete cds |
| EU305676.1 | Torque teno virus isolate LTT10 ORF1 gene, complete cds |
| EU889253.1 | Torque teno virus isolate ViPi08 nonfunctional ORF1 gene, complete sequence |
| FJ392105.1 | Torque teno virus isolate TW53A25 ORF2 gene, partial cds; and ORF1 gene, |
| | complete cds |
| FJ392107.1 | Torque teno virus isolate TW53A27 ORF2 gene, partial cds; and ORF1 gene, |
| | complete cds |
| FJ392108.1 | Torque teno virus isolate TW53A29 ORF2 gene, partial cds; and ORF1 gene, |
| | complete cds |
| FJ392111.1 | Torque teno virus isolate TW53A35 ORF2 gene, partial cds; and ORF1 gene, |
| | complete cds |
| FJ392112.1 | Torque teno virus isolate TW53A39 ORF2 gene, partial cds; and ORF1 gene, |
| | complete cds |
| FJ392113.1 | Torque teno virus isolate TW53A26 ORF2 gene, complete cds; and nonfunctional |
| | ORF1 gene, complete sequence |
| FJ392114.1 | Torque teno virus isolate TW53A30 ORF2 and ORF1 genes, complete cds |
| FJ392115.1 | Torque teno virus isolate TW53A31 ORF2 and ORF1 genes, complete cds |
| FJ392117.1 | Torque teno virus isolate TW53A37 ORF1 gene, complete cds |
| FJ426280.1 | Torque teno virus strain SIA109, complete genome |
| FR751500.1 | Torque teno virus complete genome, isolate TTV-HD23a (rheu215) |
| GU797360.1 | Torque teno virus clone 8-17, complete genome |
| HC742700.1 | Sequence 7 from Patent WO2010044889 |
| HC742710.1 | Sequence 17 from Patent WO2010044889 |
| JX134044.1 | TTV-like mini virus isolate TTMV_LY1, complete genome |
| JX134045.1 | TTV-like mini virus isolate TTMV_LY2, complete genome |
| KU243129.1 | TTV-like mini virus isolate TTMV-204, complete genome |
| KY856742.1 | TTV-like mini virus isolate zhenjiang, complete genome |
| LC381845.1 | Torque teno virus Human/Japan/KS025/2016 DNA, complete genome |
| MH648892.1 | Anelloviridae sp. isolate ctdc048, complete genome |
| MH648893.1 | Anelloviridae sp. isolate ctdh007, complete genome |
| MH648897.1 | Anelloviridae sp. isolate ctcb038, complete genome |
| MH648900.1 | Anelloviridae sp. isolate ctfc019, complete genome |
| MH648901.1 | Anelloviridae sp. isolate ctbb022, complete genome |
| MH648907.1 | Anelloviridae sp. isolate ctcf040, complete genome |
| MH648911.1 | Anelloviridae sp. isolate cthi018, complete genome |
| MH648912.1 | Anelloviridae sp. isolate ctea38, complete genome |
| MH648913.1 | Anelloviridae sp. isolate ctbg006, complete genome |
| MH648916.1 | Anelloviridae sp. isolate ctbg020, complete genome |
| MH648925.1 | Anelloviridae sp. isolate ctci019, complete genome |
| MH648932.1 | Anelloviridae sp. isolate ctid031, complete genome |
| MH648946.1 | Anelloviridae sp. isolate ctdb017, complete genome |
| MH648957.1 | Anelloviridae sp. isolate ctch017, complete genome |
| MH648958.1 | Anelloviridae sp. isolate ctbh011, complete genome |
| MH648959.1 | Anelloviridae sp. isolate ctbc020, complete genome |
| MH648962.1 | Anelloviridae sp. isolate ctif015, complete genome |
| MH648966.1 | Anelloviridae sp. isolate ctei055, complete genome |
| MH648969.1 | Anelloviridae sp. isolate ctjg000, complete genome |
| MH648976.1 | Anelloviridae sp. isolate ctcj064, complete genome |
| MH648977.1 | Anelloviridae sp. isolate ctbj022, complete genome |
| MH648982.1 | Anelloviridae sp. isolate ctbf014, complete genome |
| MH648983.1 | Anelloviridae sp. isolate ctbd027, complete genome |
| MH648985.1 | Anelloviridae sp. isolate ctch016, complete genome |
| MH648986.1 | Anelloviridae sp. isolate ctbd020, complete genome |
| MH648989.1 | Anelloviridae sp. isolate ctga035, complete genome |
| MH648990.1 | Anelloviridae sp. isolate cthf001, complete genome |
| MH648995.1 | Anelloviridae sp. isolate ctbd067, complete genome |
| MH648997.1 | Anelloviridae sp. isolate ctce026, complete genome |
| MH648999.1 | Anelloviridae sp. isolate ctfb058, complete genome |
| MH649002.1 | Anelloviridae sp. isolate ctjj046, complete genome |
| MH649006.1 | Anelloviridae sp. isolate ctcf030, complete genome |
| MH649008.1 | Anelloviridae sp. isolate ctbg025, complete genome |
| MH649011.1 | Anelloviridae sp. isolate ctbh052, complete genome |
| MH649014.1 | Anelloviridae sp. isolate ctba003, complete genome |
| MH649017.1 | Anelloviridae sp. isolate ctbb016, complete genome |
| MH649022.1 | Anelloviridae sp. isolate ctch023, complete genome |
| MH649023.1 | Anelloviridae sp. isolate ctbd051, complete genome |
| MH649028.1 | Anelloviridae sp. isolate ctbf9, complete genome |
| MH649038.1 | Anelloviridae sp. isolate ctbi030, complete genome |
| MH649039.1 | Anelloviridae sp. isolate ctca057, complete genome |
| MH649040.1 | Anelloviridae sp. isolate ctch033, complete genome |
| MH649042.1 | Anelloviridae sp. isolate ctjd005, complete genome |
| MH649045.1 | Anelloviridae sp. isolate ctdc021, complete genome |
| MH649051.1 | Anelloviridae sp. isolate ctdg044, complete genome |
| MH649056.1 | Anelloviridae sp. isolate ctcc062, complete genome |
| MH649061.1 | Anelloviridae sp. isolate ctid009, complete genome |
| MH649062.1 | Anelloviridae sp. isolate ctdc018, complete genome |
| MH649063.1 | Anelloviridae sp. isolate ctbf012, complete genome |
| MH649068.1 | Anelloviridae sp. isolate ctcc066, complete genome |
| MH649070.1 | Anelloviridae sp. isolate ctda011, complete genome |
| MH649077.1 | Anelloviridae sp. isolate ctbh034, complete genome |
| MH649083.1 | Anelloviridae sp. isolate ctdg028, complete genome |
| MH649084.1 | Anelloviridae sp. isolate ctii061, complete genome |
| MH649085.1 | Anelloviridae sp. isolate cteh021, complete genome |
| MH649092.1 | Anelloviridae sp. isolate ctbg012, complete genome |
| MH649101.1 | Anelloviridae sp. isolate ctif053, complete genome |
| MH649104.1 | Anelloviridae sp. isolate ctei657, complete genome |
| MH649106.1 | Anelloviridae sp. isolate ctca015, complete genome |
| MH649114.1 | Anelloviridae sp. isolate ctbf050, complete genome |
| MH649122.1 | Anelloviridae sp. isolate ctdc002, complete genome |
| MH649125.1 | Anelloviridae sp. isolate ctbb15, complete genome |
| MH649127.1 | Anelloviridae sp. isolate ctba013, complete genome |
| MH649137.1 | Anelloviridae sp. isolate ctbb000, complete genome |
| MH649141.1 | Anelloviridae sp. isolate ctbc019, complete genome |
| MH649142.1 | Anelloviridae sp. isolate ctid026, complete genome |
| MH649144.1 | Anelloviridae sp. isolate ctfj004, complete genome |
| MH649152.1 | Anelloviridae sp. isolate ctcj13, complete genome |
| MH649156.1 | Anelloviridae sp. isolate ctci006, complete genome |
| MH649157.1 | Anelloviridae sp. isolate ctbd025, complete genome |
| MH649158.1 | Anelloviridae sp. isolate ctbf005, complete genome |
| MH649161.1 | Anelloviridae sp. isolate ctcf045, complete genome |
| MH649165.1 | Anelloviridae sp. isolate ctcc29, complete genome |
| MH649169.1 | Anelloviridae sp. isolate ctib021, complete genome |
| MH649172.1 | Anelloviridae sp. isolate ctbh857, complete genome |
| MH649174.1 | Anelloviridae sp. isolate ctbj049, complete genome |
| MH649178.1 | Anelloviridae sp. isolate ctfc006, complete genome |
| MH649179.1 | Anelloviridae sp. isolate ctbe000, complete genome |
| MH649183.1 | Anelloviridae sp. isolate ctbb031, complete genome |
| MH649186.1 | Anelloviridae sp. isolate ctcb33, complete genome |
| MH649189.1 | Anelloviridae sp. isolate ctcc12, complete genome |
| MH649196.1 | Anelloviridae sp. isolate ctci060, complete genome |
| MH649199.1 | Anelloviridae sp. isolate ctbb017, complete genome |
| MH649203.1 | Anelloviridae sp. isolate cthc018, complete genome |
| MH649204.1 | Anelloviridae sp. isolate ctbj003, complete genome |
| MH649206.1 | Anelloviridae sp. isolate ctbg010, complete genome |
| MH649208.1 | Anelloviridae sp. isolate ctid008, complete genome |
| MH649209.1 | Anelloviridae sp. isolate ctbg056, complete genome |
| MH649210.1 | Anelloviridae sp. isolate ctda001, complete genome |
| MH649212.1 | Anelloviridae sp. isolate ctcf004, complete genome |
| MH649217.1 | Anelloviridae sp. isolate ctbe029, complete genome |
| MH649223.1 | Anelloviridae sp. isolate ctci016, complete genome |
| MH649224.1 | Anelloviridae sp. isolate ctce11, complete genome |
| MH649228.1 | Anelloviridae sp. isolate ctcf013, complete genome |
| MH649229.1 | Anelloviridae sp. isolate ctcb036, complete genome |
| MH649241.1 | Anelloviridae sp. isolate ctda027, complete genome |
| MH649242.1 | Anelloviridae sp. isolate ctbf003, complete genome |
| MH649254.1 | Anelloviridae sp. isolate ctjb007, complete genome |
| MH649255.1 | Anelloviridae sp. isolate ctbb023, complete genome |
| MH649256.1 | Anelloviridae sp. isolate ctca002, complete genome |
| MH649258.1 | Anelloviridae sp. isolate ctcg010, complete genome |
| MH649263.1 | Anelloviridae sp. isolate ctgh3, complete genome |
| MK012439.1 | Anelloviridae sp. isolate cthe000, complete genome |
| MK012440.1 | Anelloviridae sp. isolate ctjd008, complete genome |
| MK012448.1 | Anelloviridae sp. isolate ctch012, complete genome |
| MK012457.1 | Anelloviridae sp. isolate ctda009, complete genome |
| MK012458.1 | Anelloviridae sp. isolate ctcd015, complete genome |
| MK012485.1 | Anelloviridae sp. isolate ctfd011, complete genome |
| MK012489.1 | Anelloviridae sp. isolate ctba003, complete genome |
| MK012492.1 | Anelloviridae sp. isolate ctbb005, complete genome |
| MK012493.1 | Anelloviridae sp. isolate ctcj014, complete genome |
| MK012500.1 | Anelloviridae sp. isolate ctcb001, complete genome |
| MK012504.1 | Anelloviridae sp. isolate ctcj010, complete genome |
| MK012516.1 | Anelloviridae sp. isolate ctcf003, complete genome |
| NC_038336.1 | Torque teno virus 5 isolate TCHN-C1 Orf2 and Orf1 genes, complete cds |
| NC_038338.1 | Torque teno virus 11 isolate TCHN-D1 Orf2 and Orf 1 genes, complete cds |
| NC_038339.1 | Torque teno virus 13 isolate TCHN-A Orf2 and Orf1 genes, complete cds |
| NC_038340.1 | Torque teno virus 20 ORF4, ORF3, ORF2, ORF1 genes, complete cds, clone: |
| | SAa-10 |
| NC_038341.1 | Torque teno virus 21 isolate TCHN-B ORF2 and ORF1 genes, complete cds |
| NC_038342.1 | Torque teno virus 23 ORF2, ORF1 genes, complete cds, isolate: s-TTV CH65-2 |
| NC_038343.1 | Torque teno virus 24 ORF4, ORF3, ORF2, ORF1 genes, complete cds, clone: |
| | SAa-01 |
| NC_038344.1 | Torque teno virus 29 ORF2, ORF1, ORF3 genes, complete cds, isolate: TTVyon- |
| | KC009 |
| NC_038345.1 | Torque teno mini virus 10 isolate LIL-y1 ORF2, ORF1, ORF3, and ORF4 genes, |
| | complete cds |
| NC_038346.1 | Torque teno mini virus 11 isolate LIL-y2 ORF2, ORF1, and ORF3 genes, |
| | complete cds |
| NC_038347.1 | Torque teno mini virus 12 isolate LIL-y3 ORF2, ORF1, ORF3, and ORF4 genes, |
| | complete cds |
| NC_038350.1 | Torque teno midi virus 3 isolate 2PoSMA ORF2 and ORF1 genes, complete cds |
| NC_038351.1 | Torque teno midi virus 4 isolate 6PoSMA ORF2, ORF1, and ORF3 genes, |
| | complete cds |
| NC_038352.1 | Torque teno midi virus 5 DNA, complete genome, isolate: MDJHem2 |
| NC_038353.1 | Torque teno midi virus 6 DNA, complete genome, isolate: MDJHem3-1 |
| NC_038354.1 | Torque teno midi virus 7 DNA, complete genome, isolate: MDJHem3-2 |
| NC_038355.1 | Torque teno midi virus 8 DNA, complete genome, isolate: MDJN1 |
| NC_038356.1 | Torque teno midi virus 9 DNA, complete genome, isolate: MDJN2 |
| NC_038357.1 | Torque teno midi virus 10 DNA, complete genome, isolate: MDJN14 |
| NC_038358.1 | Torque teno midi virus 11 DNA, complete genome, isolate: MDJN47 |
| NC_038359.1 | Torque teno midi virus 12 DNA, complete genome, isolate: MDJN51 |
| NC_038360.1 | Torque teno midi virus 13 DNA, complete genome, isolate: MDJN69 |
| NC_038361.1 | Torque teno midi virus 14 DNA, complete genome, isolate: MDJN97 |
| NC_038362.1 | Torque teno midi virus 15 DNA, complete genome, isolate: Pt-TTMDV210 |

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more. Since the genetic code is degenerate, a homologous nucleotide sequence can include any number of silent base changes, i.e., nucleotide substitutions that nonetheless encode the same amino acid.

Proteinaceous Exterior

In some embodiments, the anellovector, e.g., synthetic anellovector, comprises a proteinaceous exterior that encloses the genetic element. The proteinaceous exterior can comprise a substantially non-pathogenic exterior protein that fails to elicit an unwanted immune response in a mammal. The proteinaceous exterior of the anellovectors typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 molecule and/or capsid protein sequence, e.g., as described herein. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to an Anellovirus ORF1 nucleic acid, e.g., as described herein.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to an Anellovirus ORF1 molecule as described herein.

In some embodiments, the ranges of amino acids with less sequence identity may provide one or more of the properties described herein and differences in cell/tissue/species specificity (e.g. tropism).

In some embodiments, the anellovector lacks lipids in the proteinaceous exterior. In some embodiments, the anellovector lacks a lipid bilayer, e.g., a viral envelope. In some embodiments, the interior of the anellovector is entirely covered (e.g., 100% coverage) by a proteinaceous exterior. In some embodiments, the interior of the anellovector is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: an arginine-rich region, jelly-roll region, N22 domain, hypervariable region, and/or C-terminal domain, e.g., of an ORF1 molecule, e.g., as described herein. In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 nucleic acid, e.g., as described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

In some embodiments, a plurality of anellovectors (e.g., a first plurality of anellovectors or a second plurality of anellovectors, e.g., as described herein) comprises multiple copies of the same anellovector. In some embodiments, a plurality of anellovectors (e.g., a first plurality of anellovectors or a second plurality of anellovectors, e.g., as described herein) comprises multiple different anellovectors.

In some embodiments, a first plurality of anellovectors comprising a proteinaceous exterior as described herein is administered to a subject. In some embodiments, a second plurality of anellovectors comprising a proteinaceous exterior described herein, is subsequently administered to the subject following administration of the first plurality. In some embodiments, the second plurality of anellovectors comprises the same proteinaceous exterior as the anellovectors of the first plurality. In some embodiments, the second plurality of anellovectors comprises a proteinaceous exterior with at least 70%, 75%, 80%, 85%, 90%, 95%, 96%, 97%, 98%, 99%, or 100% amino acid sequence identity to the proteinaceous exterior of the anellovectors of the first plurality. In some embodiments, the second plurality of anellovectors comprises an ORF1 molecule with at least 70%, 75%, 80%, 85%, 90%, 95%, 96%, 97%, 98%, 99%, or 100% amino acid sequence identity to the ORF1 molecule of the anellovectors of the first plurality. In some embodiments the second plurality of anellovectors comprises an ORF1 molecule having the same amino acid sequence as the ORF1 molecule comprised by the anellovectors of the first plurality. In some embodiments, the proteinaceous exterior of the second plurality of anellovectors comprises a polypeptide, e.g., an ORF1 molecule, having at least 70%, 75%, 80%, 85%, 90%, 95%, 96%, 97%, 98%, 99%, or 100% amino acid sequence identity to a polypeptide, e.g., an ORF1 molecule, in the proteinaceous exterior of the first plurality of anellovectors. In some embodiments, the proteinaceous exterior of the second plurality of anellovectors comprises a polypeptide, e.g., a capsid protein, having at least 70%, 75%, 80%, 85%, 90%, 95%, 96%, 97%, 98%, 99%, or 100% amino acid sequence identity to a polypeptide, e.g., a capsid protein, in the proteinaceous exterior of the first plurality of Anellovectors. In some embodiments, the second plurality of anellovectors comprises a proteinaceous exterior with at least one surface epitope in common with the anellovectors of the first plurality. In some embodiments, the second plurality of anellovectors comprises an ORF1 molecule with at least one surface epitope in common with the ORF1 of the anellovectors of the first plurality. In some embodiments, the second plurality of anellovectors comprises a proteinaceous exterior with one or more amino acid sequence difference (e.g., a conservative mutation) from the proteinaceous exterior of the anellovectors of the first plurality. In some embodiments, an antibody, e.g., an antibody within the subject, that binds to the proteinaceous exterior of the first plurality of anellovectors also binds to the proteinaceous exterior of the second plurality of of anellovectors. In some embodiments, the antibody binds with about the same affinity (e.g., having a KD of about 90-110%, e.g., 95-105%) to the proteinaceous exterior of the first plurality of anellovectors as to the proteinaceous exterior of the second plurality of anellovectors.

In some embodiments, the proteinaceous exterior of the first plurality of anellovectors comprises the same tertiary structure as the proteinaceous exterior of the second plurality of anellovectors. In some embodiments, the structure, e.g., tertiary structure, of the proteinaceous exterior of the anellovectors in the first and second plurality can be determined using cryo-electron microscopy (cryo-EM), X-ray crystallography, or nuclear magnetic resonance (NMR). In some embodiments, the structure of the proteinaceous exterior of the first plurality of anellovectors is compared to structure of the proteinaceous exterior of the second plurality of anellovectors using structural alignment and measurement of the atomic coordinates of the atoms in the protein structure, e.g., a measurement of root-mean-square-deviation (RMSD). In some embodiments, the RMSD can be calculated for the backbone of the polypeptide chain of the structures being compared, the alpha carbons of the polypeptide chain of the structures being compared, or all the atoms of the structures being compared, e.g., the proteinaceous exterior of the first plurality of anellovectors and the proteinaceous exterior of the second plurality of anellovectors. In some embodiments, an RMSD of a lower value, e.g., ≤5 Angstroms, indicates structural similarity between the proteinaceous exterior of the first plurality of anellovectors and proteinaceous exterior of the second plurality of anellovectors. In some embodiments, an RMSD of a lower value, e.g., ≤3 Angstroms, indicates high structural similarity between the proteinaceous exterior of the first plurality of anellovectors and proteinaceous exterior of the second plurality of anellovectors. In some embodiments, an RMSD of 0 Angstroms indicates that two proteins comprise the same structure, e.g., that the structure of the proteinaceous exterior of the first plurality of anellovectors is the same as the proteinaceous exterior of the second plurality of anellovectors.

### III. Nucleic Acid Constructs

The genetic element described herein may be included in a nucleic acid construct (e.g., a nucleic acid construct as described herein).

In one aspect, the invention includes a nucleic acid genetic element construct comprising a genetic element comprising (i) a sequence encoding a non-pathogenic exterior protein (e.g., an Anellovirus ORF1 molecule or a splice variant or functional fragment thereof), (ii) an exterior protein binding sequence that binds the genetic element to the non-pathogenic exterior protein, and (iii) a sequence encoding an effector.

The genetic element or any of the sequences within the genetic element can be obtained using any suitable method. Various recombinant methods are known in the art, such as, for example screening libraries from cells harboring viral sequences, deriving the sequences from a nucleic acid construct known to include the same, or isolating directly from cells and tissues containing the same, using standard techniques. Alternatively or in combination, part or all of the genetic element can be produced synthetically, rather than cloned.

In some embodiments, the nucleic acid construct includes regulatory elements, nucleic acid sequences homologous to target genes, and/or various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the nucleic acid construct is substantially non-pathogenic and/or substantially non-integrating in a host cell or is substantially non-immunogenic in a host.

In some embodiments, the nucleic acid construct is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

The anellovectors described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ anellovectors. In some embodiments, the pharmaceutical composition comprises about 10⁵-10¹⁵, 10⁵-10¹⁰, or 10¹⁰-10¹⁵ anellovectors. In some embodiments, the pharmaceutical composition comprises about 10⁸ (e.g., about 10⁵, 10⁶, 10⁷, 10⁸, 10⁹, or 10¹⁰) genomic equivalents/mL of the anellovector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the anellovector, e.g., as determined according to the method of Example 18 of PCT/US19/65995. In some embodiments, the pharmaceutical composition comprises sufficient anellovectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the anellovectors per cell to a population of the eukaryotic cells. In some embodiments, the pharmaceutical composition comprises sufficient anellovectors to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the anellovectors per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:

a) an anellovector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and

b) a pharmaceutical excipient.

Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In some embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an anellovector, Anellovirus, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs) (see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

Combinations

In one aspect, the anellovector or composition comprising an anellovector described herein may also include one or more heterologous moiety. In one aspect, the anellovector or composition comprising a anellovector described herein may also include one or more heterologous moiety in a fusion. In some embodiments, a heterologous moiety may be linked with the genetic element. In some embodiments, a heterologous moiety may be enclosed in the proteinaceous exterior as part of the anellovector. In some embodiments, a heterologous moiety may be administered with the anellovector.

In one aspect, the invention includes a cell or tissue comprising any one of the anellovectors and heterologous moieties described herein.

In another aspect, the invention includes a pharmaceutical composition comprising a anellovector and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

Viruses

In some embodiments, an anellovector or composition (e.g., as described herein) may further comprise one or more components or elements (e.g., nucleic acids or polypeptides) from a virus other than an Anellovirus, e.g., as a heterologous moiety, e.g., a single stranded DNA virus, e.g., Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the anellovector is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein.

In some embodiments, the heterologous moiety may comprise one or more viruses that are identified as lacking in the subject. For example, a subject identified as having dyvirosis may be administered a composition comprising an anellovector and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the anellovector or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective anellovector or virus under the control of regulatory sequences within the LTR. Suitable cell lines for replicating the anellovectors described herein include cell lines known in the art, e.g., A549 cells, which can be modified as described herein.

Targeting Moiety

In some embodiments, the composition or anellovector described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the anellovector or genetic element. For example, a targeting moiety may include a therapeutic that interacts with a specific molecule of interest to increase, decrease or otherwise modulate its function.

Tagging or Monitoring Moiety

In some embodiments, the composition or anellovector described herein may further comprise a tag to label or monitor the anellovector or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

Nanoparticles

In some embodiments, the composition or anellovector described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

Small Molecules

In some embodiments, the composition or anellovector described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.e.g, PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

Peptides or Proteins

In some embodiments, the composition or anellovector described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition or anellovector described herein includes a polypeptide linked to a ligand that is capable of targeting a specific location, tissue, or cell.

Oligonucleotide Aptamers

In some embodiments, the composition or anellovector described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamer-cite_note-10), hemin, interferon γ, vascular endothelial growth factor (VEGF), prostate specific antigen (PSA), dopamine, and the non-classical oncogene, heat shock factor 1 (HSF1).

Peptide Aptamers

In some embodiments, the composition or anellovector described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### V. Host Cells

The invention is further directed to a host or host cell comprising an anellovector described herein. In some embodiments, the host or host cell is a plant, insect, bacteria, fungus, vertebrate, mammal (e.g., human), or other organism or cell. In certain embodiments, as confirmed herein, provided anellovectors infect a range of different target host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the anellovector is substantially non-immunogenic in the host. The anellovector or genetic element fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an anellovector. In some embodiments, the host is a mammal, such as a human. In some embodiments, the host cell is a mammalian cell, e.g., a human cell. The amount of the anellovector in the host can be measured at any time after administration. In certain embodiments, a time course of anellovector growth in a culture is determined.

In some embodiments, the anellovector, e.g., an anellovector as described herein, is heritable. In some embodiments, the anellovector is transmitted linearly in fluids and/or cells from mother to child. In some embodiments, daughter cells from an original host cell comprise the anellovector. In some embodiments, a mother transmits the anellovector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the anellovector in a host cell has a transmission efficiency during meiosis of at 25%, 50%, 60%, 70%, 80%, 85%, 90%, 95%, or 99%. In some embodiments, the anellovector in a host cell has a transmission efficiency during mitosis of at least 25%, 50%, 60%, 70%, 80%, 85%, 90%, 95%, or 99%. In some embodiments, the anellovector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the anellovector, e.g., anellovector replicates within the host cell. In one embodiment, the anellovector is capable of replicating in a mammalian cell, e.g., human cell. In other embodiments, the anellovector is replication deficient or replication incompetent.

While in some embodiments the anellovector replicates in the host cell, the anellovector does not integrate into the genome of the host, e.g., with the host's chromosomes. In some embodiments, the anellovector has a negligible recombination frequency, e.g., with the host's chromosomes. In some embodiments, the anellovector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The anellovectors and compositions comprising anellovectors described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The anellovectors may be administered alone or formulated as a pharmaceutical composition. In some embodiments, the anellovectors may be administered in a single dose, e.g., a first plurality. In some embodiments, anellovectors may be administered in at least two doses, e.g., a first plurality, followed by a second plurality. In some embodiments, the anellovectors may be administered in multiple doses, e.g., a first plurality, a second plurality, a third plurality, optionally a fourth plurality, optionally a fifth plurality, and/or optionally further pluralities.

The anellovectors may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of an anellovector or composition comprising same, e.g., as described herein, may result in delivery of a genetic element comprised by the anellovector to a target cell, e.g., in a subject.

An anellovector or composition thereof described herein, e.g., comprising an effector (e.g., an endogenous or exogenous effector), may be used to deliver the effector to a cell, tissue, or subject. In some embodiments, the anellovector or composition thereof is used to deliver the effector to bone marrow, blood, heart, GI or skin. Delivery of an effector by administration of a anellovector composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In some embodiments, a anellovector or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the anellovector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the anellovector described herein, or a composition comprising the anellovector, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector modulates (e.g., increases or decreases) an activity or function in a cell with which the anellovector is contacted. In some embodiments, the anellovector modulates (e.g., increases or decreases) the level or activity of a molecule (e.g., a nucleic acid or a protein) in a cell with which the anellovector is contacted. In some embodiments, the anellovector decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that decreases viability of a cell, e.g., a cancer cell, with which the anellovector is contacted, e.g., by at least about 10%, 20%, 30%, 40%, 50%, 60%, 70%, 80%, 90%, 95%, 99%, or more. In some embodiments, the anellovector increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector comprises an effector, e.g., an miRNA, e.g., miR-625, that increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more.

### VII. Administration/Delivery

The composition (e.g., a pharmaceutical composition comprising an anellovector as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, the subject to which administration of the pharmaceutical compositions is contemplated is a human. In some embodiments, the subject is a neonate, e.g., between 0 and 4 weeks of age. In some embodiments, the subject is an infant, e.g., between 4 weeks of age and 1 year of age. In some embodiments, the subject is a a child, e.g., between 1 year of age and 12 years of age. In some embodiments, the subject is less than 18 years of age. In some embodiments, the subject is an adolescent, e.g., between 12 years of age and 18 years of age. In some embodiments, the subject is above the age of 18. In some embodiments, the subject is a young adult, e.g., between 18 years of age and 25 years of age. In some embodiments, the subject is an adult, e.g., between 25 years of age to 50 years of age. In some embodiments, the subject is an older adult, e.g., an adult at least 50 years of age or older.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector to a subject. The method includes administering a pharmaceutical composition comprising an anellovector as described herein to the subject. In some embodiments, the administered anellovector replicates in the subject (e.g., becomes a part of the virome of the subject).

The pharmaceutical composition may include wild-type or native viral elements and/or modified viral elements. The anellovector may include one or more Anellovirus sequences (e.g., nucleic acid sequences or nucleic acid sequences encoding amino acid sequences thereof) or a sequence with at least about 60%, 65%, 70%, 75%, 80%, 85%, 90% 95%, 96%, 97%, 98% and 99% nucleotide sequence identity thereto. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more Anellovirus sequences (e.g., an Anellovirus ORF1 nucleic acid sequence). The anellovector may comprise a nucleic acid molecule encoding an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule). The anellovector may comprise a polypeptide comprising an amino acid sequence with at least about 60%, 65%, 70%, 75%, 80%, 85%, 90% 95%, 96%, 97%, 98% and 99% sequence identity to an Anellovirus amino acid sequence (e.g., the amino acid sequence of an Anellovirus ORF1 molecule).

In some embodiments, the anellovector is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In one aspect, the invention features a method of delivering an effector to a subject, e.g., a human subject, who has previously been administered an anellovector, e.g., a first plurality of anellovectors, the method comprising administration of a second plurality of anellovectors. In another aspect, the invention features a method of delivering an effector to a subject, e.g., a human subject, the method comprising administering a first plurality of anellovectors to the subject and subsequently administering to the subject a second plurality of anellovectors. In some embodiments, the methods described herein, further comprise administration of a third, fourth, fifth, and/or further plurality of anellovectors. In some embodiments, the first and second plurality are administered via the same route of administration, e.g., intravenous administration. In some embodiments, the first and second plurality are administered via different routes of administration. In some embodiments, the first plurality of anellovectors is administered to the subject as part of a first pharmaceutical composition. In some embodiments, the second plurality of anellovectors is administered to the subject as part of a second pharmaceutical composition.

In some embodiments, the first and the second plurality comprise about the same dosage of anellovectors, e.g., wherein the first plurality and the second plurality of anellovectors comprise about the same quantity and/or concentration of anellovectors. In some embodiments, the second plurality comprises 90-110%, e.g., 95-105% of the number of anellovectors in the first plurality. In some embodiments, the first plurality comprises a greater dosage of anellovectors than the second plurality, e.g., wherein the first plurality comprises a greater quantity and/or concentration of anellovectors relative to the second plurality. In some embodiments, the first plurality comprises a lower dosage of anellovectors than the second plurality, e.g., wherein the first plurality comprises a greater quantity and/or concentration of anellovectors relative to the second plurality. In some embodiments, the subject receives repeated doses of anellovectors, wherein the repeated doses are administered over the course of at least 1, 2, 3, 4, or 5 years. In some embodiments, the repeated dose is administered about every 1, 2, 3, or 4 weeks, or about every 1, 2, 3, 4, 5, 6, 7, 8, 9, 10, 11, or 12 months.

In some embodiments, the genetic element comprised in the anellovectors of the first plurality administered to the subject are detectable in the subject at least 50, 60, 70, 80, 90, 100, 110, 120, 130, 140, or 150 days after administration thereof, e.g., by a high-resolution melting (HRM) assay, e.g., as described in Example 1. In some embodiments, the genetic element comprised in the anellovectors of the second plurality administered to the subject are detectable in the subject at least 50, 60, 70, 80, 90, 100, 110, 120, 130, 140, or 150 days after administration thereof, e.g., by a high-resolution melting (HRM) assay, e.g., as described in Example 1.

In some embodiments, the first and/or second plurality of anellovectors administered to the subject comprises an effector. In some embodiments, the first and/or second plurality comprises an exogenous effector. In some embodiments, the first and/or second plurality comprises an endogenous effector. In some embodiments, the effector of the second plurality of anellovectors is the same effector as the effector of the first plurality of anellovectors. In some embodiments, the effector of the second plurality of anellovectors is different from the effector of the first plurality of anellovectors. In some embodiments, the second plurality of anellovectors delivers about the same number of copies of the effector to the subject as the number of effectors delivered by the first plurality of anellovectors. In some embodiments, the second plurality of anellovectors delivers the effector to the subject at a level of at least about 50%, 55%, 60%, 65%, 70%, 75%, 80%, 85%, 90%, 95%, 96%, 97%, 98%, 99%, or 100% of copies of the effector delivered to the subject by the first plurality of anellovectors (e.g., wherein the effector delivered by the first plurality may be the same or different form the effector delivered by the second plurality), In some embodiments, the second plurality of anellovectors delivers delivers more copies (e.g., at least 2, 3, 4, 5, 6, 7, 8, 9, 10, 20, 30, 40, 50, 60, 70, 80, 90, 100, 500, or 1000-fold as many copies) of the effector to the subject than the first plurality of anellovectors. In some embodiments, the second plurality of anellovectors has a biological effect on the subject (e.g., knockdown of a target gene, or upregulation of a biomarker) that is no less than the biological effect of administration of the first plurality of anellovectors.

In some embodiments, identifying or selecting a subject on the basis of having received a plurality of anellovectors comprises performing an assay on a sample from the subject. In some embodiments, identifying or selecting a subject on the basis of having received a plurality of anellovectors comprises obtaining information from a third party (e.g., a laboratory), wherein the third party performed an assay on a sample from the subject. In some embodiments, identifying or selecting a subject on the basis of having received a plurality of anellovectors comprises reviewing the subject's medical history.

In some embodiments, the subject is administered the pharmaceutical composition further comprising one or more viral strains that are not represented in the viral genetic information.

In some embodiments, the pharmaceutical composition comprising an anellovector described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector is sufficient to compete with chronic or acute viral infection. In certain embodiments, the anellovector may be administered prophylactically to protect from viral infections (e.g. a provirotic). In some embodiments, the anellovector is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more). In some embodiments, treatment, treating, and cognates thereof comprise medical management of a subject (e.g., by administering an anellovector, e.g., an anellovector made as described herein), e.g., with the intent to improve, ameliorate, stabilize, prevent or cure a disease, pathological condition, or disorder. In some embodiments, treatment comprises active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder), and/or supportive treatment (treatment employed to supplement another therapy).

### VIII. Methods of Amplifying Anellovirus Sequences

The present disclosure provides, in some aspects, methods of amplifying nucleic acid molecules comprising an Anellovirus sequence. In some embodiments, such methods comprise rolling-circle amplification, e.g., a targeted rolling-circle amplification. In some embodiments, such methods may be used to identify and isolate Anellovirus sequences from a sample. In some embodiments, the present disclosure provides methods of determining the Anellovirus profile (also referred to as an anellome) of a subject. In embodiments, the Anellovirus profile of a subject comprises a compilation of Anellovirus sequences identified from a sample obtained from the subject. In embodiments, the Anellovirus profile of a subject can be used to identify the population of Anellovirus strains present in the subject, or a sample obtained therefrom.

DNA Amplification

The methods herein can be used to identify and isolate Anellovirus sequences from a sample (e.g., a sample from a subject, e.g., as described herein). In some embodiments, the present disclosure relates to a method of amplifying a circular nucleic acid molecule comprising an Anellovirus sequence. In some embodiments, a method comprises a step of providing a sample comprising a circular nucleic acid molecule comprising an Anellovirus sequence and a primer that binds to (e.g., is complementary to) at least a portion of the Anellovirus sequence. In some embodiments, a method comprises a step of contacting a circular nucleic acid molecule comprising an Anellovirus sequence with a DNA-dependent DNA polymerase molecule. In some embodiments, a method comprises rolling circle amplification of a nucleic acid molecule, or a portion thereof, wherein the nucleic acid molecule comprises an Anellovirus sequence. While may of the methods described herein (e.g., involving rolling circle amplification) are suitable for amplifying circular DNA, it is understood that methods described herein can also be used to amplify a linear template. For example, the linear template can be a fragment of an Anellovirus genome. In some embodiments, the linear template is amplified using multiple strand displacement amplification. Amplification may, in some embodiments, be exponential (e.g., using PCR amplification) or linear (e.g., using rolling circle amplification or multiple strand displacement amplification).

Rolling Circle Amplification

Rolling circle amplification is a form of DNA and/or RNA replication that facilitates replication and amplification of circular nucleic acid molecules. In some instances, rolling circle amplification is performed using a DNA polymerase (e.g., a DNA-dependent DNA polymerase) with strand displacement activity to extend one or more primers annealed to a circular nucleic acid template. In some embodiments, strand displacement activity enables displacement of the newly synthesized nucleic acid strand to allow further templating and generates a long single-stranded DNA or RNA molecule comprising a repeated sequence complimentary to the circular nucleic acid template.

In some embodiments, a method of rolling circle amplification described herein comprises a step of providing a sample comprising a circular nucleic acid molecule and one or more primers complementary to at least a portion of the circular nucleic acid molecule and a step of contacting the sample comprising the circular nucleic acid molecule and one or more primers with a DNA polymerase molecule (e.g., a DNA-dependent DNA polymerase molecule).

In some embodiments, a method of rolling circle amplification further comprises, e.g., prior to contacting of a circular nucleic acid molecule with a primer and/or a DNA polymerase, a step of enriching a sample comprising a circular nucleic acid molecule for one or more constituents of interest. In some embodiments, the one or more constituents of interest comprise nucleic acid molecules. For example, in some embodiments, the one or more constituents of interest comprise non-chromosomal nucleic acid molecules, e.g., circular non-chromosomal nucleic acid molecules and/or viral nucleic acid molecules (e.g. Anellovirus nucleic acid molecules, e.g., Anellovirus genomes, or portions thereof, e.g., comprising at least 100, 200, 300, 400, 500, 600, 700, 800, 900, 1000, 1500, 2000, 2500, or 3000 nucleotides of an Anellovirus genome).

In some embodiments, a method of rolling circle amplification further comprises a step of denaturing the circular nucleic acid molecule in the sample prior to the step of contacting the sample with a DNA-dependent DNA polymerase. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for a period of time, e.g., at least about 1, 2, 3, 4, or 5 minutes. In some embodiments, a step of denaturing the circular nucleic acid molecule comprises exposing the circular nucleic acid molecule to a temperature of at least about 80° C. In some embodiments, a step of denaturing the circular nucleic acid molecule comprises exposing the circular nucleic acid molecule to a temperature of at least about 85° C. In some embodiments, a step of denaturing the circular nucleic acid molecule comprises exposing the circular nucleic acid molecule to a temperature of at least about 90° C. In some embodiments, a step of denaturing the circular nucleic acid molecule comprises exposing the circular nucleic acid molecule to a temperature of at least about 91° C. In some embodiments, a step of denaturing the circular nucleic acid molecule comprises exposing the circular nucleic acid molecule to a temperature of at least about 92° C. In some embodiments, a step of denaturing the circular nucleic acid molecule comprises exposing the circular nucleic acid molecule to a temperature of at least about 93° C. In some embodiments, a step of denaturing the circular nucleic acid molecule comprises exposing the circular nucleic acid molecule to a temperature of at least about 94° C. In some embodiments, a step of denaturing the circular nucleic acid molecule comprises exposing the circular nucleic acid molecule to a temperature of at least about 95° C. In some embodiments, a step of denaturing the circular nucleic acid molecule comprises exposing the circular nucleic acid molecule to a temperature of at least about 96° C. In some embodiments, a step of denaturing the circular nucleic acid molecule comprises exposing the circular nucleic acid molecule to a temperature of at least about 97° C. In some embodiments, a step of denaturing the circular nucleic acid molecule comprises exposing the circular nucleic acid molecule to a temperature of at least about 98° C. In some embodiments, a step of denaturing the circular nucleic acid molecule comprises exposing the circular nucleic acid molecule to a temperature of at least about 99° C. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for at least about 1 minute. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for at least about 2 minutes. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for at least about 3 minutes. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for at least about 4 minutes. In some embodiments, a step of denaturing the circular nucleic acid molecule comprises exposing the circular nucleic acid molecule to a temperature of at least about 80, 85, 90, 91, 92, 93, 94, 95, 96, 97, 98, or 99° C. for at least about 5 minutes.

In some embodiments, a method of rolling circle amplification further comprises a step of cooling the circular nucleic acid molecule, e.g., after a step of denaturing the circular nucleic acid molecule and prior to the step of contacting the sample with a DNA-dependent DNA polymerase. In some embodiments, a step of cooling the circular nucleic acid molecule comprises cooling the circular nucleic acid molecule to a temperature of about 2, 3, 4, 5, 6, or 7° C. In some embodiments, a step of cooling the circular nucleic acid molecule comprises cooling the circular nucleic acid molecule to a temperature of about 2° C. In some embodiments, a step of cooling the circular nucleic acid molecule comprises cooling the circular nucleic acid molecule to a temperature of about 3° C. In some embodiments, a step of cooling the circular nucleic acid molecule comprises cooling the circular nucleic acid molecule to a temperature of about 4° C. In some embodiments, a step of cooling the circular nucleic acid molecule comprises cooling the circular nucleic acid molecule to a temperature of about 5° C. In some embodiments, a step of cooling the circular nucleic acid molecule comprises cooling the circular nucleic acid molecule to a temperature of about 6° C. In some embodiments, a step of cooling the circular nucleic acid molecule comprises cooling the circular nucleic acid molecule to a temperature of about 7° C.

In some embodiments, a method of rolling circle amplification further comprises one or more steps of incubating the sample after the step of contacting the sample with a DNA-dependent DNA polymerase. In some embodiments, a first incubation step comprises incubating the sample in the presence of a DNA-dependent DNA polymerase at a temperature of about 25, 26, 27, 28, 29, 30, 31, 32, 33, 34, or 35° C., for a period of time, e.g., for at least about 10, 15, 16, 17, 18, 19, 20, 21, 22, 23, 24, 25, or 30 hours. In some embodiments, a first incubation step comprises incubating the sample at a temperature of about 25° C. In some embodiments, a first incubation step comprises incubating the sample at a temperature of about 26° C. In some embodiments, a first incubation step comprises incubating the sample at a temperature of about 27° C. In some embodiments, a first incubation step comprises incubating the sample at a temperature of about 28° C. In some embodiments, a first incubation step comprises incubating the sample at a temperature of about 29° C. In some embodiments, a first incubation step comprises incubating the sample at a temperature of about 30° C. In some embodiments, a first incubation step comprises incubating the sample at a temperature of about 31° C. In some embodiments, a first incubation step comprises incubating the sample at a temperature of about 32° C. In some embodiments, a first incubation step comprises incubating the sample at a temperature of about 33° C. In some embodiments, a first incubation step comprises incubating the sample at a temperature of about 34° C. In some embodiments, a first incubation step comprises incubating the sample at a temperature of about 35° C. In some embodiments, a first incubation step comprises incubating the sample at a temperature of about 25, 26, 27, 28, 29, 30, 31, 32, 33, 34, or 35° C., for a period of time, e.g., for at least about 10 hours. In some embodiments, a first incubation step comprises incubating the sample at a temperature of about 25, 26, 27, 28, 29, 30, 31, 32, 33, 34, or 35° C., for a period of time, e.g., for at least about 15 hours. In some embodiments, a first incubation step comprises incubating the sample at a temperature of about 25, 26, 27, 28, 29, 30, 31, 32, 33, 34, or 35° C., for a period of time, e.g., for at least about 16 hours. In some embodiments, a first incubation step comprises incubating the sample at a temperature of about 25, 26, 27, 28, 29, 30, 31, 32, 33, 34, or 35° C., for a period of time, e.g., for at least about 17 hours. In some embodiments, a first incubation step comprises incubating the sample at a temperature of about 25, 26, 27, 28, 29, 30, 31, 32, 33, 34, or 35° C., for a period of time, e.g., for at least about 18 hours. In some embodiments, a first incubation step comprises incubating the sample at a temperature of about 25, 26, 27, 28, 29, 30, 31, 32, 33, 34, or 35° C., for a period of time, e.g., for at least about 19 hours. In some embodiments, a first incubation step comprises incubating the sample at a temperature of about 25, 26, 27, 28, 29, 30, 31, 32, 33, 34, or 35° C., for a period of time, e.g., for at least about 20 hours. In some embodiments, a first incubation step comprises incubating the sample at a temperature of about 25, 26, 27, 28, 29, 30, 31, 32, 33, 34, or 35° C., for a period of time, e.g., for at least about 21 hours. In some embodiments, a first incubation step comprises incubating the sample at a temperature of about 25, 26, 27, 28, 29, 30, 31, 32, 33, 34, or 35° C., for a period of time, e.g., for at least about 22 hours. In some embodiments, a first incubation step comprises incubating the sample at a temperature of about 25, 26, 27, 28, 29, 30, 31, 32, 33, 34, or 35° C., for a period of time, e.g., for at least about 23 hours. In some embodiments, a first incubation step comprises incubating the sample at a temperature of about 25, 26, 27, 28, 29, 30, 31, 32, 33, 34, or 35° C., for a period of time, e.g., for at least about 24 hours. In some embodiments, a first incubation step comprises incubating the sample at a temperature of about 25, 26, 27, 28, 29, 30, 31, 32, 33, 34, or 35° C., for a period of time, e.g., for at least about 25 hours. In some embodiments, a first incubation step comprises incubating the sample at a temperature of about 25, 26, 27, 28, 29, 30, 31, 32, 33, 34, or 35° C., for a period of time, e.g., for at least about 30 hours. In some embodiments, a second incubation step comprises incubating the sample under conditions suitable to inactivate the DNA-dependent DNA polymerase. For example, in some embodiments, a second incubation step comprises incubating the sample at a temperature of about 60, 61, 62, 63, 64, 65, 66, 67, 68, 69, or 70° C. for a period of time, e.g., for at least 5, 6, 7, 8, 9, 10, 11, 12, 13, 14, or 15 minutes. In some embodiments, a second incubation step comprises incubating the sample at a temperature of about 60° C. In some embodiments, a second incubation step comprises incubating the sample at a temperature of about 61° C. In some embodiments, a second incubation step comprises incubating the sample at a temperature of about 62° C. In some embodiments, a second incubation step comprises incubating the sample at a temperature of about 63° C. In some embodiments, a second incubation step comprises incubating the sample at a temperature of about 64° C. In some embodiments, a second incubation step comprises incubating the sample at a temperature of about 65° C. In some embodiments, a second incubation step comprises incubating the sample at a temperature of about 66° C. In some embodiments, a second incubation step comprises incubating the sample at a temperature of about 67° C. In some embodiments, a second incubation step comprises incubating the sample at a temperature of about 68° C. In some embodiments, a second incubation step comprises incubating the sample at a temperature of about 69° C. In some embodiments, a second incubation step comprises incubating the sample at a temperature of about 70° C. In some embodiments, a second incubation step comprises incubating the sample at a temperature of about 60, 61, 62, 63, 64, 65, 66, 67, 68, 69, or 70° C. for at least 5 minutes. In some embodiments, a second incubation step comprises incubating the sample at a temperature of about 60, 61, 62, 63, 64, 65, 66, 67, 68, 69, or 70° C. for at least 6 minutes. In some embodiments, a second incubation step comprises incubating the sample at a temperature of about 60, 61, 62, 63, 64, 65, 66, 67, 68, 69, or 70° C. for at least 7 minutes. In some embodiments, a second incubation step comprises incubating the sample at a temperature of about 60, 61, 62, 63, 64, 65, 66, 67, 68, 69, or 70° C. for at least 8 minutes. In some embodiments, a second incubation step comprises incubating the sample at a temperature of about 60, 61, 62, 63, 64, 65, 66, 67, 68, 69, or 70° C. for at least 9 minutes. In some embodiments, a second incubation step comprises incubating the sample at a temperature of about 60, 61, 62, 63, 64, 65, 66, 67, 68, 69, or 70° C. for at least 10 minutes. In some embodiments, a second incubation step comprises incubating the sample at a temperature of about 60, 61, 62, 63, 64, 65, 66, 67, 68, 69, or 70° C. for at least 11 minutes. In some embodiments, a second incubation step comprises incubating the sample at a temperature of about 60, 61, 62, 63, 64, 65, 66, 67, 68, 69, or 70° C. for at least 12 minutes. In some embodiments, a second incubation step comprises incubating the sample at a temperature of about 60, 61, 62, 63, 64, 65, 66, 67, 68, 69, or 70° C. for at least 13 minutes. In some embodiments, a second incubation step comprises incubating the sample at a temperature of about 60, 61, 62, 63, 64, 65, 66, 67, 68, 69, or 70° C. for at least 14 minutes. In some embodiments, a second incubation step comprises incubating the sample at a temperature of about 60, 61, 62, 63, 64, 65, 66, 67, 68, 69, or 70° C. for at least 15 minutes.

In some embodiments, in a method of rolling circle amplification, the step of contacting the circular nucleic acid molecule and one or more primers with a DNA-dependent DNA polymerase molecule occurs in a mixture having a concentration of the one or more primers of about 0.1, 0.2, 0.3, 0.4, 0.5, 0.6, 0.7, or 0.8 μM per primer, or about 0.1-0.2, 0.2-0.3, 0.3-0.4, 0.4-0.5, 0.5-0.6, 0.6-0.7, or 0.7-0.8 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.1, 0.2, 0.3, 0.4, 0.5, 0.6, 0.7, or 0.8 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.1 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.2 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.3 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.4 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.5 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.6 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.7 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.8 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.1-0.2, 0.2-0.3, 0.3-0.4, 0.4-0.5, 0.5-0.6, 0.6-0.7, or 0.7-0.8 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.1-0.2 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.2-0.3 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.3-0.4 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.4-0.5 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.5-0.6 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.6-0.7 μM per primer. In some embodiments, in a method of rolling circle amplification, the step of contacting the circular nucleic acid molecule and one or more primers with a DNA-dependent DNA polymerase molecule occurs in a mixture having a concentration of the one or more primers of about 0.7-0.8 μM per primer.

In some embodiments, in a method of rolling circle amplification, the step of contacting the circular nucleic acid molecule and one or more primers with a DNA-dependent DNA polymerase molecule occurs in a mixture having a DNA polymerase buffer suitable for the DNA-dependent DNA polymerase to synthesize DNA (e.g., a Phi29 DNA polymerase buffer).

In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 100, 150, 160, 170, 180, 190, 200, 210, 220, 230, 240, 250, or 300 ng/μL, or about 100-150, 150-175, 175-190, 190-200, 200-210, 210-225, 225-250, or 250-300 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 100, 150, 160, 170, 180, 190, 200, 210, 220, 230, 240, 250, or 300 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 100 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 150 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 160 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 170 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 180 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 190 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 200 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 210 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 220 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 230 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 240 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 250 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 300 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 100-150, 150-175, 175-190, 190-200, 200-210, 210-225, 225-250, or 250-300 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 100-150 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 150-175 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 175-190 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 190-200 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 200-210 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 210-225 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 225-250 ng/μL. In some embodiments, in a method of rolling circle amplification, the step of contacting the circular nucleic acid molecule and one or more primers with a DNA-dependent DNA polymerase molecule occurs in a mixture comprising bovine albumin serum, e.g., at a concentration of about 250-300 ng/μL.

In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.5, 0.6, 0.7, 0.8, 0.9, 1.0, 1.1, 1.2, 1.3, 1.4, 1.5, or 2 mM, or about 0.5-0.7, 0.7-0.9, 0.9-1.0, 1.0-1.1, 1.1-1.3, 1.3-1.5, or 1.5-2 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.5, 0.6, 0.7, 0.8, 0.9, 1.0, 1.1, 1.2, 1.3, 1.4, 1.5, or 2 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.5 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.6 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.7 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.8 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 0.9 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.0 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.1 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.2 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.3 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.4 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 1.5 mM. In some embodiments, in a method of rolling circle amplification, the step of contacting the circular nucleic acid molecule and one or more primers with a DNA-dependent DNA polymerase molecule occurs in a mixture comprising dNTPs, e.g., at a concentration of about 2 mM.

In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1, 1.5, 2, 2.5, or 3 U/μL, or about 1-1.5, 1.5-2, 2-2.5, or 2.5-3 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1, 1.5, 2, 2.5, or 3 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1.5 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 2 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 2.5 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 3 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1-1.5, 1.5-2, 2-2.5, or 2.5-3 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1-1.5 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 1.5-2 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 2-2.5 U/μL. In some embodiments, in a method of rolling circle amplification, the step of contacting the circular nucleic acid molecule and one or more primers with a DNA-dependent DNA polymerase molecule occurs in a mixture having Phi29 polymerase, e.g., at a concentration of about 2.5-3 U/μL.

In some embodiments, a method of rolling circle amplification does not comprise thermocycling, e.g., is performed isothermally. In some embodiments, a method of rolling circle amplification comprises displacement (e.g., partial or full displacement) of the strand synthesized by the DNA-dependent DNA polymerase from the circular nucleic acid molecule. In some embodiments, in a method of rolling circle amplification, the strand synthesized by the DNA-dependent DNA polymerase is released into the surrounding solution. In some embodiments, in a method of rolling circle amplification, the DNA-dependent DNA polymerase nicks the synthesized strand, thereby releasing the synthesized strand.

In some embodiments, in a method of rolling circle amplification, the DNA-dependent DNA polymerase synthesizes a product strand comprising a plurality of copies of the sequence of the circular nucleic acid, or a plurality of copies of a fragment thereof comprising at least 100, 200, 300, 400, 500, 600, 700, 800, 900, 1000, 1100, 1200, 1300, 1400, 1500, 1600, 1700, 1800, 1900, 2000, 2500, 3000, 3500, or 4000 contiguous nucleotides thereof. In some embodiments, a plurality of copies of the sequence of the circular nucleic acid, or the fragment thereof, are arranged in tandem within the product strand. In embodiments, the plurality of copies arranged in tandem are each separated by between 0-1, 1-5, 5-10, 10-15, 15-20, 20-25, 25-30, 30-40, 40-50, 50-60, 60-70, 70-80, 80-90, or 90-100 nucleotides (e.g., about 0, 1, 2, 3, 4, 5, 6, 7, 8, 9, 10, 15, 20, 25, 30, 35, 40, 45, 50, 60, 70, 80, 90, or 100 nucleotides). In some embodiments, in a method of rolling circle amplification, the DNA-dependent DNA polymerase synthesizes a product strand comprising one copy of the sequence of the circular nucleic acid, or a fragment thereof comprising at least 1000, 2000, 2500, 3000, 3500, or 4000 contiguous nucleotides thereof.

In some embodiments, a method of rolling circle amplification is validated by PCR, e.g., using one or more pan-Anelloviruse primers, e.g., as described in Ninomiya et al. 2008 (J. Clin. Microbiol. 46: 507-514; incorporated herein by reference with respect to the pan-Anellovirus primers and methods relating to same). In some embodiments, an amplified nucleic acid molecule prepared by a method of rolling circle amplification, e.g., as described herein, is assessed by library quality control (QC) techniques, e.g., as described herein. In embodiments, the QC techniques include assessment of library size, e.g., prior to sequencing. In embodiments, the QC techniques include assessment of library concentration, e.g., prior to sequencing. In an embodiment, an Agilent Tapestation 4200 is used (e.g., with D5000 screen tape) to assess library size and/or concentration. In embodiments, the amplified nucleic acid molecule is assessed by gel electrophoresis (e.g., by identifying the presence of a band at an expected size, e.g., at about 110, 115, 120, 121, 122, 123, 124, 125, 126, 127, 128, 129, 130, 131, 132, 133, 134, 135, 140, or 150 bp). In an embodiment, the expected size of the band is 128 bp.

Primers

The amplification methods described herein generally involve contacting a nucleic acid molecule comprising an Anellovirus sequence with a primer, thereby permitting a DNA polymerase (e.g., a DNA-dependent DNA polymerase) to initiate DNA synthesis from the primer. In some embodiments, a plurality of primers used in a method described herein is based on a degenerate sequence, e.g., comprising one or more (e.g., 1, 2, 3, 4, 5, 6, 7, 8, 9, or 10) variable positions (e.g., such that a plurality of the degenerate primers may comprise a plurality of different nucleotides at the one or more variable positions). In some embodiments, a primer used in a method described herein is a primer specific for an Anellovirus sequence, or the method uses a plurality of Anellovirus-specific primers. In embodiments, the primer comprises a nucleic acid sequence that is the reverse complement to a nucleic acid sequence comprised in an Anellovirus sequence, e.g., as described herein. In some embodiments, a plurality of primers (e.g., as described herein) are used in the methods described herein. In some embodiments, the plurality of primers comprise primers having at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more different sequences (e.g., due to degeneracy at one or more (e.g., 1, 2, 3, 4, 5, 6, 7, 8, 9, or 10) variable positions within the primers).

In some embodiments, a plurality of degenerate primers are used in the methods described herein. In some embodiments, wherein one or more primers are used in the methods described herein, a first primer has at least 70%, 75%, 80%, 85%, 90%, 95%, 96%, 97%, 98%, 99%, or 100% sequence identity to a second primer, and wherein the first primer and the second primer are not identical. In some embodiments, wherein one or more primers are used in the methods described herein, a first primer has at least 70% sequence identity to a second primer, and wherein the first primer and the second primer are not identical. In some embodiments, wherein one or more primers are used in the methods described herein, a first primer has at least 75% sequence identity to a second primer, and wherein the first primer and the second primer are not identical. In some embodiments, wherein one or more primers are used in the methods described herein, a first primer has at least 80% sequence identity to a second primer, and wherein the first primer and the second primer are not identical. In some embodiments, wherein one or more primers are used in the methods described herein, a first primer has at least 85% sequence identity to a second primer, and wherein the first primer and the second primer are not identical. In some embodiments, wherein one or more primers are used in the methods described herein, a first primer has at least 90% sequence identity to a second primer, and wherein the first primer and the second primer are not identical. In some embodiments, wherein one or more primers are used in the methods described herein, a first primer has at least 95% sequence identity to a second primer, and wherein the first primer and the second primer are not identical. In some embodiments, wherein one or more primers are used in the methods described herein, a first primer has at least 96% sequence identity to a second primer, and wherein the first primer and the second primer are not identical. In some embodiments, wherein one or more primers are used in the methods described herein, a first primer has at least 97% sequence identity to a second primer, and wherein the first primer and the second primer are not identical. In some embodiments, wherein one or more primers are used in the methods described herein, a first primer has at least 98% sequence identity to a second primer, and wherein the first primer and the second primer are not identical. In some embodiments, wherein one or more primers are used in the methods described herein, a first primer has at least 99% sequence identity to a second primer, and wherein the first primer and the second primer are not identical.

In some embodiments, a method described herein uses a plurality of primers. In some embodiments, a plurality of primers shares the same orientation relative to the circular nucleic acid molecule of the methods described herein. In some embodiments, a plurality of primers are all positive-strand primers or all negative-strand primers. In some embodiments, a plurality of primers are all positive-strand primers. In some embodiments, a plurality of primers are all negative strand primers. In some embodiments, a plurality of primers comprises at least 3, 4, 5, 6, 7, 8, 9, or 10 contiguous nucleotides in common. In some embodiments, a plurality of primers comprises at least 3 contiguous nucleotides in common. In some embodiments, a plurality of primers comprises at least 4 contiguous nucleotides in common. In some embodiments, a plurality of primers comprises at least 5 contiguous nucleotides in common. In some embodiments, a plurality of primers comprises at least 6 contiguous nucleotides in common. In some embodiments, a plurality of primers comprises at least 7 contiguous nucleotides in common. In some embodiments, a plurality of primers comprises at least 8 contiguous nucleotides in common. In some embodiments, a plurality of primers comprises at least 9 contiguous nucleotides in common. In some embodiments, a plurality of primers comprises at least 10 contiguous nucleotides in common. In some embodiments, a plurality of primers comprises at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more different primers. In some embodiments, a plurality of primers comprises at least 2 different primers. In some embodiments, a plurality of primers comprises at least 3 different primers. In some embodiments, a plurality of primers comprises at least 4 different primers. In some embodiments, a plurality of primers comprises at least 5 different primers. In some embodiments, a plurality of primers comprises at least 10 different primers. In some embodiments, a plurality of primers comprises at least 12 different primers. In some embodiments, a plurality of primers comprises at least 15, 20, 25, 30, 35, 40, 45, or 50 different primers.

In some embodiments, a primer used in a method described herein comprises a nucleic acid sequence selected from the primers listed in Table A. In some embodiments, a primer used in a method described herein comprises a nucleic acid sequence of a sense sequence listed in Table A. In some embodiments, a primer used in a method described herein comprises a nucleic acid sequence of an antisense sequence listed in Table A. In some embodiments, a primer comprises a nucleic acid sequence selected from the group consisting of CGAATGGYW, AAGGGGCAA, YTGYGGBTG, YAGAMACMM, YAARTGGTAC, SACCACWAAC, TBGTCGGTG, CACTCCGAG, GAGGAGTGC, CAGACTCCG, GTGAGTGGG, and CTTCGCCAT. In some embodiments, a primer comprises a nucleic acid sequence selected from the group consisting of WRCCATTCG, TTGCCCCTT, CAVCCRCAR, KKGTKTCTR, GTACCAYTTR, GTTWGTGGTS, CACCGACVA, CTCGGAGTG, GCACTCCTC, CGGAGTCTG, CCCACTCAC, and ATGGCGAAG. In some embodiments, a primer comprises a nucleic acid sequence selected from the group consisting of CGAATGGYW, TTGCCCCTT, YTGYGGBTG, YAGAMACMM, GTACCAYTTR, SACCACWAAC, CACCGACVA, CACTCCGAG, GCACTCCTC, CAGACTCCG, CCCACTCAC, and CTTCGCCAT. In some embodiments, a primer is CGAATGGYW. In some embodiments, a primer is AAGGGGCAA. In some embodiments, a primer is YTGYGGBTG. In some embodiments, a primer is YAGAMACMM. In some embodiments, a primer is YAARTGGTAC. In some embodiments, a primer is SACCACWAAC. In some embodiments, a primer is TBGTCGGTG. In some embodiments, a primer is CACTCCGAG. In some embodiments, a primer is GAGGAGTGC. In some embodiments, a primer is CAGACTCCG. In some embodiments, a primer is GTGAGTGGG. In some embodiments, a primer is CTTCGCCAT. In some embodiments, a primer is WRCCATTCG. In some embodiments, a primer is TTGCCCCTT. In some embodiments, a primer is CAVCCRCAR. In some embodiments, a primer is KKGTKTCTR. In some embodiments, a primer is GTACCAYTTR. In some embodiments, a primer is GTTWGTGGTS. In some embodiments, a primer is CACCGACVA. In some embodiments, a primer is CTCGGAGTG. In some embodiments, a primer is GCACTCCTC. In some embodiments, a primer is CGGAGTCTG. In some embodiments, a primer is CCCACTCAC. In some embodiments, a primer is ATGGCGAAG.

TABLE A
Exemplary sense and antisense sequences for primers

| SEQ | Sense | SEQ | Antisense |
|---|---|---|---|
| ID NO: | Sequence | ID NO: | Sequence |
| 1 | CGAATGGYW | 13 | WRCCATTCG |
| 2 | AAGGGGCAA | 14 | TTGCCCCTT |
| 3 | YTGYGGBTG | 15 | CAVCCRCAR |
| 4 | YAGAMACMM | 16 | KKGTKTCTR |
| 5 | YAARTGGTAC | 17 | GTACCAYTTR |
| 6 | SACCACWAAC | 18 | GTTWGTGGTS |
| 7 | TBGTCGGTG | 19 | CACCGACVA |
| 8 | CACTCCGAG | 20 | CTCGGAGTG |
| 9 | GAGGAGTGC | 21 | GCACTCCTC |
| 10 | CAGACTCCG | 22 | CGGAGTCTG |
| 11 | GTGAGTGGG | 23 | CCCACTCAC |
| 12 | CTTCGCCAT | 24 | ATGGCGAAG |

TABLE B
The UPAC nucleotide code, which is used herein
unless otherwise specified.

| UPAC nucleotide code | Base |
|---|---|
| A | Adenine (A) |
| C | Cytosine (C) |
| G | Guanine (G) |
| T | Thymine (T) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

In some embodiments, a primer comprises (e.g., is protected by) one or more thiophosphate modifications. In some embodiments, a primer comprises 1, 2, 3, or 4 thiophosphate modifications. In some embodiments, a primer comprises 1 thiophosphate modification. In some embodiments, a primer comprises 2 thiophosphate modifications. In some embodiments, a primer comprises 3 thiophosphate modifications. In some embodiments, a primer comprises 4 thiophosphate modifications. In some embodiments, a primer comprises a thiophosphate modification positioned between the last 2 nucleotides at the 3′ end. In some embodiments, a primer comprises a thiophosphate modification positioned between the second and third 3′-most nucleotides. In some embodiments, a primer comprises 2 thiophosphate modifications between each of the last 3 nucleotides at the 3′ end. In some embodiments, a primer comprises 3 thiophosphate modifications between each of the last 4 nucleotides at the 3′ end. In some embodiments, a primer comprises 4 thiophosphate modifications between each of the last 5 nucleotides at the 3′ end.

Samples and Target Sequences

In some embodiments, a sample is obtained from one or more subjects (e.g., one or more human subjects, e.g., one or more healthy or asymptomatic human subjects). In some embodiments, a sample is a biological sample. In some embodiments a sample is a biological sample obtained from one or more subjects (e.g., one or more human subjects, e.g., one or more healthy or asymptomatic human subjects). In some embodiments, a biological sample comprises blood or serum.

In some embodiments, a method of amplification, e.g., rolling circle amplification, is performed on a plurality of samples (e.g., at least 5, 10, 15, 20, 25, 30, 40, 50, 60, 70, 80, 90, 100, 110, 120, 125, 126, 127, 128, 129, 130, 140, 150, 160, 170, 180, 190, 200, 250, 300, 400, 500, 600, 700, 800, 900, or 1000 samples), e.g., in parallel. In some embodiments, the plurality of samples is obtained from a plurality of subjects (e.g., human subjects), e.g., at least 5, 10, 15, 20, 25, 30, 40, 50, 60, 70, 80, 90, 100, 110, 120, 125, 126, 127, 128, 129, 130, 140, 150, 160, 170, 180, 190, 200, 250, 300, 400, 500, 600, 700, 800, 900, or 1000 subjects, e.g., serially or in parallel. In some embodiments, the plurality of samples is obtained from a plurality of time points (e.g., a plurality of samples obtained from the same subject at multiple time points, or a plurality of samples obtained from a plurality of subjects at multiple time points). In some embodiments, the plurality of samples is obtained from a plurality of tissue or cell types, e.g., at least 5, 10, 15, 20, 25, 30, 40, 50, 60, 70, 80, 90, or 100 different tissue or cell types.

In some embodiments, a sample comprises at least 2, 3, 4, 5, 6, 7, 8, 9, or 10 different circular nucleic acid molecules (e.g., comprising at least 2, 3, 4, 5, 6, 7, 8, 9, or 10 different Anellovirus sequences). In some embodiments, a sample comprises at least 2 different circular nucleic acid molecules. In some embodiments, a sample comprises at least 3 different circular nucleic acid molecules. In some embodiments, a sample comprises at least 4 different circular nucleic acid molecules. In some embodiments, a sample comprises at least 5 different circular nucleic acid molecules. In some embodiments, a sample comprises at least 6 different circular nucleic acid molecules. In some embodiments, a sample comprises at least 7 different circular nucleic acid molecules. In some embodiments, a sample comprises at least 8 different circular nucleic acid molecules. In some embodiments, a sample comprises at least 9 different circular nucleic acid molecules. In some embodiments, a sample comprises at least 10 different circular nucleic acid molecules. In some embodiments, a sample comprises at least 2 different Anellovirus sequences. In some embodiments, a sample comprises at least 3 different Anellovirus sequences. In some embodiments, a sample comprises at least 4 different Anellovirus sequences. In some embodiments, a sample comprises at least 5 different Anellovirus sequences. In some embodiments, a sample comprises at least 6 different Anellovirus sequences. In some embodiments, a sample comprises at least 7 different Anellovirus sequences. In some embodiments, a sample comprises at least 8 different Anellovirus sequences. In some embodiments, a sample comprises at least 9 different Anellovirus sequences. In some embodiments, a sample comprises at least 10 different Anellovirus sequences. In some embodiments, a circular nucleic acid molecule encodes one or more elements from the genome sequence of an Anellovirus. In some such embodiments, the one or more elements comprised and/or encoded in the genome sequence of the Anellovirus comprises one or more of: a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, and/or GC rich region.

Sequencing

Nucleic acid molecules comprising Anellovirus sequences (e.g., amplified according to the methods described herein) can be sequenced according to sequencing methods known in the art. Sequencing methods that can be used include traditional Sanger sequencing as well as next generation deep sequencing methods, in which large quantities of nucleic acid molecules are sequenced in massively parallel fashion.

In some embodiments, the methods described herein further comprise sequencing circular nucleic acid molecules amplified according to methods described herein (e.g., enriched for circular nucleic acid molecules comprising Anellovirus sequences). In some embodiments, sequencing comprises next-generation sequencing (e.g., sequencing by synthesis (e.g., Illumina sequencing), pyrosequencing, reversible terminator sequencing, sequencing by ligation, or nanopore sequencing). In some embodiments, sequencing comprises Sanger sequencing. In some embodiments, sequencing comprises use of benchtop sequencing instrumentation (e.g., an Illumina iSeq 100 or an Illumina NextSeq 550). In some embodiments, two or more different sequencing methods are used in a method described herein.

In some embodiments, a plurality of sequencing reads obtained by such methods can be analyzed and assembled into larger contiguous sequences (generally referred to herein as contigs), which correspond to a larger portion of the source nucleic acid sequence (e.g., a single circular nucleic acid molecule as described herein). In some embodiments, a contig comprises an Anellovirus genome sequence, or a contiguous fragment thereof, e.g., comprising at least 50, 100, 200, 300, 400, 500, 600, 700, 800, 900, 1000, 1200, 1400, 1500, 1600, 1800, 2000, 2500, or 3000 contiguous nucleic acids thereof. In some embodiments, a contig comprises an Anellovirus sequence encoding one or more of: a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, and/or a GC rich region, or a fragment thereof (e.g., comprising at least 5, 10, 15, 20, 25, 30, 35, 40, 45, 50, 60, 70, 80, 90, 100, 110, 120, 130, 140, 150, 160, 170, 180, 190, 200 300, 400, 500, 600, 700, 800, 900, or 1000 contiguous nucleotides thereof), e.g., of an Anellovirus described herein. In some embodiments, a contig comprises a nucleic acid sequence encoding an Anellovirus ORF1 molecule, or a fragment thereof (e.g., comprising at least 5, 10, 15, 20, 25, 30, 35, 40, 45, 50, 60, 70, 80, 90, 100, 110, 120, 130, 140, 150, 160, 170, 180, 190, 200 300, 400, 500, 600, 700, 800, 900, 1000, 1200, 1400, 1600, 1800, or 2000 contiguous nucleotides thereof). In some embodiments, a contig comprises the nucleic acid sequence of an Anellovirus 5′ UTR, or a fragment thereof (e.g., comprising at least 5, 10, 15, 20, 25, 30, 35, 40, 45, 50, 60, 70, 80, 90, or 100 contiguous nucleotides thereof).

In some embodiments, the Anellovirus sequence is an Alphatorquevirus sequence (e.g., an Alphatorquevirus 5′ UTR sequence or an Alphatorquevirus ORF1 molecule-encoding sequence). In some embodiments, the Anellovirus sequence is a Betatorquevirus sequence (e.g., a Betatorquevirus 5′ UTR sequence or a Betatorquevirus ORF1 molecule-encoding sequence). In some embodiments, the Anellovirus sequence is a Gammatorquevirus sequence (e.g., a Gammatorquevirus 5′ UTR sequence or a Gammatorquevirus ORF1 molecule-encoding sequence).

Computational Analysis

In some embodiments, the methods described herein further comprise computational analysis of the sequencing results. Such computational analyses may, in some embodiments, be used to identify and/or classifying (e.g., within an Anellovirus clade described herein) one or more Anellovirus strains present in the sample comprising the nucleic acid molecules that were sequenced. The computational analyses may, in some embodiments, be used to determine an Anellovirus profile or anellome of the sample comprising the nucleic acid molecules that were sequenced. In some instances, the computational analyses may further used to compare Anellovirus profiles or anellomes from a plurality of samples (e.g., to determine the relative frequency of certain Anellovirus clades or strains in one sample versus another).

In some embodiments, computational analysis comprises identifying one or more Anellovirus sequences represented in the sequences of the amplified nucleic acid molecules. In some embodiments, computational analysis comprises determining sequence similarity of the genome sequence or one or more elements comprised and/or encoded therein within a plurality (e.g., at least 2, 3, 4, 5, 6, 7, 8, 9, 10, 15, 20, 30, 40, 50, 60, 70, 80, 90, 100, 200, 300, 400, 500, 600, 700, 800, 900, 1000, 1100, 1200, 1300, 1400, or 1500) of distinct sequences of the amplified nucleic acid molecules. In some embodiments, computational analysis comprises determining the Anellovirus sequences present in each sample, each subject, each tissue or cell type, and/or each time point of the methods described herein. In some embodiments, computational analysis comprises determining the unique Anellovirus lineages present in each sample, each subject, each tissue or cell type, and/or each time point of the methods described herein.

In some embodiments, computational analysis comprises comparing the sequences present in a sample to one or more reference sequences, e.g., from a database (e.g., GenBank). In some embodiments, computational analysis comprises comparing the sequences present in a sample to sequences from other known Anelloviruses. In some embodiments, computational analysis comprises comparing the sequences present in a sample to sequences of viruses other than Anelloviruses (e.g. human papillomavirus HPV, adeno-associated virus AAV, Dengue virus, Middle East respiratory syndrome-associated coronavirus MERS-CoV, Ebolavirus, Lassa fever virus, and influenza A virus, human immunodeficiency virus-1 HIV-1). In some embodiments, computational analysis comprises comparing the sequences present in one sample to another sample. In some embodiments, computational analysis comprises comparing the sequences present in one subject to another subject. In some embodiments, computational analysis comprises comparing the sequences present in one tissue or cell type to another tissue or cell type (e.g., in the same subject or in different subjects). In some embodiments, computational analysis comprises comparing the sequences present at one time point to the sequences present at another time point (e.g., comparing a sample from a subject at one time point with a sample from the same subject at a different time point, e.g., a later time point).

In some embodiments, computational analysis comprises performing multidimensional scaling (MDS) of the sequences, or portions thereof (e.g., portions comprising or encoding one or more of: a TATA box, cap site, transcriptional start site, 5′ UTR conserved domain, ORF1, ORF1/1, ORF1/2, ORF2, ORF2/2, ORF2/3, TAIP, three open-reading frame region, poly(A) signal, and/or GC rich region). In some embodiments, computational analysis comprises performing phylogenetic analysis, e.g., to classify a plurality of Anellovirus sequences present in one or more samples (e.g., by their sequence similarity and/or likely evolutionary history). In some embodiments, sequences are aligned and clustered into groups where members were at least 70%, 75%, 80%, 85%, 90%, or 95% identical at a nucleotide level. In some embodiments, sequences are aligned and clustered into groups where members were at least 75% identical at a nucleotide level. In some embodiments, sequences were aligned and clustered into groups where members are at least 80% identical at a nucleotide level. In some embodiments, sequences were aligned and clustered into groups where members are at least 85% identical at a nucleotide level. In some embodiments, sequences were aligned and clustered into groups where members are at least 90% identical at a nucleotide level. In some embodiments, MDS of portions of sequences (e.g. ORF1, ORF1/1, ORF1/2, ORF2, ORF2/2, and/or ORF2/3 region) are used to construct a maximum likelihood phylogenetic tree. In some embodiments, phylogenetic analysis further comprises recombination analysis. In some embodiments, phylogenetic trees and sequence alignments are used to identify mutations. In some embodiments, phylogenetic trees and sequence alignments are used to identify clusters of at least 2, 3, 4, 5, or 6 mutations that occurred within about 5, 6, 7, 8, 9, 10, 11, 12, 13, 14, or 15 nucleotides of each other. In some embodiments, phylogenetic trees and sequence alignments are used to identify clusters of at least 2 mutations that occurred within about 5 nucleotides of each other. In some embodiments, phylogenetic trees and sequence alignments are used to identify clusters of at least 2 mutations that occurred within about 7 nucleotides of each other. In some embodiments, phylogenetic trees and sequence alignments are used to identify clusters of at least 2 mutations that occurred within about 10 nucleotides of each other. In some embodiments, phylogenetic trees and sequence alignments are used to identify clusters of at least 3 mutations that occurred within about 5 nucleotides of each other. In some embodiments, phylogenetic trees and sequence alignments are used to identify clusters of at least 3 mutations that occurred within about 7 nucleotides of each other. In some embodiments, phylogenetic trees and sequence alignments are used to identify clusters of at least 3 mutations that occurred within about 9 nucleotides of each other. In some embodiments, phylogenetic trees and sequence alignments are used to identify clusters of at least 3 mutations that occurred within about 10 nucleotides of each other. In some embodiments, phylogenetic trees and sequence alignments are used to identify clusters of at least 3 mutations that occurred within about 11 nucleotides of each other. In some embodiments, phylogenetic trees and sequence alignments are used to identify clusters of at least 3 mutations that occurred within about 12 nucleotides of each other. In some embodiments, phylogenetic trees and sequence alignments are used to identify clusters of at least 3 mutations that occurred within about 15 nucleotides of each other. In some embodiments, phylogenetic trees and sequence alignments are used to identify clusters of at least 4 mutations that occurred within about 7 nucleotides of each other. In some embodiments, phylogenetic trees and sequence alignments are used to identify clusters of at least 4 mutations that occurred within about 10 nucleotides of each other. In some embodiments, phylogenetic trees and sequence alignments are used to identify clusters of at least 4 mutations that occurred within about 15 nucleotides of each other. In some embodiments, phylogenetic trees and sequence alignments are used to identify clusters of at least 5 mutations that occurred within about 10 nucleotides of each other. In some embodiments, phylogenetic trees and sequence alignments are used to identify clusters of at least 5 mutations that occurred within about 15 nucleotides of each other.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Anellovirus multiple dosing in human transfusion patients
Example 2: Recurring Anellovirus dosing in human transfusion patients
Example 3: Preparation of synthetic anellovectors
Example 4: Assembly and infection of anellovectors
Example 5: Selectivity of anellovectors
Example 6: Replication-deficient anellovectors and helper viruses
Example 7: Manufacturing process for replication-competent anellovectors
Example 8: Manufacturing process of replication-deficient anellovectors
Example 9: Production of anellovectors using suspension cells
Example 10: Utilizing anellovectors to express an exogenous protein in mice
Example 11: Functional effects of an anellovector expressing an exogenous microRNA sequence
Example 12: Preparation and production of anellovectors to express exogenous non-coding RNAs
Example 13: Expression of an endogenous miRNA from an anellovector and deletion of the endogenous miRNA
Example 14: Anellovector delivery of exogenous proteins in vivo
Example 15: In vitro circularized Anellovirus genomes
Example 16: Production of anellovectors containing chimeric ORF1 with hypervariable domains from different Torque Teno Virus strains
Example 17: Production of chimeric ORF1 containing non-TTV protein/peptides in place of hypervariable domains
Example 18: Anellovectors based on tth8 and LY2 each successfully transduced the EPO gene into lung cancer cells
Example 19: Anellovectors with therapeutic transgenes can be detected in vivo after intravenous (i.v.) administration
Example 20: In vitro circularized genome as input material for producing anellovectors in vitro
Example 21: Tandem copies of the Anellovirus genome
Example 22: Efficient replication of anellovectors from a tandem anellovector construct
Example 23: Exemplary tandem anellovector construct designs
Example 24: Transcription of genes from a tandem Anellovirus construct in mammalian cells
Example 25: ORF1 and ORF2 protein produced from a tandem Anellovirus construct in mammalian cells
Example 26: Assessment of infectivity of tandem Anellovectors
Example 27: Delivery of tandem anelloviral genomes into Sf9 insect cells via baculovirus
Example 28: Production of Anellovirus proteins in a baculovirus expression system
Example 29: Expression of Ring1 ORFs in Sf9 cells
Example 30: Expression of Ring2 ORFs in Sf9 cells
Example 31: Expression of all Ring2 ORFs simultaneously in Sf9 cells
Example 32: Co-delivery and independent expression of anellovirus genomes and recombinant Anellovirus ORFs in Sf9 cells
Example 33: Anellovirus ORF1 associates with DNA in Sf9 cells to form complexes isolated by isopycnic centrifugation
Example 34: Expression of ORF1 protein from a diverse array of Anelloviruses using baculovirus
Example 35: In vitro assembly of baculovirus constructs

### Example 1: Anellovirus Multiple Dosing in Human Transfusion Patients

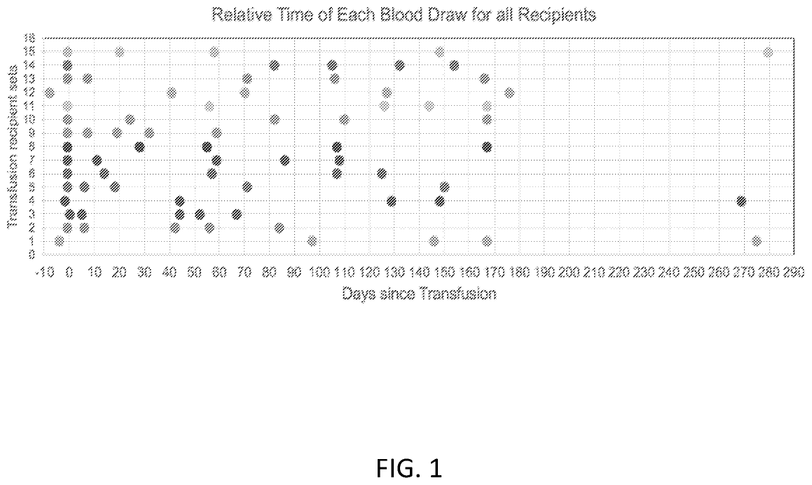

In this example, human patients receiving multiple blood transfusions were tracked for persistence of Anellovirus strains introduced from donors and relative persistence compared to host Anellovirus strains. Blood samples were taken on the date of transfusion, or shortly before, to establish each patient's original Anellovirus profile. As shown in FIG. 1, a total of fifteen human transfusion recipients were monitored for this study. To assess change in Anellovirus profiles over time after transfusion, blood samples were taken regularly up to 280 days after transfusion. Five samples were taken from each patient over the course of the study, one prior to the transfusion and four time-points post-transfusion. Generally, blood samples were taken every few weeks or months for each patient. 12/15 recipients completed all their post-transfusion blood draws within 6 months of the transfusion date.

Blood samples were assessed for the presence of Anellovirus strains. Briefly, Anellovirus sequence-containing nucleic acids were isolated from the blood samples, followed by amplification and high-throughput sequencing. Anellovirus strains were then identified in each sample, thereby constructing an Anellovirus profile specific to each patient at each time of sampling.

The patients in this example received one or more transfusions at a single transfusion event from different donors (i.e., non-matched donor transfusions). Recipient blood samples were collected at four times post-transfusion, so the Anellovirus strains introduced by each donor could be tracked over time in the transfusion recipient using the methods described above. By comparing the change in Anellovirus profiles over time, the relative persistence of donor Anelloviruses and the recipient host's original Anelloviruses could be determined.

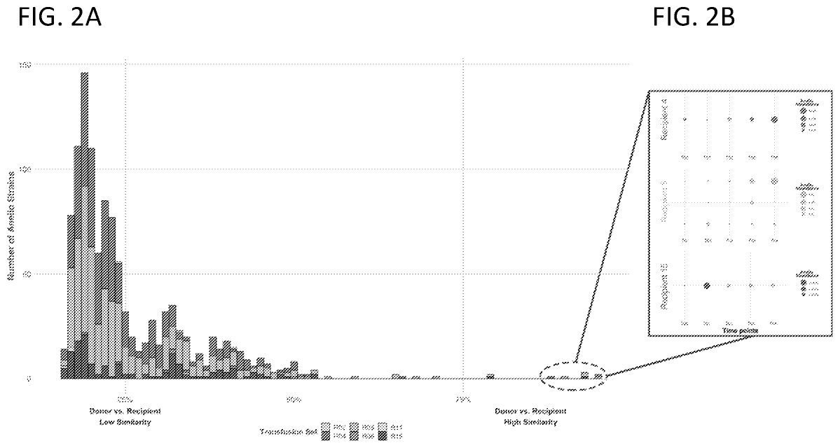

Furthermore, similarity between the Anellovirus strains introduced from each donor could also be assessed. Patients that received Anelloviruses highly similar to those already present in the patient prior to transfusion effectively received a re-dosing of Anelloviruses. These patients could then be used as a proxy for re-dosing, e.g., to infer whether re-dosed Anelloviruses induced an immune response. Here, five Anelloviruses were identified in three patients that received highly similar Anelloviruses to ones already present in the recipient pre-transfusion (i.e., amino acid similarity greater than 90% in ORF1). As shown in FIGS. 2A and 2B these patients showed proxy re-dosing in all three Anellovirus genera (i.e., Alphatorqueviruses, Betatorqueviruses, and Gammatorqueviruses).

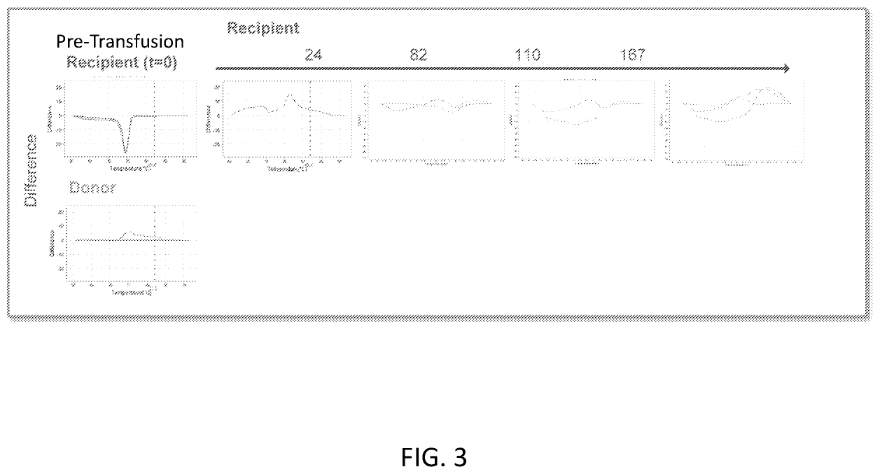

In addition, analysis of marker SNPs indicated that the proxy re-dosed strains persisted longitudinally up to 167 days post-transfusion. A High-Resolution Melting (HRM) assay was used to detect and distinguish highly similar Anellovirus strains in transfusion recipients at post-transfusion time-points. Briefly, we looked for strains from donors and recipient's pre-transfusion that had >90% pairwise identity at the nucleotide level. We then designed primers that would anneal to both strains and had at least one nucleotide difference within the amplicon. Using a saturating dye, we carried out a high-resolution melt curve which generates a unique profile based on which strain is present in the sample. As shown in FIG. 3, at 24 days after transfusion, the Anellovirus profiles of proxy re-dosed patients primarily consisted of the patient's own Anelloviruses. By 82 days post-transfusion, the Anellovirus profiles consisted of a mixture of patient and donor strains. By 110-167 days post-transfusion, the Anellovirus profiles primarily resembled that of the donors. These data demonstrated substantial persistence by the highly similar re-dosed donor Anellovirus strains, suggesting Anellovirus transmission via blood transfusion of strains that are highly similar to those already present in a patient.

### Example 2: Recurring Anellovirus Dosing in Human Transfusion Patients

In this example, human patients will receive multiple, recurring donor-matched blood transfusions. In brief, the patients will receive an initial transfusion from particular donors. Every patient will then receive subsequent blood transfusions from the same donor or donors. This will allow us to track the change in the donors' Anelloviruses in blood as well as which strains infect and persist in the recipient, thus also allow us to monitor potential recurring redosing of Anelloviruses via blood transfusion.

### Example 3: Preparation of Synthetic Anellovectors

This example demonstrates in vitro production of a synthetic anellovector.

DNA sequences from LY1 and LY2 strains of TTMiniV (Eur Respir J. 2013 August; 42(2):470-9), between the EcoRV restriction enzyme sites, were cloned into a kanamycin vector (Integrated DNA Technologies). The resultant genetic element constructs based on DNA sequences from the LY1 and LY2 strains of TTMiniV are referred to as Anellovector 1 (Anello 1) and Anellovector 2 (Anello 2) respectively, in Examples 4 and 5. Cloned constructs were transformed into 10-Beta competent E. coli. (New England Biolabs Inc.), followed by plasmid purification (Qiagen) according to the manufacturer's protocol.

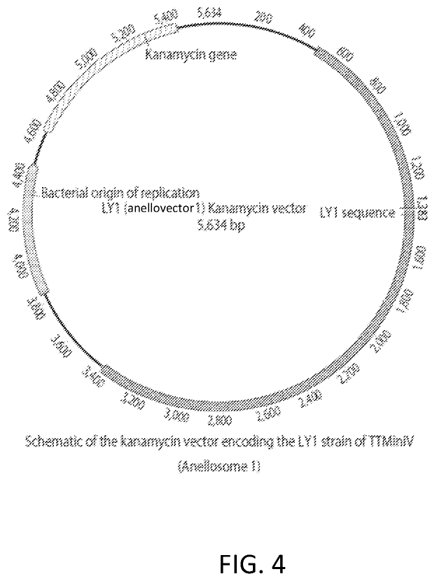

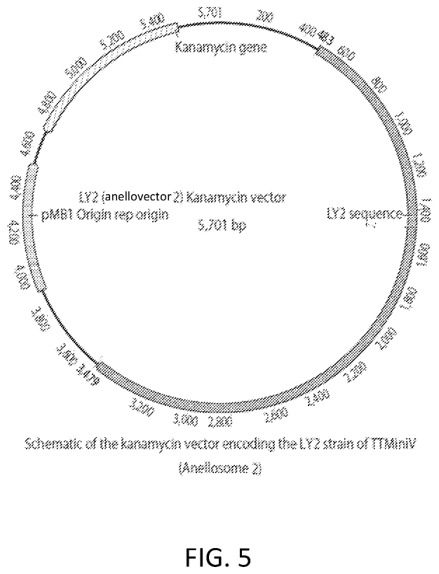

DNA constructs (FIG. 4 and FIG. 5) were linearized with EcoRV restriction digest (New England Biolabs, Inc.) at 37 degree Celsius for 6 hours, yielding double-stranded linear DNA fragments containing the TTMiniV genome, and excluding bacterial backbone elements (such as the origin of replication and selectable markers). This was followed by agarose gel electrophoresis, excision of a correctly size DNA band for the TTMiniV genome fragment (2.9 kilobase pairs), and gel purification of DNA from excised agarose bands using a gel extraction kit (Qiagen) according to the manufacturer's protocol.

In some embodiments, a method according to this example can be used to produce the constructs of anellovectors to be used in the methods of administration of anellovectors described herein.

### Example 4: Assembly and Infection of Anellovectors

This example demonstrates successful in vitro production of infectious anellovectors using synthetic DNA sequences as described in Example 3.

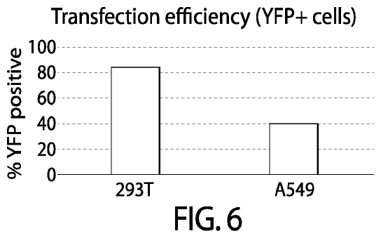

The double-stranded linearized gel-purified Anellovirus genome DNA (obtained in Example 3) was transfected into either HEK293T cells (human embryonic kidney cell line) or A549 cells (human lung carcinoma cell line), either in an intact plasmid or in linearized form, with lipid transfection reagent (Thermo Fisher Scientific). 6 ug of plasmid or 1.5 ug of linearized Anellovirus genome DNA was used for transfection of 70% confluent cells in T25 flasks. Empty vector backbone lacking the viral sequences included in the anellovector was used as a negative control. Six hours post-transfection, cells were washed with PBS twice and were allowed to grow in fresh growth medium at 37 degrees Celsius and 5% carbon dioxide. DNA sequences encoding the human Ef1alpha promoter followed by YFP gene were synthesized from IDT. This DNA sequence was blunt end ligated into a cloning vector (Thermo Fisher Scientific). The resulting vector was used as a control to assess transfection efficiency. YFP was detected using a cell imaging system (Thermo Fisher Scientific) 72 hours post transfection. The transfection efficiencies of HEK293T and A549 cells were calculated as 85% and 40% respectively (FIG. 6).

Supernatants of 293T and A549 cells transfected with anellovectors were harvested 96 hours post transfection. The harvested supernatants were spun down at 2000 rpm for 10 minutes at 4 degrees Celsius to remove any cell debris. Each of the harvested supernatants was used to infect new 293T and A549 cells, respectively, that were 70% confluent in wells of 24 well plates. Supernatants were washed away after 24 hours of incubation at 37 degrees Celsius and 5% carbon dioxide, followed by two washes of PBS, and replacement with fresh growth medium. Following incubation of these cells at 37 degrees and 5% carbon dioxide for another 48 hours, cells were individually harvested for genomic DNA extraction. Genomic DNA from each of the samples was harvested using a genomic DNA extraction kit (Thermo Fisher Scientific), according to manufacturer's protocol.

To confirm the successful infection of 293T and A549 cells by anellovectors produced in vitro, 100 ng of genomic DNA harvested as described herein was used to perform quantitative polymerase chain reaction (qPCR) using primers specific for beta-torqueviruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 42.

TABLE 42

| | Primer sequence (5′ > 3′) | |
|---|---|---|
| Target | Forward | Reverse |
| Betatorqueviruses | ATTCGAATGGCTGAGTTTATGC | CCTTGACTACGGTGGTTTCAC |
| | (SEQ ID NO: 690) | (SEQ ID NO: 693) |
| LY2 TTMiniV | CACGAATTAGCCAAGACTGGGCAC | TGCAGGCATTCGAGGGCTTGTT |
| strain | (SEQ ID NO: 691) | (SEQ ID NO: 694) |
| GAPDH | GCTCCCACTCCTGATTTCTG | TTTAACCCCCTAGTCCCAGG |
| | (SEQ ID NO: 692) | (SEQ ID NO: 695) |

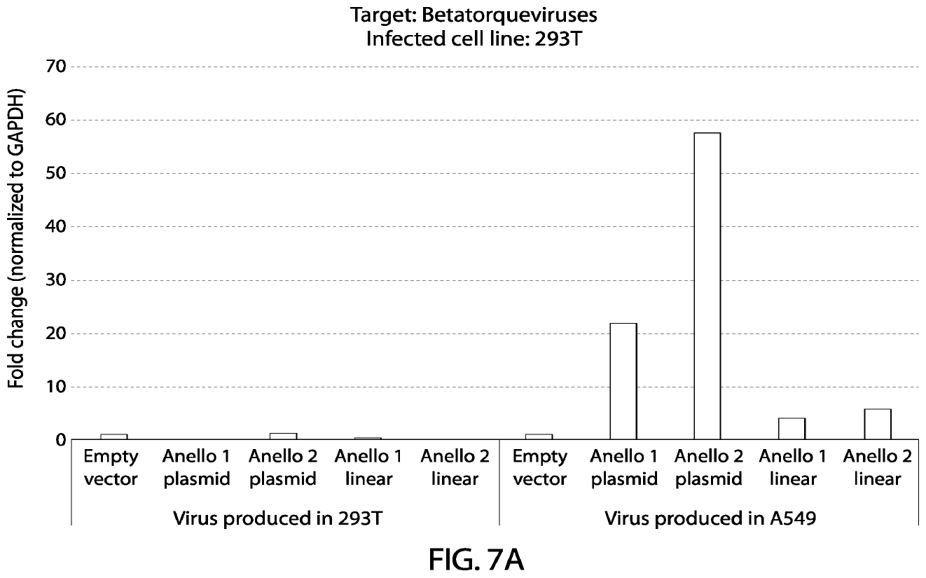

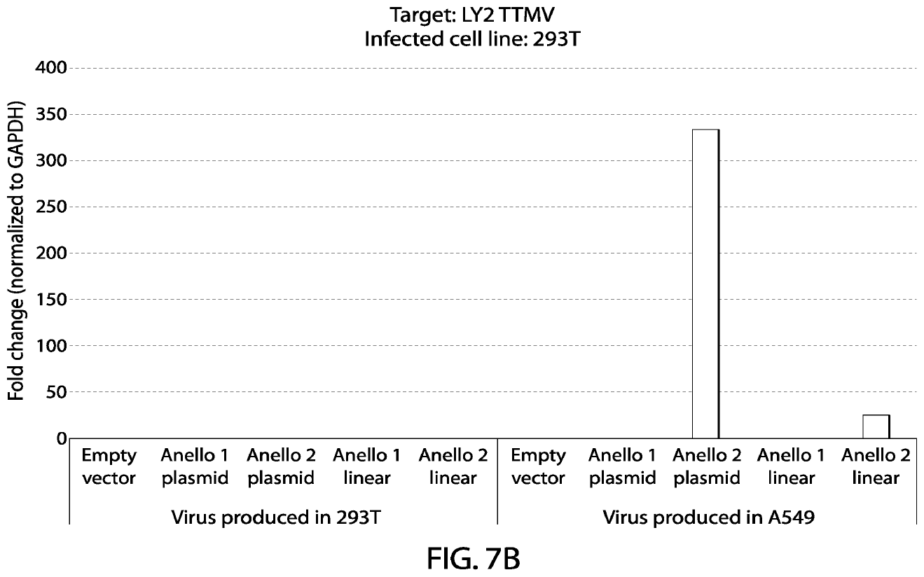

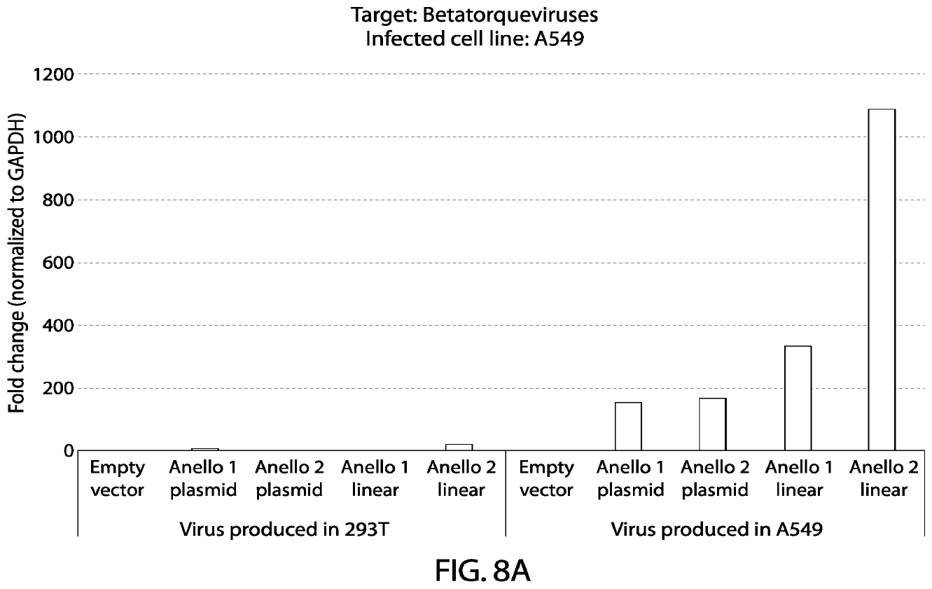

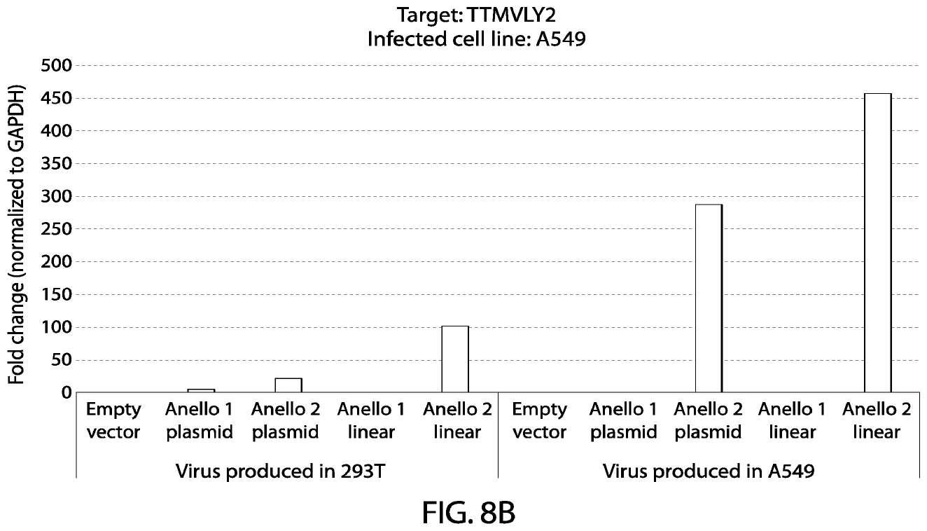

As shown in the qPCR results depicted in FIGS. 7A, 7B, 8A, and 8B, the anellovectors produced in vitro and as described in this example were infectious.

In some embodiments, a method according to this example can be used to produce the anellovectors to be used in the methods of administration of anellovectors described herein.

### Example 5: Selectivity of Anellovectors

This example demonstrates the ability of synthetic anellovectors produced in vitro to infect cell lines of a variety of tissue origins.

Supernatants with the infectious TTMiniV anellovectors (described in Example 3) were incubated with 70% confluent 293T, A549, Jurkat (an acute T cell leukemia cell line), Raji (a Burkitt's lymphoma B cell line), and Chang cell lines at 37 degrees and 5% carbon dioxide in wells of 24 well plates. Cells were washed with PBS twice, 24 hours post infection, followed by replacement with fresh growth medium. Cells were then incubated again at 37 degrees and 5% carbon dioxide for another 48 hours, followed by harvest for genomic DNA extraction. Genomic DNA from each of the samples was harvested using a genomic DNA extraction kit (Thermo Fisher Scientific), according to manufacturer's protocol.

To confirm successful infection of these cell lines by anellovectors produced in the previous Example, 100 ng of genomic DNA harvested as described herein was used to perform quantitative polymerase chain reaction (qPCR) using primers specific for beta-torqueviruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 42.

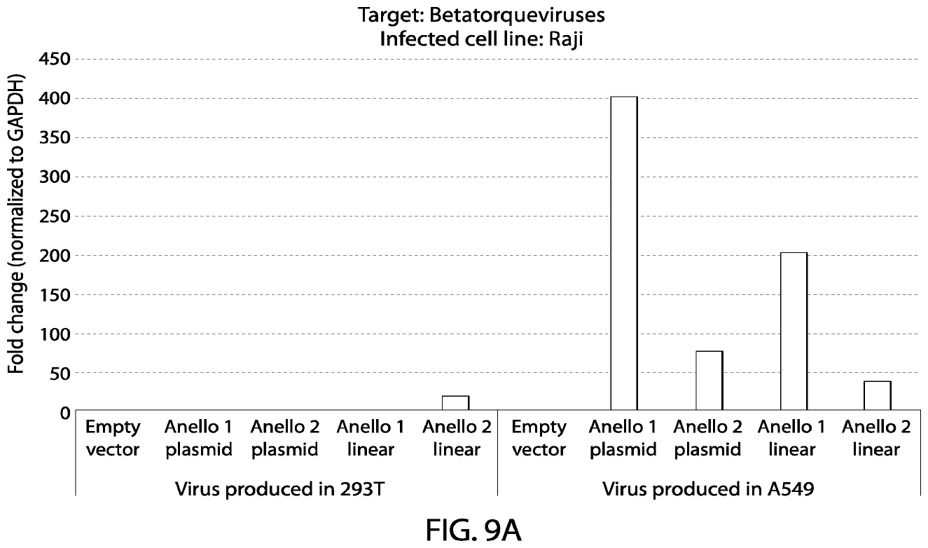

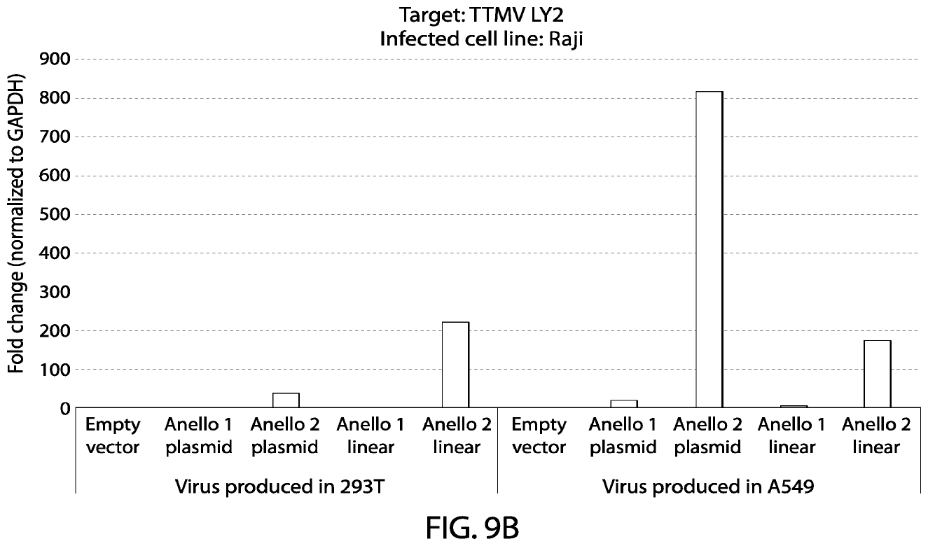

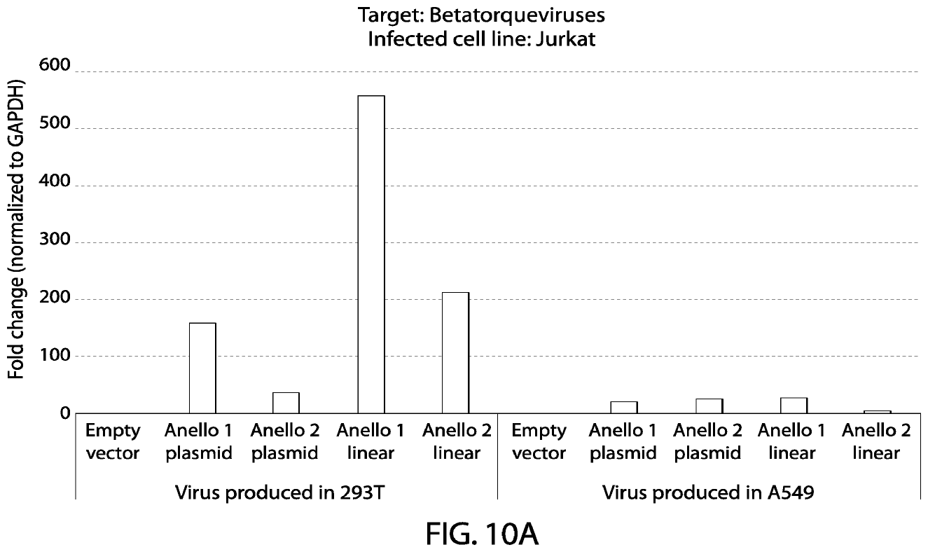

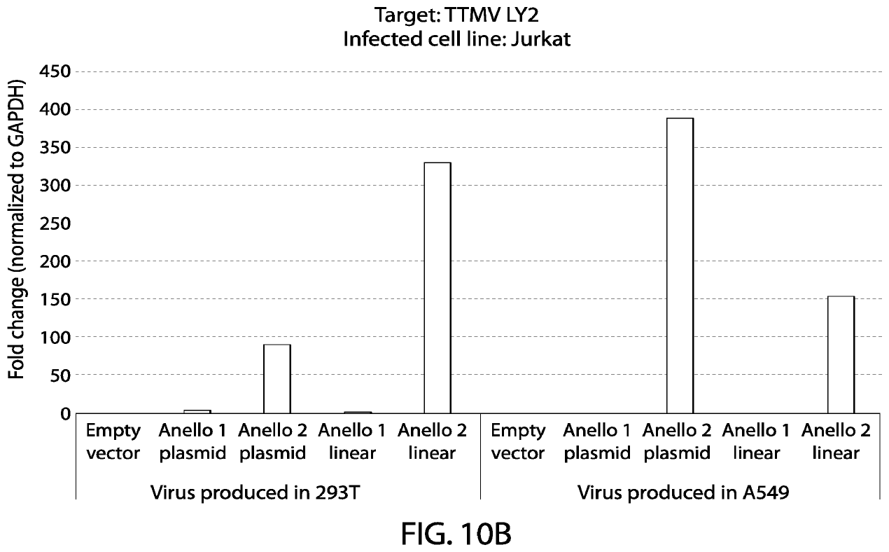

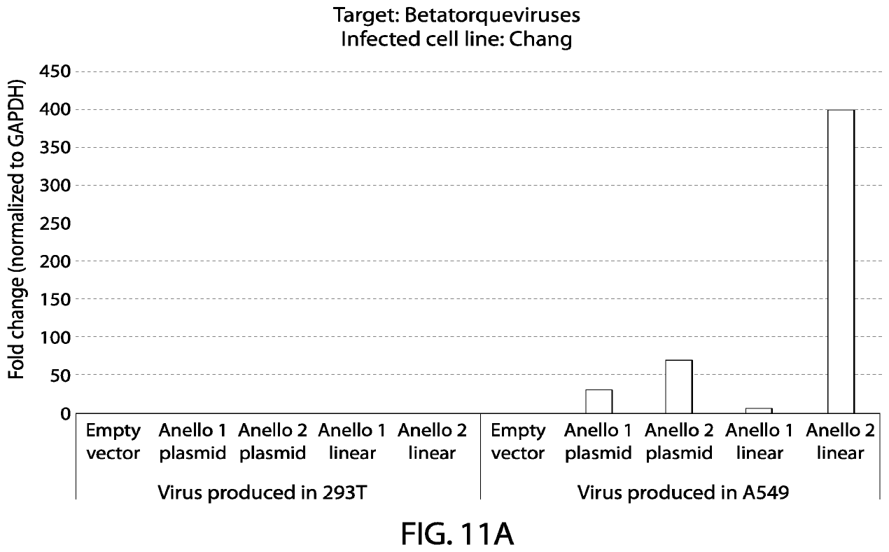

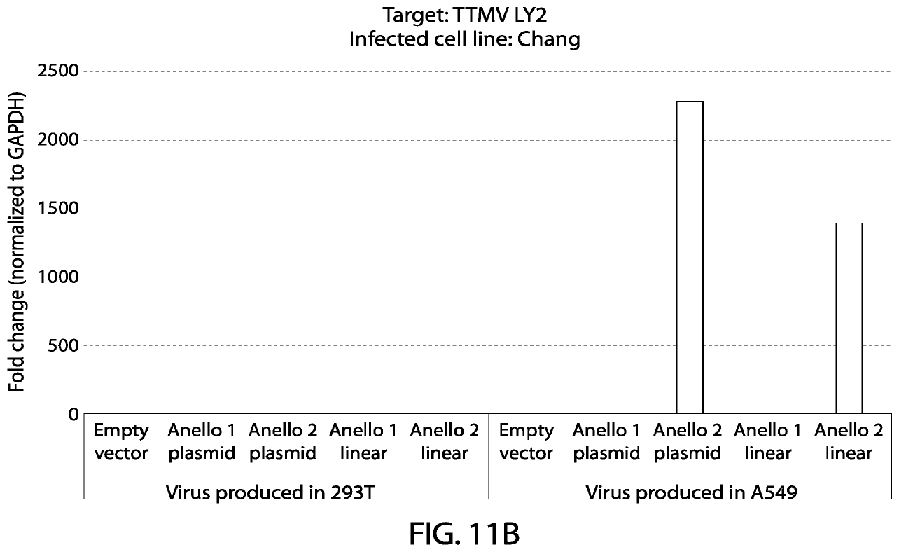

As shown in the qPCR results depicted in FIGS. 7A-11B, not only were anellovectors produced in vitro infectious, they were able to infect a variety of cell lines, including examples of epithelial cells, lung tissue cells, liver cells, carcinoma cells, lymphocytes, lymphoblasts, T cells, B cells, and kidney cells. It was also observed that a synthetic anellovector was able to infect HepG2 cells (a liver cell line), resulting in a greater than 100-fold increase relative to a control.

In some embodiments, the method of this example can be performed with anellovectors to be used in the methods of administration of anellovectors described herein.

### Example 6: Replication-Deficient Anellovectors and Helper Viruses

For replication and packaging of an anellovector, some elements can be provided in trans. These include proteins or non-coding RNAs that direct or support DNA replication or packaging. Trans elements can, in some instances, be provided from a source alternative to the anellovector, such as a helper virus, plasmid, or from the cellular genome.

Other elements are typically provided in cis. These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans.

Replication deficient viruses can be useful as helper viruses, e.g., for controlling replication of an anellovector (e.g., a replication-deficient or packaging-deficient anellovector) in the same cell. In some instances, the helper virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient helper virus would drive the amplification and packaging of the anellovector. The packaged particles collected would thus be comprised solely of therapeutic anellovector, without helper virus contamination.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

In some embodiments, a method according to this example can be used to produce the anellovectors to be used in the methods of administration of anellovectors described herein.

### Example 7: Manufacturing Process for Replication-Competent Anellovectors

This example describes a method for recovery and scaling up of production of replication-competent anellovectors. Anellovectors are replication competent when they encode in their genome all the required genetic elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic anellovector either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1).

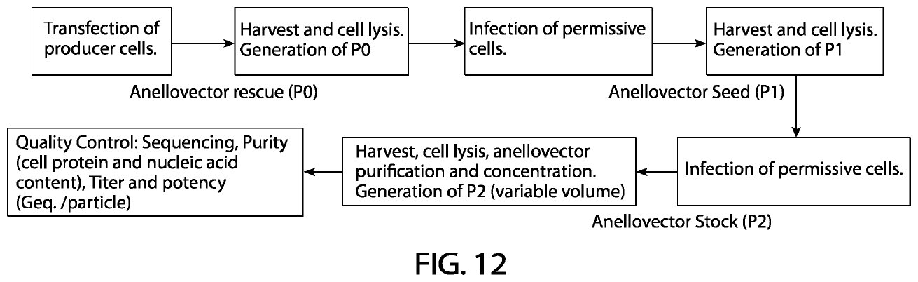

Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 12.

In some embodiments, a method according to this example can be used to produce the anellovectors to be used in the methods of administration of anellovectors described herein.

### Example 8: Manufacturing Process of Replication-Deficient Anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described in Example 7.

In some embodiments, a method according to this example can be used to produce the anellovectors to be used in the methods of administration of anellovectors described herein.

### Example 9: Production of Anellovectors Using Suspension Cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described in Example 8). The complementing plasmids can, in some instances, encode for viral proteins that have been deleted from the anellovector genome (e.g., an anellovector genome based on a viral genome, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence, e.g., as described in Example 18 of PCT/US2018/037379 (incorporated herein by reference).

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

In some embodiments, a method according to this example can be used to produce the anellovectors to be used in the methods of administration of anellovectors described herein.

### Example 10: Utilizing Anellovectors to Express an Exogenous Protein in Mice

This example describes the usage of an anellovector in which the Torque Teno Mini Virus (TTMV) genome is engineered to express the firefly luciferase protein in mice.

The plasmid encoding the DNA sequence of the engineered TTMV encoding the firefly-luciferase gene is introduced into A549 cells (human lung carcinoma cell line) by chemical transfection. 18 ug of plasmid DNA is used for transfection of 70% confluent cells in a 10 cm tissue culture plate. Empty vector backbone lacking the TTMV sequences is used as a negative control. Five hours post-transfection, cells are washed with PBS twice and are allowed to grow in fresh growth medium at 37° C. and 5% carbon dioxide.

Transfected A549 cells, along with their supernatant, are harvested 96 hours post transfection. Harvested material is treated with 0.5% deoxycholate (weight in volume) at 37° C. for 1 hour followed by endonuclease treatment. Anellovector particles are purified from this lysate using ion exchange chromatography. To determine anellovector concentration, a sample of the anellovector stock is run through a viral DNA purification kit and genome equivalents per ml are measured by qPCR using primers and probes targeted towards the anellovector DNA sequence.

A dose-range of genome equivalents of anellovectors in 1× phosphate-buffered saline is performed via a variety of routes of injection (e.g. intravenous, intraperitoneal, subcutaneous, intramuscular) in mice at 8-10 weeks of age. Ventral and dorsal bioluminescence imaging is performed on each animal at 3, 7, 10 and 15 days post injection. Imaging is performed by adding the luciferase substrate (Perkin-Elmer) to each animal intraperitoneally at indicated time points, according to the manufacturer's protocol, followed by intravital imaging.

In some embodiments, the method of this example can be performed with anellovectors to be used in the methods of administration of anellovectors described herein.

### Example 11: Functional Effects of an Anellovector Expressing an Exogenous microRNA Sequence

This example demonstrates the successful expression of an exogenous miRNA (miR-625) from anellovector genome using a native promoter.

500 ng of following plasmid DNAs were transfected into 60% confluent wells of HEK293T cells in a 24 well plate:

i) Empty plasmid backbone

ii) Plasmid containing TTV-tth8 genome in which endogenous miRNA is knocked out (KO)

iii) TTV-tth8 in which endogenous miRNA is replaced with a non-targeting scramble miRNA

iv) TTV-tth8 in which endogenous miRNA sequence is replaced with miRNA encoding miR-625

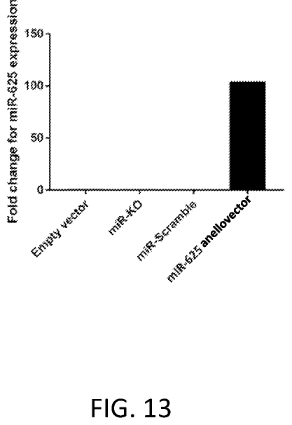

72 hours post transfection, total miRNA was harvested from the transfected cells using the Qiagen miRNeasy kit, followed by reverse transcription using miRNA Script RT II kit. Quantitative PCR was performed on the reverse transcribed DNA using primer that should specifically detect miRNA-625 or RNU6 small RNA. RNU6 small RNA was used as a housekeeping gene and data is plotted in FIG. 13 as a fold change relative to empty vector. As shown in FIG. 13, miR-625 anellovector resulted in approximately 100-fold increase in miR-625 expression, whereas no signal was detected for empty vector, miR-knockout (KO), and scrambled miR.

In some embodiments, the method of this example can be performed with anellovectors to be used in the methods of administration of anellovectors described herein.

### Example 12: Preparation and Production of Anellovectors to Express Exogenous Non-Coding RNAs

This example describes the synthesis and production of anellovectors to express exogenous small non-coding RNAs.

The DNA sequence from the tth8 strain of TTV (Jelcic et al, Journal of Virology, 2004) is synthesized and cloned into a vector containing the bacterial origin of replication and bacterial antibiotic resistance gene. In this vector, the DNA sequence encoding the TTV miRNA hairpin is replaced by a DNA sequence encoding an exogenous small non-coding RNA such as miRNA or shRNA. The engineered construct is then transformed into electro-competent bacteria, followed by plasmid isolation using a plasmid purification kit according to the manufacturer's protocols.

The anellovector DNA encoding the exogenous small non-coding RNAs is transfected into an eukaryotic producer cell line to produce anellovector particles. The supernatant of the transfected cells containing the anellovector particles is harvested at different time points post transfection. Anellovector particles, either from the filtered supernatant or after purification, are used for downstream applications, e.g., as described herein.

In some embodiments, a method according to this example can be used to produce the anellovectors to be used in the methods of administration of anellovectors described herein.

### Example 13: Expression of an Endogenous miRNA from an Anellovector and Deletion of the Endogenous miRNA

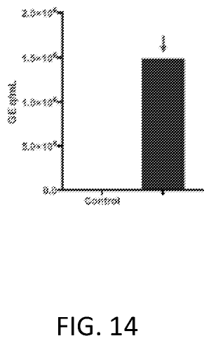

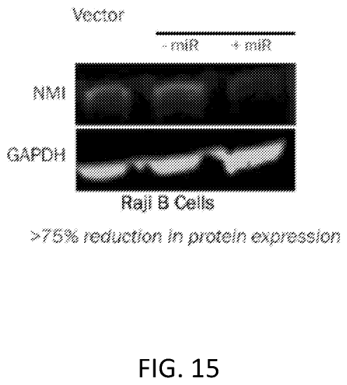

In one example, anellovectors comprising a modified TTV-tth8 genome, in which the TTV-tth8 genome was modified with a 36-nucleotide (nt) sequence (CGCGCTGCGCGCGCCGCCCAGTAGGGGGAGCCATGC (SEQ ID NO: 160)) deletion in the GC-rich region as described in Example 27 of PCT/US19/65995 (incorporated herein by reference), were used to infect Raji B cells in culture. These anellovectors comprised a sequence encoding the endogenous payload of the TTV-tth8 Anellovirus, which is a miRNA targeting the mRNA encoding n-myc interacting protein (NMI), and were produced by introducing a plasmid comprising the Anellovirus genome into a host cell. NMI operates downstream of the JAK/STAT pathway to regulate the transcription of various intracellular signals, including interferon-stimulated genes, proliferation and growth genes, and mediators of the inflammatory response. As shown in FIG. 14, viral genomes were detected in target Raji B cells. Successful knockdown of NMI was also observed in target Raji B cells compared to control cells (FIG. 15). Anellovector comprising the miRNA against NMI induced a greater than 75% reduction in NMI protein levels compared to control cells. This example demonstrates that an anellovector with a native Anellovirus miRNA can knock down a target molecule in host cells.

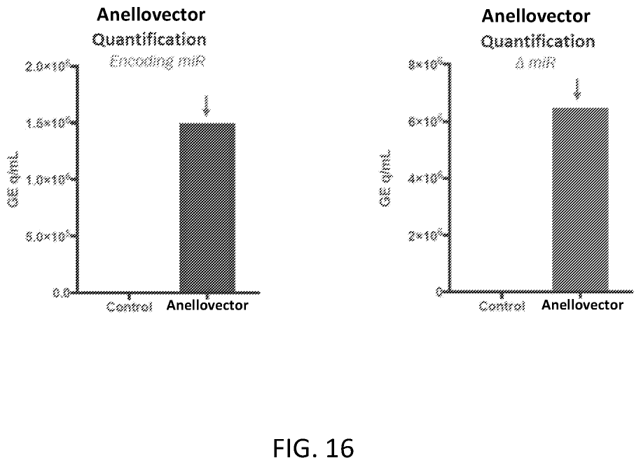

In another example, the endogenous miRNA of an Anellovirus-based anellovector was deleted. The resultant anellovector (Δ miR) was then incubated with host cells. Genome equivalents of Δ miR anellovector genetic elements was then compared to that of corresponding anellovectors in which the endogenous miRNA was retained. As shown in FIG. 16, anellovector genomes in which the endogenous miRNA were deleted were detected in cells at levels comparable to those observed for anellovector genomes in which the endogenous miRNA was still present. This example demonstrates that the endogenous miRNA of an Anellovirus-based anellovector can be mutated, or deleted entirely and the anellovector genome can still be detected in target cells.

In some embodiments, a method according to this example can be used to produce the anellovectors to be used in the methods of administration of anellovectors described herein.

### Example 14: Anellovector Delivery of Exogenous Proteins In Vivo

This example demonstrates in vivo effector function (e.g. expression of proteins) of anellovectors after administration.

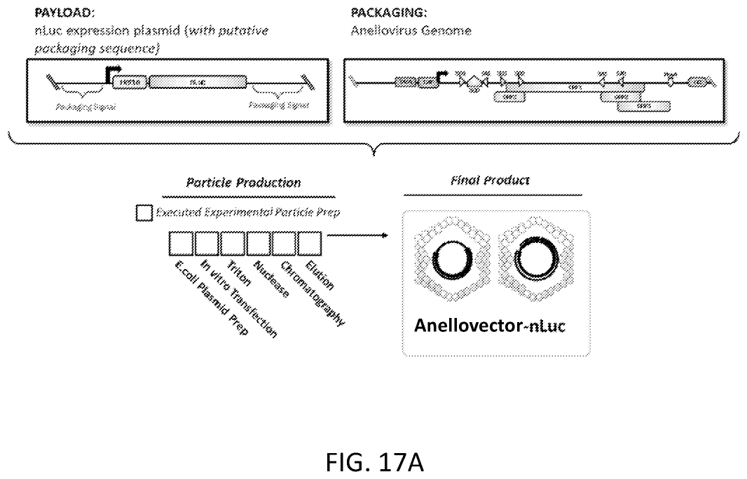

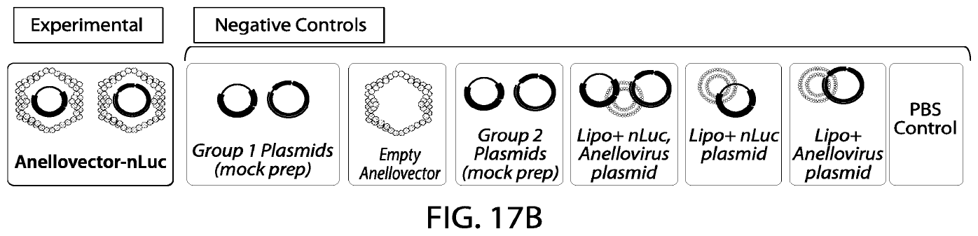

Anellovectors comprising a transgene encoding nano-luciferase (nLuc) (FIGS. 17A-17B) were prepared. Briefly, double-stranded DNA plasmids harboring the TTMV-LY2 non-coding regions and an nLuc expression cassette were transfected into HEK293T cells along with double-stranded DNA plasmids encoding the full TTMV-LY2 genome to act as trans replication and packaging factors. After transfection, cells were incubated to permit anellovector production and anellovector material was harvested and enriched via nuclease treatment, ultrafiltration/diafiltration, and sterile filtration. Additional HEK293T cells were transfected with non-replicating DNA plasmids harboring nLuc expression cassettes and TTMV-LY2 ORF transfection cassettes, but lacking non-coding domains essential for replication and packaging, to act as a “non-viral” negative control. The non-viral samples were prepared following the same protocol as the anellovector material.

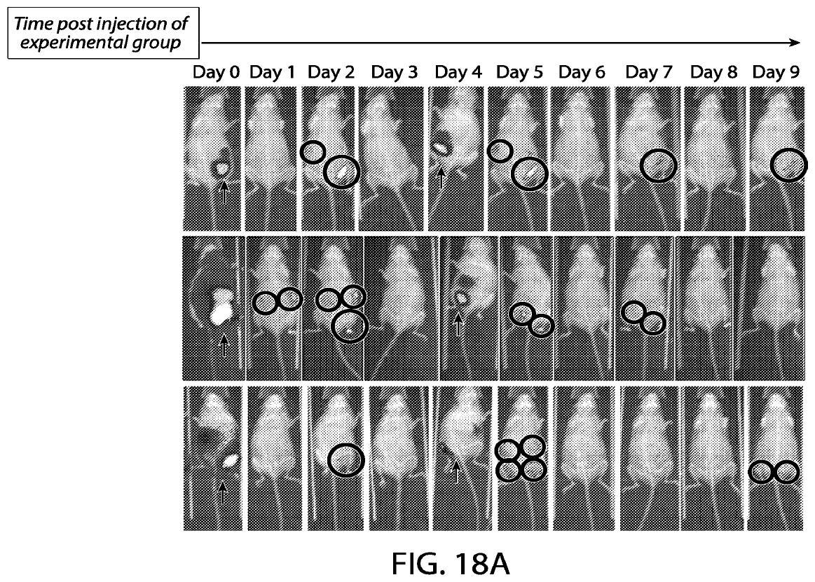

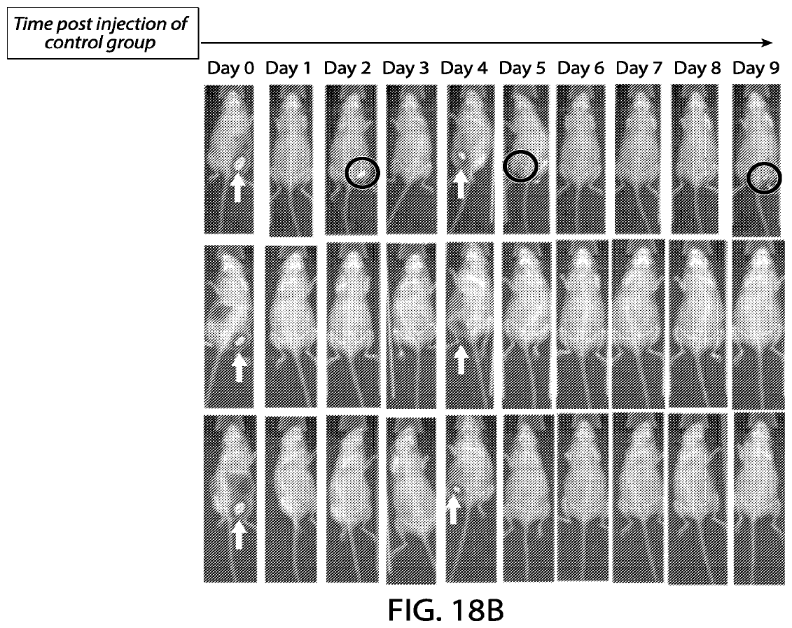

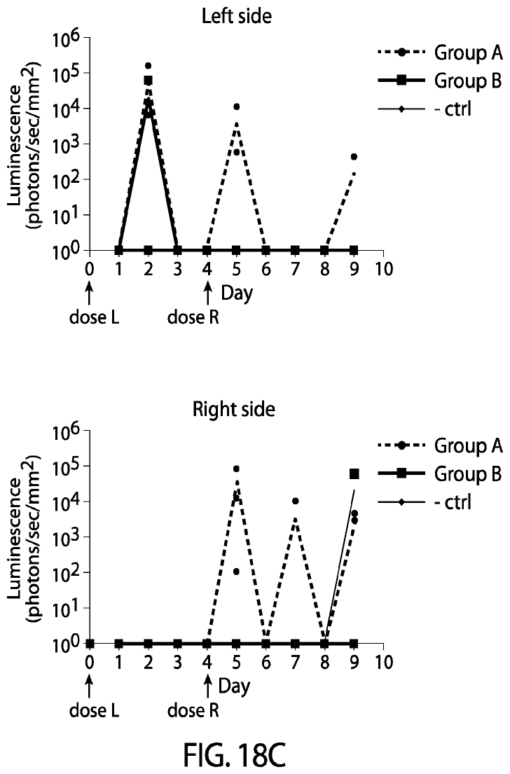

Anellovector preparation was administered to a cohort of three healthy mice intramuscularly, and monitored by IVIS Lumina imaging (Bruker) over the course of nine days (FIG. 18A). As a non-viral control, the non-replicating preparation was administered to three additional mice (FIG. 18B). Injections of 25 μL of anellovector or non-viral preparations were administered to the left hind leg on Day 0, and re-administered to the right hind leg on Day 4 (See arrows in FIGS. 18A and 18B). After 9 days of IVIS imaging, more occurrences of nLuc luminescent signal were observed in mice injected with the anellovector preparation (FIG. 18A) than the non-viral preparation (FIG. 18B), which is consistent with trans gene expression after in vivo anellovector transduction.

### Example 15: In Vitro Circularized Anellovirus Genomes

This example describes constructs comprising circular, double stranded Anelloviral genome DNA with minimal non-viral DNA. These circular viral genomes more closely match the double-stranded DNA intermediates found during wild-type Anellovirus replication. When introduced into a cell, such circular, double stranded Anelloviral genome DNA with minimal non-viral DNA can undergo rolling circle replication to produce, for example, a genetic element as described herein.

In one example, plasmids harboring TTV-tth8 variants and TTMV-LY2 were digested with restriction endonucleases recognizing sites flanking the genomic DNA. The resulting linearized genomes were then ligated to form circular DNA. These ligation reactions were done with varying DNA concentrations to optimize the intramolecular ligations. The ligated circles were either directly transfected into mammalian cells, or further processed to remove non-circular genome DNA by digesting with restriction endonucleases to cleave the plasmid backbone and exonucleases to degrade linear DNA. For TTV-tth8, XmaI endonuclease was used to linearize the DNA; the ligated circle contained 53 bp of non-viral DNA between the GC-rich region and the 5′ non-coding region. For TTMV-LY2, the type IIS restriction enzyme Esp3I was used, yielding a viral genomic DNA circle with no non-viral DNA. This protocol was adapted from previously published circularizations of TTV-tth8 (Kincaid et al., 2013, PLoS Pathogens 9(12): e1003818). To demonstrate the improvements in Anellovirus production, circularized TTV-tth8 and TTMV-LY2 were transfected into HEK293T cells. After 7 days of incubation, cells were lysed, and qPCR was performed to compare the levels of anellovirus genome between circularized and plasmid-based anelloviral genomes. Increased levels of Anelloviral genomes show that circularization of the viral DNA is a useful strategy for increasing Anellovirus production.

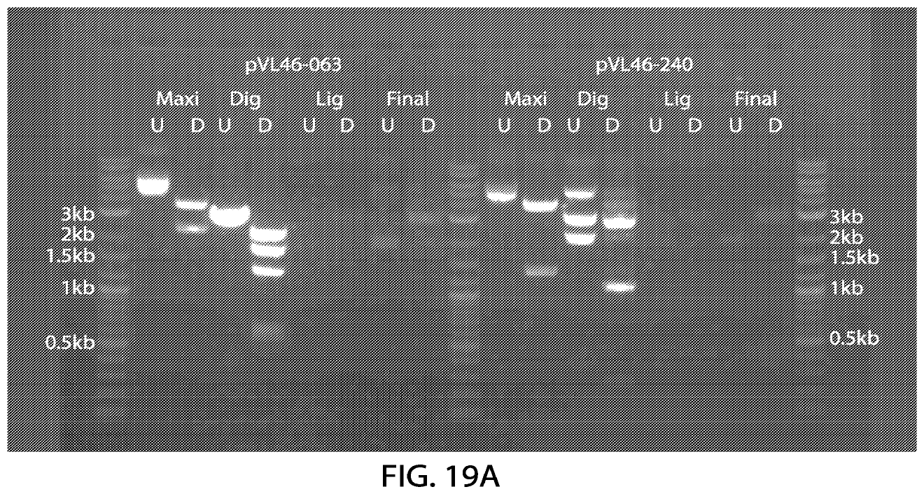

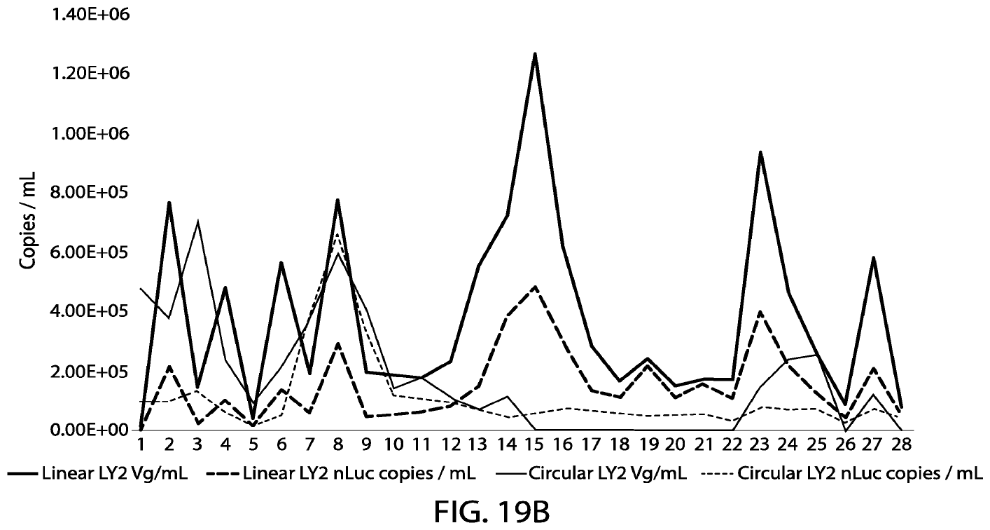

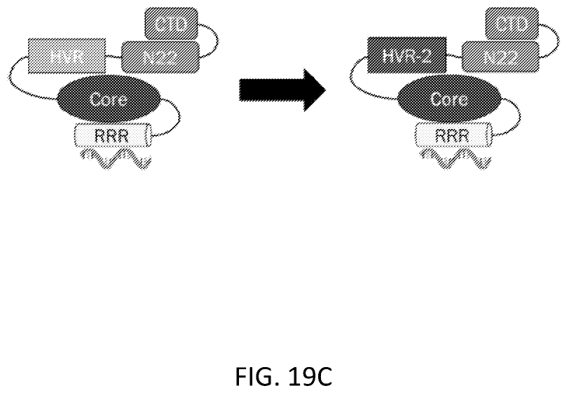

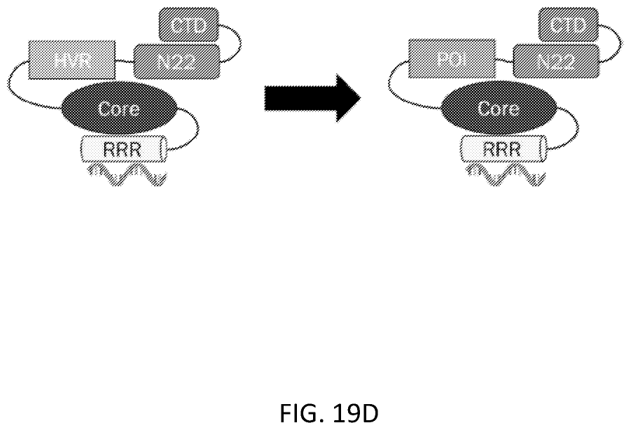

In another example, TTMV-LY2 plasmid (pVL46-240) and TTMV-LY2-nLuc were linearized with Esp3I or EcoRV-HF, respectively. Digested plasmid was purified on 1% agarose gels prior to electroelution or Qiagen column purification and ligation with T4 DNA Ligase. Circularized DNA was concentrated on a 100 kDa UF/DF membrane before transfection. Circularization was confirmed by gel electrophoresis, as shown in FIG. 19A. T-225 flasks were seeded with HEK293T at 3×10⁴ cells/cm² one day prior to lipofection with Lipofectamine 2000. Nine micrograms of circularized TTMV-LY2 DNA and 50 μg of circularized TTMV-LY2-nLuc were co-transfected one day post flask seeding. As a comparison, an additional T-225 flask was co-transfected with 50 μg of linearized TTMV-LY2 and 50 μg of linearized TTMV-LY2-nLuc.

Anellovector production proceeded for eight days prior to cell harvest in Triton X-100 harvest buffer. Generally, anellovectors can be enriched, e.g., by lysis of host cells, clarification of the lysate, filtration, and chromatography. In this example, harvested cells were nuclease treated prior to sodium chloride adjustment and 1.2 μm/0.45 μm normal flow filtration. Clarified harvest was concentrated and buffer exchanged into PBS on a 750 kDa MWCO mPES hollow fiber membrane. The TFF retentate was filtered with a 0.45 μm filter before loading on a Sephacryl S-500 HR SEC column pre-equilibrated in PBS. Anellovectors were processed across the SEC column at 30 cm/hr. Individual fractions were collected and assayed by qPCR for viral genome copy number and transgene copy number, as shown in FIG. 19B. Viral genomes and transgene copies were observed beginning at the void volume, Fraction 7, of the SEC chromatogram. A residual plasmid peak was observed at Fraction 15. Copy number for TTMV-LY2 genomes and TTMV-LY2-nLuc transgene were in good agreement for Anellovectors produced using circularized input DNA at Fraction 7-Fraction 10, indicating packaged Anellovectors containing nLuc transgene. SEC fractions were pooled and concentrated using a 100 kDa MWCO PVDF membrane and then 0.2 m filtered prior to in vivo administration.

Circularization of input Anellovector DNA resulted a threefold increase in a percent recovery of nuclease protected genomes throughout the purification process when compared to linearized Anellovector DNA, indicating improved manufacturing efficiency using the circularized input Anellovector DNA as shown in Table 46.

TABLE 46
Purification Process Yields

| | Linearized TTMV-LY2 | | Circularized TTMV-LY2 | |
|---|---|---|---|---|
| | | Total nLuc | | Total nLuc |
| | Total viral | transgene | Total viral | transgene |
| | genome | genome | genome | genome |
| Step | copies | copies | copies | copies |
| Harvest pre- | 2.78E+12 | 2.17E+12 | 1.04E+11 | 4.39E+11 |
| nuclease | | | | |
| Clarified | 9.96E+09 | 5.48E+09 | 6.55E+08 | 9.81E+08 |
| Harvest | | | | |
| TFF | 1.01E+10 | 7.66E+09 | 2.58E+08 | 3.56E+08 |
| SEC | 3.18E+07 | 8.73E+06 | 9.16E+06 | 7.75E+06 |
| UF/DF | 8.82E+06 | 3.25E+06 | 1.78E+06 | 2.73E+06 |
| Sterile | 5.60E+06 | 2.64E+06 | 8.66E+05 | 1.63E+06 |
| Filtration | | | | |
| Purification | 0.0002% | 0.0001% | 0.0006% | 0.0004% |
| Process | | | | |
| Yield (%) | | | | |

In some embodiments, a method according to this example can be used to produce the anellovectors to be used in the methods of administration of anellovectors described herein.

### Example 16: Production of Anellovectors Containing Chimeric ORF1 with Hypervariable Domains from Different Torque Teno Virus Strains

This example describes domain swapping of hypervariable regions of ORF1 to produce chimeric anellovectors containing the ORF1 arginine-rich region, jelly-roll domain, N22, and C-terminal domain of one TTV strain, and the hypervariable domain from an ORF1 protein of a different TTV strain.

The full-length genome LY2 strain of Betatorquevirus has been cloned into expression vectors for expression in mammalian cells. This genome is mutated to remove the hypervariable domain of LY2 and replace it with the hypervariable domain of a distantly related Betatorqueviruses (FIG. 19C). The plasmid containing the LY2 genome with the swapped hypervariable domain (pTTMV-LY2-HVRa-z) is then linearized and circularized using previously published methods (Kincaid et al., PLoS Pathogens 2013). HEK293T cells are transfected with the circularized genome and incubated for 5-7 days to allow anellovector production. After the incubation period anellovectors are purified from the supernatant and cell pellet of transfected cells by gradient ultracentrifugation.

To determine if the chimeric anellovectors are still infectious, the isolated viral particles are added to uninfected cells. The cells are incubated for 5-7 days to allow viral replication. After incubation the ability of the chimeric anellovectors to establish infection will be monitored by immunofluorescence, western blot, and qPCR. The structural integrity of the chimeric viruses is assessed by negative stain and cryo-electron microscopy. Chimeric anellovectors can further be tested for ability to infect cells in vivo. Establishment of the ability to produce functional chimeric anellovectors through hypervariable domain swapping could allow for engineering of viruses to alter tropism and potentially evade immune detection.

In some embodiments, a method according to this example can be used to produce the anellovectors to be used in the methods of administration of anellovectors described herein.

### Example 17: Production of Chimeric ORF1 Containing Non-TTV Protein/Peptides in Place of Hypervariable Domains

This example describes the replacement of the hypervariable regions of ORF1 with other proteins or peptides of interest to produce chimeric ORF1 protein containing the arginine-rich region, jelly-roll domain, N22, and C-terminal domain of one TTV strain, and a non-TTV protein/peptide in place of the hypervariable domain.

As shown in example 16, the hypervariable domain of LY2 is deleted from the genome and a protein or peptide of interest may be inserted into this region (FIG. 19D). Examples of types of sequences that could be introduced into this region include but are not limited to, affinity tags, single chain variable regions (scFv) of antibodies, and antigenic peptides. Mutated genomes in the plasmid (pTTMV-LY2-ΔHVR-POI) are linearized and circularized as described in example 16. Circularized genomes are transfected into HEK293T cells and incubated for 5-7 days. Following incubation, the chimeric anellovectors containing the POI are purified from the supernatant and cell pellet via ultracentrifugation and/or affinity chromatography where appropriate.

The ability to produce functional chimeric anellovectors containing POIs is assessed using a variety of techniques. First, purified virus is added to uninfected cells to determine if chimeric anellovectors can replicate and/or deliver payload to naïve cells. Additionally, structural integrity of chimeric anellovectors is assessed using electron microscopy. For chimeric anellovectors that are functional in vitro, the ability of replicate/delivery payload in vivo is also assessed.

In some embodiments, a method according to this example can be used to produce the anellovectors to be used in the methods of administration of anellovectors described herein.

### Example 18: Anellovectors Based on tth8 and LY2 Each Successfully Transduced the EPO Gene into Lung Cancer Cells

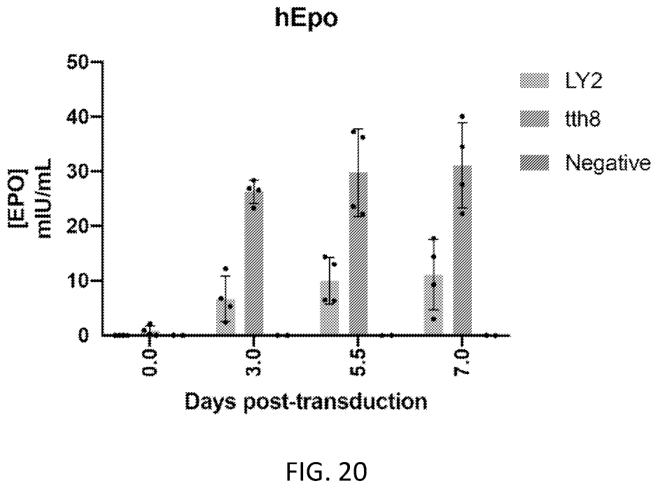

In this example, a non-small cell lung cancer line (EKVX) was transduced using two different anellovectors carrying the erythropoietin gene (EPO). The anellovectors were generated by in vitro circularization, as described herein, and included two types of anellovectors based on either an LY2 or tth8 backbone. Each of the LY2-EPO and tth8-EPO anellovectors included a genetic element that included the EPO-encoding cassette and non-coding regions of the LY2 or tth8 genome (5′ UTR, GC-rich region), respectively, but did not include Anellovirus ORFs, e.g., as described in Example 39 of PCT/US19/65995 (incorporated herein by reference). Cells were inoculated with purified anellovectors or a positive control (AAV2-EPO at high dose or at the same dose as the anellovectors) and incubated for 7 days. Anellovirus ORFs were provided in trans in a separate in vitro circularized DNA. Culture supernatant was sampled 3, 5.5, and 7 days post-inoculation and assayed using a commercial ELISA kit to detect EPO. Both LY2-EPO and tth8-EPO anellovectors successfully transduced cells, showing significantly higher EPO titers compared to untreated (negative) control cells (P<0.013 at all time points) (FIG. 20).

In some embodiments, the method of this example can be performed with anellovectors to be used in the methods of administration of anellovectors described herein.

### Example 19: Anellovectors with Therapeutic Transgenes can be Detected In Vivo after Intravenous (i.v.) Administration

In this example, anellovectors encoding human growth hormone (hGH) were detected in vivo after intravenous (i.v.) administration. Replication-deficient anellovectors, based on a LY2 backbone and encoding an exogenous hGH (LY2-hGH), were generated by in vitro circularization as described herein. The genetic element of the LY2-hGH anellovectors included LY2 non-coding regions (5′ UTR, GC-rich region) and the hGH-encoding cassette, but did not include Anellovirus ORFs, e.g., as described in Example 39 of PCT/US19/65995 (incorporated herein by reference). LY2-hGH anellovectors were administered to mice intravenously. The Anellovirus ORFs were provided in trans in a separate in vitro circularized DNA. Briefly, anellovectors (LY2-hGH) or PBS was injected intravenously at day 0 (n=4 mice/group). Anellovectors were administered to independent animal groups at 4.66E+07 anellovector genomes per mouse.

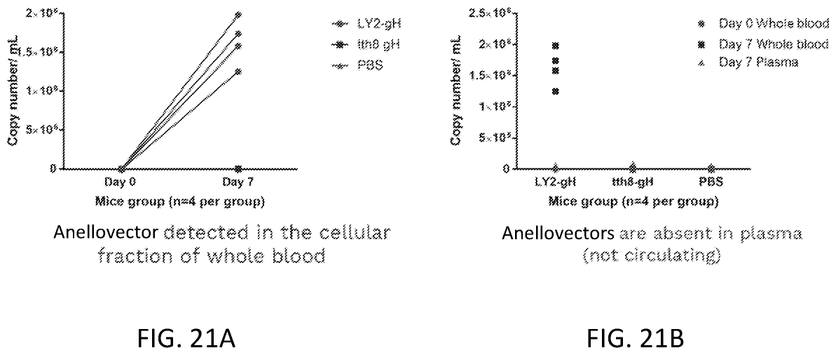

In a first example, anellovector viral genome DNA copies were detected. At day 7, blood and plasma were collected and analyzed for the hGH DNA amplicon by qPCR. LY2-hGH anellovectors were present in the cellular fraction of whole blood after 7 days post infection in vivo (FIG. 21A). Furthermore, the absence of anellovectors in plasma demonstrated the inability of these anellovectors to replicate in vivo (FIG. 21B).

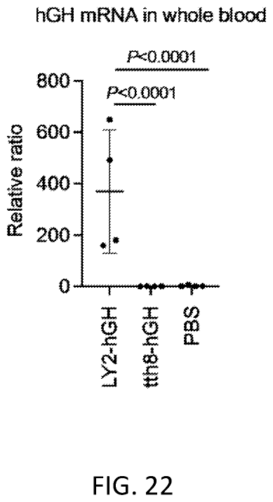

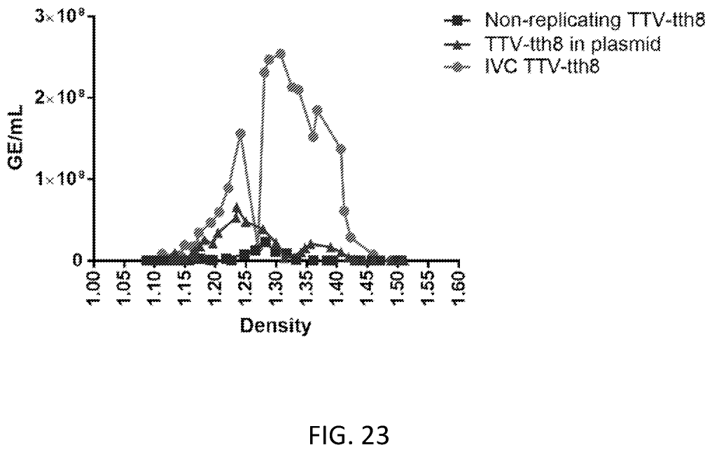

In a second example, hGH mRNA transcripts were detected after in vivo transduction. At day 7, blood was collected and analyzed for the hGH mRNA transcript amplicon by qRT-PCR. GAPDH was used as a control housekeeping gene. hGH mRNA transcripts in were measured in the cellular fraction of whole blood. mRNA from the anellovector-encoded transgene was detected in vivo (FIG. 22).

In some embodiments, the method of this example can be performed with anellovectors to be used in the methods of administration of anellovectors described herein.

### Example 20: In Vitro Circularized Genome as Input Material for Producing Anellovectors In Vitro

This example demonstrates that in vitro circularized (IVC) double stranded anellovirus DNA, as source material for an anellovector genetic element as described herein, is more robust than an anellovirus genome DNA in a plasmid to yield packaged anellovector genomes of the expected density.

1.2E+07 HEK293T cells (human embryonic kidney cell line) in T75 flasks were transfected with 11.25 ug of either, (i) in vitro circularized double stranded TTV-tth8 genome (IVC TTV-tth8), (ii) TTV-tth8 genome in a plasmid backbone, or (iii) plasmid containing just the ORF1 sequence of TTV-tth8 (non-replicating TTV-tth8). Cells were harvested 7 days post transfection, lysed with 0.1% Triton, and treated with 100 units per ml of Benzonase. The lysates were used for cesium chloride density analysis; density was measured and TTV-tth8 copy quantification was performed for each fraction of the cesium chloride linear gradient. As shown in FIG. 23, IVC TTV-tth8 yielded dramatically more viral genome copies at the expected density of 1.33 as compared to TTV-tth8 plasmid.

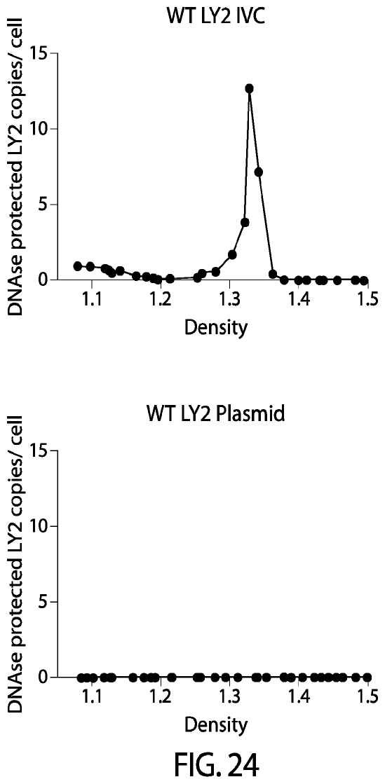

1E+07 Jurkat cells (human T lymphocyte cell line) were nucleofected with either in-vitro circularized LY2 genome (LY2 IVC) or LY2 genome in plasmid. Cells were harvested 4 days post transfection and lysed using a buffer containing 0.5% triton and 300 mM sodium chloride, followed by two rounds of instant freeze-thaw. The lysates were treated with 100 units/ml benzonase, followed by cesium chloride density analysis. Density measurement and LY2 genome quantification was performed on each fraction of the cesium chloride linear gradient. As shown in FIG. 24, transfection of in vitro circularized LY2 genome in Jurkat cells led to a sharp peak at the expected density, as compared to the transfection of plasmid containing the LY2 genome, which showed no detectable peak in FIG. 24.

In some embodiments, a method according to this example can be used to produce the anellovectors to be used in the methods of administration of anellovectors described herein.

### Example 21: Tandem Copies of the Anellovirus Genome

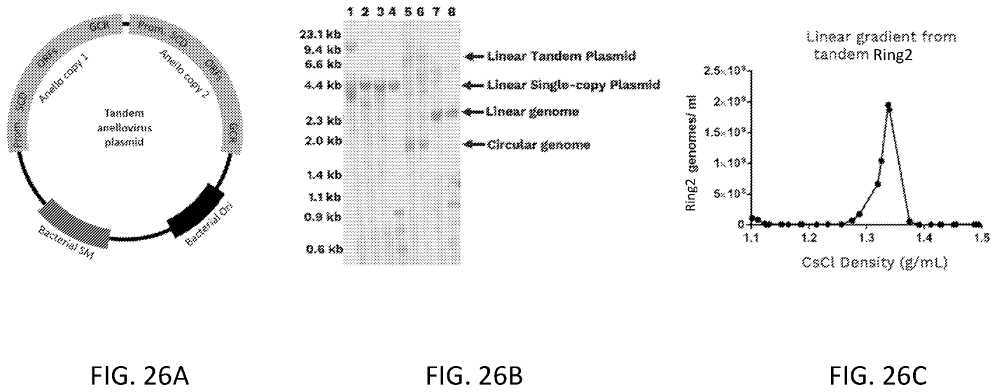

This example describes plasmid-based expression vectors harboring two copies of a single anelloviral genome, arranged in tandem such that the GC-rich region of the upstream genome is near the 5′ region of the downstream genome (FIG. 26A).

In some embodiments, anelloviruses may replicate via rolling circle, in which a replicase (Rep) protein binds to the genome at an Anellovirus Rep binding site (e.g., as described herein, e.g., comprising a 5′ UTR, e.g., comprising a hairpin loop and/or origin of replication) and initiates DNA synthesis around the circle. For anellovirus genomes contained in plasmid backbones, this typically involves either replication of the full plasmid length, which is longer than the native viral genome, or recombination of the plasmid resulting in a smaller circle comprising the genome with minimal backbone. Therefore, viral replication off of a plasmid can be inefficient. To improve viral genome replication efficiency, a plasmid was engineered with tandem copies of TTMV-LY2. Without wishing to be bound by theory, these plasmids may have presented circular permutations of the anelloviral genome, such that regardless of where the Rep protein binds, it would be able to drive replication of the viral genome from the upstream Anellovirus Rep binding site through to a downstream Anellovirus Rep displacement site (e.g., comprising a 5′ UTR, e.g., comprising a hairpin loop and/or origin of replication, e.g., as described herein).

Tandem TTMV-LY2 was assembled via Golden-gate assembly, simultaneously incorporating two copies of the genome into a backbone and leaving no extra nucleotides between the genomes. The tandem TTMV-LY2 plasmid comprised two identical copies of the anellovirus genome, starting with the first 5′NCR through the first GC-rich region, and followed immediately by the second 5′ NCR through the second GC-rich region (FIG. 26A). The plasmid also comprised a bacterial backbone with bacterial origin and selectable marker.

Plasmid harboring tandem copies of TTMV-LY2 was transfected into MOLT-4 cells via nucleofection. Plasmid with a single copy of the TTMV-LY2 genome was similarly transfected as a control. Cells were incubated for four days, then cell pellets were collected. A portion of each cell pellet was used for Southern blotting. Total DNA was isolated from the cells using a Qiagen DNeasy Blood and Tissue Kit. Four alternative digests were performed on 10 μg of each total DNA sample, using restriction endonucleases that digest the genomic DNA with different effects on the TTMV-LY2 genomes and plasmids: one digest did not cut within genomes or plasmids uncut; a second digest cut at a single within the bacterial backbone, but not the anellovirus genome; a third digest cut a single locus within the TTMV-LY2 genome, but not within the bacterial backbone; and a final digest cut within the TTMV-LY2 genome and not the bacterial backbone, but also included methylation-sensitive DpnI enzyme that will digest only input plasmid DNA produced in bacteria, and will not cut within DNA replicated in the mammalian cells. The digests were run on a 7 mm thick 1% agarose gel in 1×TAE at 0.5V/cm for 3 hours. The gel was then treated to depurinate and denature the DNA. The DNA was then transferred to a positively-charged nylon membrane via capillary transfer overnight. The DNA was crosslinked to the membrane via ultraviolet light. The blot was then probed with random-hexamer generated fragments against the TTMV-LY2 genome, incorporating Biotin-dUTP into the probes. The probes were detected using Streptavidin-conjugated IRDye-800, and imaged on a LiCor Odyssey imager.

Southern blotting demonstrated that the tandem TTMV-LY2 plasmid was capable of replicating circular double-stranded anellovirus genomes of wild-type size (FIG. 26B). For a plasmid harboring a single copy of the TTMV-LY2 genome, uncut supercoiled DNA between 4 and 10 kb was observed (lane 1), which was linearized to 5.1 kb when cut within the plasmid backbone (lane 2) or within the TTMV-LY2 genome (lane 3). No bands consistent with recovered wild-type length TTMV-LY2 genome, either circular or linear, were observed from the plasmid with a single copy of the TTMV-LY2 genome. The entire plasmid with a single copy did replicate in the MOLT-4 cells, as observed by DpnI-resistant copies digestion of the linearized plasmid (lane 4). However, no wild-type length genome was recovered from the single-copy TTMV-LY2 plasmid.

For the plasmid harboring tandem copies of TTMV-LY2 genome, the supercoiled plasmid between 4 and 10 kb was observed (lane 5), which linearized to 8.8 kb when cut in the plasmid backbone (lane 6). Importantly, an approximately 1.8 kb band consistent with a single copy of double stranded DNA TTMV-LY2 genome was observed from the uncut and backbone cut lanes, consistent with recovery of wild-type TTMV-LY2 genome (lanes 5 and 6). This when digested with an enzyme that cuts within the TTMV-LY2 genome, the 1.8 kb band was replaced with a 3.0 kb band consistent with linearized TTMV-LY2 genomic DNA (lane 7). This linearized TTMV-LY2 genome band was DpnI resistant, indicating that it was replicated within the mammalian cell, rather than being produced through recombination of the tandem DNA (lane 8). Together these data demonstrated that wild-type length TTMV-LY2 genomes were recovered from the tandem TTMV-LY2 plasmid in MOLT-4 cells.

Additional cell pellets transfected with the tandem TTMV-LY2 plasmid were lysed by freeze/thaw in the presence of 0.5% Triton, then run on a linear CsCl gradient to separate viral particles from unpackaged DNA. Fractions were taken from the linear gradient, and qPCR was performed using Taqman probes for the TTMV-LY2 genome sequence. A peak of TTMV-LY2 genomes was observed at a CsCl density between 1.30 and 1.35 g/cm³, where anellovirus-sized particles are expected to be found (FIG. 26C). This indicated that the TTMV-LY2 genomes produced in MOLT-4 cells were successfully packaged into viral particles. Overall, these data demonstrated that engineering tandem Anelloviral genomes can increase viral genome replication and can be used as a strategy for increasing Anellovirus production.

### Example 22: Efficient Replication of Anellovectors from a Tandem Anellovector Construct

In this example, a tandem Anellovector is shown to successfully undergo amplification in a mammalian host cell, such as HEK293 or MOLT-4 cells. The tandem Anellovector construct were built to include two full-length copies of an Anellovirus genome (e.g., Ring1, Ring2, or Ring4, e.g., as described herein). Each copy of the genome included, in order from 5′ to 3′, a 5′ non-coding region comprising a highly conserved domain, a region comprising the cargo sequence replacing the native anellovirus open reading frames, and a 3′ UTR comprising a GC-rich region. The 3′ end of the first genome copy and the 5′ end of the second genome copy were attached directly to each other without intervening nucleotides.

Briefly, the construct is introduced into HEK293 or MOLT-4 cells by PEI transfection reagent or nucleofection. Trans replication and packaging elements, including anellovirus ORF1, are provided in trans from separate plasmids. The transfected cells are incubated for four days at 37° C. Replication of the Anellovirus genome is measured by qPCR and Southern blot. For negative controls, plasmid harboring a single copy of the anellovector and the tandem anellovector without the trans elements are included.

### Example 23: Exemplary Tandem Anellovector Construct Designs

In the examples described below, a number of exemplary construct designs for tandem Anelloviruses were tested for capacity to undergo rolling circle amplification in MOLT-4 host cells. Without wishing to be bound by theory, it is contemplated that Anellovirus rolling circle amplification begins and ends at a replicase-binding site (e.g., a 5′ UTR, e.g., comprising a hairpin loop and/or origin of replication). In circularized single Anellovirus genomes, the same replicase-binding site can act as both the start and stop sites. Tandem Anelloviruses, as well as the alternate designs described in this example, position such replicase-binding sites at both ends of the genome to be replicated, such that the genomes effectively operate like the circularized single-copy genomes.

Constructs Having Partial Anellovirus Genomes on the 3′ End

In this example, exemplary tandem Anellovectors were designed in which a full length copy of an Anellovirus genome was positioned 5′ relative to a partial Anellovirus genome. As shown in FIG. 27A, a first alternate construct (pRTx-843) comprised, in order for 5′ to 3′, a full length copy of an Anellovirus genome (Ring2), followed by a partial Anellovirus genome consisting of a 5′ NCR, a region comprising the full set of viral open reading frames, and a 3′ NCR lacking a GC-rich region. As shown in FIG. 27A, a second alternate construct (pRTx-844) comprised, in order for 5′ to 3′, a full length copy of an Anellovirus genome (Ring2), followed by a partial Anellovirus genome consisting of a 5′ NCR and a region comprising the full set of viral open reading frames, from nucleotides 1 to 2812 of Ring2. As shown in FIG. 27A, a third alternate construct (pRTx-845) comprised, in order for 5′ to 3′, a full length copy of an Anellovirus genome (Ring2), followed by a partial Anellovirus genome consisting of a 5′ NCR and a region comprising only part of the viral open reading frames, from nucleotides 1 to 2583 of Ring2. As shown in FIG. 27A, a fourth alternate construct (pRTx-846) comprised, in order for 5′ to 3′, a full length copy of an Anellovirus genome (Ring2), followed by a partial Anellovirus genome consisting of a 5′ NCR and a region comprising only part of the viral open reading frames, from nucleotides 1 to 2264 of Ring2. As shown in FIG. 27A, a fifth alternate construct (pRTx-847) comprised, in order for 5′ to 3′, a full length copy of an Anellovirus genome (Ring2), followed by a partial Anellovirus genome consisting of a 5′ NCR and a region comprising only part of the viral open reading frames, from nucleotides 1 to 723 of Ring2. As shown in FIG. 27A, a sixth alternate construct (pRTx-848) comprised, in order for 5′ to 3′, a full length copy of an Anellovirus genome (Ring2), followed by a partial Anellovirus genome consisting of a 5′ NCR, from nucleotides 1 to 423 of Ring2. As shown in FIG. 27A, a seventh alternate construct (pRTx-849) comprised, in order for 5′ to 3′, a full length copy of an Anellovirus genome (Ring2), followed by a partial Anellovirus genome consisting of a part of a 5′ NCR, from nucleotides 1 to 267 of Ring2.

Briefly, each of the tandem constructs was introduced into MOLT-4 cells by nucleofection. Replicase proteins for rolling circle amplification were provided in cis by the complete viral genome. ORF1 protein was provided in cis by the complete viral genome.

The full length tandem Ring2 construct with two full genomes (pVL46-257) was used as a positive control for viral replication and packaging. For a negative control, a plasmid harboring a single copy of the Ring2 genomes (pVL46-240) is used. The transfected cells were incubated for 4 days at 37°, then cells were harvested for Southern blot and qPCR analysis. For Southern blot, total DNA was isolated from the cells using a Qiagen DNeasy Blood and Tissue Kit, and 10 μg of total DNA was digested with an enzyme that cuts once in the plasmid backbone and with DpnI to digest any input DNA produced in bacteria. The digests were run on a 7 mm thick 1% agarose gel in 1×TAE at 0.5V/cm for 3 hours. The gel was then treated to depurinate and denature the DNA. The DNA was then transferred to a positively-charged nylon membrane via capillary transfer overnight. The DNA was crosslinked to the membrane via ultraviolet light. The blot was then probed with random-hexamer generated fragments against the TTMV-LY2 genome, incorporating Biotin-dUTP into the probes. The probes were detected using Streptavidin-conjugated IRDye-800, and imaged on a LiCor Odyssey imager. Note that samples from plasmid pRTx-845 were not tested by Southern blot. Recovery of replicated circular double-stranded DNA Ring2 genomes was observed for pRTx-843 and 844, but not for pRTx-846-849 (FIG. 27D). Replication of the plasmid DNA was also observed for pRTx-843, 844, and 848, similar to what is observed for the single-copy genome plasmid.

Additional cell pellets were lysed using freeze/thaw and 0.5% triton. Lysates were passed over a cesium chloride step gradient and Anellovirus-containing fractions were collected. Replication of the Anellovirus genome was measured by DNase-protected qPCR. pRTx-843-846 produced similar levels of Ring2 viral genomes per cell as observed from the full tandem pRTx-257, indicating successful production of encapsidated virus (FIG. 27E). pRTx-847 also produced protected genomes, albeit fewer than observed for the full tandem, while pRTx-848 and 849 were not tested by qPCR.

Constructs Having Partial Anellovirus Genomes on the 5′ End

In this example, exemplary tandem Anellovectors were designed in which a full length copy of an Anellovirus genome is positioned 3′ relative to a partial Anellovirus genome. As shown in FIG. 27B, a series of constructs were tested, with the following partial Ring2 genomes followed by a full length Ring2 genome: pRTx-836, with a partial anellovirus genome consisting of the highly conserved 5′NCR domain, the full set of anelloviral open reading frames, and the 3′ NCR including a GC-rich region (Ring2 nucleotides 267 to 2979); pRTx-837, with a partial anellovirus genome consisting of the full set of anelloviral open reading frames and the 3′ NCR including a GC-rich region (Ring2 nucleotides 423 to 2979); pRTx-838, with a partial anellovirus genome consisting of a part of the anelloviral open reading frames and the 3′ NCR including a GC-rich region (Ring2 nucleotides 723 to 2979); pRTx-839, with a partial anellovirus genome consisting of a part of the anelloviral open reading frames and the 3′ NCR including a GC-rich region (Ring2 nucleotides 2273 to 2979); pRTx-840, with a partial anellovirus genome consisting of a part of the anelloviral open reading frames and the 3′ NCR including a GC-rich region (Ring2 nucleotides 2452 to 2979); pRTx-841, with a partial anellovirus genome consisting of the 3′ NCR including a GC-rich region (Ring2 nucleotides 2812 to 2979); and pRTx-842, with a partial anellovirus genome consisting of the GC-rich region (Ring2 nucleotides 2867 to 2979).

Briefly, each of the tandem constructs was introduced into MOLT-4 cells by nucleofection. Replicase proteins for rolling circle amplification were provided in cis by the complete viral genome. ORF1 protein was provided in cis by the complete viral genome. The full length tandem Ring2 construct with two full genomes (pVL46-257) was used as a positive control for viral replication and packaging. For a negative control, a plasmid harboring a single copy of the Ring2 genomes (pVL46-240) is used.-The transfected cells were incubated for 4 days at 37°, then cells were harvested for Southern blot and qPCR analysis. For Southern blot, total DNA was isolated from the cells using a Qiagen DNeasy Blood and Tissue Kit, and 10 μg of total DNA was digested with an enzyme that cuts once in the plasmid backbone and with DpnI to digest any input DNA produced in bacteria. The digests were run on a 7 mm thick 1% agarose gel in 1×TAE at 0.5V/cm for 3 hours. The gel was then treated to depurinate and denature the DNA. The DNA was then transferred to a positively-charged nylon membrane via capillary transfer overnight. The DNA was crosslinked to the membrane via ultraviolet light. The blot was then probed with random-hexamer generated fragments against the TTMV-LY2 genome, incorporating Biotin-dUTP into the probes. The probes were detected using Streptavidin-conjugated IRDye-800, and imaged on a LiCor Odyssey imager. Recovery of replicated circular double-stranded DNA Ring2 genomes was observed for pRTx-836 through 839, but not for pRTx-840-842 (FIG. 27D).

Additional cell pellets were lysed using freeze/thaw and 0.5% triton. Lysates were passed over a cesium chloride step gradient and Anellovirus-containing fractions were collected. Replication of the Anellovirus genome was measured by DNase-protected qPCR. pRTx-836-840 produced similar levels of Ring2 viral genomes per cell as observed from the full tandem pRTx-257, indicating successful production of encapsidated virus (FIG. 27E). Little to no protected viral genomes were observed for pRTx-841 and 842.

Constructs Having Two Partial Anellovirus Genomes

In this example, exemplary tandem Anellovectors are designed comprising two partial copies of an Anellovirus genome, arranged such that they sufficiently mimic the structure of a tandem structure to permit efficient rolling circle amplification. Six such permutations are shown in FIG. 27C: Permutation 1 comprising, from 5′ to 3′, an partial Ring2 genome starting at the 5′ NCR conserved region, with the full Ring2 open reading frames and the 3′ NCR with the GC-rich region (Ring2 nucleotides 267 to 2979), followed by a partial Ring2 genome with the 5′ NCR and highly conserved region (Ring2 nucleotides 1 to 423); Permutation 2 comprising, from 5′ to 3′, an partial Ring2 genome starting with the full Ring2 open reading frames and the 3′ NCR with the GC-rich region (Ring2 nucleotides 423 to 2979), followed by a partial Ring2 genome with the 5′ NCR with the highly conserved region and part of the open reading frame (Ring2 nucleotides 1 to 723); Permutation 3 comprising, from 5′ to 3′, an partial Ring2 genome starting with part of the Ring2 open reading frame and the 3′ NCR with the GC-rich region (Ring2 nucleotides 723 to 2979), followed by a partial Ring2 genome with the 5′ NCR and part of the anelloviral open reading frame (Ring2 nucleotides 1 to 2273); Permutation 4 comprising, from 5′ to 3′, an partial Ring2 genome starting a partial Ring2 open reading frame and the 3′ NCR with the GC-rich region (Ring2 nucleotides 2273 to 2979), followed by a partial Ring2 genome with the 5′ NCR and part of the anelloviral open reading frame (Ring2 nucleotides 1 to 2452); Permutation 5 comprising, from 5′ to 3′, an partial Ring2 genome starting a partial Ring2 open reading frame and the 3′ NCR with the GC-rich region (Ring2 nucleotides 2452 to 2979), followed by a partial Ring2 genome with the 5′ NCR and the full Ring2 open reading frame (Ring2 nucleotides 1 to 2812); and Permutation 6 comprising, from 5′ to 3′, an partial Ring2 genome starting at the 3′ NCR with the GC-rich region (Ring2 nucleotides 2812 to 2979), followed by a partial Ring2 genome with the 5′ NCR and the full Ring2 open reading frame and the 3′NCR without the GC-rich region (Ring2 nucleotides 1 to 2867).

Briefly, each of the tandem constructs is introduced into MOLT-4 cells by nucleofection. Proteins for rolling circle amplification and viral packaging, including Rep factors and Ring2 ORF1, are provided in trans from other plasmids. The transfected cells are incubated at 37° for 4 days. Replication of the Anellovirus genome is measured by qPCR and Southern blot. The full length tandem Ring2 construct with two full genomes (pVL46-257) is used as a positive control for viral replication and packaging. For a negative control, a plasmid harboring a single copy of the Ring2 genomes (pVL46-240) is used.

### Example 24: Transcription of Genes from a Tandem Anellovirus Construct in Mammalian Cells

In this example, a series of anellovector constructs were produced, based on Ring1 as the backbone (as indicated in FIG. 27F). The constructs included a tandem construct comprising a Ring1 sequence encoding an eGFP-ORF1 fusion protein (codon optimized) and a tandem Ring1 sequence. The constructs were then transfected into Jurkat cells. Transcription of Anellovirus (Ring1) ORF1 was then assessed by sequencing long RNA reads.

As shown in FIG. 27F, greater quantities of full-length Ring1 ORF1 transcripts were detected in Jurkat cells transfected with the Ring1-based tandem GFP constructs compared to Jurkat cells transfected with alternate constructs.

### Example 25: ORF1 and ORF2 Protein Produced from a Tandem Anellovirus Construct in Mammalian Cells

In this example, a series of anellovector constructs were produced, based on Anellovirus Ring2 as the backbone (as indicated in FIG. 27G). The constructs included a tandem construct comprising a first Ring2 sequence and a second Ring2 sequence in tandem. The constructs were nucleofected into MOLT4 cells (Human T lymphoblast cell line) and Ring2 ORF1 protein was then detected by Western blot. Briefly, 1E07 MOLT4 cells were nucleofected with 25 ug of either a plasmid containing the tandem Ring2 genome (Rep) or a negative control plasmid containing 149 bp of the Ring2 genome. Each of the nucleofected samples were inoculated in 25 ml growth medium (RPMI+10% FBS+0.01% Polyaxmer+1 mM Sodium Pyruvate). 1 ml of culture was pelleted from each sample everyday from day 1 to day 3 post nucleofection. The pelleted cells were lysed by resuspending the cells in 50 ul lysis buffer (0.5% Triton, 300 mM NaCl, 50 mM Tris pH 8.0), followed by 2 rounds of freeze thaw. The lysate was then clarified by spinning at 10,000×g for 30 minutes. 20 ul of the clarified lysate was used for western blot analysis to detect Ring2 ORF1 protein by using a cocktail of two rabbit polyclonal antibodies raised against Ring2 ORF1.

As shown in FIG. 27G, Ring2 ORF1 protein was detected in MOLT-4 cells nucleofected with the Ring2-based tandem GFP construct at day 2 and day 3 after nucleofection.

### Example 26: Assessment of Infectivity of Tandem Anellovectors

In this example, tandem Anellovectors are produced as proteinaceous exteriors encapsulating a genetic element encoding an exogenous gene. Tandem Anellovectors are produced, e.g., as described in any of Examples 21-24. In brief, host cells are transfected with tandem Anellovector DNA and incubated under conditions suitable for replication of the tandem Anellovector genetic element and encapsulation within proteinaceous exteriors. Encapsulated Anellovectors are then isolated from the culture, e.g., as described herein. The Anellovectors are then contacted with cells (e.g., MOLT-4 or Jurkat cells) under conditions suitable for infection of the cells.

Infectivity can be assessed, for example, using quantitative real-time PCR (qPCR) to detect Anelloviral nucleic acids in infected cells. For example, infected cells can be harvested for DNA, and qPCR is then performed using primers specific for Anellovirus-specific sequences. qPCR for primers specific to genomic DNA sequence of, for example, GAPDH can be used for normalization. qPCR can be used to quantify infectivity according to the genomic equivalents of Anelloviral DNA detected. Alternatively, infectivity can be assessed by detecting the expression of the exogenous gene or a downstream activity of the exogenous gene. For example, an exogenous fluorescent marker such as GFP or nano-luciferase can be detected, e.g., by detecting fluorescence or by an immunoassay using an antibody that recognizes the marker.

### Example 27: Delivery of Tandem Anelloviral Genomes into Sf9 Insect Cells Via Baculovirus

In this example, baculoviruses harboring tandem copies of the Ring2 genome were made and delivered to Sf9 cells. Tandem Ring2 genomes were assembled as described above. Full length Ring2 genomes were amplified via PCR adding Type IIS restriction sites and inserted into a plasmid backbone with a bacterial origin of replication and selectable marker via golden gate assembly. The resulting plasmid comprised two complete Ring2 genomes next to each other with no intervening nucleotides, arranged with the first genome from 5′ non-coding region through GC-rich region, followed by the second genome from 5′ non-coding region through the GC-rich region. The pair of genomes was flanked by AsiSI and PacI restriction enzyme sites in the plasmid backbone.

For insertion of the tandem Ring2 genomes into baculovirus, a modified pFastBac was first assembled. The modified pFastBac had the insect-cell promoter removed, and the promoter and standard multiple cloning site were replaced with a custom multiple cloning site containing AsiSI and PacI sites. The tandem Ring2 genome construct was cloned into the pFastBac plasmid via digestion with AsiSI and PacI, followed by ligation. The final pFastBac-TandemRing2 plasmid comprised the Tn7L recombination sequence, the tandem Ring2 genomes, a Gentamycin resistance gene, and the Tn7R recombination sequence, followed by the plasmid backbone with bacterial origin of replication and ampicillin-resistance marker (FIG. 27H). Inclusion of the tandem Ring2 genomes was confirmed by sequencing and PCR product analysis. The pFastBac was used to produce Bacmids harboring the tandem Ring2 genomes, followed by production of baculoviruses as described above.

Baculoviruses harboring tandem Ring2 genomes were used to infect Sf9 cells at an MOI of 1. Additionally, samples were included with Sf9 cells infected with Ring2 ORF1-expression baculoviruses alone or co-infected with the Ring2 tandem genomes baculoviruses and Ring2 ORF1-expression baculoviruses. After 3 days, Sf9 cells were pelleted by centrifugation. Total DNA was harvested using the Qiagen DNeasy Blood and Tissue Kit. 10 μg of total DNA was digested with Esp3I restriction enzyme, which cuts within the baculovirus immediately flanking the tandem Ring2 genomes (see FIG. 27I). Digested DNA was run on an agarose gel. Then DNA was chemically denatured and depurinated, and transferred to a positively-charged nylon membrane by capillary transfer. DNA was UV-crosslinked to the membrane, then hybridized with Biotin-containing probes designed against the Ring2 genome. The probes were detected with Streptavidin-IRDye800, and imaged on a LiCor Odyssey imager.

Bands consistent with the tandem Ring2 genome size were observed in all samples infected with the tandem Ring2 baculoviruses, demonstrating successful delivery of tandem Ring2 genomes to Sf9 cells (FIG. 27I). Additionally, bands consistent with a single copy of the Ring2 genome isolated from baculoviruses were observed, indicating that some DNA recombination occurred during baculovirus production, resulting in loss of one copy of the Ring2 genome in part of the baculovirus population. Approximately 50% of the baculoviruses showed single copy Ring2 genomes rather than a tandem copy. Circular Ring2 genomes were not detected from the baculoviruses (in contrast to tandem Ring2 constructs introduced into MOLT-4 cells, in which circular single-copy dsDNA genomes were detected; FIG. 27I). However, this recombination did not inhibit the successful delivery of the tandem genome copies to SF9 cells.

### Example 28: Production of Anellovirus Proteins in a Baculovirus Expression System

In this example, a baculovirus expression system from Thermofisher Scientific (Cat. no. A38841) was adapted for expression of Anellovirus proteins. Briefly, a gene of interest (e.g., a gene encoding an Anellovirus ORF as described herein) was cloned into the pFastBac plasmid, which was then transformed into DH10Bac E. coli cells harboring a baculovirus genome. The transformants were grown on indicator plates according to the manufacturer's instructions and white colonies were selected for liquid culture and extraction of bacmid DNA. Recombination of the Anellovirus ORFs into the bacmids was validated by PCR.

Validated bacmid constructs showing successful recombination of the anellovirus ORF gene were then transfected into ExpiSf9 insect cells. The cells were incubated in a 27° C. non-humidified, non-CO₂ atmosphere incubator on an orbital shaker set at 125 rpm. After 72 hours post-transfection, Passage 0 stock (P0) recombinant baculovirus was harvested from the supernatant.

ExpiSf9 cells were infected using 25-100 μL of P0 baculovirus stock to make Passage 1 (P1) baculovirus for protein production. After 96 hours (approx. 4 days) post-infection, the supernatant was collected to obtain P1 baculovirus.

P1 recombinant baculovirus was titered by preparing five 10-fold serial dilutions of the test virus in fresh ExpiSf CD Medium in 1200 μL total volume. 800 μL of Expisf9 cells at 1.25×10⁶ viable cells/mL were seeded in a deep well plate and 1000 μL of the different dilutions of the test virus were added to each well. One well was setup as a negative control. Plate was then incubated overnight at 27° C. in a non-humidified incubator on a shaking platform at 225±5 rpm. After approx. 14-16 hours of incubation, the plate was removed from the incubator and everything was transferred to microcentrifuge tubes and spun at 300×g for 5 minutes. Supernatant was aspirated and each cell pellet was resuspended in 100 μL of dilution buffer (PBS+2% Fetal Bovine Serum) containing Anti-Baculovirus Envelope gp64 APC antibody at a final concentration of 0.15 μg/mL. Tubes were incubated for 30 mins at room temperature. Samples were then washed with 1 mL PBS followed by a 10 min centrifuge spin at 300×g. Supernatant was aspirated and cell pellet was resuspended in 1 mL Dilution Buffer. Samples were analyzed on a flow cytometer using the following parameters: red laser excitation: 633-647 nm; emission: 660 nm. Samples with different viral dilutions expressing percent positive gp64 were noted and used to calculate the viral titer.

For this and following examples, a series of recombinant bacmids and baculovirus vectors was produced for expression using the method described above. As shown in Table 47 below, various ORFs from LY2, tth8, and other anellovirus strains were cloned into bacmids. The ORFs were either tagged with an N-terminal His-tag with or without a human rhinovirus 3C (HRV 3C) proteolytic cleavage site, a C-terminal His-tag, or were left untagged, as indicated.

TABLE 47
Recombinant bacmid constructs produced. “FullORF” = Full ORF-containing region, with noncoding regions removed; ORF2/3 tagged.

| Construct #/name | Strain | Ring # | ORF | Tag type | Tag position | pFastBac | Bacmid | Baculovirus | Made |
|---|---|---|---|---|---|---|---|---|---|
| tth8 ORF1 | tth8 | Ring1 | ORF1 | no-tag | NA | Yes | Yes | No | in-house |
| tth8 ORF1 N-His | tth8 | Ring1 | ORF1 | 6xHis | N-ter | Yes | Yes | Yes | in-house |
| tth8 ORF1 C-His | tth8 | Ring1 | ORF1 | 6xHis | C-ter | Yes | Yes | Yes | in-house |
| tth8 ORF2 | tth8 | Ring1 | ORF2 | no-tag | NA | Yes | Yes | Yes | in-house |
| tth8 ORF2 C-His | tth8 | Ring1 | ORF2 | 6xHis | C-ter | Yes | Yes | Yes | in-house |
| tth8 ORF1/1 | tth8 | Ring1 | ORF1/1 | no-tag | NA | Yes | Yes | Yes | in-house |
| tth8 ORF1/1 C-His | tth8 | Ring1 | ORF1/1 | 6xHis | C-ter | Yes | Yes | Yes | in-house |
| tth8 ORF1/2 | tth8 | Ring1 | ORF1/2 | no-tag | NA | Yes | Yes | Yes | in-house |
| tth8 ORF1/2 C-His | tth8 | Ring1 | ORF1/2 | 6xHis | C-ter | Yes | Yes | Yes | in-house |
| tth8 ORF2/2 | tth8 | Ring1 | ORF2/2 | no-tag | NA | Yes | Yes | Yes | in-house |
| tth8 ORF2/2 C-His | tth8 | Ring1 | ORF2/2 | 6xHis | C-ter | Yes | Yes | Yes | in-house |
| tth8 ORF2/3 | tth8 | Ring1 | ORF2/3 | no-tag | NA | Yes | Yes | Yes | in-house |
| tth8 ORF2/3 C-His | tth8 | Ring1 | ORF2/3 | 6xHis | C-ter | Yes | Yes | Yes | in-house |
| tth8 FullORF | tth8 | Ring1 | FullORF | no-tag | NA | Yes | Yes | Yes | in-house |
| tth8 FullORF C-His | tth8 | Ring1 | FullORF | 6xHis | C-ter | Yes | Yes | Yes | in-house |
| tth8 ORF2 C-His | tth8 | Ring1 | ORF2/ORF1 | 6xHis | C-ter | Yes | Yes | Yes | in-house |
| Ring 3.1 ORF1 | 6B.CD8.contig3 | Ring3.1 | ORF1 | no-tag | NA | No | No | No | in-house |
| Ring 3.1 ORF1 C-His | 6B.CD8.contig3 | Ring3.1 | ORF1 | 6xHis | C-ter | Yes | Yes | Yes | in-house |
| Ring 3.1 ORF2 | 6B.CD8.contig3 | Ring3.1 | ORF2 | no-tag | NA | No | No | No | in-house |
| Ring 3.1 ORF2 C-His | 6B.CD8.contig3 | Ring3.1 | ORF2 | 6xHis | C-ter | Yes | Yes | Yes | in-house |
| Ring 3.1 ORF2/ORF1 | 6B.CD8.contig3 | Ring3.1 | ORF2/ORF1 | 6xHis | C-ter | Yes | Yes | Yes | in-house |
| C-His | | | | | | | | | |
| LY2 FullORF | LY2 | Ring2 | FullORF | no-tag | NA | Yes | Yes | No | in-house |
| LY2 FullORF N-His | LY2 | Ring2 | FullORF | 6xHis | N-ter | Yes | Yes | Yes | in-house |
| LY2 FullORF C-His | LY2 | Ring2 | FullORF | 6xHis | C-ter | Yes | Yes | Yes | in-house |
| LY2 ORF1 | LY2 | Ring2 | ORF1 | no-tag | NA | Yes | Yes | No | in-house |
| LY2 ORF1 N-His | LY2 | Ring2 | ORF1 | 6xHis | N-ter | Yes | Yes | Yes | in-house |
| LY2 ORF1 C-His | LY2 | Ring2 | ORF1 | 6xHis | C-ter | Yes | Yes | Yes | in-house |
| LY2 ORF1(dR) | LY2 | Ring2 | ORF1 (delta- | no-tag | NA | Yes | No | No | in-house |
| | | | arginine rich | | | | | | |
| | | | region) | | | | | | |
| LY2 ORF1(dR) N-His | LY2 | Ring2 | ORF1 (delta- | 6xHis | N-ter | Yes | Yes | Yes | in-house |
| | | | arginine rich | | | | | | |
| | | | region) | | | | | | |
| LY2 ORF1(dR) C-His | LY2 | Ring2 | ORF1 (delta- | 6xHis | C-ter | Yes | Yes | Yes | in-house |
| | | | arginine rich | | | | | | |
| | | | region) | | | | | | |
| LY2 ORF1/1 | LY2 | Ring2 | ORF1/1 | no-tag | NA | Yes | Yes | No | in-house |
| LY2 ORF1/1 N-His | LY2 | Ring2 | ORF1/1 | 6xHis | N-ter | Yes | Yes | Yes | in-house |
| LY2 ORF1/1 C-His | LY2 | Ring2 | ORF1/1 | 6xHis | C-ter | Yes | Yes | Yes | in-house |
| LY2 ORF1/2 | LY2 | Ring2 | ORF1/2 | no-tag | NA | Yes | Yes | No | in-house |
| LY2 ORF1/2 N-His | LY2 | Ring2 | ORF1/2 | 6xHis | N-ter | Yes | Yes | Yes | in-house |
| LY2 ORF1/2 C-His | LY2 | Ring2 | ORF1/2 | 6xHis | C-ter | Yes | Yes | Yes | in-house |
| LY2 ORF2 | LY2 | Ring2 | ORF2 | no-tag | NA | Yes | Yes | No | in-house |
| LY2 ORF2 N-His | LY2 | Ring2 | ORF2 | 6xHis | N-ter | Yes | Yes | Yes | in-house |
| LY2 ORF2 C-His | LY2 | Ring2 | ORF2 | 6xHis | C-ter | Yes | Yes | Yes | in-house |
| LY2 ORF2/2 | LY2 | Ring2 | ORF2/2 | no-tag | NA | Yes | Yes | No | in-house |
| LY2 ORF2/2 N-His | LY2 | Ring2 | ORF2/2 | 6xHis | N-ter | Yes | Yes | Yes | in-house |
| LY2 ORF2/2 C-His | LY2 | Ring2 | ORF2/2 | 6xHis | C-ter | Yes | Yes | Yes | in-house |
| LY2 ORF2/3 | LY2 | Ring2 | ORF2/3 | no-tag | NA | Yes | Yes | No | in-house |
| LY2 ORF2/3 N-His | LY2 | Ring2 | ORF2/3 | 6xHis | N-ter | Yes | Yes | Yes | in-house |
| LY2 ORF2/3 C-His | LY2 | Ring2 | ORF2/3 | 6xHis | C-ter | Yes | Yes | Yes | in-house |
| LY2 ORF2/ORF1 C-His | LY2 | Ring2 | ORF2/ORF1 | 6xHis | C-ter | Yes | Yes | Yes | in-house |
| LY2 ORF1 HisE354 | LY2 | Ring2 | ORF1 | 6xHis | After E354 | Yes | Yes | No | in-house |
| LY2 ORF1 HisN299 | LY2 | Ring2 | ORF1 | 6xHis | After N299 | Yes | Yes | No | in-house |
| LY2 ORF1 HisL267 | LY2 | Ring2 | ORF1 | 6xHis | After L267 | Yes | Yes | No | in-house |
| tth8 ORF1 (JA20 | tth8 | Ring1 | ORF1 (with | 6xHis | C-ter | Yes | No | No | in-house |
| HVR) | | | JA20's | | | | | | |
| | | | hypervariable | | | | | | |
| | | | region) | | | | | | |
| tth8 ORF1 (TJN02 | tth8 | Ring1 | ORF1 (with | 6xHis | C-ter | Yes | No | No | in-house |
| HVR) | | | TJN02's | | | | | | |
| | | | hypervariable | | | | | | |
| | | | region) | | | | | | |
| tth8 ORF1 (TTV16 | tth8 | Ring1 | ORF1 (with | 6xHis | C-ter | Yes | No | No | in-house |
| HVR) | | | TTV16's | | | | | | |
| | | | hypervariable | | | | | | |
| | | | region) | | | | | | |
| Ring2 ORF1 (CodOpt) | LY2 | Ring2 | ORF1 (codon | no-tag | NA | Yes | Yes | Yes | Medigen |
| | | | optimized) | | | | | | |
| Ring2 ORF1 (CodOpt) | LY2 | Ring2 | ORF1 (codon | 6xHis | C-ter | Yes | Yes | Yes | Medigen |
| HRV3C-6His | | | optimized) | | | | | | |
| Ring4 ORF1 (CodOpt) | 6B.CD8.contig2 | Ring4 | ORF1 (codon | no-tag | NA | Yes | Yes | Yes | Medigen |
| | | | optimized) | | | | | | |
| RIng4 ORF1 (CodOpt) | 6B.CD8.contig2 | Ring4 | ORF1 (codon | 6xHis | C-ter | Yes | Yes | Yes | Medigen |
| HRV3C-6His | | | optimized) | | | | | | |
| RIng5.2 ORF1 (CodOpt) | CT30F | Ring5.2 | ORF1 (codon | no-tag | NA | Yes | Yes | Yes | Medigen |
| | | | optimized) | | | | | | |
| Ring5.2 ORF1 (CodOpt) | CT30F | RIng5.2 | ORF1 (codon | 6xHis | C-ter | Yes | Yes | Yes | Medigen |
| HRV3C-6His | | | optimized) | | | | | | |
| Ring6 ORF1 (CodOpt) | 190783.3 | Ring6 | ORF1 (codon | no-tag | NA | Yes | Yes | Yes | Medigen |
| | | | optimized) | | | | | | |
| Ring6 ORF1 (CodOpt) | 190783.3 | Ring6 | ORF1 (codon | 6xHis | C-ter | Yes | Yes | Yes | Medigen |
| HRV3C-6His | | | optimized) | | | | | | |
| Ring1 ORF1 (CosOpt) | tth8 | Ring1 | ORF1 (codon | 6xHis | C-ter | Yes | Yes | Yes | Medigen |
| His | | | optimized) | | | | | | |
| Rig3.1 ORF1 (CodOpt) | 6B.CD8.contig3 | Ring3.1 | ORF1 (codon | 6xHis | C-ter | Yes | Yes | Yes | Medigen |
| His | | | optimized) | | | | | | |
| Ring7 ORF1 (CodOpt) | 190783.4 | Ring7 | ORF1 (codon | 6xHis | C-ter | Yes | Yes | Yes | Medigen |
| His | | | optimized) | | | | | | |
| Ring2 (CodOpt) N-His | LY2 | Ring2 | ORF1 (codon | 6xHis | N-ter | Yes | Yes | Yes | Medigen |
| | | | optimized) | | | | | | |
| Ring2 (CodOpt) N-His | LY2 | Ring2 | ORF1 (codon | 6xHis- | N-ter | Yes | Yes | Yes | Medigen |
| (PS) | | | optimized) | PreScision | | | | | |
| | | | | Protease | | | | | |
| | | | | recognition | | | | | |
| | | | | sequence) | | | | | |
| Ring2 tandem | LY2 | Ring2 | 2x whole genome | no-tag | NA | Yes | Yes | Yes | Medigen |
| | | | (without | | | | | | |
| | | | Polyhedrin | | | | | | |
| | | | promoter) | | | | | | |
| WTLY2 | LY2 | Ring2 | whole genome | no-tag | NA | Yes | Yes | Yes | in-house |
| WTtth8 | tth8 | Ring1 | whole genome | no-tag | NA | Yes | Yes | Yes | in-house |
| WTtth8 (Reverse) | tth8 | Ring1 | whole genome | no-tag | NA | Yes | Yes | Yes | in-house |
| | | | (with Reversed | | | | | | |
| | | | 5′ Polyhedrin | | | | | | |
| | | | promoter) | | | | | | |
| LoxPWTLY2 | LY2 | Ring2 | LoxP-whole | no-tag | NA | Yes | Yes | Yes | in-house |
| | | | genome-LoxP | | | | | | |
| Cre-R | NA | NA | Cre recombinase | no-tag | NA | Yes | Yes | Yes | in-house |

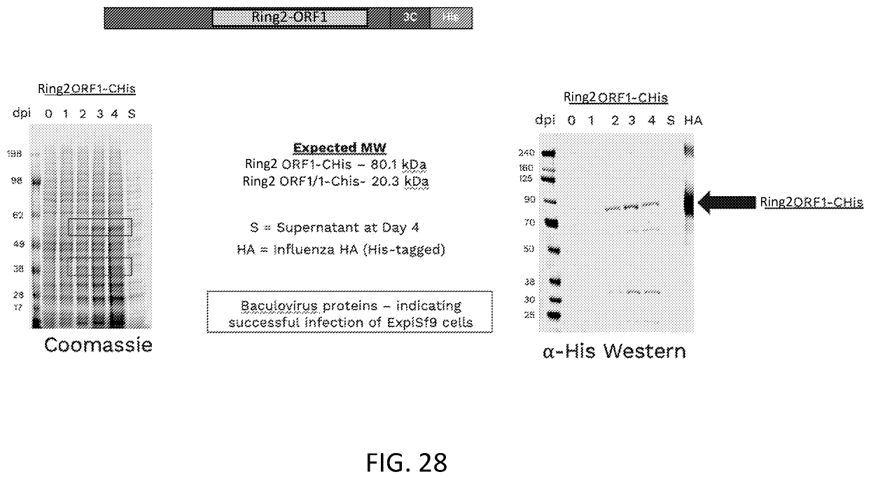

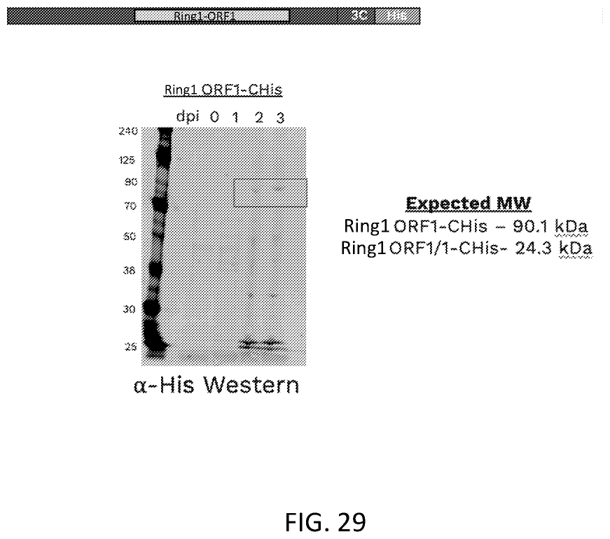

On the day before infection, ExpiSf9 cells were seeded at 5×10⁶ cells/ml in 25 ml room temperature ExpiSf9 CD Medium in 125 ml Nalgene Single-Use PETG Erlenmeyer Plain Bottom Flask [Thermofisher Scientific Catalog no: 4115-0125]. The cell viability was monitored to ensure that it was maintained at or above 95%. 100 μL of ExpiSf Enhancer solution was added to the cells in a dropwise manner. Cells were incubated with shaking overnight in a 27° C. non-humidified, air-regulated, non-CO₂ atmosphere incubator using an orbital shaker at 125±5 rpm. On Day 1, approximately 18-24 hours after adding ExpiSf Enhancer, cells were infected with the indicated baculovirus at a multiplicity of infection (MOI) of 5 and incubated under the same conditions. Cells were harvested 72 hours post infection and viability was found to range between 60 and 80%. To analyze samples, cells were lysed by adding 1× Bolt LDS sample buffer [Invitrogen Catalog No.: B0007] and 1× Bolt reducing agent [Invitrogen Catalog No.: B0009] and sonicating for 2.5 minutes. As shown in FIG. 28, C-His-tagged LY2 ORF1 was successfully expressed in infected ExpiSf9 cells by day 2 post-infection as determined by western blotting using an anti-poly-histidine antibody. In addition, baculovirus proteins were detected by Coomassie staining, indicating a successful infection.

As shown in FIG. 29, C-His-tagged tth8 ORF1 and ORF1/1 were also successfully expressed in infected ExpiSf9 cells by day 2 post-infection.

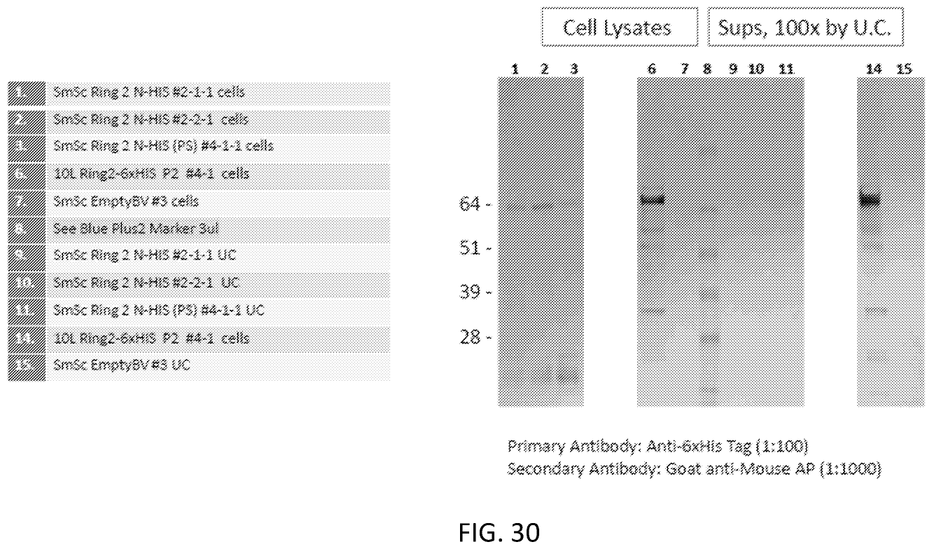

N-terminally His-tagged LY2 ORF1 expression was also detected in infected ExpiSf9 cells (FIG. 30). Here, constructs either comprised an N-terminal His-tag which was immediately followed by the wildtype ORF1 sequence (lanes 1, 2, 9, 10, or 14), or an N-terminal His-tag which was followed by a rhinovirus 3C cleavage sequence (lanes 3, 11). Samples in lanes 1 to 7 are lysates loaded directly onto the gel, whereas samples in lanes 9-15 were prepared by first pelleting protein from conditioned medium via ultracentrifugation and resuspending the pellet in a 100-fold smaller volume. Samples shown in lanes 1-3 and 9-11 were grown at a small (5 mL) scale. Samples in lanes 6 and 14 were obtained from a 10 L culture. Thus, this example shows that production of ORF1 from a plurality of strains with N or C terminal poly-histidine tags can be successfully carried out at a scale ranging from 5 mL to 10 L, and that ORF1 can be found in Sf9 lysate or culture supernatant (conditioned medium).

### Example 29: Expression of Ring1 ORFs in Sf9 Cells

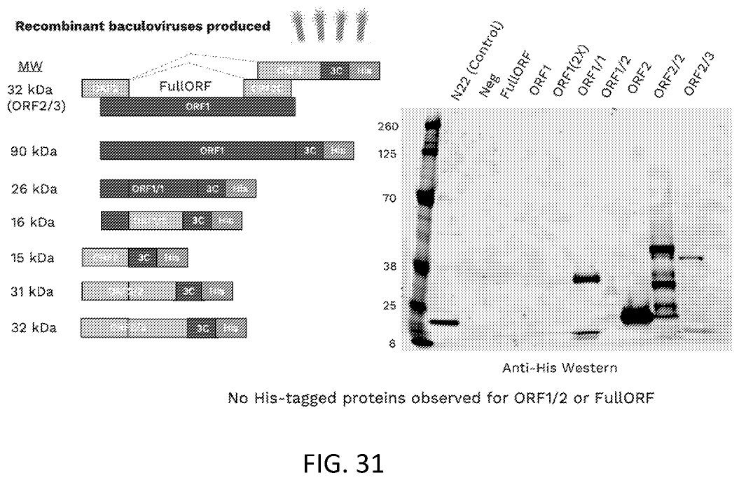

In this example, a series of recombinant baculoviruses were produced with alternate arrangements of Ring1 ORFs, each tagged with a C-terminal poly-histidine (FIG. 31). The recombinant baculovirus designs included one baculovirus construct for each of the Ring1 ORF splice variants (i.e., ORF1, ORF1/1, ORF1/2, ORF2, ORF2/2, and ORF2/3), as well as a “FullORF” construct containing the full ORF region from Ring1, driven by the baculovirus polyhedrin promoter. These baculoviruses were produced as described in Example 28.

Protein expression was then detected by western blot using anti-poly-histidine antibody. As shown in FIG. 31, His-tagged Ring1 ORFs ORF1/1, ORF1/2, ORF2, ORF2/2 and ORF2/3 were detected.

### Example 30: Expression of Ring2 ORFs in Sf9 Cells

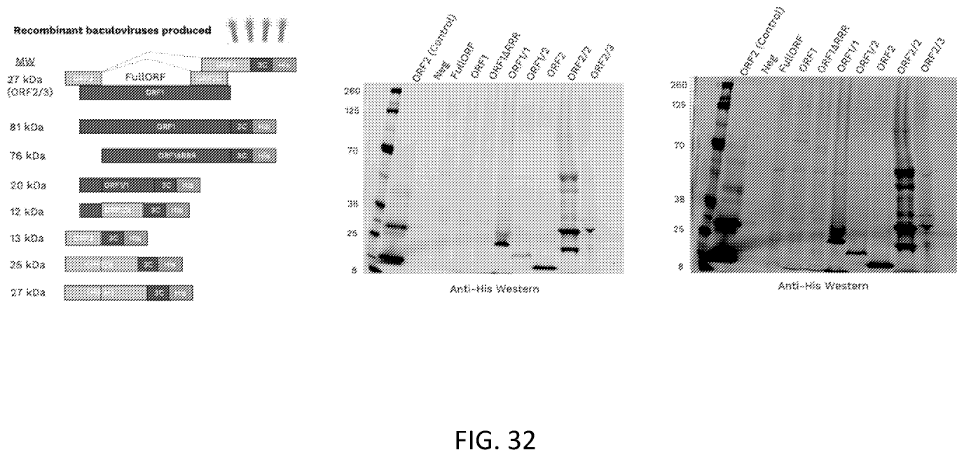

In one example, a series of recombinant baculoviruses were produced with alternate arrangements of Ring2 ORFs, each tagged with a poly-histidine tag at the C terminus (FIG. 32). The recombinant baculovirus designs included one baculovirus construct for each of the Ring2 ORF splice variants (i.e., ORF1, ORF1/1, ORF1/2, ORF2, ORF2/2, and ORF2/3), a variant in which the N-terminal arginine-rich region (RRR) is deleted (ORF1ΔRRR), as well as a “FullORF” construct containing the full ORF region from Ring2 driven by the baculovirus polyhedrin promoter. For each experimental condition, ExpiSf9 cells were infected with recombinant baculoviruses expressing individual Ring2 variants at an MOI of 5. The experimental conditions for this were as described in Examples 28 and 29.

Protein expression was then detected by western blot using anti-His. As shown in FIG. 32, His-tagged Ring2 ORFs ORF1, ORF1ΔRRR, ORF1/1, ORF1/2, ORF2, ORF2/2, and ORF2/3 were all detected.

In a further experiment as part of this example, recombinant baculoviruses comprising a Ring2 ORF1-encoding sequence and/or a Ring2 ORF2 splice variant-encoding sequence were used to infect Sf9 cells. The expression conditions tested included ORF1 alone, or co-infection of ORF1+“FullORF”, ORF1+ORF2, ORF1+ORF2/2, and ORF1+ORF2/3, as well as a negative control labeled ‘Neg’. ExpiSf9 cells were co-infected with baculoviruses at a MOI of 5 for each condition. Experimental conditions were as described in Examples 28 and 29. Protein expression of ORF1, ORF2, ORF2/2, and ORF2/3 was then assessed for each condition by western blot using either anti-His or anti-Ring2 N22. The latter is a monoclonal antibody that was obtained by immunizing mice with the N22 fragment of Ring2 ORF1 produced in E. coli, and then generating hybridomas.

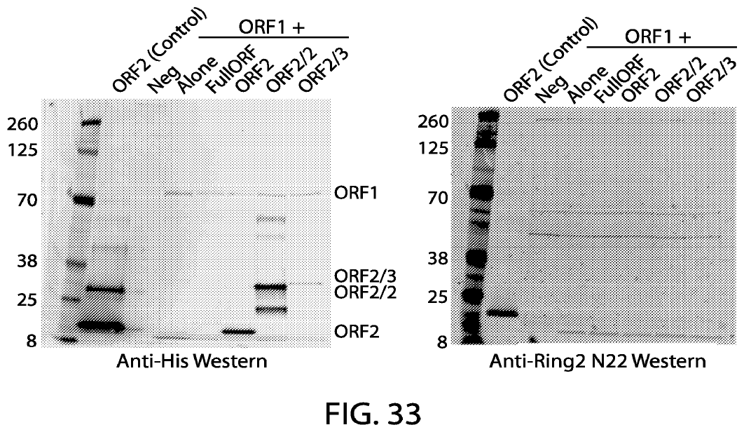

As shown in FIG. 33, both Westerns detected ORF1 as a band at ˜81 kD in each of the ORF1-infected conditions. The ORF1 band is highlighted by a dashed box in the anti-N22 Western, and is not visible in the negative control (Neg) sample. The lower molecular weight (˜10 kD) band detected by both antibodies is thought to be a C-terminal fragment of ORF1. ORF2, ORF2/2, and ORF2/3 were also detected in the corresponding samples (anti-His blot). Thus, this example illustrates that both ORF1 and individual splice variants of ORF2 can be co-expressed in insect cells.

### Example 31: Expression of all Ring2 ORFs Simultaneously in Sf9 Cells

In one example, a series of six recombinant baculoviruses were produced, each designed to express a particular Ring2 ORF (i.e., ORF1, ORF1/1, ORF1/2, ORF2, ORF2/2, and ORF2/3), each tagged with a His tag (FIG. 34), as described in Example 30. Sf9 cells were infected with various combinations of the Ring2 ORF baculoviruses—specifically, each condition involved infecting cells with all but one ORF construct, as indicated in FIG. 34. Protein expression was then detected by western blot of whole cell suspension using anti-His. As shown in FIG. 34, His-tagged Ring2 ORFs were detected in the expected pattern. Either all ORFs were detected, or all except for the omitted one.

### Example 32: Co-Delivery and Independent Expression of Anellovirus Genomes and Recombinant Anellovirus ORFs in Sf9 Cells

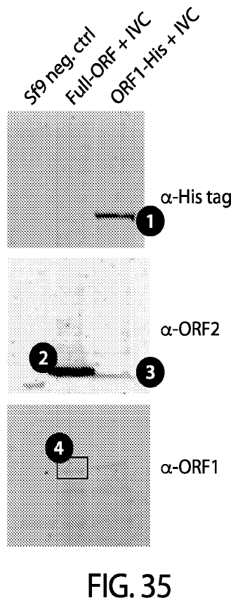

In this example, anellovirus ORFs and genomes were co-delivered in Sf9 cells by transfecting an in vitro circularized (IVC) anellovirus genome and infecting the cells with baculovirus encoding ORF1 tagged at its C-terminus with hexa-histidine (FIG. 35). Protein expression was then detected by western blot using anti-His, anti-ORF2, and anti-ORF1 monoclonal antibody targeting the N22 fragment. As shown (FIG. 35, bullet 1), His-tagged ORF1 was detected in this preparation showing successful recombinant ORF1 expression from the baculovirus vector. Consistent with this result, the same ORF1 protein was detected using the anti-ORF1 antibody (FIG. 35, bottom panel, right-most lane).

In the same sample of treated cells, the native anellovirus promoter was shown to be transcriptionally active in Sf9 cells because ORF2 expression was detected (FIG. 35, bullet 3) and could only have been produced by the IVC genome which was transfected into the cells.

In addition, Anellovirus ORFs were co-delivered and expressed in Sf9 cells using an in vitro circularized (IVC) construct and a FullORF baculovirus. Protein expression was then detected by western blot using anti-His, anti-Ring2 ORF2, and anti-Ring2 ORF1 N22. ORF1 protein was detected in the cells (FIG. 35, bullet 4) and could be the product of either the IVC or the FullORF baculovirus construct. Surprisingly, ORF2 protein was readily detected and its intensity suggests the expression is derived from the FullORF baculovirus construct (FIG. 35, bullet 2).

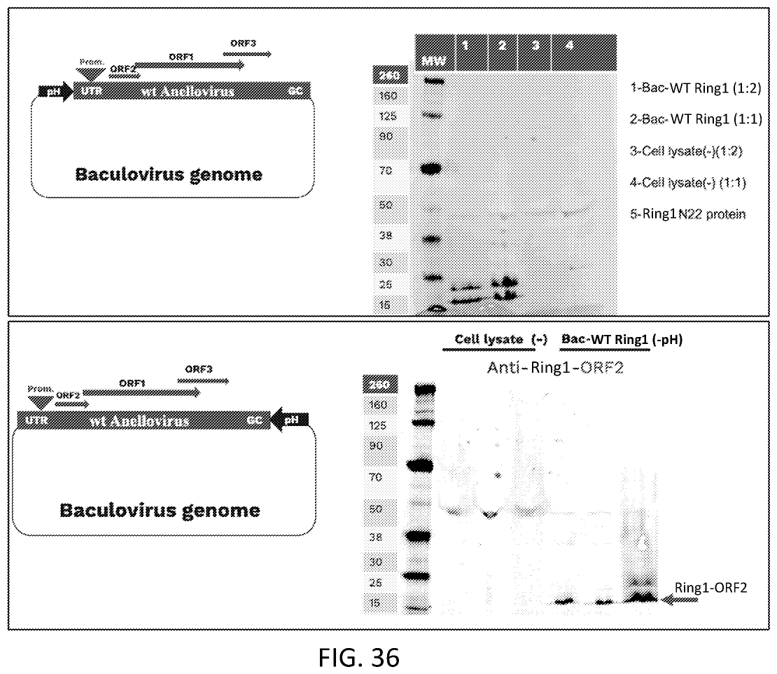

As a further test of the ability of the anellovirus genome to express its genes in insect cells, the tth8 anellovirus coding region was cloned into the pFastBac vector in both orientations. This yielded ‘FullORF’ tth8 baculovirus constructs in which the polyhedrin promoter was positioned upstream of either the sense or the anti-sense direction of the coding region. The latter configuration is highly unlikely to initiate transcription of the anellovirus genes. Consistent with our surprising observations in Ring2, expression of tth8 ORF2 was independent of the orientation of the coding region relative to the baculovirus polyhedrin promoter, suggesting that expression is driven by the anellovirus promoter (FIG. 36, bands at ˜15 and 20 kDa).

This example shows that IVC transfections and baculovirus infections can co-deliver functional anellovirus genes to Sf9 insect cells and that the native anellovirus promoter is active in these cells.

### Example 33: Anellovirus ORF1 Associates with DNA in Sf9 Cells to Form Complexes Isolated by Isopycnic Centrifugation

In this example, Sf9 cells were transfected with IVC anellovirus genome LY2, infected with a baculovirus encoding LY2 ORF1 with a C-terminal poly-histidine tag, and then fractionated to determine whether ORF1 expressed using the baculoviral expression system forms protein-DNA complexes that can be isolated in vitro.

CsCl gradients were prepared by adding 8 ml of 1.2 g/ml CsCl solution (in TN buffer; 20 mM Tris pH 8.0, 140 mM NaCl) to ultracentrifuge tubes (Ultra-Clear 17 ml—Beckman #344061) for SW32.1 Ti rotor. Tubes were underlayed with 8 ml of 40% CsCl (in TN buffer), then capped with topper and run on Gradient Master program 5-50% for 13 minutes to prepare linear gradient. The caps were removed and the gradients overlayed with 0.5 ml-2 ml of Sf9 lysate to each tube and topped off to near the top with TN buffer containing 0.001% Poloxamer-188. Ultracentrifugation was for 18.5 hours at 22,500×RPM. Fractions were collected from the gradient by piercing the bottom of the tube and allowing ˜600 ul fractions to flow into wells of a deep well block. The refractive index of each sample was measured to determine their density.

Anelloviral DNA content in the fractions was then determined by first extracting DNA from the fractions, and then by carrying out qPCR. Pure Link Viral DNA extraction Kit [Thermofisher Scientific Catalog no. 12280050] was used to purify viral DNA from 50 uL of the fractions. The samples were treated with Proteinase K and lysed using Lysis buffer by incubating at 56° C. for 15 min., washed with 99% ethanol, and transferred to a Viral Spin Column. Samples were centrifuged at 6800×g, washed twice with 500 uL Wash buffer provided with the kit and centrifuged again. 100 uL of RNase-free water was added to the column to elute the DNA.

For qPCR, 2×TaqMan Gene Expression Master Mix, 100 uM LY2 primers Forward (AGCAACAGGTAATGGAGGAC), 100 uM LY2 Reverse (TGAAGCTGGGGTCTTTAAC) along with 100 uM LY2 Probe (TCTACCTAGGTGCAAAGGGCC) were diluted in 5.83 uL Nuclease Free water for each reaction. The following conditions were used for each qPCR cycle: 50° C. hold for 2 minutes, 95° C. hold for 10 minutes followed by 40 cycles of 95° C. for 15 seconds and 60° C., for 1 minute on an Applied Biosystems Quant Studio 3 Real-Time PCR Machine. Each sample was run in triplicate and the entire assay was repeated thrice and used to plot the graph.

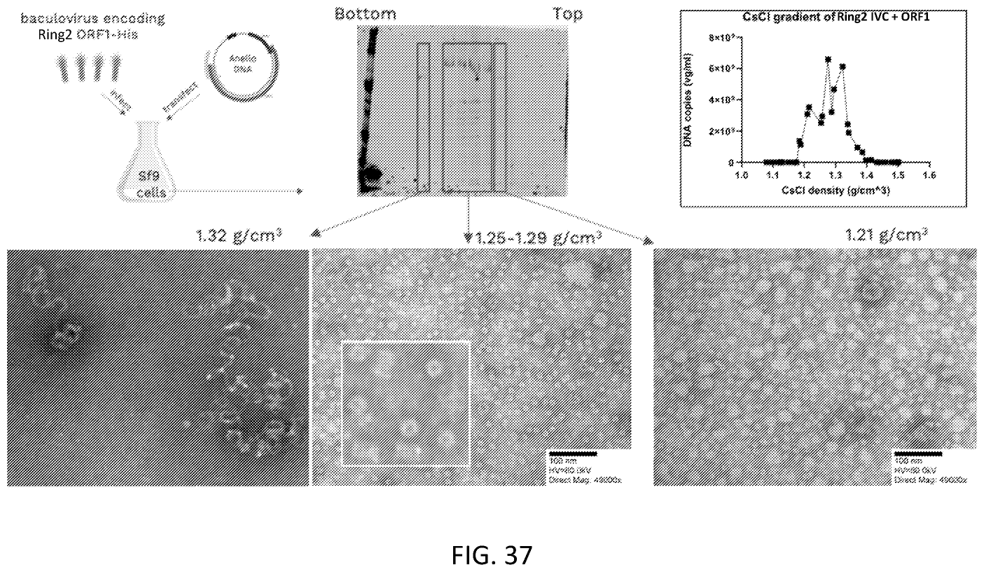

As shown in FIG. 37, isopycnic fractions were characterized by western blotting, quantitative PCR, and transmission electron microscopy. Anti-his western blotting of gradient fractions showed clear bands of the expected molecular weight for LY2 ORF1 in fractions having densities of 1.32 g/mL and 1.21 g/mL. In addition, fractions ranging from 1.25 to 1.29 g/mL had clear bands of higher and lower molecular weights than expected. Also, qPCR indicates the presence of LY2 genomic DNA in certain fractions, with peaks at approximately 1.21 g/mL, 1.29 g/mL, and 1.32 g/mL.

Negative stain transmission electron microscopy was carried out on the 1.32 g/mL and 1.21 g/mL fractions, as well as a pool of fractions ranging from 1.25 to 1.29 g/mL. The pool shows an abundance of particles, including several having the appearance of proteasomes. The presence of proteasomes may explain the western blot bands at low and high molecular weights. The former may be due to proteolytic degradation and the latter due to ubiquitylated ORF1, or ORF1 fragments covalently associated with proteasome proteins in the course of degradation. The 1.21 g/mL fraction shows particles of various sizes, including several which appear to be consistent with lipid-based particles. The 1.32 g/mL fraction shows remarkable DNA-like structures that stain differently than naked DNA, suggesting association with macromolecules such as protein.

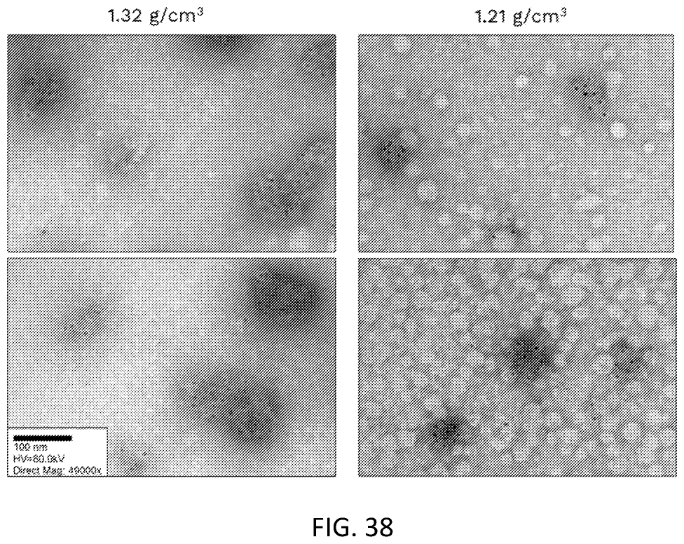

To determine if LY2 ORF1 is associated with the structures observed in the electron micrographs, immunogold detection using an anti-poly-histidine antibody was carried out. FIG. 38 shows gold label accumulating on the structures observed in the 1.32 and 1.21 g/mL fractions, consistent with the presence of ORF1-His in association with the DNA seen in the 1.32 g/mL fraction, and in the particles seen in the 1.21 g/mL fraction.

Taken together, these results show that ORF1 expressed in Sf9 cells can associate with DNA to form complexes having a density consistent with anellovirus particles.

### Example 34: Expression of ORF1 Protein from a Diverse Array of Anelloviruses Using Baculovirus

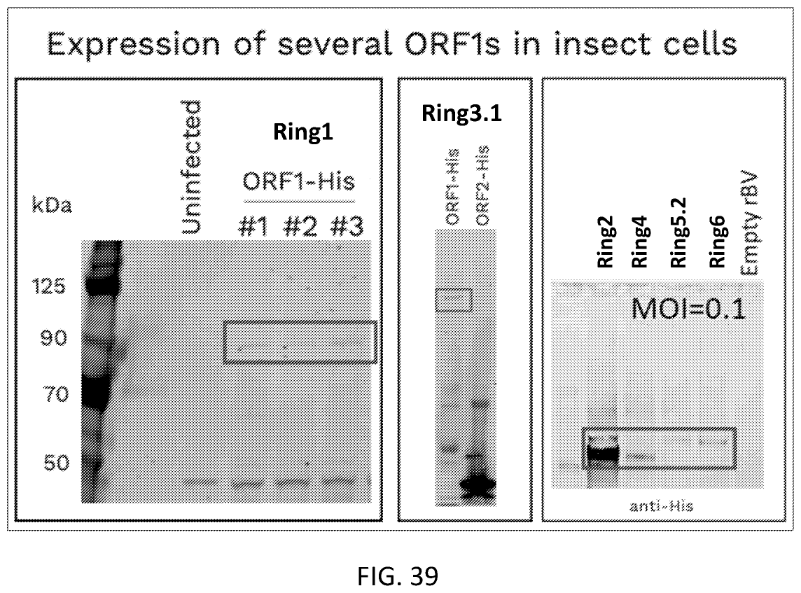

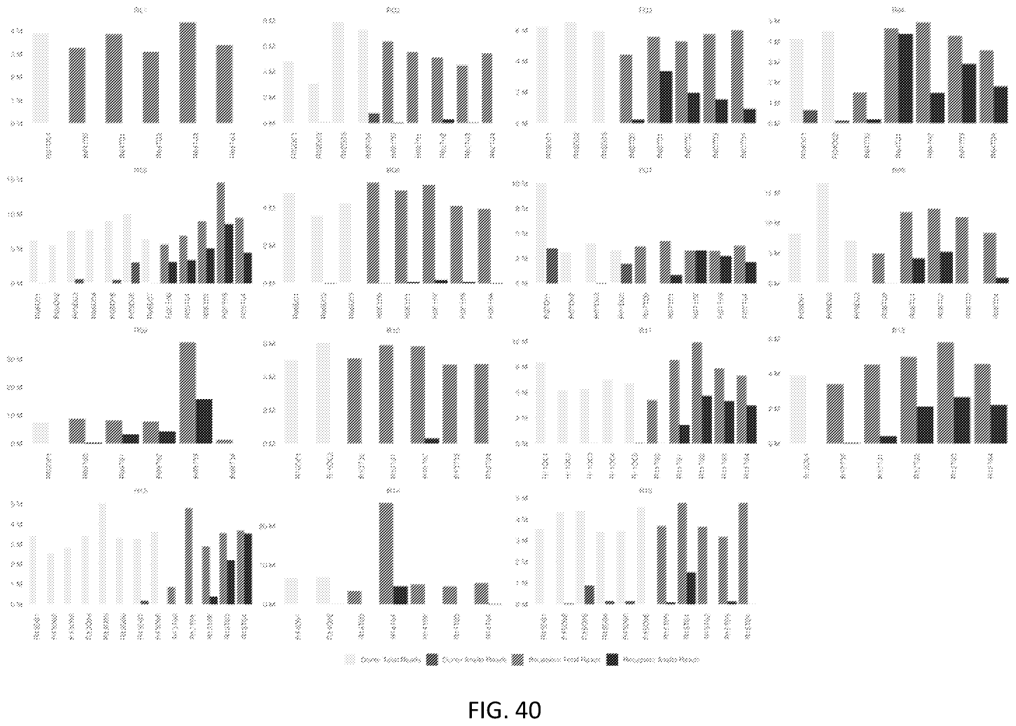

In this example, Sf9 cells were infected with baculoviruses engineered to express C-terminal His-tagged ORF1 proteins from anellovirus strains Ring3.1, Ring4, Ring5.2, Ring6, as well as Ring1 and Ring2. As shown in FIG. 39, ORF1 protein originating from each of the Anellovirus strains were successfully expressed in Sf9 cells. As shown in Table Y, Anellovirus ORF1 from the strains representing all three genera (Alphatorquevirus, Betatorquevirus, and Gammatorquevirus), were tested and their expression level is seen in FIGS. 28, 29, 30, and 39. In general, we find that the level of expression in this system is highest for ORF1 from Betatorqueviruses, intermediate from Gammatorqueviruses, and lowest from Alphatorqueviruses.

TABLE Y
Strains for which recombinant ORF1 expression was successful

| Name | Genus |
|---|---|
| Ring1 | Alpha |
| Ring2 | Beta |
| Ring3.1 | Gamma |
| Ring4 | Gamma |
| Ring5.2 | Alpha |
| Ring6 | Alpha |
| HLH | Beta |
| ctgh3 | Beta |
| LY1 | Beta |

### Example 35: In Vitro Assembly of Baculovirus Constructs

In this example, baculovirus constructs suitable for expression of Anellovirus proteins (e.g., ORF1) are generated by in vitro assembly.

DNA encoding Anellovirus ORF1 (wildtype protein, chimeric protein or fragments thereof) which may be untagged or contain tags fused N-terminally, C-terminally, or harbor mutations within the ORF1 protein itself to introduce a tag to aid in purification and/or identity determination through immunostaining assays (such as, but not limited to, ELISA or Western Blot) is expressed in insect cell lines (Sf9 and/or HighFive). Anellovirus ORF1 may be expressed alone or in combination with any number of helper proteins including, but not limited to, Anellovirus ORF2 and/or ORF3 proteins.

Protein is purified using developed purification techniques potentially including but not limited to chelating purification, heparin purification, gradient sedimentation purification and/or size exclusion purification. ORF1 is evaluated for its ability to form capsomers or VLPs and used in subsequent steps for nucleic acid encapsidation.

In one example, DNA encoding Ring2 ORF1 fused to an N-terminal HIS₆-tag (HIS-ORF1) was codon optimized for insect expression and cloned into the baculovirus expression vector pFASTbac system to generate a baculovirus expressing Ring2 ORF-HIS recombinant protein using the Bac-to-BAC expression system according to manufacturer's method (ThermoFisher Scientific). 10 liters of insect cells (Sf9) were infected with Ring2 HIS-ORF1 baculovirus and the cells were harvested 3-days post-infection by centrifugation. The cells were lysed, and the lysate was purified using a chelating resin column using standard art in the field. The elution fraction containing HIS-ORF1 was dialyzed and treated with DNAse to digest host cell DNA. The resulting material was purified again using a chelating resin column and fractions containing ORF1 were retained for nucleic acid encapsidation and viral vector purification.

Nucleic acid encapsidation and viral vector purification: Ring ORF1 (wildtype protein, chimeric protein or fragments thereof) is treated with conditions sufficient to dissociate VLPs or viral capsids to enable reassembly with nucleic acid cargo. Nucleic acid cargo can be defined as double stranded DNA, single stranded DNA, or RNA which encodes a gene of interest that one wants to deliver as a therapeutic agent. Potential conditions sufficient to dissociate VLPs or viral capsids can, but are not limited to, buffers of different pH, conditions of defined conductivity (salt content), conditions containing detergents (such as SDS, Tween, Triton), conditions containing chaotropic agents (such as Urea) or conditions involving defined temperature and time (reannealing temperatures). Nucleic acid cargo of defined concentration is combined with Ring ORF1 of defined concentration and treated with conditions sufficient to permit nucleic acid encapsidation. The resulting particle, defined as viral vector, is subsequently purified, e.g., using developed standard viral purification procedures.

In one example, single stranded circular DNA of a GFP-expression plasmid is added to a solution of Ring 2 HIS-ORF1 and the resulting sample is treated with 0.1% SDS in 50 mM Tris pH 8 buffer at 37 C for 30 minutes. The resulting solution is further purified using a heparin column and the viral vector eluted from the column using a gradient of increasing NaCl concentration. The integrity of the viral vector is tested by transducing the cell lines EKVX and HEK293, and observing GFP production in at least one of the cell lines by fluorescence microscopy, demonstrating encapsidation of the nucleic acid cargo by the ORF1 protein to form the viral vector.

### Example 36: Generation of an Anellovirus Genomic Dataset

In this study, in-depth sequencing of blood transfusion donor(s)-recipient pairs was coupled with public genomic resources for large-scale assembly of novel Anellovirus genomes to characterize global and personal Anellovirus diversity through time. A targeted Anellovirus sequencing method was developed that allows for high-scale profiling of Anelloviruses directly from human samples, and was used to study longitudinal samples from a blood transfusion cohort consisting of donor(s)-recipient pairs. A large-scale sequence dataset was assembled and investigated for the kinetics and transmissibility of the anellome within and between individuals. The results showed that the breadth of the anellome (used herein to refer to the set of Anellovirus strains or variants present in a subject or population, and/or the relative quantity of such Anellovirus strains or variants therein) is greater than previously understood and that individuals harbor and transmit a multitude of unique anellomes that can persist for at least several months. In addition, it is shown that Anellovirus diversity is linked to extensive recombination.

In brief, blood and serum samples from the National Heart, Lung & Blood Institute's (NHLBI) longitudinal Transfusion-transmitted Viruses Study (TTVS) were screened to identify new Anellovirus sequences. Serum samples were obtained from the TTVS (Accession #HLB01910909a). Nucleic acids were extracted from 200 μl serum using a purelink viral DNA/RNA kit from Invitrogen. The samples were processed according to manufacturer's protocol with an increase to 60 min for the proteinase K incubation. Samples were eluted in 50 ul of nuclease-free water.

An amplification method was developed and employed that specifically targeted Anellovirus genome sequence to increase the yield of Anellovirus genomic sequences identified in each subject of the TTVS cohort. As a result, the method was capable of finding tens to hundreds of novel Anellovirus lineages. The amplification method employed multiply-primed rolling-circle amplification, which utilized degenerate amplification primers that cover conserved regions of the Anellovirus genome. Degenerate amplification primers were designed to cover well-conserved regions based on alignments of Anelloviridae genomes that were generated from published genomes on pubmed and metagenomic databases (see Table 1 below). Primers were protected by two thiophosphate modifications between each of the last three nucleotides at the 3′. The targeted rolling-circle amplification method contained a premix of Anello-specific primers according to 12 sequences shown in Table 1 at a final concentration of 0.4 μM each, 1×phi29 DNA polymerase buffer (NEB), 2 μl DNA sample, and dH₂0 in a final volume of 10 μl. The DNA mixture was then denatured at 95° C. for 3 minutes and cooled to 4° C., before being put on ice. The denatured sample was then added to 10 μl of the amplification solution which contained the Anello specific primers at a final concentration of 0.4 μM each, 1×phi29 DNA polymerase buffer (NEB), 200 ng/μl bovine albumin serum, 1 mM dNTPs, and 2 U/μl phi29 polymerase, and dH₂0 in a final conc of 10 μl. The sample was incubated at 30° C. for 20 hours followed by inactivation of the enzyme at 65° C. for 10 minutes. The final product was then diluted to 50 μl by adding nuclease-free water to reduce viscosity of the samples, and the concentration of DNA was assessed by Qubit. A Nextera Flex (Illumina ect) kit was used to prepare the samples for sequencing following the manufacturers protocol for 100-500 ng input. Library QC was carried out by using D5000 screen tape on an Agilent Tapestation 4200.

TABLE 1
Exemplary Anellovirus-specific degenerate primers

| Primer Name | Sequence |
|---|---|
| TTV-RCA-1 | CGAATGG*Y*W |
| TTV-RCA-2 | TTGCCCC*T*T |
| TTV-RCA-3 | YTGYGGB*T*G |
| TTV-RCA-4 | YAGAMAC*M*M |
| TTV-RCA-5 | GTACCAYT*T*R |
| TTV-RCA-6 | SACCACWA*A*C |
| TTV-RCA-7 | CACCGAC*V*A |
| TTV-RCA-8 | CACTCCG*A*G |
| TTV-RCA-9 | GCACTCC*T*C |
| TTV-RCA-10 | CAGACTC*C*G |
| TTV-RCA-11 | CCCACTC*A*C |
| TTV-RCA-12 | CTTCGCC*A*T |

* = Thiophosphate Bond

All libraries were sequenced on either an illumina iSeq 100 or a NextSeq 550. An initial read-out of Anellovirus content in raw sequencing reads were generated using kraken (Wood and Salzberg, 2014) using default parameters against a custom in-house constructed Anellovirus database. These resultant classified sequences were further verified using NCBI's BLASTn (Camacho et al., 2009), using default parameters, to confirm that the output from kraken were valid Anellovirus sequences. Raw sequencing reads were subjected to quality control utilizing FastQC (Andrews, n.d.) on each paired-end read set to measure various statistics in regard to each sequencing run. Each of the FastQC generated reports were aggregated into a single report using MultiQC (Ewels et al., 2016). Metrics from these reports influenced parameter selection to quality control steps further downstream during analysis.

Low quality sequence data and common adapters were removed using bbduk (Bushnell, 2014) with the following parameters: ktrim=r, k=23, mink=11, tpe=t, tbo=t, qtrim=rl, trimq=20, minlength=50, maxns=2. The supplied contaminant file was assembled by pulling target contaminant sequences from NCBI Genbank covering several bacterial species as well as human genetic elements to be removed. An accession list containing specific sequences is provided in the supplementary data.

Next, human sequences were removed in two passes using both NextGenMap (Sedlazeck et al., 2013) and BWA (Li, 2013) against the GRCh37/hg19 build of the human reference genome. NextGenMap was run with parameters --affine, -s 0.7, -p and BWA was run with default parameters. Mapped reads output in SAM file format were converted to paired-end FASTQ format using both SAMtools (Li et al., 2009) and Picard's (Broad Institute, 2018) SamToFastq utility configured with the parameter VALIDATION_STRINGENCY=“silent”. rRNA contaminants and common laboratory bacterial contaminants were removed using bbmap (Bushnell, 2014) with the following parameters: minid=0.95, bwr=0.16, bw=12, quickmatch=t, fast=t, minhits=2. An accounting of all reference sequences screened against can be found in the provided supplementary data. Finally, we de-duplicated the short read data passing all QC and decontamination steps to speed-up and aid in genome assembly quality using clumpify (Bushnell, 2014) with configured with parameter the dedupe=t.

Trimmed, decontaminated and de-duplicated sequencing data were assembled using metaSPAdes (Nurk et al., 2017) skipping the error correction module via the use of the --only-assembler parameter. Assembled contigs were filtered using PRINSEQ lite (Schmieder and Edwards, 2011) configured with parameters out_format 1, -lc_method dust and lc_threshold 20. Contigs assembled from each sample were clustered at 99.5% similarity to remove any duplicate sequences using the usearch software's cluster_fast algorithm (Edgar, 2010).

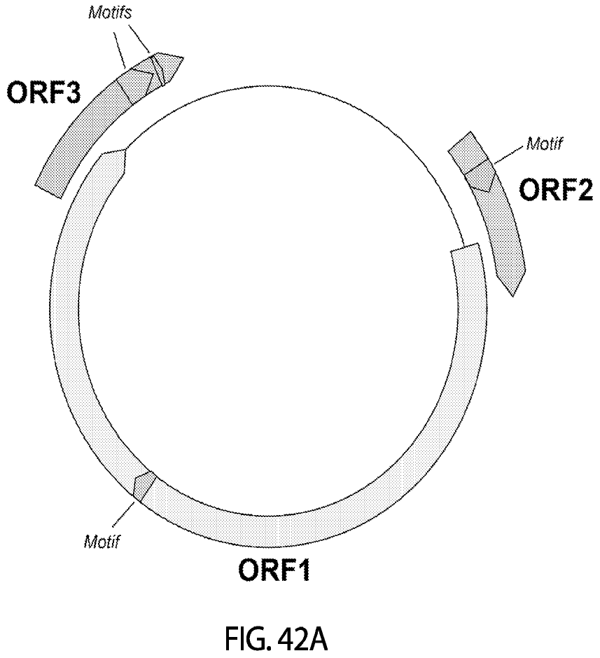

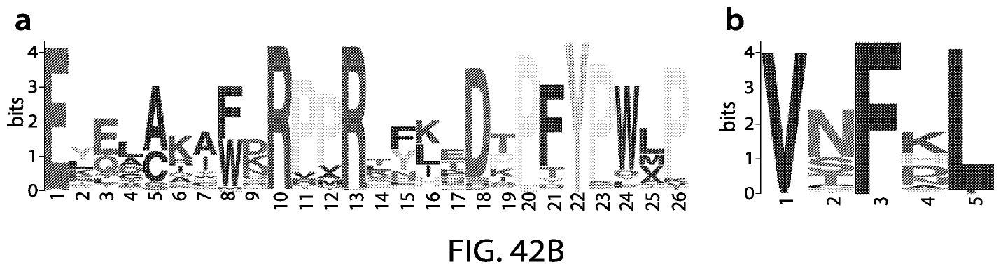

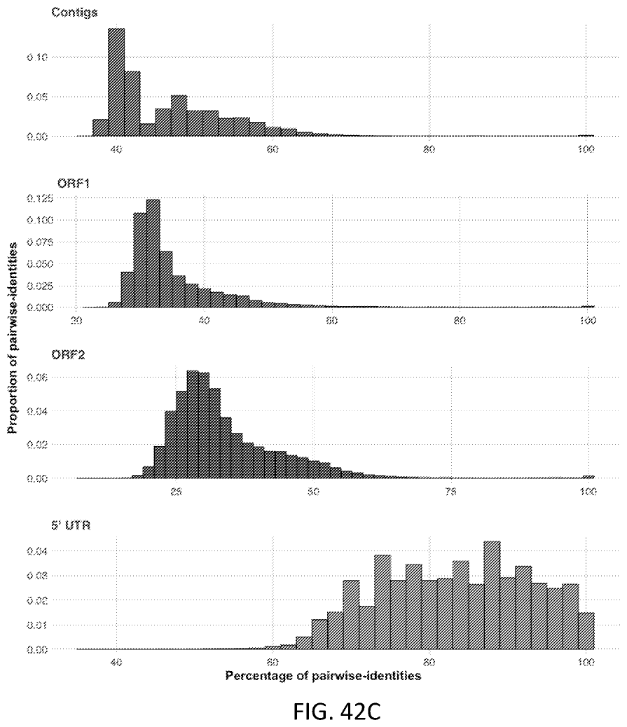

ORF sequences were called from assembled contigs using orfm (Woodcroft et al., 2016) with parameters configured to print stop codons (-p) and print ORF's in the same frame as a stop codon (-s) and constrained to ORF sequences no shorter than 50 amino acids (-m 150). Predicted ORF sequences were further filtered using seqkit's seq and grep utilities (Shen et al., 2016) to subdivide ORF sequences into ORF1, ORF2 and ORF3. ORF1 sequences were identified by filtering ORF sequences using seqkit grep for those no shorter than 600 amino acids (-m 600) and using seqkit grep to search just sequence data (-s), enable regex pattern searching (-r) and by querying for the conserved motif YNPX²DXGX²N (-p “YNP.{2}D.G.{2}”). ORF2 sequences were identified using conserved motif WX⁷HX³CXCX⁵H previously identified in literature (Takahashi et al., 2000) through seqkit's grep utility (-p “W.{7}H.{3}C.C.{5}H”). In addition to ORF1 and ORF2, a third open reading frame (ORF3) was predicted near the 3′ end of ORF1 in 471 Anellovirus genomes in the TTVS dataset. ORF3 uses a STOP codon downstream from the one used by ORF1 and its reading frame is different from that of ORF1 and ORF2. A protein in the ORF3 reading frame, labelled ORF2/3, has previously been characterized in human Anelloviruses (Qiu et al., 2005) and studies on Anelloviruses infecting other species such as seals, cats and gorillas (Hrazdilovi et al., 2016) (Fahsbender et al. 2017; Zhang et al. 2016; Hrazdilova et al. 2016) have shown evidence for ORF3. Parsing the 471 ORF3 sequences (median length: 68aa, minimum length: 50aa, maximum length: 159aa) through MEME (Bailey et al. 1994) revealed the presence of two previously unknown and highly conserved motifs located near the 3′ end of ORF3. Motif 1 (26 aa) was observed in 467 out of the 471 sequences (99%) while Motif 2 (5 aa) was observed in 463 out of the 471 sequences (98%) (FIG. 42B). ORF sequences identified as ORF1, ORF2 or ORF3 frequently contained peptides upstream of the canonical start codon as per the functionality of orfm. These sequences were trimmed to the proper start and stop codons via an in-house written python script that searched for the first methionine located from the 5′ end and in the cases of ORF1 the start codon was predicted by first locating the arginine-rich region and locating the first methionine upstream. In some cases, a non-canonical start codon was predicted as the ORF1 start codon by searching for the amino acids threonine-proline-tryptophan or threonine-alanine-tryptophan just upstream of the arginine-rich region.

Estimates of the proportion of individual Anellovirus lineages in each sample/longitudinal timepoint for donor-recipient datasets were estimated by identifying the unique set of lineages present across each donor sample by clustering ORF1 sequences at 97.5% similarity using the usearch software (Edgar, 2010) and the cluster_fast algorithm. These unique donor-derived Anellovirus lineages were then searched for in recipient longitudinal samples by mapping the derived short read sequencing data against them using the Novoalign software (Novocraft, n.d.) with the following parameters: -H 15, -l 30, -t 500, -r Random, -g 50, -x 6, -F STDFQ.

The resulting BAM mapping files were used to calculate relative Anellovirus proportion estimates for each donor lineage by custom script using the formula below:

$$Anellovirus Lineage Relative Proportion = \frac{Mapped Anello Reads}{Total Mapped Anello Reads} \times 100$$

The relative proportion of all donor lineages in each donor-recipient dataset were collated together into one tab-delimited file for further downstream analysis.

Steam graph figures depicting Anellovirus proportion shifts over time in subjects were generated with R (R Core Team, 2013) using the ggplot2 (Wickham, 2016, p. 2) and ggTimeSeries package.

High resolution curve analysis was performed in a QuantStudio 3.0 thermal cycler (Applied Biosystems, Thermo) using the MeltDoctor HRM mastermix (Applied Biosystems, Thermo) in a 10 μl reaction volume. All specimens were tested in triplicate and their melting profiles were analyzed using High Resolution melt v3.1 software and the HRM algorithm provided according to the manufacturer. ORF1 regions of strains in recipients and donors (>95% pairwise identity) were cloned and Sanger sequenced prior to performing the high resolution melt. Characterization of different alleles in the samples was determined based on their melt curves.

To validate the new Anellovirus sequence discovery method, the difference in Anellovirus genomic sequence yield between a standard rolling-circle amplification (RCA) method (Niel et al., 2005) and the new discovery method described herein was measured. Compared to standard RCA, the new discovery method led to a 1,046 to 52,812-fold increase in Anellovirus coverage measured from serum samples from our TTVS cohort (Table 2).

TABLE 2
Benchmarking of the novel discovery method

| | | Total | TTV | Percent | |
|---|---|---|---|---|---|
| Sample | RCA Type | Reads | Reads | TTV | Fold Change |
| R04D01 | Random | 722,717 | 0 | 0.000% | ∞ |
| | Hexamer | | | | |
| | TTV-RCA | 642,901 | 85,530 | 13.46% | |
| R04D02 | Random | 823,172 | 19 | 0.002% | 1,046 |
| | Hexamer | | | | |
| | TTV-RCA | 622,596 | 15,026 | 2.41% | |
| R04T00 | Random | 798,299 | 1 | 0.000% | 41,894 |
| | Hexamer | | | | |
| | TTV-RCA | 304,654 | 15,988 | 5.25% | |
| R04T01 | Random | 759,930 | 168 | 0.022% | 3,083 |
| | Hexamer | | | | |
| | TTV-RCA | 785,323 | 535,237 | 68.16% | |
| R04T02 | Random | 649,627 | 5 | 0.001% | 21,073 |
| | Hexamer | | | | |
| | TTV-RCA | 595,526 | 96,592 | 16.22% | |
| R04T03 | Random | 732,105 | 6 | 0.001% | 52,812 |
| | Hexamer | | | | |
| | TTV-RCA | 528,421 | 228,715 | 43.28% | |
| R04T04 | Random | 434,485 | 11 | 0.003% | 10,060 |
| | Hexamer | | | | |
| | TTV-RCA | 409,314 | 104,244 | 25.47% | |

Anellovirus presence in longitudinal samples was determined in order to quantify the number of Anellovirus lineages at each timepoint and to measure the diversity found in each subject. The new discovery method was applied to 128 samples from 67 individuals. There were a total of 53 healthy volunteer donors (21 females, 32 males) ranging in age from 17 and 62 (median age: 34) and 1S recipients, whose details are provided in Table 3. 75 longitudinal recipient samples were examined as well. Samples ranged across five time points (one pre-transfusion, four post-transfusion). Sequence reads from both donors and recipients are plotted in FIG. 40, which shows total reads from donor and recipient samples as well as Anellovirus reads. In total, 300.1 Gbp of sequence data was recovered, of which 159.6 Gbp were derived from Anelloviruses. From the sequence data, 1,656 high-quality Anellovirus contigs (median length=2,916 bp mi length=2,190 bp, max length=4,917 bp) were identified. Previously identified Anellovirus genomes were taken from the NCBI GenBank repository (Benson et al., 2012; incorporated herein by reference in its entirety) to create a baseline of known sequences for comparison. Sequences from the repository were filtered by size, and non-human and published Anellovirus sequences were removed to produce a set of 445 curated sequences. A combined dataset containing 2,101 Anellovirus sequences was established for further downstream analysis.

TABLE 3
Recipient Demographics

| Recipient | Gender | Age | Surgical Procedure |
|---|---|---|---|
| R01 | Female | 62 | Knee replacement |
| R02 | Male | 27 | Carotid endarterctomy |
| R03 | Male | 57 | CABG |
| R04 | Male | 54 | Knee replacement |
| R05 | Female | 64 | Aortic valve replacement |
| R06 | Male | 50 | CABG |
| R07 | Female | 59 | Mitral Valve replacement |
| R08 | Male | 46 | CABG |
| R09 | Female | 38 | Cervical Surgery |
| R10 | Male | 20 | Aortic valve Replacement |
| R11 | Male | 61 | CABG |
| R12 | Female | 20 | Caesarean-section |
| R13 | Male | 57 | Aortic Aneurysm Repair |
| R14 | Female | 65 | Resection of Recto-sigmoid |
| R15 | Female | 21 | CABG |

### Example 37: Phylogenetic Analysis of Anellovirus Genomes

In this study, diversity of human Anelloviruses was evaluated through homology and phylogenetic analyses on the ORF1 sequences from the Anellovirus sequence dataset described in Example 36. From the set of 1,177 novel Anellovirus sequences, 1,177 ORF1 sequences were isolated via identification of a novel amino acid motif YNPX²DXGX²N found in the N22 region. The 35% sequence similarity cut-off suggested by the International Committee on Taxonomy of Viruses (ICTV) (Adams et al., 2016) was too restrictive to fully characterize the subspecies of the dataset, so the 1,177 ORF1 sequences were defined as being distinct Anellovirus lineages where there are at least 97.5% similarity. 813 unique ORF1 sequences were accordingly classified as belonging to distinct Anellovirus lineages. Of these 813 ORF1 sequences, 767 (94%) were classified as unique based on sequence dissimilarity greater than or equal to 25% of all Anellovirus sequences found in the NCBI RefSeq non-redundant proteins (nr) database (O'Leary et al., 2016).

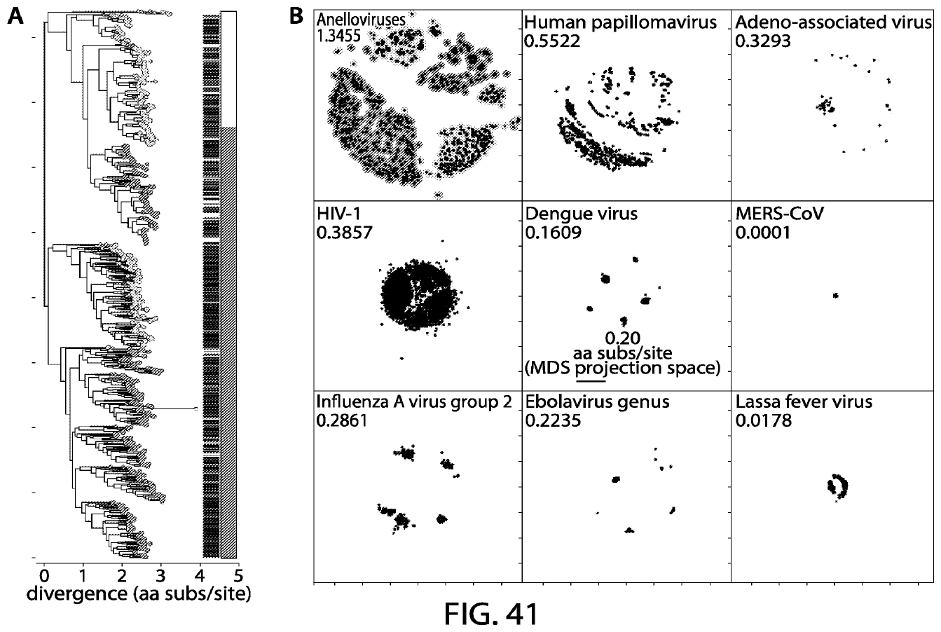

Human Anelloviruses have been taxonomically classified into three broad genera, Alphatorqueviruses, Betatorqueviruses, and Gammatorqueviruses. Publicly available and newly described Anellovirus sequences were split into the three genera, with 689 Alphatorquevirus sequences, 619 Betatorquevirus sequences, and 271 Gammatorquevirus sequences, and trimmed to the ORF1 region. ORF1 sequences were translated and aligned using MAFFT (FFT-NS-i×1000 setting), and pairwise distances between amino acid sequences computed. All three alignments (alpha, beta and gamma) were then consensus-aligned using MAFFT (G-INS-i setting). A maximum-likelihood phylogeny of all 2,101 Anellovirus capsid proteins (1,177 from TTVS cohort and 449 downloaded from NCBI GenBank) was constructed using RAxML (CAT sequence evolution model, BLOSSUM62 substitution matrix), and revealed that the sequences from the TTVS cohort fell into the three genera, providing increases of 28%, 27% and 15% in the number of sequences in the Alphatorquevirus, Betatorquevirus, and Gammatorquevirus genera, respectively (FIG. 41, Panel A).

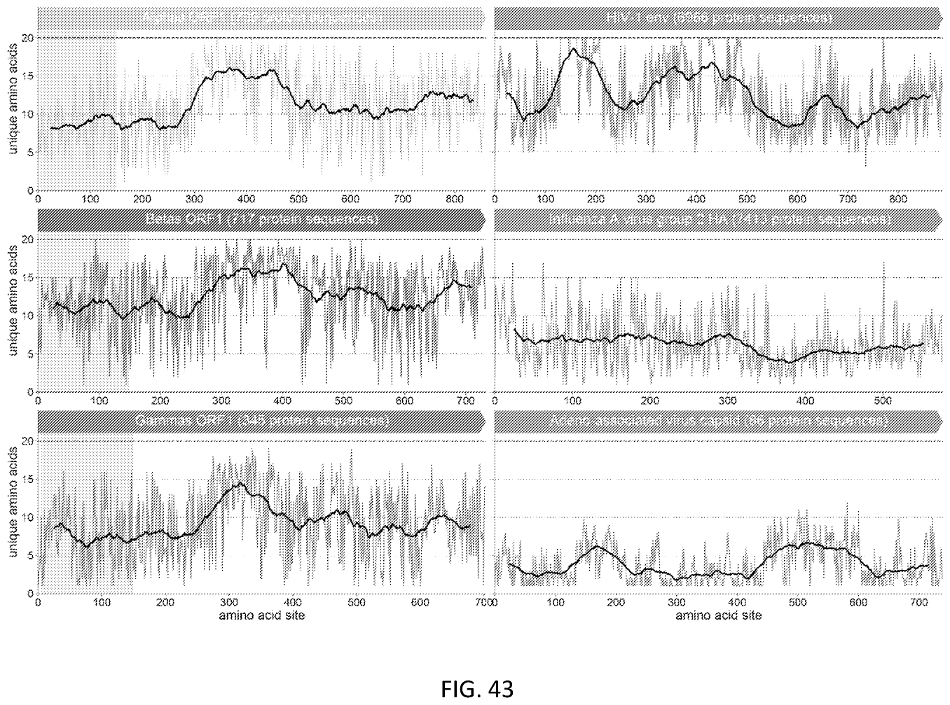

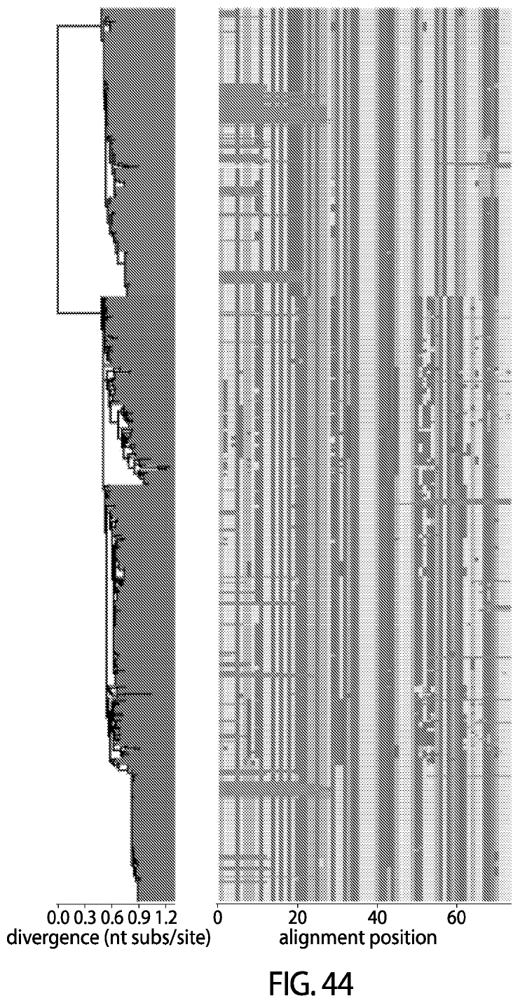

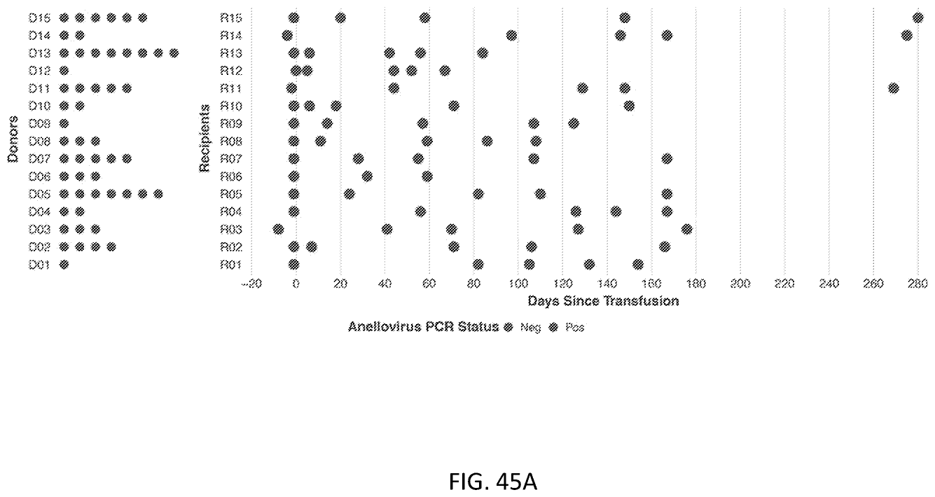

Phylogenetic analysis operates under the assumption that the organisms being analyzed follow clonal evolution models. However, as recombination may play a significant role in genetic variation that has been observed, a clonal model may not be sufficient to analyze the sequence dataset established in these studies. To further characterize the extent of Anellovirus diversity, the Anellovirus ORF1 sequences were analyzed using multidimensional scaling (MDS). Further, diversity of Anellovirus ORF1 sequences was compared to diversity found in eight other suitable candidate surface proteins: DNA viruses (Anellovirus, human papillomavirus (HPV), adeno-associated virus (AAV)); negative sense single-stranded RNA viruses not known to recombine (Influenza A virus group 2, Ebolavirus, Lassa virus); and positive sense single-stranded RNA viruses known to recombine (HIV1, Dengue virus, MERS coronavirus). Viruses were selected across three different groups each exhibiting slow or fast rates of evolution, single or double stranded molecules and known or not known to recombine to provide comparisons against viruses with a wide level of diversity. Genbank datasets for human papillomavirus (HPV, diversity encompasses HPV type 41) late protein (L1), adeno-associated virus (AAV, all diversity found in humans) capsid protein, Dengue virus (all known serotypes) envelope protein, Middle East respiratory syndrome-associated coronavirus (MERS-CoV, all known diversity) spike protein (S), Ebolavirus (genus-level) glycoprotein (GP) protein, and Lassa fever (all known diversity) virus glycoprotein complex (GPC) protein were downloaded from GenBank. Additional datasets for influenza A virus group 2 haemagluttinin (HA) sequences were downloaded from the Influenza Research Database and human immunodeficiency virus-1 (HIV-1) env sequences were obtained from the Los Alamos National Laboratory pre-made alignment sequence database. Sequences were translated and aligned using MAFFT (auto setting) and down-sampled to 3000 sequences. Sequences were binned into four groups (full contigs, ORF1 capsid, ORF2, and 5′ UTR) and analyzed for pairwise genetic distances across lineages. The distribution of the percentage of pairwise-identities across all sequences is depicted by group in FIG. 42. Site diversity of the viral protein sequences was probed via analysis of the number of unique amino acids at each position. This analysis of amino acid diversity is illustrated in the plots of FIG. 43. As an illustrative example, phylogeny of the 5′ UTR region for each category of Anellovirus is depicted in FIG. 44, highlighting the nucleotide alignment across sequences.

To account for potential non-clonal evolution in Anelloviruses, Anellovirus diversity was examined using, for example, multidimensional scaling (MDS). MDS was applied to all viral protein sequences to project them into two dimensions using Scikit-learn. Agglomerative clustering was additionally applied to pairwise amino acid distances of Anelloviruses using Scikit-learn to identify 10 (arbitrarily chosen for ease of comparison) clusters. MDS-projected sequences were visualized using matplotlib and colored by assigned cluster in the case of Anelloviruses.

The results of the MDS analysis, plotted in Panel B of FIG. 41, indicate that Anelloviruses occupy a large amount of projected space. Projecting the MDS results for the eight comparator viruses on the same scale as the Anellovirus analysis indicated that Anellovirus diversity was significantly higher (3 to 4 times larger) than the viruses selected for comparison. Even compared to viruses that are known to accrue mutations rapidly, such as influenza and HIV, and viruses that are known to recombine, such as MERS-CoV, Anelloviruses occupied more of the MDS' 2D projected space. Measuring the area of the convex hull that encompasses all MDS coordinates (sequences) of all surface proteins provided a single measure of viral diversity from each virus. This measurement for the Anelloviruses of the present study was more than double the virus with the closest value; in comparison to viruses that are known to have high levels of diversity, Anelloviruses exhibited a measurement three to four times larger. The observed growth in sequences in each of these genera was between 15% to 28%. Also determined were the phylogenetic branch length contribution per new sequence in the Betatorque—(0.114 substitutions per amino acid site per sequence) and Gammatorque—(0.148) genera compared to Alphatorqueviruses (0.039).

### Example 38. Anellovirus Co-Infection in Blood Transfusion Donors and Recipients

In this study, the samples described in Example 36 were surveyed to measure the amount of Anellovirus present at various longitudinal timepoints after infusion. PCR and sequencing were used to survey the Anellovirus lineages present at each timepoint and to characterize the anellome in each subject. All fifteen blood transfusion recipients in the cohort were found to contain co-infections with numerous Anellovirus lineages.

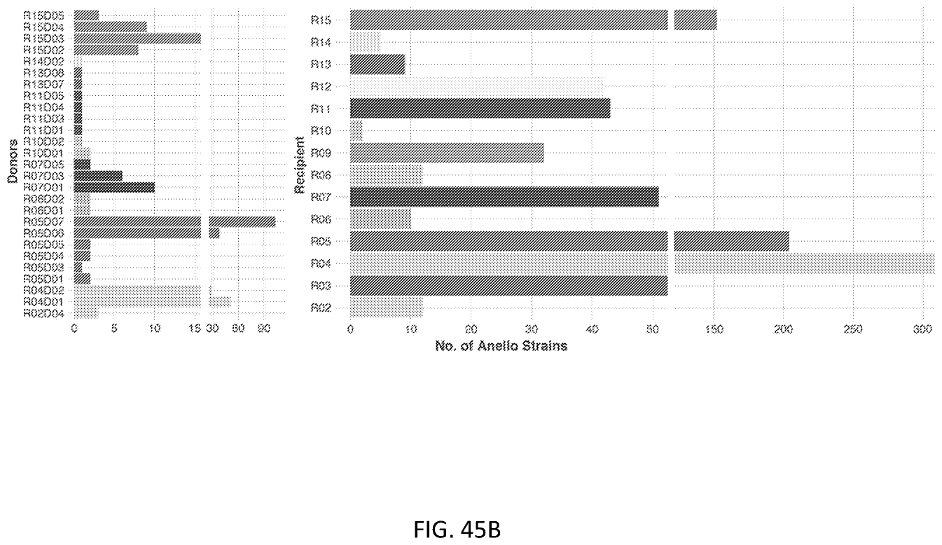

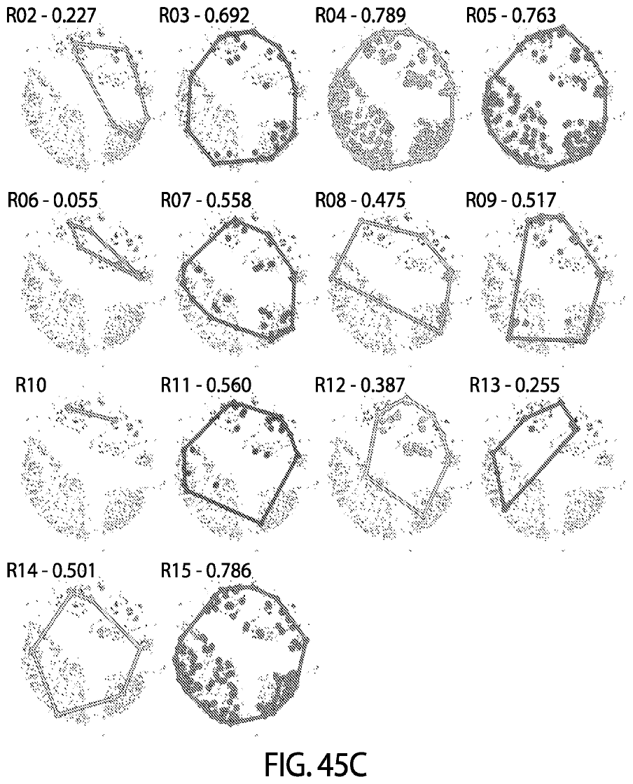

Pan-Anellovirus PCR assays were used to quickly assess the presence or absence of Anellovirus DNA in all donor and recipient samples. The presence of Anelloviridae in serum samples was tested by PCR using pan-Anellovirus primers developed by Ninomiya 2008 (incorporated herein by reference in its entirety). Briefly, 10 μl of sample was added to 1×PCR Master Mix (Sigma Aldrich PCR Master kit #11636103001) and the 4 degenerate primers at a final concentration of 1 μM each in a final volume of 25 ul. Positive samples were identified by the presence of a 128 bp band in a 2% Agarose gel. FIG. 45A shows the results of the PCR analysis; the absence or presence Anelloviruses in the donor samples used for each recipient are denoted, as well as on which days Anelloviruses were detected in each recipient.

Anelloviruses were detected in 53% of donor samples (33/53) and 86% (65/75) of recipient samples. At least one positive sample was detected in each donor-recipient transfusion set; while Anelloviruses were not detected in four of the donor sets (the blood donors for recipients 1, 8, 9, and 14), there was subsequent detection of Anellovirus in at least one sample from each of these recipients. In total, Anelloviruses were found in 76% (98/128) of our samples by PCR with detection rates supporting those previously observed in whole blood or plasma samples.

Targeted deep sequencing in conjunction with the new amplification method described above was used to measure the number of Anelloviruses in each subject and at each time-point in this study. The unique number of Anellovirus capsid protein sequences was analyzed, and each was isolated as a unique marker gene identifiable using the YNPX²DXGX²N amino acid motif described herein. FIG. 45B depicts a plot indicating the number of Anellovirus strains in a subset of the donors and recipients. The majority of subjects in the cohort contained a median of 6 distinct Anellovirus lineages per subject, with individual blood transfusion recipients containing a median of 27 lineages and three subjects containing over 100 unique lineages across all five time-points recorded. A plurality of subjects were identified as containing over 20 unique lineages each. The median number of lineages increased over four-fold when examining just transfusion recipients. These findings indicate that the increase in abundance of anellovirus lineages in transfusion recipients was elevated by transmission of lineages from blood donors.

Having identified substantial Anellovirus diversity, we next asked whether or not the diversity found in ORF1 sequences were limited to specific regions or widespread across the entire gene. We plotted the number of unique amino acids seen across the entire ORF1 sequence delineated into the three genera for the sequences isolated from the transfusion cohort (an alignment of 1,861 sequences). In addition, these findings were compared against those found by examining collections of HIV-1 env, Influenza A virus group 2 HA and the AAV capsid proteins (FIG. 43). It was found that on average the number of unique amino acids seen across the ORF1 sequence varied widely but, in many cases, all 26 amino acids were present at multiple sites. Out of the three Anellovirus genera compared we noted that amino acid diversity per site was greater in Alphatorqueviruses and Betatorqueviruses and that the number of unique amino acids was lower at the 5′ end of the gene potentially near the arginine-rich region and jelly-roll domain. The maximal amount of amino acid diversity followed these two features in the presumed hypervariable regions. We also observed that the amino acid diversity in Anelloviruses were higher than or equal to those found in the HIV, Influenza and AAV viruses used as a comparison point. Overall, it was found that the average unique amino acids per site was elevated across the entire ORF1 sequence and greater than those found in the two of the three viruses (AAV and influenza A virus) selected for comparison (FIG. 43). It was observed that the ORF1 amino acid diversity of the three anellovirus genera are each greater than all the currently described surface proteins in two out of the three viruses compared against, with only HIV-1 env exhibiting equal or greater diversity primarily driven by its hypervariable loops.

### Example 39. Analysis of Diversity of a Personal Anellome

In this study, the diversity of Anelloviruses that comprise the anellome in individuals described in Example 36 was evaluated. This analysis probed the extent to which diversity is (or is not) restricted in evolutionary space within individuals and the extent to which each personal anellome is unique. Each subject was found to have a distinct anellome, with few lineages shared across subjects. The breadth of total diversity within each individual spanned the breadth seen within the full dataset. The results indicate a higher prevalence than previously reported by lower sensitivity studies and indicate that Anelloviruses may inhabit nearly all healthy individuals.

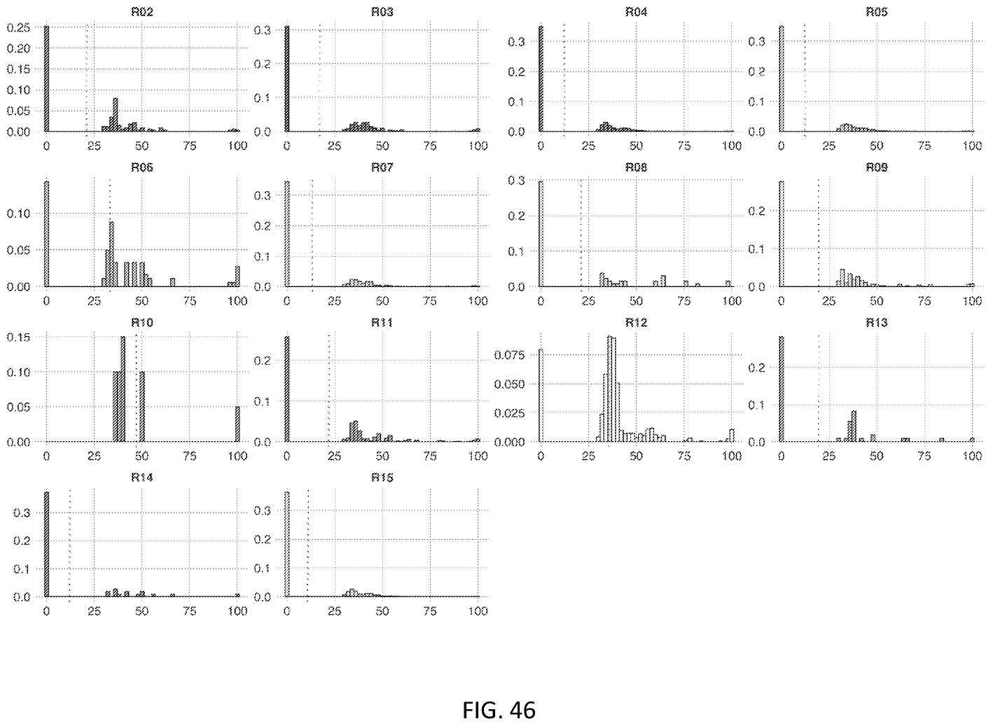

Diversity within each donor/recipient subject set was examined by analyzing the similarity of Anellovirus lineages via pairwise comparisons using the average amino acid identity (AAI) of each ORF1 sequence (FIG. 46). Relatively little similarity was observed between Anellovirus lineages isolated from each donor-recipient subject, with a mean pairwise AAI across all donor-recipient sets at 12.1%. In subjects with a higher number of Anellovirus lineages (e.g. subjects 4, 5, and 15), lower mean pairwise AAI values were observed relative to subject sets with fewer Anellovirus lineages. Lower mean pairwise AAI values were observed than those in subject sets with fewer Anellovirus lineages. We also observed that in individuals with fewer lineages the breadth of diversity was just as wide (recipients 3, 7, 11, 14), suggesting that even in subjects with smaller anellomes the diversity of lineages found can still be quite high. To support these observations, we summarized the total occupied area of the MDS' project 2D space, using the same single summary statistic computed in our global analysis and found that these values supported our observations. We observed the highest values in transfusion sets 4, 5 and 15, agreeing with our assessment that these sets contained the most diversity in the TTVS cohort. These results points to a rich diversity of Anellovirus lineages within a subject, as measured by ORF1 sequence similarity.

We next searched through the full assembled contigs, ORF2 sequences and 5′UTR sequences, conducting the same pairwise sequence similarity comparisons across the full dataset to analyze whether the diversity extended beyond what we observed in the ORF1 protein (FIG. 42C). The 5′UTR region of the Anellovirus sequence demonstrated high conservation and had similarity levels higher than those found in other Anellovirus features. These findings indicate that the 5′UTR could act as a suitable discriminator in classifying Anellovirus lineages at a higher specificity than the currently endorsed ORF1 model.

MDS analysis, following the methods described in Example 37, was used to measure, visualize and compare diversity of individuals in recipient subject set (FIG. 45C). The per-subject analyses were projected over the same 2D space used for the data set of the total anellome, depicted in Panel B of FIG. 41. The results of this study indicate that in the cases where a large enough sample size of Anellovirus lineages (i.e., 40 or more lineages) were present, the diversity encompassed the majority of the projected MDS space and mirrored the same organized found when examining the full dataset. Similarly, in those subjects that contained a smaller proportion of lineages, a spread across the projected diversity space was still observed, with lineages covering multiple genera in the majority of cases. The same diversity statistic was computed for the full dataset to represent the amount of the projected space occupied by Anellovirus lineages found in each subject set. Again, the subjects with a higher number of Anellovirus lineages were highest in this statistic. The subject sets with the largest number of Anellovirus lineages (4, 5, 15) all exhibited the greatest amount of diversity, and the largest diversity statistic was found in the set with the highest number of lineages. The difference in the statistic between the three subject sets with the highest number of lineages was 4.

### Example 40: Persistence of Anelloviruses Transmitted Via Blood Transfusion

Anelloviruses can be detected in multiple biological sample types, and transmission may occur through routes such as saliva, breast milk, semen, oral-fecal, mucous members, skin, or blood. In this study, the transmission dynamics of Anelloviruses in a blood transfusion cohort was probed by tracking the transmission of strains between donors and recipients over time by sequence similarity and by proportion of reads that mapped to donor strains.

The results indicated that Anellovirus transmission occurred consistently in multiple subjects. The majority of blood transfusion recipients had several lineages that were transmitted from one or more donors. Fully assembled putative Anellovirus genomes of donors and recipients were characterized, and the proportions of transmitted lineages present in recipients was measured by both comparing the presence of fully assembled putative anellovirus genomes and measuring the proportions of transmitted lineages present via mapping of Anellovirus sequencing reads. The data evidenced that transmitted lineages were present up to at least 270 days after the transfusion event.

Unique Anellovirus lineages in the blood transfusion recipients were categorized as resident to that particular recipient or transmitted from the donor. To produce the set of Anellovirus lineages unique to each donor, pairwise comparisons between ORF1 sequences of lineages isolated from donors and lineages isolated from recipients pre-transfusion at a 95% similarity cut-off was used. Donor recipients were searched for traces of these unique sets of donor Anellovirus lineages at several time points post-transfusion and extracted in cases where significant sequence homology in the ORF1 sequence was indicative of a transmitted lineage. Transmission of at least one Anellovirus lineage was observed in 8/15 recipient subjects (median=5, min=0, max=53), and identified a total of 133 transmitted lineages across all donor/recipient subject sets. In addition, 6 lineages were identified as present in recipient samples pre-transfusion, but also increased via transmission from donors, consistent with the possibility of re-dosing of anellovirus lineages. FIG. 41A depicts a plot of the abundance of Anelloviruses in transfusion recipients over time; these plots follow the change in both transmitted lineages and resident lineages over time.

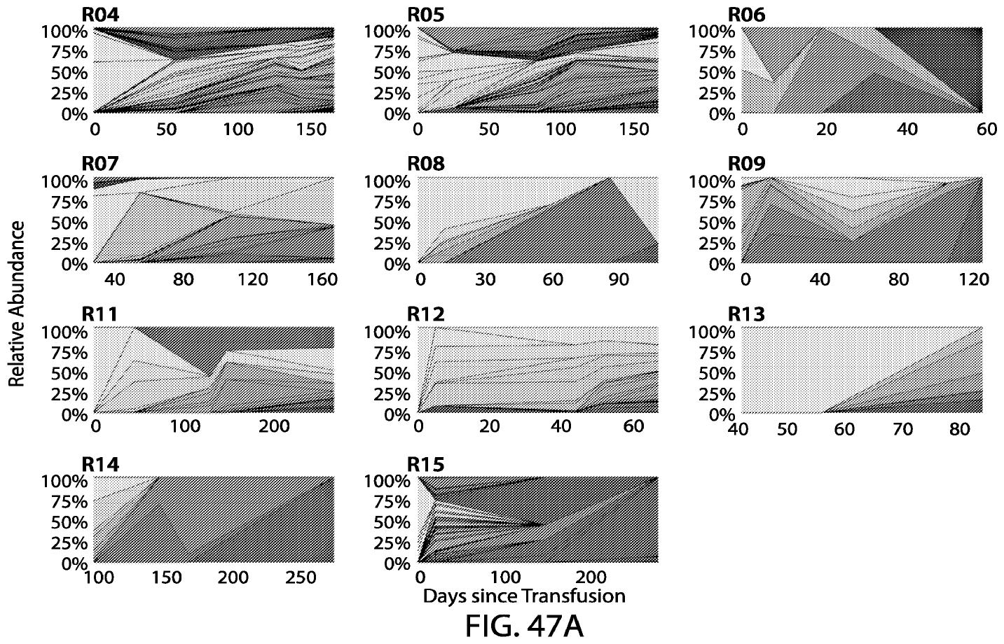

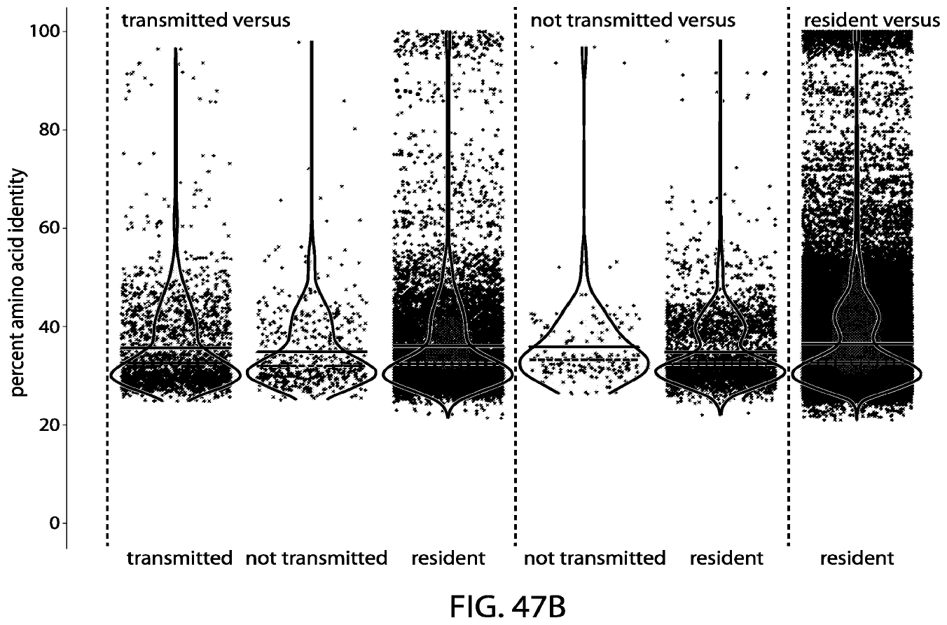

The proportion of Anellovirus sequencing reads per sample was measured and attributed to each Anellovirus lineage in order to approximate shifts in the anellome longitudinally. This proportion was calculated by mapping decontaminated and QC-filtered metagenomic reads to the coding sequences of each ORF1 protein in each sample. Recipient time point samples were queried post-transfusion for the presence of transmitted lineages from paired donors primarily by sequence similarity homology. Utilizing the unique set of donor lineages for each donor-recipient set, the four post-transfusion time points were searched for Anellovirus lineages with a sequence similarity of 95% or greater over 90% of a donor lineage. In recipients where at least one anellovirus lineage was transmitted, marked shifts in the proportion of resident Anelloviruses were observed (FIG. 47A). Recipients 4, 5, 6, 11 and 15 all exhibited a steady increase in the proportion of transmitted lineages over the 50 days following the initial date of transfusion with some donor lineages present at the latest time point sampled in all three subjects. In the majority of recipients, transmitted Anellovirus lineages persisted over the course of the longitudinal study with 29 lineages detected greater than 100 days from the date of transfusion (median=88 days).

### Example 41: Independent Transmission of Anelloviruses

In this study, the ability of Anelloviruses to transmit independently of sequence determinants, such as sequence similarity over small or large tracts or conserved motifs shared across lineages, was probed. For example, this study assessed whether Anellovirus lineages are more likely to transmit if they are highly similar (or dissimilar) to lineages in a recipient's anellome. The sequences from the sets of Anellovirus lineages that transmitted, did not transmit, and were resident to recipients were compared, and sequence similarity to resident lineages recipient anellomes in the majority of cases had no impact on whether or not a lineage is more transmissible.

Anellovirus lineages were divided into the three previously defined categories (i.e., transmitted, not transmitted, and resident) using ORF1 sequences and their similarity/dissimilarity to lineages identified in donor and recipient samples. In addition to the 123 Anellovirus lineages that transmitted between donor and recipient samples (see Example 40), 43 Anellovirus lineages were identified that were unique to donors but were not transmitted to their respective recipient samples.

The two sets of donor-derived Anellovirus lineages were compared to a number of recipient resident lineages (in the range of tens to hundreds of lineages), in a pairwise fashion in every permutation, to measure whether or not sequence similarity played a role in transmissibility of lineages (FIG. 47B). The percent amino acid similarity between the comparisons of these three categories of Anelloviruses differed relatively little. In all six of the comparisons, the median percentage amino acid identity was 32.44%. A small set of Anellovirus lineages shared high sequence similarity with resident lineages and were transmitted to recipients, indicating that while Anelloviruses may not generally require similarity to a recipient's anellome, it may aid in transmission in some cases. For example, in the comparison of transmitted and resident lineages, 79 of the transmitted lineages were >80% similar to a resident lineage in the corresponding recipient.

### Example 42: Recombination in Anelloviruses as a Mechanism to Increase Diversity

In this study, the mechanism for generating diversity of ORF1 sequences was probed by searching for and evaluating recombination in ORF1 genes. By unlinking loci on the same genome, recombination leaves numerous signals in sequence data—for example, excessive repeat mutations (homoplasies) caused by phylogenetic methods assuming strictly clonal evolution, inconsistent phylogenetic tree topologies between different parts of the genome, and decreasing statistical association between loci with increasing distance between them. Due to the difficulty of aligning Anellovirus sequences, recombination inference of this study was limited to the best possible alignments that could be identified. Translationally aligned sequences of the three Anellovirus genera were grouped into clusters where all members were at least 80% identical to another member at nucleotide level, resulting in 28 clusters with more than 10 members (23 clusters of Alphatorquevirus, four of Betatorquevirus and one of Gammatorquevirus). A single representative of each genus was chosen for a closer analysis, giving clusters with 23 Alphatorquevirus, 11 Betatorquevirus, and 10 Gammatorquevirus sequences. Sequences within each cluster were realigned using MAFFT (a more accurate E-INS-i setting) to improve the alignments. Then each alignment was split into 500 nucleotide fragments and phylogenies inferred from each fragment using PhyML (HKY+Γ₄ substitution model) and midpoint-rooted. Phylogenies derived from neighboring fragments were displayed in a tangled chain where each taxon is tracked through successive trees. Inconsistency of the topologies of 500 nucleotide fragment trees along the ORF1 sequence are depicted in Panel A of FIG. 48.

The same cluster alignments, undivided, were used to infer single trees using PhyML (HKY+Γ₄ substitution model). Each tree and alignment were then used to reconstruct the mutations that occurred across the tree using ClonalFrameML with kappa set to 2.0. For every mutation that was reconstructed to only occur once in the tree, the branch where the mutation occurred was marked with ticks, and every mutation that was inferred to occur more than once in the tree was indicated by a line connecting the mutation to its identical counterparts (i.e., reversions are considered separately) elsewhere in the tree. Reconstructing mutations on a single tree and highlighting those that occur only once in the tree (i.e. synapomorphies, marked with ticks on branches) versus those that occur multiple times (i.e., homoplasies, indicated with lines connecting branches where the same mutation happened) revealed an abundance of repeat mutations, even at relatively low levels of divergence, indicative of recombination (FIG. 48, Panel B).

Next, the relationship between the physical distance of polymorphic nucleotide sites and linkage disequilibrium between them was assessed in translation-aligned sequences within each genus. Decay of linkage disequilibrium (LD) was evaluated using the χ²_{df} statistic, which behaves identically to the more common r² statistic for biallelic loci. To this end, the genus-wide alignments with 689 Alphatorquevirus, 619 Betatorquevirus, and 271 Gammatorquevirus sequences were used. Alignment columns with fewer than 10% valid sites (A, C, T or G) were ignored, as were sites where the minority variant was lower than 5% frequency. LD measured between pairs of variable sites was plotted against the distance between sites with mean LD calculated in windows 100 nucleotides long (FIG. 48, Panel C). Panel C of FIG. 48 shows the relationship between physical distance of polymorphic nucleotide sites and a measure of linkage disequilibrium between sites in translation-aligned sequences within each genus. With recombination, the probability of recombination occurring increases with increasing distance between sites with highest linkage disequilibrium observed between neighboring sites and increasing with physical distance. There are two extremes where such a relationship is not expected—no recombination at all and free recombination. For non-recombining genomes, only repeat mutations can reduce linkage disequilibrium from the baseline of 1.0, and for free recombination, linkage disequilibrium is 0.0 between adjacent loci. The plots of linkage disequilibrium of the three genera showed that each genus exhibited a linkage disequilibrium close to zero, which indicates that, on a large scale, Anellovirus loci effectively evolved independently.

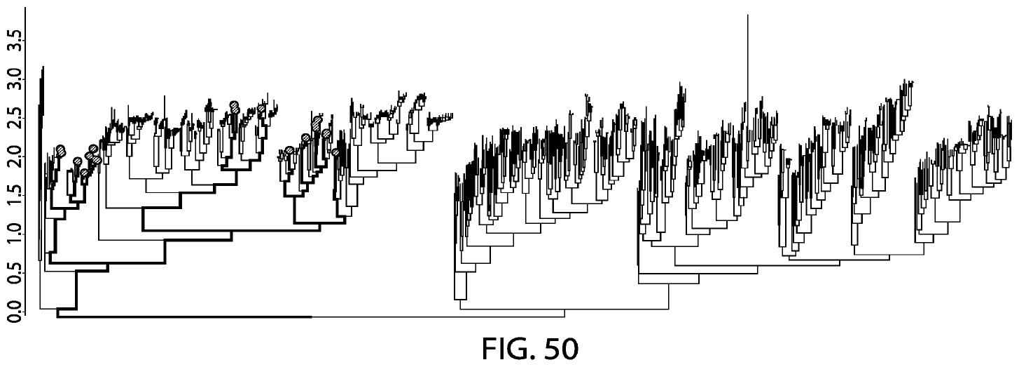

As complete circularized genomes were available for a number of Anelloviruses, the degree of reticulate evolution in non-coding parts of the genome was probed. To this end, complete genomes of each genus (22 Alphatorqueviruses, 467 Betatorqueviruses, and 23 Gammatorqueviruses) were aligned and non-coding regions were extracted, followed by ancestral state reconstruction using ClonalFrameML. To identify putative recombination tracts, sequences were analyzed for repeat mutations (homoplasies) occurring in clusters of at least three mutations within ten nucleotides of each other. Such clusters of mutations identified within non-coding genomic regions of Alphatorqueviruses are depicted in FIG. 49. FIG. 50 highlights the phylogenic positions these recombination tracts, indicating that the mutations span the entirety of Alphatorquevirus diversity. These results demonstrate frequent recombination in the study cohort as well as in public data. The Examples provided herein showed frequent coinfections with multiple distinct lineages of Anelloviruses that would provide opportunity for recombination to occur within individuals. Evidence for recombination was clearest at low levels of divergence, either between closely related ORF1 sequences from donor-recipient pairs, within-patient sequence clusters, or more conserved regions of the Anellovirus genome. These data suggest that strictly clonal models of evolution (e.g., a phylogenetic tree) may not be able to adequately infer the relationships or distances between Anellovirus sequences, and that few or no regions of the Anellovirus genomes may be entirely free of recombination.

### CONCLUSION

In summary, the present study explored the anellomes of 15 blood transfusion recipients and their matching donors and identified a dynamic landscape that suggests that each individual harbored a distinctive set of Anelloviruses. This was done by utilizing an Anellovirus-targeted amplification method coupled with deep sequencing to identify unique anellovirus lineages across 112 samples. By using the Anellovirus ORF1 sequence as a backbone of exploration, as well as a unique marker feature, the diversity in each subject was assessed at a substantially deeper level than would have been possible by analyzing complete Anellovirus genomes. Recovering complete Anellovirus genomes is hindered by the high GC content in the non-coding regions. Classifying anelloviruses using the current ICTV cut-off values collapses down the majority of diversity found within samples and therefore may benefit from subspecies/lineage definitions complemented by experimental evidence to demarcate species boundaries.

Over 200 transmitted Anellovirus lineages were identified in blood transfusion recipients and Anellovirus transmission was observed in 6/15 (40%) of recipients (FIG. 47A). The similarity of donor lineages to the host anellome seemed to have little effect on transmission success (FIG. 47B). In fact, instances of donor lineages that successfully transmitted despite high sequence similarity (>90%) to resident lineages were observed, indicative of re-infection and that therapeutic anellovectors (e.g., as described herein) can be effectively re-dosed. Anellovirus transmission via other non-iatrogenic routes, such as respiratory and fecal-oral, is also contemplated here, based on the ubiquitous acquisition of anelloviruses in the first year of life (REF).

Targeted anellovirus sequencing enabled the differentiation and tracking of hundreds of unique Anellovirus lineages over time. A high prevalence of co-infections was found, with multiple anellovirus lineages (16/16 recipients). Both resident and transmitted Anellovirus lineages were observed that persisted for the duration of this study (up to 270 days post-transfusion). Without wishing to be bound by theory, the persistence of newly transmitted lineages via blood transfusion further indicates that an intravenously delivered therapeutic could be a vehicle for delivery.

The characteristics of Anelloviruses and their key features described here suggest a model of the anellome wherein new lineages cycle in and out of the space cohabited by a diverse milieu of resident lineages. Their ability to infect independent of sequence similarity and absent disease associations suggest low immunogenicity and confers long-lasting infections, which thus permit co-infections with multiple strains and facilitate frequent recombination. Characteristics of Anelloviruses such as their ubiquity and persistence in humans, and low immunogenicity and pathogenicity, are consistent with the observation that recombination facilitates Anellovirus diversification.

Without wishing to be bound by theory, the diversity observed in Anelloviruses from the blood of the subjects in the transfusion cohort provide viral templates (e.g., as described herein) that can be utilized to deliver therapeutic payloads. Anelloviruses reconfigured to carry therapeutic payloads (e.g., anellovectors as described herein) may have the advantages of being resistant to antecedent antibodies and of having tissue tropism. This may allow redosing of replication-deficient Anelloviruses and reduction of the high doses necessary in current delivery formats that can result in toxicity.

What is claimed is:

1. A method of delivering an exogenous effector to a human subject who has previously been administered a first plurality of anellovectors, said method comprising:
administering to the subject a second plurality of anellovectors, wherein:
(i) the first plurality of anellovectors comprises:
(a) a proteinaceous exterior that comprises an ORF1 molecule;
(b) a genetic element comprising a promoter element and a nucleic acid sequence (e.g., a DNA sequence) encoding an exogenous effector, and
(ii) the second plurality of anellovectors comprises:
(a) a proteinaceous exterior comprising an ORF1 molecule having at least 90% amino acid sequence identity to the ORF1 molecule in the proteinaceous exterior of the first plurality; and
(b) a genetic element comprising a promoter element and a nucleic acid sequence (e.g., a DNA sequence) encoding the exogenous effector;
thereby delivering the effector to the subject.

2. The method of claim 1, which comprises administering to the subject the first plurality of anellovectors.

3. The method of claim 1 or 2, wherein the ORF1 molecule of the second plurality of anellovectors has at least 95%, 96%, 97%, 98%, or 99% amino acid sequence identity to the ORF1 molecule in the proteinaceous exterior of the first plurality of anellovectors.

4. The method of any of the preceding claims, wherein the second plurality of anellovectors is administered to the subject at least 1, 2, 3, or 4 weeks, or 1, 2, 3, 4, 5, 6, 7, 8, 9, 10, 11, or 12 months after the administration of the first plurality of anellovectors to the subject.

5. The method of any of the preceding claims, which further comprises administering to the subject a third, fourth, fifth, and/or further plurality of anellovectors comprising:
(a) a proteinaceous exterior comprising an ORF1 molecule having at least 90% amino acid sequence identity to the ORF1 molecule in the proteinaceous exterior of the first plurality and
(b) a genetic element comprising a promoter element and a nucleic acid sequence (e.g., a DNA sequence) encoding the exogenous effector.

6. The method of any of the preceding claims, wherein the second plurality of anellovectors comprises comprises 90-110%, e.g., 95-105% of the number of anellovectors in the first plurality.

7. The method of any of the preceding claims, wherein the first plurality and the second plurality are administered via the same route of administration, e.g., intravenous administration.

8. The method of any of claims 1-6, wherein the first plurality and the second plurality are administered via different routes of administration.

9. The method of any of the preceding claims, wherein the second plurality of anellovectors comprises the same proteinaceous exterior as the anellovectors of the first plurality.

10. The method of any of the preceding claims, wherein the second plurality of anellovectors comprises an ORF1 molecule having the same amino acid sequence as the ORF1 molecule comprised by the anellovectors of the first plurality.

11. The method of any of the preceding claims, wherein the effector of the first plurality of anellovectors is an exogenous effector.

12. The method of any of the preceding claims, wherein the genetic element comprised in the anellovectors of the first plurality is detectable in the subject at least 50, 60, 70, 80, 90, 100, 110, 120, 130, 140, or 150 days after administration thereof, e.g., by a high-resolution melting (HRM) assay.

13. The method of any of the preceding claims, wherein the genetic element comprised in the anellovectors of the second plurality is detectable in the subject at least 50, 60, 70, 80, 90, 100, 110, 120, 130, 140, or 150 days after administration thereof, e.g., by a high-resolution melting (HRM) assay.

14. The method of any of the preceding claims, wherein the genetic element of the first and/or second plurality of anellovectors comprises an Anellovirus 5′ UTR (e.g., nucleotides 170-240 of SEQ ID NO: 16, nucleotides 323-393 of SEQ ID NO: 54, or nucleotides 185-254 of SEQ ID NO: 886), or a nucleic acid sequence having at least 80%, 85%, 90%, 95%, 96%, 97%, 98%, 99%, or 100% sequence identity thereto.

15. The method of any of the preceding claims, wherein the genetic element of the first and/or second plurality of anellovectors comprises the nucleic acid sequence of nucleotides 323-393 of SEQ ID NO: 41, or a nucleic acid sequence having at least 80%, 85%, 90%, 95%, 96%, 97%, 98%, 99%, or 100% sequence identity thereto.

16. The method of any of the preceding claims, wherein the genetic element of the first and/or second plurality of anellovectors comprises a sequence of at least 100 nucleotides in length, which consists of G or C at at least 80% of the positions.

17. The method of any of the preceding claims, wherein the first and/or second plurality of anellovectors comprises a nucleic acid sequence encoding an amino acid sequence chosen from ORF1, ORF2, ORF2/2, ORF2/3, ORF1/1, or ORF1/2 of Table 12, or an amino acid sequence having at least 80%, 85%, 90%, 95%, 96%, 97%, 98%, 99%, or 100% sequence identity thereto.

18. The method of any of the preceding claims, wherein the first and/or second plurality of anellovectors does not comprise a polypeptide having at least 80%, 85%, 90%, 95%, 96%, 97%, 98%, 99%, or 100% sequence identity to an Anellovirus ORF2, ORF2/2, ORF2/3, ORF1/1, or ORF1/2.

19. The method of any of the preceding claims, wherein the anellovectors of the first and/or second plurality are replication defective.

20. The method of any of the preceding claims, wherein the effector comprises:
(i) an intracellular nucleic acid (e.g., an miRNA or siRNA);
(ii) a secreted polypeptide chosen from an antibody molecule, an enzyme, a hormone, a cytokine molecule, a complement inhibitor, a growth factor, or a growth factor inhibitor, or a functional variant of any of the foregoing; or
(iii) a polypeptide that, when mutated, causes a human disease, or a functional variant of said polypeptide.

21. A primer comprising a nucleic acid sequence according to any of SEQ ID NOs: 1, 3, 4, 6, 8, 10, 12, 14, 17, 19, 21, or 23.

22. A mixture comprising a plurality of different primers comprising a nucleic acid sequence according to any 2, 3, 4, 5, 6, 7, 8, 9, 10, 11, or all of SEQ ID NOs: 1, 3, 4, 6, 8, 10, 12, 14, 17, 19, 21, or 23.

23. The mixture of claim 22, wherein each primer of the plurality is 9 nucleotides in length.

24. The mixture of claim 22, wherein each primer comprises one or more (e.g., 1 or 2) thiophosphate linkages.

25. A method of amplifying a circular DNA molecule comprising an Anellovirus sequence, the method comprising:
(a) providing a sample comprising a circular DNA molecule comprising an Anellovirus sequence and a first primer having at least 7, 8, or 9 nucleotides complementary to a portion of the Anellovirus sequence; and
(b) contacting the circular DNA molecule with a DNA-dependent DNA polymerase molecule;
wherein the contacting results in linear amplification (e.g., rolling circle amplification or multiple strand displacement amplification) of the DNA molecule, or a portion thereof.

26. The method of claim 25, wherein the sample comprises a plurality of primers having at least 7, 8, or 9 nucleotides complementary to a portion of the Anellovirus sequence.

27. The method of claim 26, wherein primers of the plurality comprise a nucleic acid sequence according to SEQ ID NO: 1, 3, 4, 6, 8, 10, 12, 14, 17, 19, 21, or 23, or any combination thereof.

28. The method of any of claims 25-27, wherein the DNA-dependent DNA polymerase molecule comprises Phi29.


SEQUENCE LISTING (1013 sequences)

SEQ ID NO: 1 - DNA, 9 bp, Artificial Sequence
  Description of Artificial Sequence: Synthetic primer
cgaatggyw

SEQ ID NO: 2 - DNA, 9 bp, Artificial Sequence
  Description of Artificial Sequence: Synthetic primer
aaggggcaa

SEQ ID NO: 3 - DNA, 9 bp, Artificial Sequence
  Description of Artificial Sequence: Synthetic primer
ytgyggbtg

SEQ ID NO: 4 - DNA, 9 bp, Artificial Sequence
  Description of Artificial Sequence: Synthetic primer
yagamacmm

SEQ ID NO: 5 - DNA, 10 bp, Artificial Sequence
  Description of Artificial Sequence: Synthetic primer
yaartggtac

SEQ ID NO: 6 - DNA, 10 bp, Artificial Sequence
  Description of Artificial Sequence: Synthetic primer
saccacwaac

SEQ ID NO: 7 - DNA, 9 bp, Artificial Sequence
  Description of Artificial Sequence: Synthetic primer
tbgtcggtg

SEQ ID NO: 8 - DNA, 9 bp, Artificial Sequence
  Description of Artificial Sequence: Synthetic primer
cactccgag

SEQ ID NO: 9 - DNA, 9 bp, Artificial Sequence
  Description of Artificial Sequence: Synthetic primer
gaggagtgc

SEQ ID NO: 10 - DNA, 9 bp, Artificial Sequence
  Description of Artificial Sequence: Synthetic primer
cagactccg

SEQ ID NO: 11 - DNA, 9 bp, Artificial Sequence
  Description of Artificial Sequence: Synthetic primer
gtgagtggg

SEQ ID NO: 12 - DNA, 9 bp, Artificial Sequence
  Description of Artificial Sequence: Synthetic primer
cttcgccat

SEQ ID NO: 13 - DNA, 9 bp, Artificial Sequence
  Description of Artificial Sequence: Synthetic primer
wrccattcg

SEQ ID NO: 14 - DNA, 9 bp, Artificial Sequence
  Description of Artificial Sequence: Synthetic primer
ttgcccctt

SEQ ID NO: 15 - DNA, 9 bp, Artificial Sequence
  Description of Artificial Sequence: Synthetic primer
cavccrcar

SEQ ID NO: 16 - DNA, 3753 bp, Alphatorquevirus sp.
tgctacgtcactaacccacgtgtcctctacaggccaatcgcagtctatgtcgtgcacttcctgggcatggtctacataattatataaatgcttgcacttccgaatggctgagtttttgctgcccgtccgcggagaggagccacggcaggggatccgaacgtcctgagggcgggtgccggaggtgagtttacacaccgaagtcaaggggcaattcgggctcaggactggccgggctttgggcaaggctcttaaaaatgcacttttctcgaataagcagaaagaaaaggaaagtgctactgctttgcgtgccagcagctaagaaaaaaccaactgctatgagcttctggaaacctccggtacacaatgtcacggggatccaacgcatgtggtatgagtcctttcaccgtggccacgcttctttttgtggttgtgggaatcctatacttcacattactgcacttgctgaaacatatggccatccaacaggcccgagaccttctgggccaccgggagtagaccccaacccccacatccgtagagccaggcctgccccggccgctccggagccctcacaggttgattcgagaccagccctgacatggcatggggatggtggaagcgacggaggcgctggtggttccggaagcggtggacccgtggcagacttcgcagacgatggcctcgatcagctcgtcgccgccctagacgacgaagagtaaggaggcgcagacggtggaggagggggagacgaaaaacaaggacttacagacgcaggagacgctttagacgcaggggacgaaaagcaaaacttataataaaactgtggcaacctgcagtaattaaaagatgcagaataaagggatacataccactgattataagtgggaacggtacctttgccacaaactttaccagtcacataaatgacagaataatgaaaggccccttcgggggaggacacagcactatgaggttcagcctctacattttgtttgaggagcacctcagacacatgaacttctggaccagaagcaacgataacctagagctaaccagatacttgggggcttcagtaaaaatatacaggcacccagaccaagactttatagtaatatacaacagaagaacccctctaggaggcaacatctacacagcaccctctctacacccaggcaatgccattttagcaaaacacaaaatattagtaccaagtttacagacaagaccaaagggtagaaaagcaattagactaagaatagcaccccccacactctttacagacaagtggtactttcaaaaggacatagccgacctcacccttttcaacatcatggcagttgaggctgacttgcggtttccgttctgctcaccacaaactgacaacacttgcatcagcttccaggtccttagttccgtttacaacaactacctcagtattaatacctttaataatgacaactcagactcaaagttaaaagaatttttaaataaagcatttccaacaacaggcacaaaaggaacaagtttaaatgcactaaatacatttagaacagaaggatgcataagtcacccacaactaaaaaaaccaaacccacaaataaacaaaccattagagtcacaatactttgcacctttagatgccctctggggagaccccatatactataatgatctaaatgaaaacaaaagtttgaacgatatcattgagaaaatactaataaaaaacatgattacataccatgcaaaactaagagaatttccaaattcataccaaggaaacaaggccttttgccacctaacaggcatatacagcccaccatacctaaaccaaggcagaatatctccagaaatatttggactgtacacagaaataatttacaacccttacacagacaaaggaactggaaacaaagtatggatggacccactaactaaagagaacaacatatataaagaaggacagagcaaatgcctactgactgacatgcccctatggactttactttttggatatacagactggtgtaaaaaggacactaataactgggacttaccactaaactacagactagtactaatatgcccttatacctttccaaaattgtacaatgaaaaagtaaaagactatgggtacatcccgtactcctacaaattcggagcgggtcagatgccagacggcagcaactacataccctttcagtttagagcaaagtggtaccccacagtactacaccagcaacaggtaatggaggacataagcaggagcgggccctttgcacctaaggtagaaaaaccaagcactcagctggtaatgaagtactgttttaactttaactggggcggtaaccctatcattgaacagattgttaaagaccccagcttccagcccacctatgaaatacccggtaccggtaacatccctagaagaatacaagtcatcgacccgcgggtcctgggaccgcactactcgttccggtcatgggacatgcgcagacacacatttagcagagcaagtattaagagagtgtcagaacaacaagaaacttctgaccttgtattctcaggcccaaaaaagcctcgggtcgacatcccaaaacaagaaacccaagaagaaagctcacattcactccaaagagaatcgagaccgtgggagaccgaggaagaaagcgagacagaagccctctcgcaagagagccaagaggtccccttccaacagcagttgcagcagcagtaccaagagcagctcaagctcagacagggaatcaaagtcctcttcgagcagctcataaggacccaacaaggggtccatgtaaacccatgcctacggtaggtcccaggcagtggctgtttccagagagaaagccagccccagctcctagcagtggagactgggccatggagtttctcgcagcaaaaatatttgataggccagttagaagcaaccttaaagataccccttactacccatatgttaaaaaccaatacaatgtctactttgaccttaaatttgaataaacagcagcttcaaacttgcaaggccgtgggagtttcactggtcggtgtctacctctaaaggtcactaagcactccgagcgtaagcgaggagtgcgaccctcccccctggaacaacttcttcggagtccggcgctacgccttcggctgcgccggacacctcagaccccccctccacccgaaacgcttgcgcgtttcggaccttcggcgtcgggggggtcgggagctttattaaacggactccgaagtgctcttggacactgagggggtgaacagcaacgaaagtgagtggggccagacttcgccataaggcctttatcttcttgccatttgtcagtgtccggggtcgccataggcttcgggctcgtttttaggccttccggactacaaaaatcgccattttggtgacgtcacggccgccatcttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcctctttttcaaaaaaaagcggaagtgccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 17 - protein, 127 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEE

SEQ ID NO: 18 - protein, 268 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEELLKTPASSPPMKYPVPVTSLEEYKSSTRGSWDRTTRSGHGTCADTHLAEQVLRECQNNKKLLTLYSQAQKSLGSTSQNKKPKKKAHIHSKENRDRGRPRKKARQKPSRKRAKRSPSNSSCSSSTKSSSSSDRESKSSSSSS

SEQ ID NO: 19 - protein, 276 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEEPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 20 - protein, 167 aa, Alphatorquevirus sp.
MSFWKPPVHNVTGIQRMWPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 21 - protein, 743 aa, Alphatorquevirus sp.
MAWGWWKRRRRWWFRKRWTRGRLRRRWPRSARRRPRRRRVRRRRRWRRGRRKTRTYRRRRRFRRRGRKAKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSINTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGRISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFNWGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 22 - protein, 194 aa, Alphatorquevirus sp.
MAWGWWKRRRRWWFRKRWTRGRLRRRWPRSARRRPRRRRIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 23 - protein, 113 aa, Alphatorquevirus sp.
MAWGWWKRRRRWWFRKRWTRGRLRRRWPRSARRRPRRRRAQKSLGSTSQNKKPKKKAHIHSKENRDRGRPRKKARQKPSRKRAKRSPSNSSCSSSTKSSSSSDRESKSSSSSS

SEQ ID NO: 24 - DNA, 9 bp, Artificial Sequence
  Description of Artificial Sequence: Synthetic primer
atggcgaag

SEQ ID NO: 25-53 - skipped (29 entries)

SEQ ID NO: 54 - DNA, 2979 bp, Betatorquevirus sp.
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61-104 - skipped (44 entries)

SEQ ID NO: 105 - DNA, 71 bp, Artificial Sequence
  Description of Artificial Sequence: Synthetic oligonucleotide
cgggtgccgkaggtgagtttacacaccgmagtcaaggggcaattcgggctcrggactggccgggcyhtggg

SEQ ID NO: 106 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctwtggg

SEQ ID NO: 107 - DNA, 71 bp, Artificial Sequence
  Description of Artificial Sequence: Synthetic oligonucleotide
cgggtgccgtaggtgagtttacacaccgcagtcaaggggcaattcgggctcgggactggccgggctatggg

SEQ ID NO: 108 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccctggg

SEQ ID NO: 109 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctttggg

SEQ ID NO: 110 - DNA, 71 bp, Artificial Sequence
  Description of Artificial Sequence: Synthetic oligonucleotide
cgggtgccggaggtgagtttacacaccgcagtcaaggggcaattcgggctcgggactggccgggctatggg

SEQ ID NO: 111 - DNA, 71 bp, Artificial Sequence
  Description of Artificial Sequence: Synthetic oligonucleotide
cgggtgccggaggtgagtttacacaccgaagtcaaggggcaattcgggctcaggactggccgggctttggg

SEQ ID NO: 112 - DNA, 71 bp, Artificial Sequence
  Description of Artificial Sequence: Synthetic oligonucleotide
cgggtgccggaggtgagtttacacaccgcagtcaaggggcaattcgggctcgggactggccgggcyhtggg

SEQ ID NO: 113 - DNA, 71 bp, Artificial Sequence; identical to SEQ ID NO: 107
  Description of Artificial Sequence: Synthetic oligonucleotide

SEQ ID NO: 114 - DNA, 70 bp, Artificial Sequence
  Description of Artificial Sequence: Synthetic oligonucleotide
cgggtgccggaggtgagtttacacaccgcagtcaaggggcaattcgggctcgggactggccgggcccggg

SEQ ID NO: 115 - DNA, 71 bp, Artificial Sequence; identical to SEQ ID NO: 111
  Description of Artificial Sequence: Synthetic oligonucleotide

SEQ ID NO: 116 - DNA, 69 bp, Artificial Sequence
  Description of Artificial Sequence: Synthetic oligonucleotide
cgggtgccggaggtgagtttacacaccgcagtcaaggggcaattcgggctcgggaggccgggccatggg

SEQ ID NO: 117 - DNA, 71 bp, Artificial Sequence
  Description of Artificial Sequence: Synthetic oligonucleotide
cgggtgccggaggtgagtttacacaccgcagtcaaggggcaattcgggctcgggactggccgggccccggg

SEQ ID NO: 118 - DNA, 71 bp, Artificial Sequence; identical to SEQ ID NO: 110
  Description of Artificial Sequence: Synthetic oligonucleotide

SEQ ID NO: 119 - DNA, 71 bp, Artificial Sequence
  Description of Artificial Sequence: Synthetic oligonucleotide
cgggtgccgaaggtgagtttacacaccgcagtcaaggggcaattcgggctcgggactggccgggctatggg

SEQ ID NO: 120 - DNA, 117 bp, Artificial Sequence
  Description of Artificial Sequence: Synthetic polynucleotide
  misc_feature (10)..(10): May or may not be present
  misc_feature (12)..(12): May or may not be present
  misc_feature (30)..(32): May or may not be present
  misc_feature (34)..(34): May or may not be present
  misc_feature (43)..(46): May or may not be present
  misc_feature (52)..(54): May or may not be present
  misc_feature (70)..(71): May or may not be present
  misc_feature (89)..(90): May or may not be present
  misc_feature (103)..(103): May or may not be present
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121 - DNA, 169 bp, Artificial Sequence
  Description of Artificial Sequence: Synthetic polynucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
  modified_base (97)..(98): a, c, t, g, unknown or other
gccgccgcggcggcggsggngnsgcgcgctdcgcgcgcsnnncrccrgggggnnnncwgcsncncccccccccgcgcatgcgcgggkcccccccccnncggggggctccgccccccggcccccccccgtgctaaacccaccgcgcatgcgcgaccacgcccccgccgcc

SEQ ID NO: 122 - DNA, 79 bp, Artificial Sequence
  Description of Artificial Sequence: Synthetic oligonucleotide
  modified_base (20)..(20): a, c, t, g, unknown or other
  modified_base (22)..(22): a, c, t, g, unknown or other
  modified_base (40)..(42): a, c, t, g, unknown or other
  modified_base (53)..(56): a, c, t, g, unknown or other
  modified_base (62)..(62): a, c, t, g, unknown or other
  modified_base (64)..(64): a, c, t, g, unknown or other
gccgccgcggcggcggsggngnsgcgcgctdcgcgcgcsnnncrccrgggggnnnncwgcsncncccccccccgcgcat

SEQ ID NO: 123 - DNA, 31 bp, Artificial Sequence
  Description of Artificial Sequence: Synthetic oligonucleotide
  modified_base (18)..(19): a, c, t, g, unknown or other
gcgcgggkcccccccccnncggggggctccg

SEQ ID NO: 124 - DNA, 59 bp, Artificial Sequence
  Description of Artificial Sequence: Synthetic oligonucleotide
ccccccggcccccccccgtgctaaacccaccgcgcatgcgcgaccacgcccccgccgcc

SEQ ID NO: 125 - DNA, 156 bp, Artificial Sequence
  Description of Artificial Sequence: Synthetic polynucleotide
gcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 126 - DNA, 7 bp, Artificial Sequence
  Description of Artificial Sequence: Synthetic oligonucleotide
gcggcgg

SEQ ID NO: 127 - DNA, 7 bp, Artificial Sequence
  Description of Artificial Sequence: Synthetic oligonucleotide
gggggcg

SEQ ID NO: 128 - DNA, 6 bp, Artificial Sequence
  Description of Artificial Sequence: Synthetic oligonucleotide
gccgcg

SEQ ID NO: 129 - DNA, 25 bp, Artificial Sequence
  Description of Artificial Sequence: Synthetic oligonucleotide
ttcgcgcgccgcccaccagggggtg

SEQ ID NO: 130 - DNA, 5 bp, Artificial Sequence
  Description of Artificial Sequence: Synthetic oligonucleotide
ctgcg

SEQ ID NO: 131 - DNA, 17 bp, Artificial Sequence
  Description of Artificial Sequence: Synthetic oligonucleotide
cgcccccccccgcgcat

SEQ ID NO: 132 - DNA, 17 bp, Artificial Sequence
  Description of Artificial Sequence: Synthetic oligonucleotide
gcgcggggccccccccc

SEQ ID NO: 133 - DNA, 72 bp, Artificial Sequence
  Description of Artificial Sequence: Synthetic oligonucleotide
gggggggctccgcccccccggcccccccccgtgctaaacccaccgcgcatgcgcgaccacgcccccgccgcc

SEQ ID NO: 134 - DNA, 115 bp, Artificial Sequence
  Description of Artificial Sequence: Synthetic polynucleotide
cggcggcggcggcgcgcgcgctgcgcgcgcgcgccgggggggcgccagcgcccccccccccgcgcatgcacgggtccccccccccacggggggctccgccccccggccccccccc

SEQ ID NO: 135 - DNA, 14 bp, Artificial Sequence
  Description of Artificial Sequence: Synthetic oligonucleotide
cggcggcggcggcg

SEQ ID NO: 136 - DNA, 17 bp, Artificial Sequence
  Description of Artificial Sequence: Synthetic oligonucleotide
cgcgcgctgcgcgcgcg

SEQ ID NO: 137 - DNA, 19 bp, Artificial Sequence
  Description of Artificial Sequence: Synthetic oligonucleotide
cgccgggggggcgccagcg

SEQ ID NO: 138 - DNA, 17 bp, Artificial Sequence
  Description of Artificial Sequence: Synthetic oligonucleotide
cccccccccccgcgcat

SEQ ID NO: 139 - DNA, 31 bp, Artificial Sequence
  Description of Artificial Sequence: Synthetic oligonucleotide
gcacgggtccccccccccacggggggctccg

SEQ ID NO: 140 - DNA, 17 bp, Artificial Sequence
  Description of Artificial Sequence: Synthetic oligonucleotide
ccccccggccccccccc

SEQ ID NO: 141 - DNA, 121 bp, Artificial Sequence
  Description of Artificial Sequence: Synthetic polynucleotide
ccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 142 - DNA, 37 bp, Artificial Sequence
  Description of Artificial Sequence: Synthetic oligonucleotide
ccgtcggcgggggggccgcgcgctgcgcgcgcggccc

SEQ ID NO: 143 - DNA, 84 bp, Artificial Sequence
  Description of Artificial Sequence: Synthetic oligonucleotide
ccgggggaggcacagcctcccccccccgcgcgcatgcgcgcgggtccccccccctccggggggctccgccccccggcccccccc

SEQ ID NO: 144 - DNA, 104 bp, Artificial Sequence
  Description of Artificial Sequence: Synthetic polynucleotide
cggcggcggcgcgcgcgctacgcgcgcgcgccggggggctgccgccccccccccgcgcatgcgcggggcccccccccgcggggggctccgccccccggcccccc

SEQ ID NO: 145 - DNA, 11 bp, Artificial Sequence
  Description of Artificial Sequence: Synthetic oligonucleotide
cggcggcggcg

SEQ ID NO: 146 - DNA, 17 bp, Artificial Sequence
  Description of Artificial Sequence: Synthetic oligonucleotide
cgcgcgctacgcgcgcg

SEQ ID NO: 147 - DNA, 10 bp, Artificial Sequence
  Description of Artificial Sequence: Synthetic oligonucleotide
cgccgggggg

SEQ ID NO: 148 - DNA, 7 bp, Artificial Sequence
  Description of Artificial Sequence: Synthetic oligonucleotide
ctgccgc

SEQ ID NO: 149 - DNA, 15 bp, Artificial Sequence
  Description of Artificial Sequence: Synthetic oligonucleotide
cccccccccgcgcat

SEQ ID NO: 150 - DNA, 17 bp, Artificial Sequence
  Description of Artificial Sequence: Synthetic oligonucleotide
gcgcggggccccccccc

SEQ ID NO: 151 - DNA, 13 bp, Artificial Sequence
  Description of Artificial Sequence: Synthetic oligonucleotide
gcggggggctccg

SEQ ID NO: 152 - DNA, 14 bp, Artificial Sequence
  Description of Artificial Sequence: Synthetic oligonucleotide
ccccccggcccccc

SEQ ID NO: 153 - DNA, 122 bp, Artificial Sequence
  Description of Artificial Sequence: Synthetic polynucleotide
gccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 154 - DNA, 19 bp, Artificial Sequence
  Description of Artificial Sequence: Synthetic oligonucleotide
gccgccgcggcggcggggg

SEQ ID NO: 155 - DNA, 41 bp, Artificial Sequence
  Description of Artificial Sequence: Synthetic oligonucleotide
gcggcgcgctgcgcgcgccgcccagtagggggagccatgcg

SEQ ID NO: 156 - DNA, 15 bp, Artificial Sequence
  Description of Artificial Sequence: Synthetic oligonucleotide
cccccccccgcgcat

SEQ ID NO: 157 - DNA, 17 bp, Artificial Sequence
  Description of Artificial Sequence: Synthetic oligonucleotide
gcgcggggccccccccc

SEQ ID NO: 158 - DNA, 13 bp, Artificial Sequence
  Description of Artificial Sequence: Synthetic oligonucleotide
gcggggggctccg

SEQ ID NO: 159 - DNA, 17 bp, Artificial Sequence
  Description of Artificial Sequence: Synthetic oligonucleotide
ccccccggccccccccg

SEQ ID NO: 160 - DNA, 36 bp, Artificial Sequence
  Description of Artificial Sequence: Synthetic oligonucleotide
cgcgctgcgcgcgccgcccagtagggggagccatgc

SEQ ID NO: 161 - DNA, 78 bp, Artificial Sequence
  Description of Artificial Sequence: Synthetic oligonucleotide
ccgccatcttaagtagttgaggcggacggtggcgtgagttcaaaggtcaccatcagccacacctactcaaaatggtgg

SEQ ID NO: 162 - DNA, 172 bp, Artificial Sequence
  Description of Artificial Sequence: Synthetic polynucleotide
cttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcc

SEQ ID NO: 163 - DNA, 36 bp, Artificial Sequence; identical to SEQ ID NO: 160
  Description of Artificial Sequence: Synthetic oligonucleotide

SEQ ID NO: 164 - DNA, 36 bp, Artificial Sequence
  Description of Artificial Sequence: Synthetic oligonucleotide
gcgctdcgcgcgcgcgccggggggctgcgccccccc

SEQ ID NO: 165 - DNA, 36 bp, Artificial Sequence
  Description of Artificial Sequence: Synthetic oligonucleotide
gcgcttcgcgcgccgcccactagggggcgttgcgcg

SEQ ID NO: 166 - DNA, 36 bp, Artificial Sequence
  Description of Artificial Sequence: Synthetic oligonucleotide
gcgctgcgcgcgccgcccagtagggggcgcaatgcg

SEQ ID NO: 167 - DNA, 36 bp, Artificial Sequence
  Description of Artificial Sequence: Synthetic oligonucleotide
gcgctgcgcgcgcggcccccgggggaggcattgcct

SEQ ID NO: 168 - DNA, 36 bp, Artificial Sequence
  Description of Artificial Sequence: Synthetic oligonucleotide
gcgctgcgcgcgcgcgccgggggggcgccagcgccc

SEQ ID NO: 169 - DNA, 36 bp, Artificial Sequence
  Description of Artificial Sequence: Synthetic oligonucleotide
gcgcttcgcgcgcgcgccggggggctccgccccccc

SEQ ID NO: 170 - DNA, 36 bp, Artificial Sequence
  Description of Artificial Sequence: Synthetic oligonucleotide
gcgcttcgcgcgcgcgccggggggctgcgccccccc

SEQ ID NO: 171 - DNA, 36 bp, Artificial Sequence
  Description of Artificial Sequence: Synthetic oligonucleotide
gcgctacgcgcgcgcgccggggggctgcgccccccc

SEQ ID NO: 172 - DNA, 36 bp, Artificial Sequence
  Description of Artificial Sequence: Synthetic oligonucleotide
gcgctacgcgcgcgcgccggggggctctgccccccc

SEQ ID NO: 173-184 - skipped (12 entries)

SEQ ID NO: 185 - protein, 743 aa, Alphatorquevirus sp.; identical to SEQ ID NO: 21

SEQ ID NO: 186 - protein, 68 aa, Alphatorquevirus sp.
MAWGWWKRRRRWWFRKRWTRGRLRRRWPRSARRRPRRRRVRRRRRWRRGRRKTRTYRRRRRFRRRGRK

SEQ ID NO: 187 - protein, 212 aa, Alphatorquevirus sp.
AKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSI

SEQ ID NO: 188 - protein, 133 aa, Alphatorquevirus sp.
NTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGR

SEQ ID NO: 189 - protein, 166 aa, Alphatorquevirus sp.
ISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFN

SEQ ID NO: 190 - protein, 164 aa, Alphatorquevirus sp.
WGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 191-214 - skipped (24 entries)

SEQ ID NO: 215 - protein, 666 aa, Betatorquevirus sp.; identical to SEQ ID NO: 58

SEQ ID NO: 216 - protein, 38 aa, Betatorquevirus sp.
MPYYYRRRRYNYRRPRWYGRGWIRRPFRRRFRRKRRVR

SEQ ID NO: 217 - protein, 208 aa, Betatorquevirus sp.
PTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 218 - protein, 128 aa, Betatorquevirus sp.
ISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAG

SEQ ID NO: 219 - protein, 163 aa, Betatorquevirus sp.
TMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFK

SEQ ID NO: 220 - protein, 129 aa, Betatorquevirus sp.
WGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 221-226 - skipped (6 entries)

SEQ ID NO: 227 - protein, 220 aa, Artificial Sequence
  Description of Artificial Sequence: Synthetic polypeptide
  MOD_RES (29)..(31): Any amino acid
  SITE (29)..(31): This region may encompass 0-3 residues
  MOD_RES (100)..(100): Any amino acid
  MOD_RES (125)..(129): Any amino acid
  SITE (125)..(129): This region may encompass 1-5 residues
  MOD_RES (181)..(181): Any amino acid
  MOD_RES (211)..(211): Any amino acid
LVLTQWQPNTVRRCYIRGYLPLIICGENXXXTTSRNYATHSDDTIQKGPFGGGMSTTTFSLRVLYDEYQRFMNRWTYSNEDLDLARYLGCKFTFYRHPDXDFIVQYNTNPPFKDTKLTAPSIHPXXXXXGMLMLSKRKILIPSLKTRPKGKHYVKVRIGPPKLFEDKWYTQSDLCDVPLVXLYATAADLQHPFGSPQTDNPCVTFQVLGSXYNKHLSISP

SEQ ID NO: 228 - protein, 172 aa, Artificial Sequence
  Description of Artificial Sequence: Synthetic polypeptide
  MOD_RES (38)..(38): Any amino acid
  MOD_RES (44)..(46): Any amino acid
  SITE (44)..(46): This region may encompass 0-3 residues
  MOD_RES (77)..(77): Any amino acid
  MOD_RES (79)..(79): Any amino acid
  MOD_RES (98)..(101): Any amino acid
  SITE (98)..(101): This region may encompass 0-4 residues
SNFEFPGAYTDITYNPLTDKGVGNMVWIQYLTKPDTIXDKTQSXXXKCLIEDLPLWAALYGYVDFCEKETGDSAIIXNXGRVLIRCPYTKPPLYDKTXXXXNKGFVPYSTNFGNGKMPGGSGYVPIYWRARWYPTLFHQKEVLEDIVQSGPFAYKDEKPSTQLVMKYCFNFN

SEQ ID NO: 229 - protein, 258 aa, Artificial Sequence
  Description of Artificial Sequence: Synthetic polypeptide
  MOD_RES (20)..(22): Any amino acid
  SITE (20)..(22): This region may encompass 0-3 residues
  MOD_RES (25)..(25): Any amino acid
  MOD_RES (78)..(78): Any amino acid
  MOD_RES (89)..(89): Any amino acid
  MOD_RES (91)..(91): Any amino acid
  MOD_RES (95)..(98): Any amino acid
  SITE (95)..(98): This region may encompass 1-4 residues
  MOD_RES (107)..(120): Any amino acid
  SITE (107)..(120): This region may encompass 2-14 residues
  MOD_RES (129)..(129): Any amino acid
  MOD_RES (139)..(168): Any amino acid
  SITE (139)..(168): This region may encompass 0-30 residues
  MOD_RES (201)..(204): Any amino acid
  SITE (201)..(204): This region may encompass 0-4 residues
  MOD_RES (219)..(258): Any amino acid
  SITE (219)..(258): This region may encompass 0-40 residues
WGGNPISQQVVRNPCKDSGXXXSGXGRQPRSVQVVDPKYMGPEYTFHSWDWRRGLFGEKAIKRMSEQPTDDEIFTGGXPKRPRRDPPTXQXPEEXXXXQKESSSFRXXXXXXXXXXXXXXPWESSSQEXESESQEEEEXXXXXXXXXXXXXXXXXXXXXXXXXXXXXXEQTVQQQLRQQLREQRRLRVQLQLLFQQLLKTXXXXQAGLHINPLLLSQAXXXXXXXXXXXXXXXXXXXXXXXXXXXXXXXXXXXXXXXX

SEQ ID NO: 230 - protein, 214 aa, Artificial Sequence
  Description of Artificial Sequence: Synthetic polypeptide
  MOD_RES (136)..(136): Any amino acid
  MOD_RES (138)..(141): Any amino acid
  SITE (138)..(141): This region may encompass 1-4 residues
  MOD_RES (179)..(179): Any amino acid
LKQWQPSTIRKCKIKGYLPLFQCGKGRISNNYTQYKESIVPHHEPGGGGWSIQQFTLGALYEEHLKLRNWWTKSNDGLPLVRYLGCTIKLYRSEDTDYIVTYQRCYPMTATKLTYLSTQPSRMLMNKHKIIVPSKXTXXXXNKKKKPYKKIFIKPPSQMQNKWYFQQDIANTPLLQLTXTACSLDRMYLSSDSISNNITFTSLNTNFFQNPNFQ

SEQ ID NO: 231 - protein, 187 aa, Artificial Sequence
  Description of Artificial Sequence: Synthetic polypeptide
  MOD_RES (1)..(10): Any amino acid
  SITE (1)..(10): This region may encompass 4-10 residues
  MOD_RES (38)..(45): Any amino acid
  SITE (38)..(45): This region may encompass 1-8 residues
  MOD_RES (94)..(94): Any amino acid
  MOD_RES (100)..(102): Any amino acid
  SITE (100)..(102): This region may encompass 1-3 residues
  MOD_RES (112)..(112): Any amino acid
  MOD_RES (114)..(115): Any amino acid
  SITE (114)..(115): This region may encompass 0-2 residues
  MOD_RES (124)..(139): Any amino acid
  SITE (124)..(139): This region may encompass 3-16 residues
  MOD_RES (154)..(154): Any amino acid
XXXXXXXXXXTPLYFECRYNPFKDKGTGNKVYLVSNNXXXXXXXXTGWDPPTDPDLIIEGFPLWLLLWGWLDWQKKLGKIQNIDTDYILVIQSXYYIPPXXXKLPYYVPLDXDXXFLHGRSPYXXXXXXXXXXXXXXXXPSDKQHWHPKVRFQXETINNIALTGPGTPKLPNQKSIQAHMKYKFYFK

SEQ ID NO: 232 - protein, 163 aa, Artificial Sequence
  Description of Artificial Sequence: Synthetic polypeptide
  MOD_RES (34)..(34): Any amino acid
  MOD_RES (65)..(65): Any amino acid
  MOD_RES (77)..(78): Any amino acid
  MOD_RES (86)..(87): Any amino acid
  MOD_RES (96)..(96): Any amino acid
  MOD_RES (102)..(106): Any amino acid
  SITE (102)..(106): This region may encompass 0-5 residues
  MOD_RES (125)..(125): Any amino acid
  MOD_RES (135)..(135): Any amino acid
  MOD_RES (138)..(163): Any amino acid
  SITE (138)..(163): This region may encompass 0-26 residues
WGGCPAPMETITDPCKQPKYPIPNNLLQTTSLQXPTTPIETYLYKFDERRGLLTKKAAKRIKKDXTTETTLFTDTGXXTSTTLPTXXQTETTQEEXTSEEEXXXXXETLLQQLQQLRRKQKQLRXRILQLLQLLXLLXXXXXXXXXXXXXXXXXXXXXXXXXX

SEQ ID NO: 233 - protein, 203 aa, Artificial Sequence
  Description of Artificial Sequence: Synthetic polypeptide
  MOD_RES (79)..(79): Any amino acid
  MOD_RES (104)..(104): Any amino acid
  MOD_RES (116)..(116): Any amino acid
  MOD_RES (120)..(121): Any amino acid
  MOD_RES (125)..(125): Any amino acid
  MOD_RES (170)..(170): Any amino acid
TIPLKQWQPESIRKCKIKGYGTLVLGAEGRQFYCYTNEKDEYTPPKAPGGGGFGVELFSLEYLYEQWKARNNIWTKSNXYKDLCRYTGCKITFYRHPTTDFIVXYSRQPPFEIDKXTYMXXHPQXLLLRKHKKIILSKATNPKGKLKKKIKIKPPKQMLNKWFFQKQFAXYGLVQLQAAACBLRYPRLGCCNENRLITLYYLN

SEQ ID NO: 234 - protein, 162 aa, Artificial Sequence
  Description of Artificial Sequence: Synthetic polypeptide
  MOD_RES (12)..(12): Any amino acid
  MOD_RES (20)..(20): Any amino acid
  MOD_RES (23)..(23): Any amino acid
  MOD_RES (30)..(30): Any amino acid
  MOD_RES (58)..(58): I or L
  MOD_RES (84)..(84): Any amino acid
  MOD_RES (90)..(90): Any amino acid
  MOD_RES (95)..(95): Any amino acid
  MOD_RES (105)..(105): Any amino acid
  MOD_RES (111)..(111): I or L
  MOD_RES (113)..(113): Any amino acid
  MOD_RES (154)..(154): Any amino acid
  MOD_RES (156)..(156): Any amino acid
LPIVVARYNPAXDTGKGNKXWLXSTLNGSXWAPPTTDKDLIIEGLPLWLALYGYWSYXKKVKKDKGILQSHMFVVKSPAIQPLXTATTQXTFYPXIDNSFIQGKXPYDEPXTXNQKKLWYPTLEHQQETINAIVESGPYVPKLDNQKNSTWELXYXYTFYFK

SEQ ID NO: 235 - protein, 177 aa, Artificial Sequence
  Description of Artificial Sequence: Synthetic polypeptide
  MOD_RES (16)..(16): Any amino acid
  MOD_RES (26)..(26): Any amino acid
  MOD_RES (33)..(33): Any amino acid
  MOD_RES (73)..(73): Any amino acid
  MOD_RES (81)..(82): Any amino acid
  SITE (81)..(82): This region may encompass 0-2 residues
  MOD_RES (90)..(90): Any amino acid
  MOD_RES (94)..(94): Any amino acid
  MOD_RES (119)..(124): Any amino acid
  SITE (119)..(124): This region may encompass 1-6 residues
  MOD_RES (168)..(177): Any amino acid
  SITE (168)..(177): This region may encompass 1-10 residues
WGGPQIPDQPVEDPKXQGTYPVPDTXQQTIQIXNPLKQKPETMFHDWDYRRGIITSTALKRMQENLETDSSFXSDSEETPXXKKKKRLTXELPXPQEETEEIQSCLLSLCEESTCQEEXXXXXXENLQQLIHQQQQQQQQLKHNILKLLSDLKZKQRLLQLQTGILEXXXXXXXXXX

SEQ ID NO: 236-299 - skipped (64 entries)

SEQ ID NO: 300 - RNA, 76 nt, Artificial Sequence
  Description of Artificial Sequence: Synthetic oligonucleotide
gccauuuuaaguagcugacgucaaggauugacguaaagguuaaaggucauccucggcggaagcuacacaaaauggu

SEQ ID NO: 301 - RNA, 78 nt, Artificial Sequence
  Description of Artificial Sequence: Synthetic oligonucleotide
gcguacgucacaagucacguggaggggacccgcuguaacccggaaguaggccccgucacgugacuuaccacgugugua

SEQ ID NO: 302 - RNA, 77 nt, Artificial Sequence
  Description of Artificial Sequence: Synthetic oligonucleotide
gccauuuuaaguagcugacgucaaggauugacgugaagguuaaaggucauccucggcggaagcuacacaaaauggug

SEQ ID NO: 303 - RNA, 78 nt, Artificial Sequence
  Description of Artificial Sequence: Synthetic oligonucleotide
gcacacgucauaagucacgugguggggacccgcuguaacccggaaguaggccccgucacgugauuugucacgugugua

SEQ ID NO: 304 - RNA, 66 nt, Artificial Sequence
  Description of Artificial Sequence: Synthetic oligonucleotide
cuuccgggucauaggucacaccuacgucacaagucacguggggaggguuggcguauagcccggaag

SEQ ID NO: 305 - RNA, 68 nt, Artificial Sequence
  Description of Artificial Sequence: Synthetic oligonucleotide
gccggggggcugccgccccccccggggaaaggggggggccccccccggggggggguuugccccccggc

SEQ ID NO: 306 - RNA, 78 nt, Artificial Sequence
  Description of Artificial Sequence: Synthetic oligonucleotide
auacgucaucagucacgugggggaaggcgugccuaaacccggaagcauccucguccacgugacugugacguguguggc

SEQ ID NO: 307 - RNA, 73 nt, Artificial Sequence
  Description of Artificial Sequence: Synthetic oligonucleotide
cauuuuaaguaaggcggaagcagcucggcguacacaaaauggcggcggagcacuuccggcuugcccaaaaugg

SEQ ID NO: 308 - RNA, 71 nt, Artificial Sequence
  Description of Artificial Sequence: Synthetic oligonucleotide
gucacaagucacguggggaggguuggcguuuaacccggaagccaauccucuuacguggccugucacgugac

SEQ ID NO: 309 - RNA, 70 nt, Artificial Sequence
  Description of Artificial Sequence: Synthetic oligonucleotide
cgaccgcgucccgaaggcggguacccgaggugaguuuacacaccgagguuaagggccaauucgggcuugg

SEQ ID NO: 310 - RNA, 59 nt, Artificial Sequence
  Description of Artificial Sequence: Synthetic oligonucleotide
cgcgguaucguagccgacgcggaccccguuuucggggcccccgcggggcucucggcgcg

SEQ ID NO: 311 - RNA, 78 nt, Artificial Sequence
  Description of Artificial Sequence: Synthetic oligonucleotide
cgccauuuugugauacgcgcguccccucccggcuuccguacaacgucaggcggggcguggccguaucagaaaauggcg

SEQ ID NO: 312 - RNA, 77 nt, Artificial Sequence
  Description of Artificial Sequence: Synthetic oligonucleotide
gcuacgucauaagucacgugacugggcagguacuaaacccggaaguauccucggucacguggccugucacguaguug

SEQ ID NO: 313 - RNA, 80 nt, Artificial Sequence
  Description of Artificial Sequence: Synthetic oligonucleotide
ggcusugacgucaaagucacgugggraggguggcguuaaacccggaagucauccucgucacgugaccugacgucacagcc

SEQ ID NO: 314 - RNA, 66 nt, Artificial Sequence
  Description of Artificial Sequence: Synthetic oligonucleotide
gcccguccgcggcgagagcgcgagcgaagcgagcgaucgagcgucccgugggcgggugccgaaggu

SEQ ID NO: 315 - RNA, 80 nt, Artificial Sequence
  Description of Artificial Sequence: Synthetic oligonucleotide
gguugugacgucaaagucacguggggagggcggcguuaaacccggaagucauccucgucacgugaccugacgucacggcc

SEQ ID NO: 316 - RNA, 67 nt, Artificial Sequence
  Description of Artificial Sequence: Synthetic oligonucleotide
gcccguccgcggcgagagcgcgagcgaagcgagcgaucgagcgucccgugggcgggugccguaggug

SEQ ID NO: 317 - RNA, 67 nt, Artificial Sequence; identical to SEQ ID NO: 316
  Description of Artificial Sequence: Synthetic oligonucleotide

SEQ ID NO: 318 - RNA, 80 nt, Artificial Sequence
  Description of Artificial Sequence: Synthetic oligonucleotide
ggcugugacgucaaagucacguggggagggcggcguuaaacccggaagucauccucgucacgugaccugacgucacggcc

SEQ ID NO: 319 - RNA, 79 nt, Artificial Sequence
  Description of Artificial Sequence: Synthetic oligonucleotide
agaccacgugguaagucacgugggggcagcugcuguaaacccggaaguagcugacccgcgugacuggucacgugaccug

SEQ ID NO: 320 - RNA, 78 nt, Artificial Sequence
  Description of Artificial Sequence: Synthetic oligonucleotide
cgccauuuuauaauacgcgcguccccucccggcuuccguacuacgucaggcggggcguggccguauuagaaaauggug

SEQ ID NO: 321 - RNA, 72 nt, Artificial Sequence
  Description of Artificial Sequence: Synthetic oligonucleotide
uaaguaaggcggaaccaggcugucacccugugucaaaggucaagggacagccuuccggcuugcacaaaaugg

SEQ ID NO: 322 - RNA, 78 nt, Artificial Sequence
  Description of Artificial Sequence: Synthetic oligonucleotide
ugccuacgucauaagucacguggggacggcugcuguaaacacggaaguagcugacccgcgugacuugucacgugagca

SEQ ID NO: 323 - RNA, 72 nt, Artificial Sequence
  Description of Artificial Sequence: Synthetic oligonucleotide
uuguguaaggcggaacaggcugacaccccgugucaaaggucaggggucagccuccgcuuugcaccaaauggu

SEQ ID NO: 324 - RNA, 79 nt, Artificial Sequence
  Description of Artificial Sequence: Synthetic oligonucleotide
uaccuacgucauaagucacgugggaagagcugcugugaaccuggaaguagcugacccgcguggcuugucacgugagugc

SEQ ID NO: 325 - RNA, 75 nt, Artificial Sequence
  Description of Artificial Sequence: Synthetic oligonucleotide
uuuuccuggcccguccgcggcgagagcgcgagcgaagcgagcgaucgggcgucccgagggcgggugccggaggug

SEQ ID NO: 326 - RNA, 68 nt, Artificial Sequence
  Description of Artificial Sequence: Synthetic oligonucleotide
aaagugaguggggccagacuucgccauagggccuuuaacuuccgggugcgucugggggccgccauuuu

SEQ ID NO: 327 - RNA, 73 nt, Artificial Sequence
  Description of Artificial Sequence: Synthetic oligonucleotide
gugacguuacucucacgugaugggggcgugcucuaacccggaagcauccucgaccacgugacugugacgucac

SEQ ID NO: 328 - RNA, 75 nt, Artificial Sequence
  Description of Artificial Sequence: Synthetic oligonucleotide
agcgucuacuacguacacuuccugggguguguccugccacuguauauaaaccagaggggugacgaaugguagagu

SEQ ID NO: 329 - RNA, 73 nt, Artificial Sequence
  Description of Artificial Sequence: Synthetic oligonucleotide
gugacgucaaagucacguggugacggccauuuuaacccggaaguggcuguugucacgugacuugacgucacgg

SEQ ID NO: 330 - RNA, 62 nt, Artificial Sequence
  Description of Artificial Sequence: Synthetic oligonucleotide
gcuuuagacgccauuuuaggcccucgcgggcacccguaggcgcguuuuaaugacgucacggc

SEQ ID NO: 331 - RNA, 73 nt, Artificial Sequence
  Description of Artificial Sequence: Synthetic oligonucleotide
cacccguaggcgcguuuuaaugacgucacggcagccauuuugucgugacguuugagacacgugaugggggcgu

SEQ ID NO: 332 - RNA, 80 nt, Artificial Sequence
  Description of Artificial Sequence: Synthetic oligonucleotide
gucgugacguuugagacacgugaugggggcgugccuaaacccggaagcaucccuggucacgugacucugacgucacggcg

SEQ ID NO: 333 - RNA, 77 nt, Artificial Sequence
  Description of Artificial Sequence: Synthetic oligonucleotide
cgaaagugaguggggccagacuucgccauaaggccuuuaacuuccgggugcgugugggggccgccauuuuagcuucg

SEQ ID NO: 334 - RNA, 76 nt, Artificial Sequence
  Description of Artificial Sequence: Synthetic oligonucleotide
cugugacgucaaagucacguggggagggcggcguguaacccggaagucauccucgucacgugaccugacgucacgg

SEQ ID NO: 335 - RNA, 73 nt, Artificial Sequence
  Description of Artificial Sequence: Synthetic oligonucleotide
cuguccgccaucuugugacuuccuuccgcuuuuucaaaaaaaaagaggaaguaugacguagcggcgggggggc

SEQ ID NO: 336 - RNA, 67 nt, Artificial Sequence
  Description of Artificial Sequence: Synthetic oligonucleotide
gguagaguuuuuuccgcccguccgcagcgaggacgcgagcgcagcgagcggccgagcgacccguggg

SEQ ID NO: 337 - RNA, 80 nt, Artificial Sequence
  Description of Artificial Sequence: Synthetic oligonucleotide
gcugugacguuucagucacguggggagggaacgccuaaacccggaagcgucccuggucacgugauugugacgucacggcc

SEQ ID NO: 338 - RNA, 63 nt, Artificial Sequence
  Description of Artificial Sequence: Synthetic oligonucleotide
ccgccauuuugugacuuccuuccgcuuuuucaaaaaaaaagaggaagugugacguagcggcgg

SEQ ID NO: 339 - RNA, 78 nt, Artificial Sequence
  Description of Artificial Sequence: Synthetic oligonucleotide
gacugugacgucaaagucacguggggagggcggcguguaacccggaagucauccucgucacgugaccugacgucacgg

SEQ ID NO: 340 - RNA, 73 nt, Artificial Sequence
  Description of Artificial Sequence: Synthetic oligonucleotide
cuguccgccaucuugugacuuccuuccgcuuuuucaaaaaaaaagaggaaguaugacguggcggcgggggggc

SEQ ID NO: 341 - RNA, 80 nt, Artificial Sequence
  Description of Artificial Sequence: Synthetic oligonucleotide
gguugugacgucaaagucacguggggagggcggcguguaacccggaagucauccucgucacgugaccugacgucacggcc

SEQ ID NO: 342 - RNA, 65 nt, Artificial Sequence
  Description of Artificial Sequence: Synthetic oligonucleotide
cccgccaucuugugacuuccuuccgcuuuuucaaaaaaaaagaggaagugugacguagcggcggg

SEQ ID NO: 343 - RNA, 67 nt, Artificial Sequence; identical to SEQ ID NO: 316
  Description of Artificial Sequence: Synthetic oligonucleotide

SEQ ID NO: 344 - RNA, 78 nt, Artificial Sequence
  Description of Artificial Sequence: Synthetic oligonucleotide
gacugugacgucaaagucacguggggaggagggcguguaacccggaagucauccucgucacgugaccugacgucacgg

SEQ ID NO: 345 - RNA, 62 nt, Artificial Sequence
  Description of Artificial Sequence: Synthetic oligonucleotide
ucgcgucuuagugacgucacggcagccaucuugguccugacgucacugucacguggggaggg

SEQ ID NO: 346 - RNA, 76 nt, Artificial Sequence
  Description of Artificial Sequence: Synthetic oligonucleotide
ugacgucacugucacguggggagggaacacgugaacccggaagugucccuggucacgugacaugacgucacggccg

SEQ ID NO: 347 - RNA, 78 nt, Artificial Sequence
  Description of Artificial Sequence: Synthetic oligonucleotide
cgccauuuuaaguaagcauggcgggcggugaugucaaauguuaaaggucacagccggucaugcuugcacaaaauggcg

SEQ ID NO: 348 - RNA, 78 nt, Artificial Sequence
  Description of Artificial Sequence: Synthetic oligonucleotide
cgccauuuuaaguaagcauggcgggcggugacgugcaaugucaaaggucacagccugucaugcuugcacaaaauggcg

SEQ ID NO: 349 - RNA, 72 nt, Artificial Sequence
  Description of Artificial Sequence: Synthetic oligonucleotide
ccaucuuaaguaguugaggcggacgguggcgucgguucaaaggucaccaucagccacaccuacucaaaaugg

SEQ ID NO: 350 - RNA, 67 nt, Artificial Sequence
  Description of Artificial Sequence: Synthetic oligonucleotide
gccugucaugcuugcacaaaauggcggacuuccgcuuccgggucgccgccauauuuggucacgugac

SEQ ID NO: 351 - RNA, 76 nt, Artificial Sequence; identical to SEQ ID NO: 300
  Description of Artificial Sequence: Synthetic oligonucleotide

SEQ ID NO: 352 - RNA, 76 nt, Artificial Sequence; identical to SEQ ID NO: 300
  Description of Artificial Sequence: Synthetic oligonucleotide

SEQ ID NO: 353 - RNA, 78 nt, Artificial Sequence
  Description of Artificial Sequence: Synthetic oligonucleotide
gcauacgucacaagucacgugggggggacccgcuguaacccggaaguaggccccgucacgugacuuaccacgugugua

SEQ ID NO: 354 - RNA, 76 nt, Artificial Sequence
  Description of Artificial Sequence: Synthetic oligonucleotide
gccauuuuaaguagcugacgucaaggauugacgugaagguuaaaggucauccucggcggaagcuacacaaaauggu

SEQ ID NO: 355 - RNA, 78 nt, Artificial Sequence; identical to SEQ ID NO: 303
  Description of Artificial Sequence: Synthetic oligonucleotide

SEQ ID NO: 356 - RNA, 76 nt, Artificial Sequence
  Description of Artificial Sequence: Synthetic oligonucleotide
gccauuuuaagucagcucuggggaggcgugacuuccaguucaaaggucauccucaccauaacuggcacaaaauggc

SEQ ID NO: 357 - RNA, 76 nt, Artificial Sequence; identical to SEQ ID NO: 300
  Description of Artificial Sequence: Synthetic oligonucleotide

SEQ ID NO: 358 - RNA, 78 nt, Artificial Sequence
  Description of Artificial Sequence: Synthetic oligonucleotide
gcauacgucacaagucacguggaggggacacgcuguaacccggaaguaggccccgucacgugacuuaccacgugugua

SEQ ID NO: 359 - RNA, 79 nt, Artificial Sequence
  Description of Artificial Sequence: Synthetic oligonucleotide
gcgccauguuaaguggcugucgccgaggauugacgucacaguucaaaggucauccucgacgguaaccgcaaacauggcg

SEQ ID NO: 360 - RNA, 76 nt, Artificial Sequence
  Description of Artificial Sequence: Synthetic oligonucleotide
caugcgucauaagucacaugacagggguccacuuaaacacggaaguaggccccgacaugugacucgucacgugugu

SEQ ID NO: 361 - RNA, 73 nt, Artificial Sequence
  Description of Artificial Sequence: Synthetic oligonucleotide
uggcagcacuuccgaauggcugaguuuuccacgcccguccgcggagagggagccacggaggugaucccgaacg

SEQ ID NO: 362 - RNA, 78 nt, Artificial Sequence
  Description of Artificial Sequence: Synthetic oligonucleotide
gccauuuuaagucagcgcuggggaggcaugacuguaaguucaaaggucauccucaccggaacugacacaaaauggccg

SEQ ID NO: 363 - RNA, 80 nt, Artificial Sequence
  Description of Artificial Sequence: Synthetic oligonucleotide
gccaucuuaaguggcugucgccgaggauugacgucacaguucaaaggucauccucggcgguaaccgcaaagauggcgguc

SEQ ID NO: 364 - RNA, 76 nt, Artificial Sequence
  Description of Artificial Sequence: Synthetic oligonucleotide
auacgucauaagucacaugucuagggguccacuuaaacacggaaguaggccccgacaugugacucgucacgugugu

SEQ ID NO: 365 - RNA, 77 nt, Artificial Sequence
  Description of Artificial Sequence: Synthetic oligonucleotide
ccauuuuaaguaaggcggaagcagcugucccuguaacaaaauggcggcgacagccuuccgcuuugcacaaaauggag

SEQ ID NO: 366 - RNA, 80 nt, Artificial Sequence
  Description of Artificial Sequence: Synthetic oligonucleotide
gccaucuuaaguggcugucgcugaggauugacgucacaguucaaaggucauccucggcgguaaccgcaaagauggcgguc

SEQ ID NO: 367 - RNA, 76 nt, Artificial Sequence
  Description of Artificial Sequence: Synthetic oligonucleotide
cauacgucauaagucacaugacaggaguccacuuaaacacggaaguaggccccgacaugugacucgucacgugugu

SEQ ID NO: 368 - RNA, 80 nt, Artificial Sequence
  Description of Artificial Sequence: Synthetic oligonucleotide
cgccaucuuaaguggcugucgccgaggauuggcgucacaguucaaaggucauccucggcgguaaccgcaaagauggcggu

SEQ ID NO: 369 - RNA, 76 nt, Artificial Sequence
  Description of Artificial Sequence: Synthetic oligonucleotide
cauacgucauaagucacaugacagggguccacuuaaacacggaaguaggccccgacaugugacucgucacgugugu

SEQ ID NO: 370 - RNA, 78 nt, Artificial Sequence
  Description of Artificial Sequence: Synthetic oligonucleotide
gcauacgucacaagucacgugggggggacccgcuguaacccggaaguaggccccgucacgugacuuaccacguggugu

SEQ ID NO: 371 - RNA, 77 nt, Artificial Sequence
  Description of Artificial Sequence: Synthetic oligonucleotide
ccgccauuuuaggcuguugccgggcguuugacuuccguguuaaaggucaaacacccagcgacaccaaaaaauggccg

SEQ ID NO: 372 - RNA, 77 nt, Artificial Sequence
  Description of Artificial Sequence: Synthetic oligonucleotide
cuacgucauaagucacgugacagggaggggcgacaaacccggaagucauccucgcccacgugacuuaccacguggug

SEQ ID NO: 373 - RNA, 77 nt, Artificial Sequence
  Description of Artificial Sequence: Synthetic oligonucleotide
gccauuuuaaguaggugacguccaggacugacguaaaguucaaaggucauccucggcggaaccuauacaaaauggcg

SEQ ID NO: 374 - RNA, 76 nt, Artificial Sequence
  Description of Artificial Sequence: Synthetic oligonucleotide
cuacgucauaagucacguggggacggcuguacuuaaacacggaaguaggccccgucacgugauuuaccacguggug

SEQ ID NO: 375 - RNA, 73 nt, Artificial Sequence
  Description of Artificial Sequence: Synthetic oligonucleotide
gccauuuuaaguaaggcggaagagcucuagcuauacaaaauggcggcggagcacuuccgcuuugcccaaaaug

SEQ ID NO: 376 - RNA, 77 nt, Artificial Sequence
  Description of Artificial Sequence: Synthetic oligonucleotide
gccauuuuaaguagcugacgucaaggauugacguagagguuaaaggucauccucggcggaagcuacacaaaauggug

SEQ ID NO: 377 - RNA, 78 nt, Artificial Sequence; identical to SEQ ID NO: 353
  Description of Artificial Sequence: Synthetic oligonucleotide

SEQ ID NO: 378 - RNA, 80 nt, Artificial Sequence
  Description of Artificial Sequence: Synthetic oligonucleotide
ggcgccauuuuaaguaagcauggcgggcggcgacgucacaugucaaaggucaccgcacuuccgugcuugcacaaaauggc

SEQ ID NO: 379 - RNA, 73 nt, Artificial Sequence
  Description of Artificial Sequence: Synthetic oligonucleotide
ugcuacgucaucgagacacguggugccagcagcuguaaacccggaagucgcugacacacgugucuugucacgu

SEQ ID NO: 380 - RNA, 78 nt, Artificial Sequence
  Description of Artificial Sequence: Synthetic oligonucleotide
gccauuuuaaguaagcaccgccuagggaugacguauaaguucaaaggucauccucagccggaacuuacacaaaauggu

SEQ ID NO: 381 - RNA, 72 nt, Artificial Sequence
  Description of Artificial Sequence: Synthetic oligonucleotide
acgucauaugucacguggggaggcccugcugcgcaaacgcggaaguaggccccgucacgugucauaccacgu

SEQ ID NO: 382 - RNA, 77 nt, Artificial Sequence
  Description of Artificial Sequence: Synthetic oligonucleotide
ccauuuuaaguaaggcggaagcagcuccacuuucucacaaaauggcggcggggcacuuccggcuugcccaaaauggc

SEQ ID NO: 383 - RNA, 72 nt, Artificial Sequence
  Description of Artificial Sequence: Synthetic oligonucleotide
ccauuuuaaguaaggcggaaguuucuccacuauacaaaauggcggcggagcacuuccggcuugcccaaaaug

SEQ ID NO: 384 - RNA, 72 nt, Artificial Sequence
  Description of Artificial Sequence: Synthetic oligonucleotide
ccaucuuaaguaguugaggcggacgguggcgugaguucaaaggucaccaucagccacaccuacucaaaaugg

SEQ ID NO: 385 - RNA, 76 nt, Artificial Sequence
  Description of Artificial Sequence: Synthetic oligonucleotide
cgccaucuuaaguaguugaggcggacgguggcgugaguucaaaggucaccaucagccacaccuacucaaaauggug

SEQ ID NO: 386 - RNA, 73 nt, Artificial Sequence
  Description of Artificial Sequence: Synthetic oligonucleotide
uuucggaccuucggcgucgggggggucgggggcuuuacuaaacagacuccgagaugccauuggacacugaggg

SEQ ID NO: 387 - RNA, 76 nt, Artificial Sequence
  Description of Artificial Sequence: Synthetic oligonucleotide
ccauuuuaaguaggugccguccagcacugcuguuccggguuaaagggcauccucggcggaaccuauacaaaauggc

SEQ ID NO: 388 - RNA, 73 nt, Artificial Sequence
  Description of Artificial Sequence: Synthetic oligonucleotide
cuacgucaucgaugacguggggaggcguacuaugaaacgcggaaguaggccccgcuacgucaucaucacgugg

SEQ ID NO: 389 - RNA, 73 nt, Artificial Sequence
  Description of Artificial Sequence: Synthetic oligonucleotide
ccauuuuaaguaaggcggaagagcugcucuauauacaaaauggcggaggagcacuuccggcuugcccaaaaug

SEQ ID NO: 390 - RNA, 75 nt, Artificial Sequence
  Description of Artificial Sequence: Synthetic oligonucleotide
ugccuacguaacaagucacguggggaggguuggcguauaacccggaagucaauccucccacguggccugucacgu

SEQ ID NO: 391 - RNA, 72 nt, Artificial Sequence
  Description of Artificial Sequence: Synthetic oligonucleotide
uaaguaaggcggaaccaggcugucaccccgugucaaaggucaggggucagccuuccgcuuuacacaaaaugg

SEQ ID NO: 392 - RNA, 72 nt, Artificial Sequence; identical to SEQ ID NO: 391
  Description of Artificial Sequence: Synthetic oligonucleotide

SEQ ID NO: 393 - RNA, 80 nt, Artificial Sequence
  Description of Artificial Sequence: Synthetic oligonucleotide
gcagccauuuuaagucagcuucggggagggucacgcaaaguucaaaggucauccucaccggaacugguacaaaauggccg

SEQ ID NO: 394 - RNA, 74 nt, Artificial Sequence
  Description of Artificial Sequence: Synthetic oligonucleotide
ugcuacgucauaagugacguagcuggugucugcuguaaacacggaaguaggccccgccacgucacuugucacgu

SEQ ID NO: 395 - RNA, 23 nt, Artificial Sequence
  Description of Artificial Sequence: Synthetic oligonucleotide
aguagcugacgucaaggauugac

SEQ ID NO: 396 - RNA, 22 nt, Artificial Sequence
  Description of Artificial Sequence: Synthetic oligonucleotide
caagucacguggaggggacccg

SEQ ID NO: 397 - RNA, 25 nt, Artificial Sequence
  Description of Artificial Sequence: Synthetic oligonucleotide
aaguagcugacgucaaggauugacg

SEQ ID NO: 398 - RNA, 23 nt, Artificial Sequence
  Description of Artificial Sequence: Synthetic oligonucleotide
auaagucacgugguggggacccg

SEQ ID NO: 399 - RNA, 26 nt, Artificial Sequence
  Description of Artificial Sequence: Synthetic oligonucleotide
uggggaggguuggcguauagcccgga

SEQ ID NO: 400 - RNA, 24 nt, Artificial Sequence
  Description of Artificial Sequence: Synthetic oligonucleotide
ccccccccggggggggguuugccc

SEQ ID NO: 401 - RNA, 25 nt, Artificial Sequence
  Description of Artificial Sequence: Synthetic oligonucleotide
aucagucacgugggggaaggcgugc

SEQ ID NO: 402 - RNA, 22 nt, Artificial Sequence
  Description of Artificial Sequence: Synthetic oligonucleotide
aaguaaggcggaagcagcucgg

SEQ ID NO: 403 - RNA, 21 nt, Artificial Sequence
  Description of Artificial Sequence: Synthetic oligonucleotide
agucacguggggaggguuggc

SEQ ID NO: 404 - RNA, 22 nt, Artificial Sequence
  Description of Artificial Sequence: Synthetic oligonucleotide
cccgaaggcggguacccgaggu

SEQ ID NO: 405 - RNA, 23 nt, Artificial Sequence
  Description of Artificial Sequence: Synthetic oligonucleotide
uaucguagccgacgcggaccccg

SEQ ID NO: 406 - RNA, 26 nt, Artificial Sequence
  Description of Artificial Sequence: Synthetic oligonucleotide
auuuugugauacgcgcguccccuccc

SEQ ID NO: 407 - RNA, 21 nt, Artificial Sequence
  Description of Artificial Sequence: Synthetic oligonucleotide
aagucacgugacugggcaggu

SEQ ID NO: 408 - RNA, 26 nt, Artificial Sequence
  Description of Artificial Sequence: Synthetic oligonucleotide
ugacgucaaagucacgugggragggu

SEQ ID NO: 409 - RNA, 23 nt, Artificial Sequence
  Description of Artificial Sequence: Synthetic oligonucleotide
gaucgagcgucccgugggcgggu

SEQ ID NO: 410 - RNA, 28 nt, Artificial Sequence
  Description of Artificial Sequence: Synthetic oligonucleotide
ugacgucaaagucacguggggagggcgg

SEQ ID NO: 411 - RNA, 23 nt, Artificial Sequence
  Description of Artificial Sequence: Synthetic oligonucleotide
gaucgagcgucccgugggcgggu

SEQ ID NO: 412 - RNA, 23 nt, Artificial Sequence
  Description of Artificial Sequence: Synthetic oligonucleotide
gaucgagcgucccgugggcgggu

SEQ ID NO: 413 - RNA, 28 nt, Artificial Sequence
  Description of Artificial Sequence: Synthetic oligonucleotide
ugacgucaaagucacguggggagggcgg

SEQ ID NO: 414 - RNA, 26 nt, Artificial Sequence
  Description of Artificial Sequence: Synthetic oligonucleotide
acgugguaagucacgugggggcagcu

SEQ ID NO: 415 - RNA, 25 nt, Artificial Sequence
  Description of Artificial Sequence: Synthetic oligonucleotide
auuuuauaauacgcgcguccccucc

SEQ ID NO: 416 - RNA, 22 nt, Artificial Sequence
  Description of Artificial Sequence: Synthetic oligonucleotide
aagggacagccuuccggcuugc

SEQ ID NO: 417 - RNA, 25 nt, Artificial Sequence
  Description of Artificial Sequence: Synthetic oligonucleotide
cauaagucacguggggacggcugcu

SEQ ID NO: 418 - RNA, 24 nt, Artificial Sequence
  Description of Artificial Sequence: Synthetic oligonucleotide
uaaggcggaacaggcugacacccc

SEQ ID NO: 419 - RNA, 28 nt, Artificial Sequence
  Description of Artificial Sequence: Synthetic oligonucleotide
uacgucauaagucacgugggaagagcug

SEQ ID NO: 420 - RNA, 22 nt, Artificial Sequence
  Description of Artificial Sequence: Synthetic oligonucleotide
ucgggcgucccgagggcgggug

SEQ ID NO: 421 - RNA, 25 nt, Artificial Sequence
  Description of Artificial Sequence: Synthetic oligonucleotide
aaagugaguggggccagacuucgcc

SEQ ID NO: 422 - RNA, 22 nt, Artificial Sequence
  Description of Artificial Sequence: Synthetic oligonucleotide
cucucacgugaugggggcgugc

SEQ ID NO: 423 - RNA, 28 nt, Artificial Sequence
  Description of Artificial Sequence: Synthetic oligonucleotide
ucuacuacguacacuuccuggggugugu

SEQ ID NO: 424 - RNA, 22 nt, Artificial Sequence
  Description of Artificial Sequence: Synthetic oligonucleotide
uggcuguugucacgugacuuga

SEQ ID NO: 425 - RNA, 24 nt, Artificial Sequence
  Description of Artificial Sequence: Synthetic oligonucleotide
agacgccauuuuaggcccucgcgg

SEQ ID NO: 426 - RNA, 25 nt, Artificial Sequence
  Description of Artificial Sequence: Synthetic oligonucleotide
ugucgugacguuugagacacgugau

SEQ ID NO: 427 - RNA, 30 nt, Artificial Sequence
  Description of Artificial Sequence: Synthetic oligonucleotide
ugacguuugagacacgugaugggggcgugc

SEQ ID NO: 428 - RNA, 22 nt, Artificial Sequence
  Description of Artificial Sequence: Synthetic oligonucleotide
agugaguggggccagacuucgc

SEQ ID NO: 429 - RNA, 30 nt, Artificial Sequence
  Description of Artificial Sequence: Synthetic oligonucleotide
ugugacgucaaagucacguggggagggcgg

SEQ ID NO: 430 - RNA, 27 nt, Artificial Sequence
  Description of Artificial Sequence: Synthetic oligonucleotide
aaaagaggaaguaugacguagcggcgg

SEQ ID NO: 431 - RNA, 21 nt, Artificial Sequence
  Description of Artificial Sequence: Synthetic oligonucleotide
agcgagcggccgagcgacccg

SEQ ID NO: 432 - RNA, 24 nt, Artificial Sequence
  Description of Artificial Sequence: Synthetic oligonucleotide
uucagucacguggggagggaacgc

SEQ ID NO: 433 - RNA, 24 nt, Artificial Sequence
  Description of Artificial Sequence: Synthetic oligonucleotide
aaaagaggaagugugacguagcgg

SEQ ID NO: 434 - RNA, 30 nt, Artificial Sequence; identical to SEQ ID NO: 429
  Description of Artificial Sequence: Synthetic oligonucleotide

SEQ ID NO: 435 - RNA, 24 nt, Artificial Sequence
  Description of Artificial Sequence: Synthetic oligonucleotide
aaaagaggaaguaugacguggcgg

SEQ ID NO: 436 - RNA, 28 nt, Artificial Sequence
  Description of Artificial Sequence: Synthetic oligonucleotide
ugacgucaaagucacguggggagggcgg

SEQ ID NO: 437 - RNA, 29 nt, Artificial Sequence
  Description of Artificial Sequence: Synthetic oligonucleotide
aaaaaagaggaagugugacguagcggcgg

SEQ ID NO: 438 - RNA, 23 nt, Artificial Sequence
  Description of Artificial Sequence: Synthetic oligonucleotide
gaucgagcgucccgugggcgggu

SEQ ID NO: 439 - RNA, 30 nt, Artificial Sequence
  Description of Artificial Sequence: Synthetic oligonucleotide
ugugacgucaaagucacguggggaggaggg

SEQ ID NO: 440 - RNA, 21 nt, Artificial Sequence
  Description of Artificial Sequence: Synthetic oligonucleotide
uugguccugacgucacuguca

SEQ ID NO: 441 - RNA, 27 nt, Artificial Sequence
  Description of Artificial Sequence: Synthetic oligonucleotide
cgucacugucacguggggagggaacac

SEQ ID NO: 442 - RNA, 24 nt, Artificial Sequence
  Description of Artificial Sequence: Synthetic oligonucleotide
uaaguaagcauggcgggcggugau

SEQ ID NO: 443 - RNA, 22 nt, Artificial Sequence
  Description of Artificial Sequence: Synthetic oligonucleotide
aaguaagcauggcgggcgguga

SEQ ID NO: 444 - RNA, 24 nt, Artificial Sequence
  Description of Artificial Sequence: Synthetic oligonucleotide
uaaguaguugaggcggacgguggc

SEQ ID NO: 445 - RNA, 29 nt, Artificial Sequence
  Description of Artificial Sequence: Synthetic oligonucleotide
ucaugcuugcacaaaauggcggacuuccg

SEQ ID NO: 446 - RNA, 23 nt, Artificial Sequence
  Description of Artificial Sequence: Synthetic oligonucleotide
aguagcugacgucaaggauugac

SEQ ID NO: 447 - RNA, 23 nt, Artificial Sequence
  Description of Artificial Sequence: Synthetic oligonucleotide
aguagcugacgucaaggauugac

SEQ ID NO: 448 - RNA, 23 nt, Artificial Sequence
  Description of Artificial Sequence: Synthetic oligonucleotide
acaagucacgugggggggacccg

SEQ ID NO: 449 - RNA, 25 nt, Artificial Sequence
  Description of Artificial Sequence: Synthetic oligonucleotide
aaguagcugacgucaaggauugacg

SEQ ID NO: 450 - RNA, 23 nt, Artificial Sequence
  Description of Artificial Sequence: Synthetic oligonucleotide
auaagucacgugguggggacccg

SEQ ID NO: 451 - RNA, 26 nt, Artificial Sequence
  Description of Artificial Sequence: Synthetic oligonucleotide
aagucagcucuggggaggcgugacuu

SEQ ID NO: 452 - RNA, 23 nt, Artificial Sequence
  Description of Artificial Sequence: Synthetic oligonucleotide
aguagcugacgucaaggauugac

SEQ ID NO: 453 - RNA, 22 nt, Artificial Sequence
  Description of Artificial Sequence: Synthetic oligonucleotide
caagucacguggaggggacacg

SEQ ID NO: 454 - RNA, 27 nt, Artificial Sequence
  Description of Artificial Sequence: Synthetic oligonucleotide
uguuaaguggcugucgccgaggauuga

SEQ ID NO: 455 - RNA, 22 nt, Artificial Sequence
  Description of Artificial Sequence: Synthetic oligonucleotide
uaagucacaugacaggggucca

SEQ ID NO: 456 - RNA, 22 nt, Artificial Sequence
  Description of Artificial Sequence: Synthetic oligonucleotide
cggagagggagccacggaggug

SEQ ID NO: 457 - RNA, 23 nt, Artificial Sequence
  Description of Artificial Sequence: Synthetic oligonucleotide
aagucagcgcuggggaggcauga

SEQ ID NO: 458 - RNA, 28 nt, Artificial Sequence
  Description of Artificial Sequence: Synthetic oligonucleotide
ucuuaaguggcugucgccgaggauugac

SEQ ID NO: 459 - RNA, 24 nt, Artificial Sequence
  Description of Artificial Sequence: Synthetic oligonucleotide
aagucacaugucuagggguccacu

SEQ ID NO: 460 - RNA, 23 nt, Artificial Sequence
  Description of Artificial Sequence: Synthetic oligonucleotide
aaguaaggcggaagcagcugucc

SEQ ID NO: 461 - RNA, 29 nt, Artificial Sequence
  Description of Artificial Sequence: Synthetic oligonucleotide
aucuuaaguggcugucgcugaggauugac

SEQ ID NO: 462 - RNA, 24 nt, Artificial Sequence
  Description of Artificial Sequence: Synthetic oligonucleotide
uaagucacaugacaggaguccacu

SEQ ID NO: 463 - RNA, 22 nt, Artificial Sequence
  Description of Artificial Sequence: Synthetic oligonucleotide
aaguggcugucgccgaggauug

SEQ ID NO: 464 - RNA, 22 nt, Artificial Sequence
  Description of Artificial Sequence: Synthetic oligonucleotide
uaagucacaugacaggggucca

SEQ ID NO: 465 - RNA, 23 nt, Artificial Sequence
  Description of Artificial Sequence: Synthetic oligonucleotide
acaagucacgugggggggacccg

SEQ ID NO: 466 - RNA, 28 nt, Artificial Sequence
  Description of Artificial Sequence: Synthetic oligonucleotide
auuuuaggcuguugccgggcguuugacu

SEQ ID NO: 467 - RNA, 23 nt, Artificial Sequence
  Description of Artificial Sequence: Synthetic oligonucleotide
auaagucacgugacagggagggg

SEQ ID NO: 468 - RNA, 21 nt, Artificial Sequence
  Description of Artificial Sequence: Synthetic oligonucleotide
aaguaggugacguccaggacu

SEQ ID NO: 469 - RNA, 24 nt, Artificial Sequence
  Description of Artificial Sequence: Synthetic oligonucleotide
cauaagucacguggggacggcugu

SEQ ID NO: 470 - RNA, 26 nt, Artificial Sequence
  Description of Artificial Sequence: Synthetic oligonucleotide
uaaguaaggcggaagagcucuagcua

SEQ ID NO: 471 - RNA, 23 nt, Artificial Sequence
  Description of Artificial Sequence: Synthetic oligonucleotide
aguagcugacgucaaggauugac

SEQ ID NO: 472 - RNA, 23 nt, Artificial Sequence
  Description of Artificial Sequence: Synthetic oligonucleotide
acaagucacgugggggggacccg

SEQ ID NO: 473 - RNA, 24 nt, Artificial Sequence
  Description of Artificial Sequence: Synthetic oligonucleotide
uaaguaagcauggcgggcggcgac

SEQ ID NO: 474 - RNA, 25 nt, Artificial Sequence
  Description of Artificial Sequence: Synthetic oligonucleotide
aucgagacacguggugccagcagcu

SEQ ID NO: 475 - RNA, 30 nt, Artificial Sequence
  Description of Artificial Sequence: Synthetic oligonucleotide
ucauccucagccggaacuuacacaaaaugg

SEQ ID NO: 476 - RNA, 26 nt, Artificial Sequence
  Description of Artificial Sequence: Synthetic oligonucleotide
auaugucacguggggaggcccugcug

SEQ ID NO: 477 - RNA, 26 nt, Artificial Sequence
  Description of Artificial Sequence: Synthetic oligonucleotide
aaguaaggcggaagcagcuccacuuu

SEQ ID NO: 478 - RNA, 24 nt, Artificial Sequence
  Description of Artificial Sequence: Synthetic oligonucleotide
aaguaaggcggaaguuucuccacu

SEQ ID NO: 479 - RNA, 24 nt, Artificial Sequence
  Description of Artificial Sequence: Synthetic oligonucleotide
uaaguaguugaggcggacgguggc

SEQ ID NO: 480 - RNA, 23 nt, Artificial Sequence
  Description of Artificial Sequence: Synthetic oligonucleotide
uaaguaguugaggcggacggugg

SEQ ID NO: 481 - RNA, 27 nt, Artificial Sequence
  Description of Artificial Sequence: Synthetic oligonucleotide
gaccuucggcgucgggggggucggggg

SEQ ID NO: 482 - RNA, 19 nt, Artificial Sequence
  Description of Artificial Sequence: Synthetic oligonucleotide
auccucggcggaaccuaua

SEQ ID NO: 483 - RNA, 26 nt, Artificial Sequence
  Description of Artificial Sequence: Synthetic oligonucleotide
aucgaugacguggggaggcguacuau

SEQ ID NO: 484 - RNA, 24 nt, Artificial Sequence
  Description of Artificial Sequence: Synthetic oligonucleotide
uggcggaggagcacuuccggcuug

SEQ ID NO: 485 - RNA, 25 nt, Artificial Sequence
  Description of Artificial Sequence: Synthetic oligonucleotide
aacaagucacguggggaggguuggc

SEQ ID NO: 486 - RNA, 21 nt, Artificial Sequence
  Description of Artificial Sequence: Synthetic oligonucleotide
aggggucagccuuccgcuuua

SEQ ID NO: 487 - RNA, 21 nt, Artificial Sequence
  Description of Artificial Sequence: Synthetic oligonucleotide
aggggucagccuuccgcuuua

SEQ ID NO: 488 - RNA, 24 nt, Artificial Sequence
  Description of Artificial Sequence: Synthetic oligonucleotide
uaagucagcuucggggagggucac

SEQ ID NO: 489 - RNA, 27 nt, Artificial Sequence
  Description of Artificial Sequence: Synthetic oligonucleotide
ucauaagugacguagcuggugucugcu

SEQ ID NO: 490 - RNA, 23 nt, Artificial Sequence
  Description of Artificial Sequence: Synthetic oligonucleotide
cauccucggcggaagcuacacaa

SEQ ID NO: 491 - RNA, 23 nt, Artificial Sequence
  Description of Artificial Sequence: Synthetic oligonucleotide
ggccccgucacgugacuuaccac

SEQ ID NO: 492 - RNA, 24 nt, Artificial Sequence
  Description of Artificial Sequence: Synthetic oligonucleotide
ucauccucggcggaagcuacacaa

SEQ ID NO: 493 - RNA, 23 nt, Artificial Sequence
  Description of Artificial Sequence: Synthetic oligonucleotide
ggccccgucacgugauuugucac

SEQ ID NO: 494 - RNA, 27 nt, Artificial Sequence
  Description of Artificial Sequence: Synthetic oligonucleotide
ccgggucauaggucacaccuacgucac

SEQ ID NO: 495 - RNA, 28 nt, Artificial Sequence
  Description of Artificial Sequence: Synthetic oligonucleotide
ggcugccgccccccccggggaaaggggg

SEQ ID NO: 496 - RNA, 22 nt, Artificial Sequence
  Description of Artificial Sequence: Synthetic oligonucleotide
auccucguccacgugacuguga

SEQ ID NO: 497 - RNA, 21 nt, Artificial Sequence
  Description of Artificial Sequence: Synthetic oligonucleotide
gagcacuuccggcuugcccaa

SEQ ID NO: 498 - RNA, 20 nt, Artificial Sequence
  Description of Artificial Sequence: Synthetic oligonucleotide
caauccucuuacguggccug

SEQ ID NO: 499 - RNA, 24 nt, Artificial Sequence
  Description of Artificial Sequence: Synthetic oligonucleotide
cgagguuaagggccaauucgggcu

SEQ ID NO: 500 - RNA, 23 nt, Artificial Sequence
  Description of Artificial Sequence: Synthetic oligonucleotide
gggcccccgcggggcucucggcg

SEQ ID NO: 501 - RNA, 27 nt, Artificial Sequence
  Description of Artificial Sequence: Synthetic oligonucleotide
gcggggcguggccguaucagaaaaugg

SEQ ID NO: 502 - RNA, 19 nt, Artificial Sequence
  Description of Artificial Sequence: Synthetic oligonucleotide
ccucggucacguggccugu

SEQ ID NO: 503 - RNA, 26 nt, Artificial Sequence
  Description of Artificial Sequence: Synthetic oligonucleotide
ccucgucacgugaccugacgucacag

SEQ ID NO: 504 - RNA, 25 nt, Artificial Sequence
  Description of Artificial Sequence: Synthetic oligonucleotide
ccguccgcggcgagagcgcgagcga

SEQ ID NO: 505 - RNA, 27 nt, Artificial Sequence
  Description of Artificial Sequence: Synthetic oligonucleotide
auccucgucacgugaccugacgucacg

SEQ ID NO: 506 - RNA, 25 nt, Artificial Sequence
  Description of Artificial Sequence: Synthetic oligonucleotide
ccguccgcggcgagagcgcgagcga

SEQ ID NO: 507 - RNA, 25 nt, Artificial Sequence
  Description of Artificial Sequence: Synthetic oligonucleotide
ccguccgcggcgagagcgcgagcga

SEQ ID NO: 508 - RNA, 27 nt, Artificial Sequence
  Description of Artificial Sequence: Synthetic oligonucleotide
auccucgucacgugaccugacgucacg

SEQ ID NO: 509 - RNA, 25 nt, Artificial Sequence
  Description of Artificial Sequence: Synthetic oligonucleotide
cugacccgcgugacuggucacguga

SEQ ID NO: 510 - RNA, 26 nt, Artificial Sequence
  Description of Artificial Sequence: Synthetic oligonucleotide
cggggcguggccguauuagaaaaugg

SEQ ID NO: 511 - RNA, 29 nt, Artificial Sequence
  Description of Artificial Sequence: Synthetic oligonucleotide
aguaaggcggaaccaggcugucacccugu

SEQ ID NO: 512 - RNA, 24 nt, Artificial Sequence
  Description of Artificial Sequence: Synthetic oligonucleotide
uagcugacccgcgugacuugucac

SEQ ID NO: 513 - RNA, 19 nt, Artificial Sequence
  Description of Artificial Sequence: Synthetic oligonucleotide
ggucagccuccgcuuugca

SEQ ID NO: 514 - RNA, 28 nt, Artificial Sequence
  Description of Artificial Sequence: Synthetic oligonucleotide
gcugacccgcguggcuugucacgugagu

SEQ ID NO: 515 - RNA, 25 nt, Artificial Sequence
  Description of Artificial Sequence: Synthetic oligonucleotide
ggcccguccgcggcgagagcgcgag

SEQ ID NO: 516 - RNA, 27 nt, Artificial Sequence
  Description of Artificial Sequence: Synthetic oligonucleotide
uccgggugcgucugggggccgccauuu

SEQ ID NO: 517 - RNA, 21 nt, Artificial Sequence
  Description of Artificial Sequence: Synthetic oligonucleotide
auccucgaccacgugacugug

SEQ ID NO: 518 - RNA, 30 nt, Artificial Sequence
  Description of Artificial Sequence: Synthetic oligonucleotide
auaaaccagaggggugacgaaugguagagu

SEQ ID NO: 519 - RNA, 23 nt, Artificial Sequence
  Description of Artificial Sequence: Synthetic oligonucleotide
caaagucacguggugacggccau

SEQ ID NO: 520 - RNA, 26 nt, Artificial Sequence
  Description of Artificial Sequence: Synthetic oligonucleotide
guaggcgcguuuuaaugacgucacgg

SEQ ID NO: 521 - RNA, 28 nt, Artificial Sequence
  Description of Artificial Sequence: Synthetic oligonucleotide
uaggcgcguuuuaaugacgucacggcag

SEQ ID NO: 522 - RNA, 29 nt, Artificial Sequence
  Description of Artificial Sequence: Synthetic oligonucleotide
aucccuggucacgugacucugacgucacg

SEQ ID NO: 523 - RNA, 26 nt, Artificial Sequence
  Description of Artificial Sequence: Synthetic oligonucleotide
gcgugugggggccgccauuuuagcuu

SEQ ID NO: 524 - RNA, 29 nt, Artificial Sequence
  Description of Artificial Sequence: Synthetic oligonucleotide
ucauccucgucacgugaccugacgucacg

SEQ ID NO: 525 - RNA, 29 nt, Artificial Sequence
  Description of Artificial Sequence: Synthetic oligonucleotide
cgccaucuugugacuuccuuccgcuuuuu

SEQ ID NO: 526 - RNA, 22 nt, Artificial Sequence
  Description of Artificial Sequence: Synthetic oligonucleotide
uagaguuuuuuccgcccguccg

SEQ ID NO: 527 - RNA, 23 nt, Artificial Sequence
  Description of Artificial Sequence: Synthetic oligonucleotide
gucccuggucacgugauugugac

SEQ ID NO: 528 - RNA, 26 nt, Artificial Sequence
  Description of Artificial Sequence: Synthetic oligonucleotide
cauuuugugacuuccuuccgcuuuuu

SEQ ID NO: 529 - RNA, 29 nt, Artificial Sequence
  Description of Artificial Sequence: Synthetic oligonucleotide
ucauccucgucacgugaccugacgucacg

SEQ ID NO: 530 - RNA, 30 nt, Artificial Sequence
  Description of Artificial Sequence: Synthetic oligonucleotide
ccgccaucuugugacuuccuuccgcuuuuu

SEQ ID NO: 531 - RNA, 27 nt, Artificial Sequence
  Description of Artificial Sequence: Synthetic oligonucleotide
auccucgucacgugaccugacgucacg

SEQ ID NO: 532 - RNA, 30 nt, Artificial Sequence
  Description of Artificial Sequence: Synthetic oligonucleotide
cgccaucuugugacuuccuuccgcuuuuuc

SEQ ID NO: 533 - RNA, 25 nt, Artificial Sequence
  Description of Artificial Sequence: Synthetic oligonucleotide
ccguccgcggcgagagcgcgagcga

SEQ ID NO: 534 - RNA, 29 nt, Artificial Sequence
  Description of Artificial Sequence: Synthetic oligonucleotide
ucauccucgucacgugaccugacgucacg

SEQ ID NO: 535 - RNA, 23 nt, Artificial Sequence
  Description of Artificial Sequence: Synthetic oligonucleotide
cuuagugacgucacggcagccau

SEQ ID NO: 536 - RNA, 25 nt, Artificial Sequence
  Description of Artificial Sequence: Synthetic oligonucleotide
gucccuggucacgugacaugacguc

SEQ ID NO: 537 - RNA, 24 nt, Artificial Sequence
  Description of Artificial Sequence: Synthetic oligonucleotide
cacagccggucaugcuugcacaaa

SEQ ID NO: 538 - RNA, 22 nt, Artificial Sequence
  Description of Artificial Sequence: Synthetic oligonucleotide
acagccugucaugcuugcacaa

SEQ ID NO: 539 - RNA, 24 nt, Artificial Sequence
  Description of Artificial Sequence: Synthetic oligonucleotide
caccaucagccacaccuacucaaa

SEQ ID NO: 540 - RNA, 28 nt, Artificial Sequence
  Description of Artificial Sequence: Synthetic oligonucleotide
cgggucgccgccauauuuggucacguga

SEQ ID NO: 541 - RNA, 23 nt, Artificial Sequence
  Description of Artificial Sequence: Synthetic oligonucleotide
cauccucggcggaagcuacacaa

SEQ ID NO: 542 - RNA, 23 nt, Artificial Sequence
  Description of Artificial Sequence: Synthetic oligonucleotide
cauccucggcggaagcuacacaa

SEQ ID NO: 543 - RNA, 23 nt, Artificial Sequence
  Description of Artificial Sequence: Synthetic oligonucleotide
ggccccgucacgugacuuaccac

SEQ ID NO: 544 - RNA, 24 nt, Artificial Sequence
  Description of Artificial Sequence: Synthetic oligonucleotide
ucauccucggcggaagcuacacaa

SEQ ID NO: 545 - RNA, 23 nt, Artificial Sequence
  Description of Artificial Sequence: Synthetic oligonucleotide
ggccccgucacgugauuugucac

SEQ ID NO: 546 - RNA, 25 nt, Artificial Sequence
  Description of Artificial Sequence: Synthetic oligonucleotide
gucauccucaccauaacuggcacaa

SEQ ID NO: 547 - RNA, 23 nt, Artificial Sequence
  Description of Artificial Sequence: Synthetic oligonucleotide
cauccucggcggaagcuacacaa

SEQ ID NO: 548 - RNA, 23 nt, Artificial Sequence
  Description of Artificial Sequence: Synthetic oligonucleotide
ggccccgucacgugacuuaccac

SEQ ID NO: 549 - RNA, 25 nt, Artificial Sequence
  Description of Artificial Sequence: Synthetic oligonucleotide
auccucgacgguaaccgcaaacaug

SEQ ID NO: 550 - RNA, 21 nt, Artificial Sequence
  Description of Artificial Sequence: Synthetic oligonucleotide
ggccccgacaugugacucguc

SEQ ID NO: 551 - RNA, 27 nt, Artificial Sequence
  Description of Artificial Sequence: Synthetic oligonucleotide
agcacuuccgaauggcugaguuuucca

SEQ ID NO: 552 - RNA, 22 nt, Artificial Sequence
  Description of Artificial Sequence: Synthetic oligonucleotide
auccucaccggaacugacacaa

SEQ ID NO: 553 - RNA, 26 nt, Artificial Sequence
  Description of Artificial Sequence: Synthetic oligonucleotide
cauccucggcgguaaccgcaaagaug

SEQ ID NO: 554 - RNA, 22 nt, Artificial Sequence
  Description of Artificial Sequence: Synthetic oligonucleotide
uaggccccgacaugugacucgu

SEQ ID NO: 555 - RNA, 21 nt, Artificial Sequence
  Description of Artificial Sequence: Synthetic oligonucleotide
acagccuuccgcuuugcacaa

SEQ ID NO: 556 - RNA, 27 nt, Artificial Sequence
  Description of Artificial Sequence: Synthetic oligonucleotide
cauccucggcgguaaccgcaaagaugg

SEQ ID NO: 557 - RNA, 23 nt, Artificial Sequence
  Description of Artificial Sequence: Synthetic oligonucleotide
uaggccccgacaugugacucguc

SEQ ID NO: 558 - RNA, 20 nt, Artificial Sequence
  Description of Artificial Sequence: Synthetic oligonucleotide
uccucggcgguaaccgcaaa

SEQ ID NO: 559 - RNA, 21 nt, Artificial Sequence
  Description of Artificial Sequence: Synthetic oligonucleotide
ggccccgacaugugacucguc

SEQ ID NO: 560 - RNA, 23 nt, Artificial Sequence
  Description of Artificial Sequence: Synthetic oligonucleotide
ggccccgucacgugacuuaccac

SEQ ID NO: 561 - RNA, 28 nt, Artificial Sequence
  Description of Artificial Sequence: Synthetic oligonucleotide
ucaaacacccagcgacaccaaaaaaugg

SEQ ID NO: 562 - RNA, 22 nt, Artificial Sequence
  Description of Artificial Sequence: Synthetic oligonucleotide
ccucgcccacgugacuuaccac

SEQ ID NO: 563 - RNA, 20 nt, Artificial Sequence
  Description of Artificial Sequence: Synthetic oligonucleotide
ccucggcggaaccuauacaa

SEQ ID NO: 564 - RNA, 22 nt, Artificial Sequence
  Description of Artificial Sequence: Synthetic oligonucleotide
gccccgucacgugauuuaccac

SEQ ID NO: 565 - RNA, 28 nt, Artificial Sequence
  Description of Artificial Sequence: Synthetic oligonucleotide
gcggcggagcacuuccgcuuugcccaaa

SEQ ID NO: 566 - RNA, 23 nt, Artificial Sequence
  Description of Artificial Sequence: Synthetic oligonucleotide
cauccucggcggaagcuacacaa

SEQ ID NO: 567 - RNA, 23 nt, Artificial Sequence
  Description of Artificial Sequence: Synthetic oligonucleotide
ggccccgucacgugacuuaccac

SEQ ID NO: 568 - RNA, 25 nt, Artificial Sequence
  Description of Artificial Sequence: Synthetic oligonucleotide
caccgcacuuccgugcuugcacaaa

SEQ ID NO: 569 - RNA, 24 nt, Artificial Sequence
  Description of Artificial Sequence: Synthetic oligonucleotide
ucgcugacacacgugucuugucac

SEQ ID NO: 570 - RNA, 30 nt, Artificial Sequence
  Description of Artificial Sequence: Synthetic oligonucleotide
cauuuuaaguaagcaccgccuagggaugac

SEQ ID NO: 571 - RNA, 26 nt, Artificial Sequence
  Description of Artificial Sequence: Synthetic oligonucleotide
guaggccccgucacgugucauaccac

SEQ ID NO: 572 - RNA, 25 nt, Artificial Sequence
  Description of Artificial Sequence: Synthetic oligonucleotide
ggcggggcacuuccggcuugcccaa

SEQ ID NO: 573 - RNA, 26 nt, Artificial Sequence
  Description of Artificial Sequence: Synthetic oligonucleotide
cggcggagcacuuccggcuugcccaa

SEQ ID NO: 574 - RNA, 24 nt, Artificial Sequence
  Description of Artificial Sequence: Synthetic oligonucleotide
caccaucagccacaccuacucaaa

SEQ ID NO: 575 - RNA, 23 nt, Artificial Sequence
  Description of Artificial Sequence: Synthetic oligonucleotide
accaucagccacaccuacucaaa

SEQ ID NO: 576 - RNA, 28 nt, Artificial Sequence
  Description of Artificial Sequence: Synthetic oligonucleotide
gacuccgagaugccauuggacacugagg

SEQ ID NO: 577 - RNA, 18 nt, Artificial Sequence
  Description of Artificial Sequence: Synthetic oligonucleotide
aguaggugccguccagca

SEQ ID NO: 578 - RNA, 27 nt, Artificial Sequence
  Description of Artificial Sequence: Synthetic oligonucleotide
aaguaggccccgcuacgucaucaucac

SEQ ID NO: 579 - RNA, 23 nt, Artificial Sequence
  Description of Artificial Sequence: Synthetic oligonucleotide
aaggcggaagagcugcucuauau

SEQ ID NO: 580 - RNA, 24 nt, Artificial Sequence
  Description of Artificial Sequence: Synthetic oligonucleotide
caauccucccacguggccugucac

SEQ ID NO: 581 - RNA, 26 nt, Artificial Sequence
  Description of Artificial Sequence: Synthetic oligonucleotide
aaggcggaaccaggcugucaccccgu

SEQ ID NO: 582 - RNA, 26 nt, Artificial Sequence
  Description of Artificial Sequence: Synthetic oligonucleotide
aaggcggaaccaggcugucaccccgu

SEQ ID NO: 583 - RNA, 24 nt, Artificial Sequence
  Description of Artificial Sequence: Synthetic oligonucleotide
cauccucaccggaacugguacaaa

SEQ ID NO: 584 - RNA, 26 nt, Artificial Sequence
  Description of Artificial Sequence: Synthetic oligonucleotide
uaggccccgccacgucacuugucacg

SEQ ID NO: 585-689 - skipped (105 entries)

SEQ ID NO: 690 - DNA, 22 bp, Artificial Sequence
  Description of Artificial Sequence: Synthetic primer
attcgaatggctgagtttatgc

SEQ ID NO: 691 - DNA, 24 bp, Artificial Sequence
  Description of Artificial Sequence: Synthetic primer
cacgaattagccaagactgggcac

SEQ ID NO: 692 - DNA, 20 bp, Artificial Sequence
  Description of Artificial Sequence: Synthetic primer
gctcccactcctgatttctg

SEQ ID NO: 693 - DNA, 21 bp, Artificial Sequence
  Description of Artificial Sequence: Synthetic primer
ccttgactacggtggtttcac

SEQ ID NO: 694 - DNA, 22 bp, Artificial Sequence
  Description of Artificial Sequence: Synthetic primer
tgcaggcattcgagggcttgtt

SEQ ID NO: 695 - DNA, 20 bp, Artificial Sequence
  Description of Artificial Sequence: Synthetic primer
tttaaccccctagtcccagg

SEQ ID NO: 696-742 - skipped (47 entries)

SEQ ID NO: 743 - DNA, 117 bp, Artificial Sequence; identical to SEQ ID NO: 120
  Description of Artificial Sequence: Synthetic polynucleotide
  misc_feature (10)..(10): May or may not be present
  misc_feature (12)..(12): May or may not be present
  misc_feature (30)..(32): May or may not be present
  misc_feature (34)..(34): May or may not be present
  misc_feature (43)..(46): May or may not be present
  misc_feature (52)..(54): May or may not be present
  misc_feature (70)..(71): May or may not be present
  misc_feature (89)..(90): May or may not be present
  misc_feature (103)..(103): May or may not be present

SEQ ID NO: 744-800 - skipped (57 entries)

SEQ ID NO: 801 - DNA, 156 bp, Artificial Sequence; identical to SEQ ID NO: 125
  Description of Artificial Sequence: Synthetic polynucleotide

SEQ ID NO: 802 - DNA, 150 bp, Artificial Sequence
  Description of Artificial Sequence: Synthetic polynucleotide
ccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 803 - DNA, 122 bp, Artificial Sequence; identical to SEQ ID NO: 153
  Description of Artificial Sequence: Synthetic polynucleotide

SEQ ID NO: 804 - DNA, 111 bp, Artificial Sequence
  Description of Artificial Sequence: Synthetic polynucleotide
cggcccagcggcggcgcgcgcgcttcgcgcgcgcgccggggggctccgcccccccccgcgcatgcgcggggcccccccccgcggggggctccgccccccggtccccccccg

SEQ ID NO: 805 - DNA, 115 bp, Artificial Sequence
  Description of Artificial Sequence: Synthetic polynucleotide
cggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 806 - DNA, 104 bp, Artificial Sequence; identical to SEQ ID NO: 144
  Description of Artificial Sequence: Synthetic polynucleotide

SEQ ID NO: 807 - DNA, 108 bp, Artificial Sequence
  Description of Artificial Sequence: Synthetic polynucleotide
ggcggcggcgcgcgcgctacgcgcgcgcgccggggagctctgcccccccccgcgcatgcgcgcgggtcccccccccgcggggggctccgccccccggtcccccccccg

SEQ ID NO: 808-828 - skipped (21 entries)

SEQ ID NO: 829 - protein, 11 aa, Artificial Sequence
  Description of Artificial Sequence: Synthetic peptide
  MOD_RES (4)..(5): Any amino acid
  MOD_RES (7)..(7): Any amino acid
  MOD_RES (9)..(10): Any amino acid
YNPXXDXGXXN

SEQ ID NO: 830-885 - skipped (56 entries)

SEQ ID NO: 886 - DNA, 3176 bp, Gammatorquevirus sp.
taaaatggcgggagccaatcattttatactttcactttccaattaaaaatggccacgtcacaaacaaggggtggagccatttaaactatataactaagtggggtggcgaatggctgagtttaccccgctagacggtgcagggaccggatcgagcgcagcgaggaggtccccggctgcccatgggcgggagccgaggtgagtgaaaccaccgaggtctaggggcaattcgggctagggcagtctagcggaacgggcaagaaacttaaaacaatatttgttttacagatggttagtatatcctcaagtgatttttttaagaaaacgaaatttaatgaggagacgcagaaccaagtatggatgtctcaaattgctgactctcatgataatatctgcagttgctggcatccatttgctcaccttcttgcttccatatttcctcctggccacaaagatcgtgatcttactattaaccaaattcttctaagagattataaagaaaaatgccattctggtggagaagaaggagaaaattctggaccaacaacaggtttaattacaccaaaagaagaagatatagaaaaagatggcccagaaggcgccgcagaagaagaccatacagacgccctgttcgccgccgccgtagaaaacttcgaaaggtaaagagaaaaaaaaaatctttaattgttagacaatggcaaccagacagtataagaacttgtaaaattataggacagtcagctatagttgttggggctgaaggaaagcaaatgtactgttatactgtcaataagttaattaatgtgcccccaaaaacaccatatgggggaggctttggagtagaccaatacacactgaaatacttatatgaagaatacagatttgcacaaaacatttggacacaatctaatgtactgaaagacttatgcagatacataaatgttaagctaatattctacagagacaacaaaacagactttgtcctttcctatgacagaaacccaccttttcaactaacaaaatttacatacccaggagcacacccacaacaaatcatgcttcaaaaacaccacaaattcatactatcacaaatgacaaagcctaatggaagactaacaaaaaaactcaaaattaaacctcctaaacaaatgctttctaaatggttcttttcaaaacaattctgtaaataccctttactatctcttaaagcttctgcactagaccttaggcactcttacctaggctgctgtaatgaaaatccacaggtatttttttattatttaaaccatggatactacacaataacaaactggggagcacaatcctcaacagcatacagacctaactccaaggtgacagacacaacatactacagatacaaaaatgacagaaaaaatattaacattaaaagccatgaatacgaaaaaagtatatcatatgaaaacggttattttcaatctagtttcttacaaacacagtgcatatataccagtgagcgtggtgaagcctgtatagcagaaaaaccactaggaatagctatttacaatccagtaaaagacaatggagatggtaatatgatataccttgtaagcactctagcaaacacttgggaccagcctccaaaagacagtgctattttaatacaaggagtacccatatggctaggcttatttggatatttagactactgtagacaaattaaagctgacaaaacatggctagacagtcatgtactagtaattcaaagtcctgctatttttacttacccaaatccaggagcaggcaaatggtattgtccactatcacaaagttttataaatggcaatggtccgtttaatcaaccacctacactgctacaaaaagcaaagtggtttccacaaatacaataccaacaagaaattattaatagctttgtagaatcaggaccatttgttcccaaatatgcaaatcaaactgaaagcaactgggaactaaaatataaatatgtttttacatttaagtggggtggaccacaattccatgaaccagaaattgctgaccctagcaaacaagagcagtatgatgtccccgatactttctaccaaacaatacaaattgaagatccagaaggacaagaccccagatctctcatccatgattgggactacagacgaggctttattaaagaaagatctcttaaaagaatgtcaacttacttctcaactcatacagatcagcaagcaacttcagaggaagacattcccaaaaagaaaaagagaattggaccccaactcacagtcccacaacaaaaagaagaggagacactgtcatgtctcctctctctctgcaaaaaagataccttccaagaaacagagacacaagaagacctccagcagctcatcaagcagcagcaggagcagcagctcctcctcaagagaaacatcctccagctcatccacaaactaaaagagaatcaacaaatgcttcagcttcacacaggcatgttaccttaaccagatttaaacctggatttgaagagcaaacagagagagaattagcaattatatttcataggccccctagaacctacaaagaggaccttccattctatccctggctaccacctgcaccccttgtacaatttaaccttaacttcaaaggctaggccaacaatgtacacttagtaaagcatgtttattaaagcacaacccccaaaataaatgtaaaaataaaaaaaaaaaaaaaaaaataaaaaattgcaaaaattcggcgctcgcgcgcatgtgcgcctctggcgcaaatcacgcaacgctcgcgcgcccgcgtatgtctctttaccacgcacctagattggggtgcgcgcgctagcgcgcgcaccccaatgcgccccgccctcgttccgacccgcttgcgcgggtcggaccacttcgggctcgggggggcgcgcctgcggcgcttttttactaaacagactccgagccgccatttggccccctaagctccgcccccctcatgaatattcataaaggaaaccacataattagaattgccgaccacaaactgccatatgctaattagttccccttttacaaagtaaaaggggaagtgaacatagccccacacccgcaggggcaaggccccgcacccctacgtcactaaccacgcccccgccgccatcttgggtgcggcagggcgggggc

SEQ ID NO: 887 - protein, 124 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFER

SEQ ID NO: 888 - protein, 271 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFESGVDHNSMNQKLLTLANKSSMMSPILSTKQYKLKIQKDKTPDLSSMIGTTDEALLKKDLLKECQLTSQLIQISKQLQRKTFPKRKRELDPNSQSHNKKKRRHCHVSSLSAKKIPSKKQRHKKTSSSSSSSSRSSSSSSRETSSSSSTN

SEQ ID NO: 889 - protein, 267 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFERSASNFRGRHSQKEKENWTPTHSPTTKRRGDTVMSPLSLQKRYLPRNRDTRRPPAAHQAAAGAAAPPQEKHPPAHPQTKRESTNASASHRHVTLTRFKPGFEEQTERELAIIFHRPPRTYKEDLPFYPWLPPAPLVQFNLNFKG

SEQ ID NO: 890 - protein, 50 aa, Gammatorquevirus sp.
MRRRRTKYGCLKLLTLMIISAVAGIHLLTFLLPYFLLATKIVILLLTKFF

SEQ ID NO: 891 - protein, 662 aa, Gammatorquevirus sp.
MPFWWRRRRKFWTNNRFNYTKRRRYRKRWPRRRRRRRPYRRPVRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYTITNWGAQSSTAYRPNSKVTDTTYYRYKNDRKNINIKSHEYEKSISYENGYFQSSFLQTQCIYTSERGEACIAEKPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 892 - protein, 215 aa, Gammatorquevirus sp.
MPFWWRRRRKFWTNNRFNYTKRRRYRKRWPRRRRRRRPYRRPVRRRRRKLRKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 893 - protein, 129 aa, Gammatorquevirus sp.
MPFWWRRRRKFWTNNRFNYTKRRRYRKRWPRRRRRRRPYRRPVRRRRRKLRKISKQLQRKTFPKRKRELDPNSQSHNKKKRRHCHVSSLSAKKIPSKKQRHKKTSSSSSSSSRSSSSSSRETSSSSSTN

SEQ ID NO: 894-924 - skipped (31 entries)

SEQ ID NO: 925 - protein, 662 aa, Gammatorquevirus sp.; identical to SEQ ID NO: 891

SEQ ID NO: 926 - protein, 58 aa, Gammatorquevirus sp.
MPFWWRRRRKFWTNNRFNYTKRRRYRKRWPRRRRRRRPYRRPVRRRRRKLRKVKRKKK

SEQ ID NO: 927 - protein, 202 aa, Gammatorquevirus sp.
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYL

SEQ ID NO: 928 - protein, 79 aa, Gammatorquevirus sp.
NHGYYTITNWGAQSSTAYRPNSKVTDTTYYRYKNDRKNINIKSHEYEKSISYENGYFQSSFLQTQCIYTSERGEACIAE

SEQ ID NO: 929 - protein, 160 aa, Gammatorquevirus sp.
KPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFK

SEQ ID NO: 930 - protein, 163 aa, Gammatorquevirus sp.
WGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 931-948 - skipped (18 entries)

SEQ ID NO: 949 - protein, 21 aa, Artificial Sequence
  Description of Artificial Sequence: Synthetic peptide
  MOD_RES (1)..(1): W or F
  MOD_RES (2)..(8): Any amino acid
  MOD_RES (10)..(12): Any amino acid
  MOD_RES (14)..(14): Any amino acid
  MOD_RES (16)..(20): Any amino acid
XXXXXXXXHXXXCXCXXXXXH

SEQ ID NO: 950 - protein, 203 aa, Unknown
  Description of Unknown: Anelloviridae family sequence
KIIIRQWQPNYTRRCNIVGYMPLLICGENTVATNYATHSDDSYYPGPFGGGMTTDKFTLRILYDEYKRFMNYWTSSNEDLDLCRYLGCTLYCFRHPEVDFIIIINTSPPFLDTEITGPSIHPGMMALNKRSRWIPSIKNRPGRKHYIKIKVGAPRMFTDKWYPQTDLCDMTLLTIFASAADMQYPFGSPLTDTIVVSFQVLQS

SEQ ID NO: 951 - protein, 203 aa, Unknown
  Description of Unknown: Anelloviridae family sequence
KIIIRQWQPNYRRRCNIVGYLPILICGGNTVSRNYATHSDDTNYPGPFGGGMTTDKFSLRILYDEYKRFMNYWTASNEDLDLCRYLGCTFYFFRHPEVDFIIKINTMPPFLDTTITAPSIHPGLMALDKRARWIPSLKNRPGKKHYIKIRVGAPKMFTDKWYPQTDLCDMTLLTIYATAADMQYPFGSPLTDTVVVNSQVLQS

SEQ ID NO: 952 - protein, 203 aa, Unknown
  Description of Unknown: Anelloviridae family sequence
KIIIKQWQPNFIRRCYIIGCLPLVFCGENTTAQNYATHSDDMISKGPYGGGMTTTKFTLRILYDEFTRFMNFWTVSNEDLDLCRYVGCKLIFFKHPTVDFMVQINTQPPFLDTSLTAASIHPGIMMLSKRRILIPSLKTRPSRKHRVVVRVGAPRLFQDKWYPQSDLCDTVLLSIFATARDLQYPFGSPLTDNPCVNFQILGP

SEQ ID NO: 953 - protein, 203 aa, Unknown
  Description of Unknown: Anelloviridae family sequence
KIIIKQWQPNFIRRCYIIGYLPLIFCGENTTANNFATHSDDMIAKGPWGGGMTTTKFTLRILYDEFTRFMNFWTVSNEDLDLCRYVSCKLIFFKHPTVDFIVRINTEPPFLDTNLTAAQIHPGIMMLSKKHILIPSLKTRPSRKHRVVVRVGPPRLFQDKWYPQSDLCDTVLLSVFATACDLQYPFGSPLTDNPCVNFQILGH

SEQ ID NO: 954 - protein, 203 aa, Unknown
  Description of Unknown: Anelloviridae family sequence
KLIIKQWQPNFIRHCYIIGYMPLIICGENTFSHNYATHSDDMLSTGPYGGGMTTTKFTLRILFDEYQRHLNFWTVSNQDLDLARYLGTKIIFFRHPTVDFVVQIHTQPPFQDTEITAPSIHPGMLILSKKHILIPSLKTRPSKKHYVKVRVGAPRLFQDKWYPQSELCDVTLLVIYATACDLQYPFGSPQTDNVCVNFQILGQ

SEQ ID NO: 955 - protein, 204 aa, Unknown
  Description of Unknown: Anelloviridae family sequence
TLILRQWQPDCIRHCKITGWMPLIICGKGSTQFNYITHADDITPRGASYGGNFTNMTFSLEAIYEQFLYHRNRWSASNHDLELCRYKGTTLKLYRHPEVDYIVTYSRTGPFEISHMTYLSTHPMLMLLNKHHIVVPSLKTKPRGRKAIKVRIRPPKLMNNKWYFTRDFCNIGLFQLWATGLELRNPWLRMSTLSPCIGFNVLKN

SEQ ID NO: 956 - protein, 204 aa, Unknown
  Description of Unknown: Anelloviridae family sequence
TLVLRQWQPDVIRHCKITGRMPLIICGKGSTQFNYITHADDITPRGASYGGNFTNMTFSLEAIYEQFLYHRNRWSASNHDLELCRYKGTTLKLYRHPDVDYIVTYSRTGPFEISHMTYLSTHPLLMLLNKHHIVVPSLKTKPRGRKAIKVRIRPPKLMNNKWYFTRDFCNIGLFQLWATGLELRNPWLRMSTLSPCIGFNVLKN

SEQ ID NO: 957 - protein, 203 aa, Unknown
  Description of Unknown: Anelloviridae family sequence
KLVLTQWNPQTVRKCVIRGFLPLFFCGQGAYHRNFTDHYDDVFPKGPSGGGHGSMVFNLSFLYQEFKKHHNKWSRSNLDFDLVRYKGTVIKLYRHQDFDYIVWISRTPPFQESLLTVMTHQPSVMLQAKKCIIVKSYRTHPGGKPYVTAKVRPPRLLTDKWYFQSDFCNVPLFSLQFALAELRFPICSPQTDTNCINFLVLDD

SEQ ID NO: 958 - protein, 203 aa, Unknown
  Description of Unknown: Anelloviridae family sequence
KLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSS

SEQ ID NO: 959 - protein, 204 aa, Unknown
  Description of Unknown: Anelloviridae family sequence
RLVLTQWSPQTVRNCSIRGIVPMVICGHTKAGRNYAIHSEDFTTQIQPFGGSFSTTTWSLKVLWDEHQKFQNRWSYPNTQLDLARYRGVTFWFYRDQKTDYIVQWSRNPPFKLNKYSSAMYHPGMMMQAKRKLVVPSFQTRPKGKKRYRVTIKPPNMFADKWYTQEDLCPVPLVQIVVSAASLLHPFCPPQTNNPCITFQVLKD

SEQ ID NO: 960 - protein, 205 aa, Unknown
  Description of Unknown: Anelloviridae family sequence
KLVLTQWHPNTMRRCLIKGIVPLVICGHTRWNYNYALHSKDYTEEGRYPHGGALSTTTWSLKVLYDEHLKHHDFWGYPNNQLDLARYKGAKFTFYRHKKTDFIIFFNRKPPFKLNKYSCASYHPGMLMQQRHKILLPSYETKPKGRPKITVRIKPPTLLEDKWYTQQDLCDVNLLQLVVTAADFRHPLCSPQTNTPTTTFQVLKD

SEQ ID NO: 961 - protein, 204 aa, Unknown
  Description of Unknown: Anelloviridae family sequence
RLILRQWQPATRRRCTITGYLPIVFCGHTRGNKNYALHSDDYTPQGQPFGGALSTTSFSLKVLFDQHQRGLNKWSFPNDQLDLARYRGCKFIFYRTKQTDWVGQYDISEPYKLDKYSCPNYHPGNMIKAKHKFLIPSYDTNPRGRQKIIVKIPPPDLFVDKWYTQEDLCSVNLVSLAVSAASFLHPFGSPQTDNPCYTFQVLKE

SEQ ID NO: 962 - protein, 204 aa, Unknown
  Description of Unknown: Anelloviridae family sequence
KLILTQWNPAIVKRCNIKGGLPIIICGEPRAAFNYGYHMEDYTPQPFPFGGGMSTVTFSLKALYDQYLKHQNRWTFSNDQLDLARYRGCKLRFYRSPVCDFIVHYNLIPPLKMNQFTSPNTHPGLLMLSKHKIIIPSFQTRPGGRRFVKIRLNPPKLFEDKWYTQQDLCKVPLVSITATAADLRYPFCSPQTNNPCTTFQVLRK

SEQ ID NO: 963 - protein, 203 aa, Unknown
  Description of Unknown: Anelloviridae family sequence
KIVLTQWNPQTTRKCIIRGMMPVLWAGMGTGGRNYAVRSDDYVVNKGFGGSFATETFSLKVLYDQFQRGFNRWSHTNEDLDLARYRGCRWTFYRHKDTDFIVYFTNNPPMKTNQFSAPLTTPGMLMRSKYKVLIPSFQTRPKGRKTVTVKIRPPKLFQDKWYTQQDLCSVPLVQLNVTAADFTHPFGSPLTETPCVEFQVLGD

SEQ ID NO: 964 - protein, 204 aa, Unknown
  Description of Unknown: Anelloviridae family sequence
KLNIKEWQPITIRKTKVKGLYPAFLCNDQRIGNNAIQYLDSIAPHHFPGGGGFGIIQFTLQGLYEQFIKATNWWTQTNCSLPLIRYNFCKLKFYRTANVDYVVKIIRCYPLKATHDLYMQTQPSILMRDKHSILIPCMKNGRNRKTYKTIKVRPPTQMTNGWFFQKDLCNFPLLVILATAASFDRYYTNSKAKSTTIGFISLNT

SEQ ID NO: 965 - protein, 204 aa, Unknown
  Description of Unknown: Anelloviridae family sequence
KIRISEWQPTKIIKSKVKGIYPCFLSNHKRLSNNFVQWIDSTTAHLMPGGGGFGIIQFTLNGLYELFCKAQNWWTKSNCNLPLVRFCGTTLKFWAAENYDYVVHIQRCYPMCCTDLMYMSCQPFIMMMTKNTIFVPCTKNKPRAKRYKKIFVKPPAQMTTQWYFQSQLAKTGLIIIRTAACSLDRIYTSSTASSTTIGLVSLHT

SEQ ID NO: 966 - protein, 204 aa, Unknown
  Description of Unknown: Anelloviridae family sequence
TIRLRQWNPRTTRLCKIKGHIPLIICGRDRQIFNWMQYYDSIGPVEQSGGGGWSSIVFSLGALYQQFKRLMNWWTKDNDGLPLVQYRGCKFKFYKSWDCDYIVTAQTCPPMTDTEYKHLDSHPYRQLMNKRPIIVPNLVRKPSKKTYKIKRFPPPSLLQNKWYFQQDFCATNLLMLTTSAASFDQFWLPNDEISQVITFHSLNT

SEQ ID NO: 967 - protein, 204 aa, Unknown
  Description of Unknown: Anelloviridae family sequence
KIILKQFQPQTIKRVKIKRLECLFHCNAKKIYFNLQMYETSTVPPHLPGGGGWSCKVFTLNALYDAFKHCRNVWTGSNHNLPLVRYMGCKFTLYQSETQDYVFKYQNHYPMVSTIESYNACQPSMLMMDNRTKKIPSKKTKHKRKPYTIVKIRPPAQMQNKWYFTHDIANTPLLLIYSAACSFDNYYISTDSESTNISIPLLRT

SEQ ID NO: 968 - protein, 204 aa, Unknown
  Description of Unknown: Anelloviridae family sequence
TITVKQFQPPNVRKCKIKGLMNLIYFNQKRLIFNSVMYENSLVPEEHPGGGGFSVIKLSLETMYDSHLRCHNWWTTSNEDLPLVRYSGMTVKLYQSKYTDYVFKYQNYLPGTSTYLTYPACQPSMMMMAKNSVIVPSLETKRRRKPYKKIHIKPTSQLQTKWYFQTDLNKTPLAIFYTAACSLTSYYLSPDWESNNISILHLNT

SEQ ID NO: 969 - protein, 204 aa, Unknown
  Description of Unknown: Anelloviridae family sequence
TIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 970 - protein, 204 aa, Unknown
  Description of Unknown: Anelloviridae family sequence
KLTLKEWQPKTIRKSCIKGLHCLFIVTEDTISRNYRMYEHSFTGEHWPSGGGFSVTKYTLDGLYEQHQLDRNWWTKPNTNMPLVRYLGCKITFYQSWEVDYVCNINLTWPMVATNLLYLSCHPNFMMMNHKAIFVPSKITKRLRRGKKTVRLRPPHQMINKWYFAKELAKTGLVMLTAAAASFDHYYIGSDKLSNNCTFTSLNP

SEQ ID NO: 971 - protein, 204 aa, Unknown
  Description of Unknown: Anelloviridae family sequence
KIPIYQWQPDSIRKCKIKGVGTLVLGAHGKQFVCYTDVETRAPPPKAPGGGGFGCKQFSLQYLYEEYRFRNNIWTHTNINLDLVRYLRAAFTFYRHPDIDFIINYDRQPPFYLDKFTYPLCHPQNLLLGKHKIILLSKASKPNGKVKKRIIIKPPKQMITKWFFQEQFTTQPLLSLRAAAASFQYPHIGCCMPNRVVTFSALNP

SEQ ID NO: 972 - protein, 204 aa, Unknown
  Description of Unknown: Anelloviridae family sequence
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNH

SEQ ID NO: 973 - protein, 204 aa, Unknown
  Description of Unknown: Anelloviridae family sequence
TLIVRQWQPDSIVLCKIKGYDSIIWGAEGTQFQCSTHEMYEYTRQKYPGGGGFGVQLYSLEYLYDQWKLRNNIWTKTNQLKDLCRYLKCVMTFYRHQHIDFVIVYERQPPFEIDKLTYMKYHPYMLLQRKHKIILPSQTTNPRGKLKKKKTIKPPKQMLSKWFFQQQFAKYDLLLIAAAACSLRYPRIGCCNENRMITLYCLNT

SEQ ID NO: 974 - protein, 204 aa, Unknown
  Description of Unknown: Anelloviridae family sequence
KITIKQWQPDSVKKCKIKGYSTLVMGAQGKQYNCYTNQASDYVQPKAPQGGGFGCEVFNLKWLYQEYTAHRNIWTKTNEYTDLCRYTGAQIILYRHPDVDFIVSWDNQPPFLLNKYTYPELQPQNLLLARRKRIILSQKSNPKGKLRIKLRIPPPKQMITKWFFQRDFCDVNLFKLCASAASFRYPGISHGAQSTIFSAYALNT

SEQ ID NO: 975 - protein, 204 aa, Unknown
  Description of Unknown: Anelloviridae family sequence
FLKLVQWQPDRIRKCTIKGRGTLVMGAHGRQMICFTNVKDRLTPSRAPAGGGFGVERITLAHLYEEWKARNNVWTATNENTDLGRYTGCKISFYRHLDTDFIIKYSRNPPFNLEKYSYMYMQPQELLLGKHKRILLSKKTNPKGKLKTTLKIGPPKLLTNKWLLQSELAKQDLVEISAAAASFTYPTIGCCNENRILNLYSINT

SEQ ID NO: 976 - DNA, 9 bp, Artificial Sequence
  Description of Artificial Sequence: Synthetic primer
kkgtktctr

SEQ ID NO: 977 - DNA, 10 bp, Artificial Sequence
  Description of Artificial Sequence: Synthetic primer
gtaccayttr

SEQ ID NO: 978 - DNA, 10 bp, Artificial Sequence
  Description of Artificial Sequence: Synthetic primer
gttwgtggts

SEQ ID NO: 979 - DNA, 9 bp, Artificial Sequence
  Description of Artificial Sequence: Synthetic primer
caccgacva

SEQ ID NO: 980 - DNA, 9 bp, Artificial Sequence
  Description of Artificial Sequence: Synthetic primer
ctcggagtg

SEQ ID NO: 981 - DNA, 9 bp, Artificial Sequence
  Description of Artificial Sequence: Synthetic primer
gcactcctc

SEQ ID NO: 982 - DNA, 9 bp, Artificial Sequence
  Description of Artificial Sequence: Synthetic primer
cggagtctg

SEQ ID NO: 983 - DNA, 9 bp, Artificial Sequence
  Description of Artificial Sequence: Synthetic primer
cccactcac

SEQ ID NO: 984 - DNA, 10 bp, Artificial Sequence
  Description of Artificial Sequence: Synthetic primer
gtaccactta

SEQ ID NO: 985 - DNA, 10 bp, Artificial Sequence
  Description of Artificial Sequence: Synthetic primer
gtaccacttg

SEQ ID NO: 986 - DNA, 10 bp, Artificial Sequence
  Description of Artificial Sequence: Synthetic primer
gtaccattta

SEQ ID NO: 987 - DNA, 10 bp, Artificial Sequence
  Description of Artificial Sequence: Synthetic primer
gtaccatttg

SEQ ID NO: 988 - DNA, 10 bp, Artificial Sequence
  Description of Artificial Sequence: Synthetic primer
gaccacaaac

SEQ ID NO: 989 - DNA, 10 bp, Artificial Sequence
  Description of Artificial Sequence: Synthetic primer
gaccactaac

SEQ ID NO: 990 - DNA, 10 bp, Artificial Sequence
  Description of Artificial Sequence: Synthetic primer
caccacaaac

SEQ ID NO: 991 - DNA, 10 bp, Artificial Sequence
  Description of Artificial Sequence: Synthetic primer
caccactaac

SEQ ID NO: 992 - protein, 22 aa, Artificial Sequence
  Description of Artificial Sequence: Synthetic peptide
  MOD_RES (6)..(7): Any amino acid
  MOD_RES (9)..(9): Any amino acid
  MOD_RES (15)..(16): Any amino acid
YNCSPXXDXGASKRXXNTSVAK

SEQ ID NO: 993 - DNA, 51 bp, Artificial Sequence
  Description of Artificial Sequence: Synthetic oligonucleotide
  modified_base (45)..(45): a, c, t, g, unknown or other
  See specification as filed for detailed description of substitutions and preferred embodiments
aggtgagtgaaaccaccgaagtcaaggggcaattcgggctagggncagtct

SEQ ID NO: 994 - DNA, 50 bp, Artificial Sequence
  Description of Artificial Sequence: Synthetic oligonucleotide
aggtgagtttacacaccgcagtcaaggggcaattcgggctcgggactggc

SEQ ID NO: 995 - DNA, 50 bp, Artificial Sequence
  Description of Artificial Sequence: Synthetic oligonucleotide
aggtgagtgaaaccaccgaagtcaaggggcaattcgggctagatcagtct

SEQ ID NO: 996 - DNA, 50 bp, Artificial Sequence
  Description of Artificial Sequence: Synthetic oligonucleotide
aggtgagtgaaaccaccgaggtctaggggcaattcgggctagggcagtct

SEQ ID NO: 997 - DNA, 20 bp, Artificial Sequence
  Description of Artificial Sequence: Synthetic primer
agcaacaggtaatggaggac

SEQ ID NO: 998 - DNA, 19 bp, Artificial Sequence
  Description of Artificial Sequence: Synthetic primer
tgaagctggggtctttaac

SEQ ID NO: 999 - DNA, 21 bp, Artificial Sequence
  Description of Artificial Sequence: Synthetic probe
tctacctaggtgcaaagggcc

SEQ ID NO: 1000 - DNA, 9 bp, Artificial Sequence
  Description of Artificial Sequence: Synthetic primer
cgaatggyw

SEQ ID NO: 1001 - DNA, 9 bp, Artificial Sequence
  Description of Artificial Sequence: Synthetic primer
ttgcccctt

SEQ ID NO: 1002 - DNA, 9 bp, Artificial Sequence
  Description of Artificial Sequence: Synthetic primer
ytgyggbtg

SEQ ID NO: 1003 - DNA, 9 bp, Artificial Sequence
  Description of Artificial Sequence: Synthetic primer
yagamacmm

SEQ ID NO: 1004 - DNA, 10 bp, Artificial Sequence
  Description of Artificial Sequence: Synthetic primer
gtaccayttr

SEQ ID NO: 1005 - DNA, 10 bp, Artificial Sequence
  Description of Artificial Sequence: Synthetic primer
saccacwaac

SEQ ID NO: 1006 - DNA, 9 bp, Artificial Sequence
  Description of Artificial Sequence: Synthetic primer
caccgacva

SEQ ID NO: 1007 - DNA, 9 bp, Artificial Sequence
  Description of Artificial Sequence: Synthetic primer
cactccgag

SEQ ID NO: 1008 - DNA, 9 bp, Artificial Sequence
  Description of Artificial Sequence: Synthetic primer
gcactcctc

SEQ ID NO: 1009 - DNA, 9 bp, Artificial Sequence
  Description of Artificial Sequence: Synthetic primer
cagactccg

SEQ ID NO: 1010 - DNA, 9 bp, Artificial Sequence
  Description of Artificial Sequence: Synthetic primer
cccactcac

SEQ ID NO: 1011 - DNA, 9 bp, Artificial Sequence
  Description of Artificial Sequence: Synthetic primer
cttcgccat

SEQ ID NO: 1012 - protein, 21 aa, Artificial Sequence
  Description of Artificial Sequence: Synthetic peptide
  MOD_RES (2)..(8): Any amino acid
  MOD_RES (10)..(12): Any amino acid
  MOD_RES (14)..(14): Any amino acid
  MOD_RES (16)..(20): Any amino acid
WXXXXXXXHXXXCXCXXXXXH

SEQ ID NO: 1013 - protein, 6 aa, Artificial Sequence
  Description of Artificial Sequence: Synthetic 6xHis tag
HHHHHH